(19) United States
(12) Patent Application Publication
(10) Pub. No.: US 2022/0072098 A1
(43) Pub. Date: Mar. 10, 2022

(54) BOLAAMPHIPHILIC COMPOUNDS, COMPOSITIONS AND USES THEREOF

(71) Applicant: Lauren Sciences LLC, New York, NY (US)

(72) Inventors: Charles LINDER, Rehovot (IL); Eliahu HELDMAN, Rehovot (IL); Sarina GRINBERG, Meitar (IL)

(21) Appl. No.: 17/351,373

(22) Filed: Jun. 18, 2021

Related U.S. Application Data
(63) Continuation of application No. 16/188,232, filed on Nov. 12, 2018, now abandoned.
Application No. 16/188,232 is a continuation of application No. 15/099,956, filed on Apr. 15, 2016, now abandoned.
Application No. 15/099,956 is a continuation-in-part of application No. 14/328,419, filed on Jul. 10, 2014, now abandoned.
Application No. 14/328,419 is a continuation-in-part of application No. PCT/US13/57956, filed on Sep. 4, 2013.
(60) Provisional application No. 61/696,789, filed on Sep. 4, 2012.
(60) Provisional application No. 61/845,185, filed on Jul. 11, 2013.
(60) Provisional application No. 61/915,908, filed on Dec. 13, 2013.
(60) Provisional application No. 62/148,511, filed on Apr. 16, 2015.
(60) Provisional application No. 62/258,773, filed on Nov. 23, 2015.

(51) Int. Cl. A61K 38/18; A61K 31/675; A61K 9/51; A61K 9/00; A61K 47/54; A61K 47/69

(52) U.S. Cl. CPC A61K 38/185; A61K 31/675; A61K 47/6929; A61K 9/0019; A61K 47/545; A61K 9/5123

(57) ABSTRACT

Bolaamphiphilic compounds are provided according to formula I:

HG²-L¹-HG¹ I

where HG¹, HG² and L¹ are as defined herein. Provided bolaamphilphilic compounds and the pharmaceutical compositions thereof are useful for delivering GDNF or NGF into animal or human brain.

### CROSS REFERENCES TO RELATED APPLICATIONS

This is a continuation of U.S. patent application Ser. No. 16/188,232, filed Nov. 12, 2018, which is a continuation of U.S. patent application Ser. No. 15/099,956, filed Apr. 15, 2016, which is a continuation-in-part of U.S. patent application Ser. No. 14/328,419, filed on Jul. 10, 2014, which is a continuation-in-part of International Application PCT/US13/57956, filed on Sep. 4, 2013, which claims priority to U.S. Patent Application 61/696,789, filed on Sep. 4, 2012. U.S. patent application Ser. No. 14/328,419 also claims the benefit of U.S. Patent Application No. 61/845,185, filed on Jul. 11, 2013, and U.S. Patent Application No. 61/915,908, filed on Dec. 13, 2013. This application also claims the benefit of U.S. Patent Application No. 62/148,511, filed on Apr. 16, 2015, and U.S. Patent Application No. 62/258,773, filed on Nov. 23, 2015. The contents of each of the above-referenced applications are incorporated by reference herein.

### FIELD

Provided herein are nanovesicles comprising bolaamphiphilic compounds, and complexes thereof with neurotrophins (NTFs), such as glial cell derived growth factor (GDNF) or nerve growth factor (NGF), and pharmaceutical compositions thereof. Also provided are methods of delivering NTFs into the human brain and animal brain using the compounds, complexes and pharmaceutical compositions provided herein. In particular, the present disclosure is further directed to compounds, compositions, and method of the treatement of neurological diseases including, for illustrative purposes Parkinson's disease, Alzheimers and amyotrophic lateral sclerosis (ALS). Also provided are methods of delivering NTFs into the human brain and animal brain using the compounds, complexes and pharmaceutical compositions provided herein.

### BACKGROUND

Many studies using cell cultures and animal models of Parkinson's disease (PD), Alzeimer's disease (AD), or amyotrophic lateral scelerosis (ALS), and in some cases human PD and AD patients and human ALS patients, demonstrate that neurotrophins (NTFs), such as glial cell derived growth factor (GDNF) or nerve growth factor (NGF), have good potential as therapeutic agents in neurodegenerative diseases, including, e.g., PD or AD treatment [1]. However, GDNF or NGF do not permeate through the blood-brain barrier (BBB), thus they have to be delivered directly into the brain in order to exert its therapeutic action. Nevertheless, attempts to deliver GDNF directly into the brain (e.g., intraputamenal injection) had little benefit, most probably because its distribution within the brain was restricted to only 2-9% of the area receiving the GDNF [2]. Also, convection-enhanced delivery of GDNF resulted in a great deal of variability in its distribution within the injected site [3]. The variability in GDNF distribution, and its limited diffusion throughout the brain, is most probably due to its binding to the extracellular matrix [4]. This implies that a delivery system which is capable of distributing GDNF uniformly within the brain, and not concentrating it at a small site (that might cause toxicity), should increase the probability that all affected neurons are exposed to GDNF's therapeutic activity and, thus, increase GDNF's efficacy in the treatment of PD.

The brain capillary endothelial cells (BCECs) that form the BBB play important role in brain physiology by maintaining selective permeability and preventing passage of various compounds from the blood into the brain. One consequence of the highly effective barrier properties of the BBB is the limited penetration of therapeutic agents into the brain, which makes treatment of many brain diseases extremely challenging.

A delivery system that uses the intense capillary network that supplies blood to the brain, should deliver GDNF or NGF to a wide area within the brain, provided that the delivery system is capable of crossing the BBB and releasing the NTF there. Targeting to specific sites within the brain is alos greatly facilitated by an efficient penetration through the BBB into the brain after systemic administration.

Efforts to improve the permeation of GDNF across the BBB have been attempted, but have not proven therapeutically successful.

Efforts to improve the permeation of biologically active compounds across the BBB using amphphilic vesicles have been attempted.

For example, complexation of the anionic carboxyfluorescein (CF) (a fluorescent marker) with single headed amphiphiles of opposite charge in cationic vesicles, formed by mixing single-tailed cationic and anionic surfactants has been reported (Danoff et al. 2007). In addition to complexation, a certain portion of the CF is passively encapsulated within the core of the formed vesicles. The present disclosure employing bolaamphilies, includes embodiments in which a portion of the active agent may be complexed to the head groups of the bolaamphiphiles and another fraction of the active agents are encapsulated within the core of the vesicles. In many embodiments, the major portion of the active agent is encapsulated by complexation with the head groups.

Furthermore, WO 02/055011 and WO 03/047499, both of the same applicant of the present disclosure, disclose amphiphilic derivatives composed of at least one fatty acid chain derived from natural vegetable oils such as vemonia oil, lesquerella oil and castor oil, in which functional groups such as epoxy, hydroxy and double bonds were modified into polar and ionic headgroups.

Additionally, WO 10/128504 reports a series of amphiphiles and bolamphiphiles (amphiphiles with two head groups) useful for targeted drug delivery of insulin, insulin analogs, TNF, GDNF, DNA, RNA (including siRNA), enkephalin class of analgesics, and others.

These synthetic bolaamphiphiles (bolas) have recently been shown to form nanovesicles that interact with and encapsulate a variety of small and large molecules including peptides, proteins and plasmid DNAs delivering them across biological membranes. These bolaamphiphiles are a unique class of compounds that have two hydrophilic headgroups placed at each ends of a hydrophobic domain. Bolaamphiphiles can form vesicles that consist of monolayer membrane that surrounds an aqueous core. Vesicles made from natural bolaamphiphiles, such as those extracted from archaebacteria (archaesomes), are very stable and, therefore, might be employed for targeted drug delivery. However, bolaamphiphiles from archaebacteria are heterogeneous and cannot be easily extracted or chemically synthesized.

Thus, there remains a need to make new compositions and for novel and optimized methods to deliver NTF, such as glial cell derived growth factor (GDNF) or nerve growth factor (NGF), into the brain. The compounds, compositions, and methods described herein are directed toward this end.

### SUMMARY OF THE INVENTION

In certain aspects, provided herein are pharmaceutical compositions comprising of a bolaamphiphile complex.

In further aspects, provided herein are novelnano-sized vesicles comprising of bolaamphiphilic compounds.

In further aspects, provided herein are novel nano-sized vesicles comprising of bolaamphiphilic compounds which are capable of encapsulating NTF, GDNF or NGF. In certain aspects, the vesicles comprise bolaamphiphilic compounds capable of encapsulating a neurotrophic factor selected from among Glial cell-derived neurotrophic factor (GDNF), Nerve Growth factor (NGF), Brain-Derived Neurotrophic Factor (BDNF), Neurotrophin-3 (NT-3), Neurotrophin-4/5 (NT-4/5), as well as combinations of two or more thereof.

In further aspects, provided herein are novel nano-sized bola vesicles that encapsulate GDNF or NGF and are capable of delivering the encapsulated material into the brain. In other aspects, the encapsulated neurotrophic factor is from among Glial cell-derived neurotrophic factor (GDNF), Nerve Growth factor (NGF), Brain-Derived Neurotrophic Factor (BDNF), Neurotrophin-3 (NT-3), Neurotrophin-4/5 (NT-4/5), as well as combinations of two or more thereof.

In further aspects, provided herein are novel nano-sized bola vesicles that encapsulate GDNF or NGF and are capable of delivering the encapsulated material to the brain, specifically to dopaminergic neurons. In a further aspect there are submicron vesicles with a monolayer membrane or bilayer membrane encapsulating an inner core, with GDNF, NGF Brain-Derived Neurotrophic Factor (BDNF), Neurotrophin-3 (NT-3), Neurotrophin-4/5 (NT-4/5), or combination of two or more thereof.

In certain embodiments, the present disclosure describes the use of GDNF for the treatment of amyotrophic lateral sclerosis (ALS) and for the treatment of Alzheimer's disease in a patient in need thereof.

In certain embodiments, the present disclosure describes treatment of neurodegenerative disease in a patient in need thereof comprising delivery of one or more neurotrophic factors (neurotrophins) using the vesicles and vesicle delivery systems described herein. In particular aspects of these embodiments, the neurotrophic factor is selected from among Glial cell-derived neurotrophic factor (GDNF), Nerve Growth factor (NGF), Brain-Derived Neurotrophic Factor (BDNF), Neurotrophin-3 (NT-3), Neurotrophin-4/5 (NT-4/5), and combinations of two or more thereof In particular aspects of these embodiments, the neurodegenerative disease may be Alzheimer's disease, Parkinson's disease, Amyotrophic lateral sclerosis (ALS), Huntingdon's disease, neurodegeneration associated with aging, and combinations thereof.

in certain embodiments therefore, the present disclosure describes vesicles and their use as delivery systems for neurotropic factors that can be administered systemically, e.g., intravenously and/or orally, that can pass intact through different biological barriers, such as but not limited to the blood brain barrier, and deliver their contents to targeted to sites within the brain and/or the peripheral nervous affected by the neurodegenerative disease of a patient in need of such treatment. Although such neurodegenerative diseases are currently incurable and involve debilitating conditions resulting fr©m progressive degeneration and/or death of nerve cells affecting movement (ataxias), or mental functioning (dementias), delivery systems and bolavesicle carriers described herein can be used to ameliorate or reverse these effects, to prevent their occurrence, to mitigate the frequency and/or intensity of flare-ups, to substantially arrest progression of such effects, and/or to diminish the symptoms thereof.

In further aspects, provided herein are novel nano-sized bola vesicles that encapsulate GDNF or NGF and are capable of delivering the encapsulated material into brain regions affected in neurological disorders, In one particular embodiment, the neurological disorder is Parkinson's disease (PD) or Alzheimer's disease (AD).

In certain aspects, provided herein are novel bolaamphiphile complexes comprising bolaamphiphilic compounds and a compound active against PD. In one embodiment, the compound active against AD is GDNF.

In certain aspects, provided herein are novel bolaamphiphile complexes comprising bolaamphiphilic compounds and a compound active against AD. In one embodiment, the compound active against PD is NGF.

In other particular aspects, the present disclosure provides bolaamphiphile complexes comprising bolaamphiphilic compounds and active agents that are protein neutrophic factors (e.g., NTF), and neurotropins for the treatment of neurodegenerative diseases such as Parkinson's disease (PD), amyotrophic lateral sclerosis (ALS) and Alzheimer's disease, as well as Huntingdon's disease and neurodegeneration associated with aging.

In other embodiments, the present disclosure is directed to vesicles containing protein/peptide antibodies and the use thereof for the treatment of Alzheimer's disease. These methods can be employed for the prevention and treatment of Alzheimer's disease, and can exhibit improved pharmacokinetics with therapeutic amounts delivered to the relevant sites in the brain affected by Alzheimer's disease. The vesicles of the present invention are drug delivery systems that can overcome prior art limitations including poor pharmacokinetics since the proteinaceous agents are readily metabolized in vivo, have poor penetrability through biological barriers and have a large bio-distribution. In contrast, the vesicles disclosed herein can be administered via enteral administration (absorption of the drug through the gastrointestinal tract) or parenteral administration (generally injection, infusion, or implantation). Topical applications are also encompassed within the present disclosure.

In one aspect of these embodiments, the mode of administration for many applications is intravenous injections and/or oral administration, where the antibody or antibody fragment may be encapsulated with the vesicle's core and/or within the encapsulating membrane and the encapsulation may include complexation with the bolaamphiphiles or additives comprising the vesicles.

Thus the present disclosure provides delivery in the described vesicles of protein antibodies to the CNS for the treatment of Alzheimer's disease. In one embodiment, the present disclosure provides formulation of encapsulated anti-tau antibodies that can strongly decrease tau accumulation and/or prevent the accumulation of tau proteins as a therapy for patients with Alzheimer's disease and other neurodegenerative disorders.

In another embodiment, the present disclosure provides vesicles with a specific antibody or antibody fragment against soluble aggregates of the AP peptide, responsible for the toxicity and cell death characteristic of Alzheimer's disease. In one embodiment the whole antibody does not have to be used; instead, an antibody fragment or a recombinant antibody consisting of the active part of the antibody responsible for the binding of Aβ oligomers is delivered to the target site. In one aspect of this embodiment, the present disclosure provides vesicles with both specific and nonspecific antibodies against Aβ-peptide and their use in a systemic treatment for patients with Alzheimer's disease.

In another embodiment, the present disclosure provides vesicles for the delivery to the CNS and sites affected by Alzheimer's disease with anti-Aβ antibodies for the removal of brain Aβ peptide. In one aspect of this embodiment, the present disclosre provides encapsulation of bapineuzumab, which is composed of humanized anti-Aβ monoclonal antibodies that has been shown to reduce AP burden in the brain of AD patients.

In still another embodiment for the treatment of Alzheimer, the present disclosure provides vesicles with encapsulated or complexed human immune globulin intravenous (IGIV [GAMMAGARD]) and their use in the treatment of Alzheimer's patients in need thereof

In another embodiment, the present disclosure provides vesicles comprising either recombinant or naturally occurring antibodies directed against beta-amyloid (Abeta and Abeta91-42) and the use thereof for the treatment of Alzhiemer's disease via systemic administration which in illustrative example is IV administration or oral administration.

In further aspects, provided herein are novel formulations of GDNF or NGF with bolaamphiphilic compounds or with bolaamhphile vesicles.

In another aspect, provided here are methods of delivering GDNF or NGF agents into animal or human brain. In one embodiment, the method comprises the step of administering to the animal or human a pharmaceutical composition comprising of a bolaamphiphile complex; and wherein the bolaamphiphile complex comprises a bolaamphiphilic compound and GDNF. In another embodiment, the complex comprises bolaamphiphilic compound and NGF. In other aspects, the administered composition comprises a neurotrophic factor selected from among Glial cell-derived neurotrophic factor (GDNF), Nerve Growth factor (NGF), Brain-Derived Neurotrophic Factor (BDNF), Neurotrophin-3 (NT-3). Neurotrophin-4/5 (N 1-4/5). as well as combinations of two or more thereof.

In a further embodiment, the present disclsosure provides compositions and methods for the delivery of the protein Activin to the CNS using bolaamphile vesicles of the present disclosure. In certain aspects of this embodiment, the Activin is at least one of Activin A, Activin B, Activin AB, and combinations thereof. In one specific aspect, the Activin is Activin A.

In one embodiment, the bolaamphiphilic compound consists of two hydrophilic headgroups linked through a long hydrophobic chain. In another embodiment, the hydrophilic headgroup is an amino containing group. In a specific embodiment, the hydrophilic headgroup is a tertiary or quaternary amino containing group.

In one particular embodiment, the bolaamphiphilic compound is a compound according to formula I:

HG²-L¹-HG¹ I

or a pharmaceutically acceptable salt, solvate, hydrate, prodrug, stereoisomer, tautomer, isotopic variant, or N-oxide thereof, or a combination thereof; wherein:

each HG¹ and HG² is independently a hydrophilic head group; and

L¹ is alkylene, alkenyl, heteroalkylene, or heteroalkenyl linker; unsubstituted or substituted with C₁-C₂₀ alkyl, hydroxyl, or oxo.

In one embodiment, the pharmaceutically acceptable salt is a quaternary ammonium salt.

In one embodiment, with respect to the bolaamphiphilic compound of formula I, the bolaamphiphilic compound is a compound according to formula II, III, IV, V, or VI:

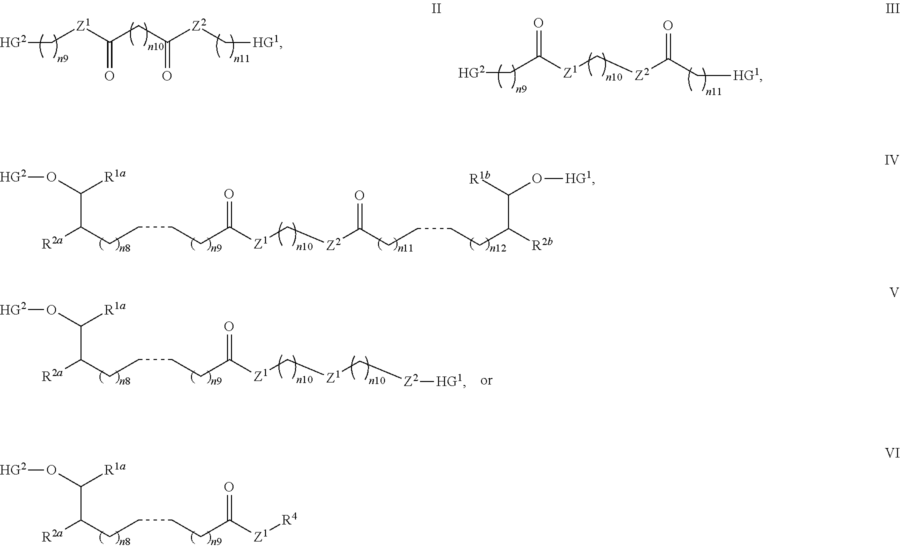

or a pharmaceutically acceptable salt, solvate, hydrate, prodrug, stereoisomer, tautomer, isotopic variant, or N-oxide thereof, or a combination thereof; wherein:

each HG¹ and HG² is independently a hydrophilic head group;

each Z¹ and Z² is independently —C(R³)₂—, —N(R³)— or —O—;

each R^{1a}, R^{1b}, R³ and R⁴ is independently H or C₁-C₈ alkyl;

each R^{2a} and R^{2b} is independently H , C₁-C₈ alkyl, OH, alkoxy, or O-HG¹ or O-HG²;

each n8, n9, n11, and n12 is independently an integer from 1-20;

n10 is an integer from 2-20; and
each dotted bond is independently a single or a double bond.

In one embodiment, with respect to the bolaamphiphilic compound of formula I, II, III, IV, V, or VI, each HG¹ and HG² is independently selected from:

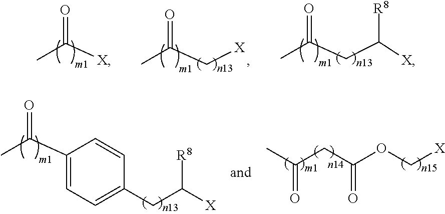

wherein:
X is —NR^{5a}R^{5b}, or —N⁺R^{5a}R^{5b}R^{5c}; each R^{5a}, and R^{5b} is independently H or substituted or unsubstituted C₁-C₂₀ alkyl or R^{5a} and R^{5b} may join together to form an N containing substituted or unsubstituted heteroaryl, or substituted or unsubstituted heterocyclyl; each R^{5c} is independently substituted or unsubstituted C₁-C₂₀ alkyl; each R⁸ is independently H, substituted or unsubstituted C₁-C₂₀ alkyl, alkoxy, or carboxy;
m1 is 0 or 1; and

each n13, n14, and n15 is independently an integer from 1-20.

In certain embodiments, this disclosure provides novel monlayer nanovescicles. In particular aspects of these embodiments, the nanovesicles comprise bolaamphiphilic compounds with head groups faciliatating penetration of the bood brain barrier. In other aspects, the nanovesicles comprise bolaamphiphilic compounds with head groups that facilitate targeting to dopaminergic neurons in the brain. * In still another aspect the vesicles formed from the bolaamphiphiles contain additives that help to stabilize the vesicles, by stabilizing the vesicle's membranes, such as but not limited to cholesterol derivcatives such as cholesteryl hemisuccinate and cholesterol itself and combinations such as cholesteryl hemisuccinate and cholesterol. In another embodiment the vesicles comprise the bolaamphiphiles, vesicle membrane stabilizing additives, stearyl amine, and GDNF and NGF. In still another embodiments the vesicles in addition to these components have another addiitves which decorates the outer vesicle memrbanes with groups or pendants that enhance penetration though biological barriers such as the BBB and groups for targeting. A non limiting example of such additives may be alkyl conjugates of chitosan or bolaamphiphiles where one of the head groups is chitiosan.

In certain embodiments, the present disclosure provides nanovesicles that comprise bolaamphiphilic compounds with chitosan head groups.

In certain embodiments, the present disclosure provides nanovesicles that comprise bolaamphiphilic compounds with head groups that can function as ligands for the dopamine transporter.

In particular, embodiments, the present disclosure provides nanovesicles that comprise bolaamphiphilic compounds with head groups that can function as ligands for the dopamine transporter as well as with bolaamphiphilic compounds with that comprise chitosan head groups.

In certain embodimetns, the present disclosure provides monlayer nanovesicles comprising the bolaamphiphilic compound designated herein as GLH-55a, the bolaamphiphilic compound designated herein as GLH-57, as well as encapsulated GDNF.

In other embodiments, the present disclosure provides a method of treatment of a neurotrophic disease comprising administration of an effective amount of monlayer nanovesicles of the disclosure comprising an encapsulated active agent. In particular aspects of this embodiment, the neutrophic disese is Parkinson's disease, and the administered monlayer nanovesicles comprise the bolaamphiphilic compound designated herein as GLH-55a, the bolaamphiphilic compound designated herein as GLH-57, as well as encapsulated GDNF.

The present disclosure further provides compositions and methods for controlling the rate of release of vesicle-encapsulated materials by varying the length of alkyl chains adjacent to hydrolysable head groups of bolaamphiphilic vesicles. In one aspect of this embodiment, the head groups are acetylcholine head groups.

Other objects and advantages will become apparent to those skilled in the art from a consideration of the ensuing detailed description.

### BRIEF DESCRIPTION OF THE DRAWINGS

FIG. 1A: TEM micrograph of vesicles from GLH-20 and FIG. 1B: their size distribution determined by DLS.

FIG. 2A: Head group hydrolysis by AChE of GLH-19 (blue) and GLH-20 (red), FIG. 2B: release of CF from GLH-19 vesicles and FIG. 2C: release of CF from GLH-20 vesicles.

FIG. 3A: CF accumulation in brain after i.v. injection of encapsulated and non-encapsulated CF. Only GLH-20 vesicles allow accumulation of CF in the brain. FIG. 3B: CS improves GLH-20 vesicles' penetration into the brain.

FIG. 4A: Analgesia after i.v. injection of enkephalin non-encapsulated and encapsulated in vesicles. Analgesia (compared with morphine, which was used as a positive control) is obtained only when enkephalin is encapsulated in GLH-20 vesicles, the head groups of which are hydrolyzed by ChE. FIG. 4B: The vesicles do not disrupt the BBB since non-encapsulated enkephalin co-injected with empty vesicles (extravesicular enkephalin) did not cause analgesia. **Significantly different from free leu-enkephalin (t-test, P<0.01). ***Significantly different from free leu-enkephalin (t-test, P<0.001).

FIG. 5A: Fluorescence in mouse cerebral cortex after i.v. injection of albumin-FITC (non-encapsulated); FIG. 5B: Fluorescence in mouse cerebral cortex after i.v. injection of albumin-FITC encapsulated in GLH-20 vesicles.

FIG. 6A: Mass spectra of GLH-20(A) and FIG. 6B: Mass spectra of GLH-19.

FIG. 7: FT-IR spectra of original (CS) (spectrum a) and LMWCS (spectrum b).

FIG. 8: ¹H NMR of the anhydroecgonine methyl ester, compound 4.

FIG. 9A: HMQC of the anhydroecgonine methyl ester 4 and FIG. 9B: ¹H COSY NMR of the anhydroecgonine methyl ester 4.

FIG. 10A depicts compound 5, β-CFT. FIG. 10B depicts the ¹H NMR spectra of compound 5, β-CFT.

FIG. 11A: ¹H-NMR spectra of the demethylated β-CFT fluoronortropane 7 and FIG. 11B: ¹³C-NMR spectra of the demethylated β-CFT fluoronortropane 7.

FIG. 12A: ¹H-NMR spectrum of GLH-57, FIG. 12B: enlargement of the section 2.6-3.5 ppm of the ¹H-NMR spectrum of GLH-57.

FIG. 13: CryoTEM micrographs of vesicles made from the basic bolas. Vesicles were prepared by film hydration followed by probe sonication from a formulation containing 10 mg/ml bola, 2.1 mg/ml cholesteryl hemisuccinate and 1.6 mg/ml cholesterol. (Panel A) empty GLH-19 vesicles; (Panel B) GLH-19 vesicles loaded with 2 mg/ml trypsinogen; (Panel C) empty GLH-20 vesicles; (Panel D) GLH-20 vesicles loaded with 2 mg/ml trypsinogen; (Panel E) GLH-19 vesicles loaded with CF; (Panel F) GLH-20 vesicles loaded with CF

FIG. 14: CryoTEM micrographs of vesicles made from a mixture of GLH-19 and GLH-20. Vesicles were prepared by film hydration followed by sonication from a formulation containing GLH-19 and GLH-20 at a ratio of 2:1, respectively (total of 10 mg/ml bolas), cholesterol (1.6 mg/ml) and cholesteryl hemisuccinate (2.1 mg/ml). (Panel A) empty vesicles; (Panel B) vesicles loaded with 2 mg/ml trypsinogen.

FIG. 15: CryoTEM of empty vesicles made from a mixture of 10 mg/ml GLH-19 and GLH-20 (2:1) together with 1 mg/ml GLH-55a (Panel A), or 0.8 mg/ml GLH-57 (Panel B), or 1 mg/ml GLH55a and 0.8 mg/ml GLH-57 (Panel C). (Bar=50nm)

FIG. 16: CryoTEM of vesicles made from a mixture of GLH-19, GLH-20, GLH-55a, and GLH-57, as described in FIG. 15 (Panel C), and loaded with 40 μg/ml GDNF (Bar=50 nm)

FIG. 17A: Representative data from DLS measurements of size distribution by intensity for vesicles made from GLH-19; FIG. 17B: Representative data from DLS measurements of size distribution by intensity for vesicles made from GLH-20; and FIG. 17C: Representative data from DLS measurements of size distribution by intensity for vesicles made from a mixture of GLH-19 and GLH-20 at a ratio of 2:1. Vesicles were prepared by film hydration followed by sonication from 10 mg/ml bolas, 2.1 mg/ml cholesteryl hemisuccinate and 1.6 mg/ml cholesterol. Each sample was measured by the DLS 3 times, and each profile shows the three measurements overlaid.

FIG. 18: Size distribution of GLH-20 vesicles, with and without encapsulated trypsinogen. Vesicles were prepared by film hydration followed by sonication from 10 mg/ml GLH-20, 2.1 mg/ml cholesteryl hemisuccinate and 1.6 mg/ml cholesterol, in presence and absence of trypsinogen. Size distribution was measured by DLS.

FIG. 19: Stability of GLH-20 vesicles in storage. Encapsulation of CF was determined after diluting the vesicles to reduce the extravesicular CF concentration, then, the vesicles were disrupted by Triton X100 and the fluorescence of released CF was measured at various times as indicated. Encapsulation was normalized using encapsulation at time 0 as 100%

FIG. 20: Stability of bolaamphiphilic vesicles in whole serum. Vesicles were prepared from GLH-19 or GLH-20 or from mixtures of both bolas using two ratios as shown. Vesicles were added to the serum in a ratio of 1:10 (vesicles:serum). Percent CF encapsulation was determined by fluorescence measurements as described in FIG. 14. Encapsulation was normalized using encapsulation at time 0 as 100%

FIG. 21: Release of CF from bolavesicles in response to AChE. Vesicles were prepared from either GLH-20 alone (plus the standard additives) (Panel A), or a mixture of GLH-19 and GLH-20 (plus the standard additives) (Panel B) and both loaded with CF. The vesicles were placed in a cuvette, and fluorescence was measured as a function of time until stable reading was achieved. Then, 2 units of AChE was added to each vesicle preparation, and the fluorescence measurement continued. The release of the encapsulated CF causes increase in the fluorescence. About 7 minutes after the addition of the AChE, triton X100 was added (to a final concentration of 0.15%), to fully disrupt the vesicles and to release the remaining CF for the determination of the total CF that was encapsulated.

FIG. 22: Elution profile of a vesicle formulation that contained encapsulated (peak 1) and free trypsinogen (peak 2). The vesicles were applied on Sephadex G50 column and eluted with PBS.

FIG. 23: Quantification of encapsulated trypsinogen using the data obtained from the experiment described in FIG. 17.

FIG. 24: Encapsulation of trypsinogen following vesicle preparation by film hydration and sonication or by extrusion. Upper graph (Panel C) shows the overlap of the elution profiles obtained running each vesicle preparation on the Sephadex G50 column. The lower graphs show the quantification of encapsulation for sonicated vesicles (Panel A) and extruded vesicles (Panel B).

FIG. 25: Encapsulation of AlexaFluor®-488-labeled trypsinogen in bolaamphiphilc vesicles. Vesicles were made by film hydration followed by sonication from a mixture of 10 mg/ml GLH-19 and GLH-20 (2:1) with 2.1 mg/ml choesteryl hemisuccinate and 1.6 mg/ml cholesterol. Trypsinogen was labeled with AlexaFluor®-488, as described in the method section, and was included in the formulation at a concentration of 0.2 mg/ml. Vesicles were placed on Sephadex G50 column and eluted with either Tris buffer, pH=7.3 (Panel A); or PBS, pH 7.3 (Panel B). Percent encapsulation was quantified for (Panel B) and is shown in (Panel C). The calculated percent encapsulation was 31%.

FIG. 26A: Encapsulation efficiencies of trypsinogen and GDNF. Vesicles were prepared by film hydration followed by sonication from a mixture of GLH-19 and GLH-20 at a concentration of 10 mg/ml with 1.6 mg/ml cholesterol and 2.1 mg/ml cholesteryl hemisuccinate. The formulations contained 50 μg/ml trypsinogen, FIG. 26B: Encapsulation efficiencies of trypsinogen and GDNF. Vesicles were prepared by film hydration followed by sonication from a mixture of GLH-19 and GLH-20 at a concentration of 10 mg/ml with 1.6 mg/ml cholesterol and 2.1 mg/ml cholesteryl hemisuccinate. The formulations contained 100 μg/ml trypsinogen and FIG. 26C: Encapsulation efficiencies of trypsinogen and GDNF. Vesicles were prepared by film hydration followed by sonication from a mixture of GLH-19 and GLH-20 at a concentration of 10 mg/ml with 1.6 mg/ml cholesterol and 2.1 mg/ml cholesteryl hemisuccinate. The formulations contained 12.5 μg/ml GDNF. All proteins were labeled with AlexaFluor®-488. After encapsulation, the vesicles were eluted from a Sephadex G50 column by PBS and the fluorescence of each fraction was determined.

FIG. 27A: The effect of the encapsulation process on GDNF integrity and activity. Analysis of GDNF on PAGE, where lane 1 is empty vesicles; lane 2 is GDNF encapsulated by the method of film hydration followed by sonication; lane 3 is encapsulated GDNF which was incubated before the PAGE at 40oC for one hour; and lane 4 is free GDNF. FIG. 27B: Test of GDNF activity using SH-SYSY neuroblastoma cells where lane 1 is control untreated cells; lane 2 is cells treated with free GDNF; lane 3 is cells treated with empty vesicles; lane 4 is cells treated with free GDNF added to empty vesicles; and lane 5 is cells treated with GDNF encapsulated in bolavesicles by the method of film hydration followed by sonication

FIG. 28A: Uptake of CF-loaded vesicles into PC12 cells in culture. FIG. 28B: Uptake of CF-loaded vesicles into SH-5Y5Y neuroblastoma cells in culture. FIG. 28C: Uptake of CF-loaded vesicles into HeLa cells in culture. Vesicles were made from 10 mg/ml GLH-19:GLH-20 (2:1) without (uncoated vesicles) and with 0.8 mg/ml GLH-57, a bola that contains DAT ligand as the head group (DAT-vesicles). Cells were incubated for 1 h with the vesicles, and tested by flow cytometry. A shift to the right of the peak indicates fluorescent cells due to uptake of the vesicles.

FIG. 29: Accumulation of CF in the brain following i.v. administration. Vesicles were made by film hydration followed by sonication from a 10 mg/ml mixture of GLH-19 and GLH-20 (2:1), 1 mg/ml CS-fatty acid (vernolate) conjugate, 2.1 mg/ml cholesteryl hemisuccinate and 1.6 mg/ml cholesterol in absence (empty vesicles) and in presence of 0.2/ml CF (CF-loaded vesicles). Mice were pretreated with 0.5 mg/kg (i.m.) pyridostigmine and 15 min afterward the mice were injected i.v. with either free CF, or empty vesicles and then CF, or CF-loaded vesicles. The total amounts of the CF that were injected in each case were identical (10 mg/kg). 30 min after the injection, the animals were sacrificed, perfused with 10 ml PBS and the brains removed and homogenized, deproteinized by 5% tricholoroacetic acid and fluorescence determined in the supernatants obtained following centrifugation. Each bar represents an average value obtained from 5 mice+/−SEM.

FIG. 30: CF concentration in the brain after delivering it encapsulated in vesicles with CS surface groups. Vesicles were prepared as described in FIG. 24, except that in one case, 1 mg/ml GLH-55a was used in the vesicle formulation to provide CS surface groups (vesicles with CS-bola), and in the other case, 1 mg/ml CS-fatty acid conjugate was used. Conditions of this experiment were similar to those presented in FIG. 24.

FIG. 31: Distribution of CF in the brain after injecting CF-loaded vesicles with and without surface DAT ligand. Vesicles were prepared by film hydration followed by sonication from a 10 mg/ml mixture of GLH-19 and GLH-20 (2:1), 1 mg/ml GLH-55a (a bola with CS head group), 2.1 mg/ml cholesteryl hemisuccinate, 1.6 mg/ml cholesterol, 0.2 mg/ml CF and without (vesicle CS bola) or with GLH-57 (vesicles DAT CS bola). Mice were pretreated with 0.5 mg/kg (i.m.) pyridostigmine (to inhibit peripheral ChE) and 15 min afterward the vesicles were injected i.v. After 30 min the mice were sacrificed, perfused with 10 ml PBS and the brain removed and dissected into cortex, striatum and cerebellum. The tissues were weighed, homogenized and deproteinated by trichloroacetic acid, centrifuged and fluorescence was determined in the homogenates. The amount of the CF in each brain region (cerebellum (Panel A); cortex (Panel B); striatum (Panel C)) was calculated from a calibration curve of CF, taking into consideration the weight of the tissue and the dilution done during the homogenization. Each bar represent an average value obtained from 5 mice+/−SEM. The comparative data are depicted in Panel D.

FIG. 32A: Representative histofluorescence slides of brain tissue of control untreated mice; FIG. 32B: Representative histofluorescence slide showing AlxaFlour-488-labeld trypsinogen in brain tissue of mice injected with 200 μg of free trypsinogen labeled with AlexaFluor®-488; FIG. 32C: Representative histofluorescence slide showing AlxaFlour-488-labeld trypsinogen in brain tissue of mice injected with 200 μg of encapsulated trypsinogen labeled with AlexaFluor®-488; FIG. 32D: Representative histofluorescence slides of liver tissue of control untreated mice; FIG. 32E: Representative histofluorescence slide showing AlxaFlour-488-labeld trypsinogen in liver tissue of mice injected with 200 μg of free trypsinogen labeled with AlexaFluor®-488; FIG. 32F: Representative histofluorescence slide showing AlxaFlour-488-labeld trypsinogen in liver tissue of mice injected with 200 μg of encapsulated trypsinogen labeled with AlexaFluor®-488; FIG. 32G: Representative histofluorescence slide of kidney tissue of control untreated mice; FIG. 32H: Representative histofluorescence slide showing AlxaFlour-488-labeld trypsinogen in kidney tissue of mice injected with 200 μg of free trypsinogen labeled with AlexaFluor®-488; FIG. 32I: Representative histofluorescence slide showing AlxaFlour-488-labeld trypsinogen in kidney tissue of mice injected with 200 μg of encapsulated trypsinogen labeled with AlexaFluor®-488.

FIG. 33: Distribution of trypsinogen labeled with AlexaFluor®-488 in brain, kidney and liver after the injection (i.v.) of the labeled protein in its free form or encapsulated in vesicles. For the quantification, data obtained in the experiment described in FIG. 27 were used. Each bar represent an average value of 5 mice+/−SEM

FIG. 34: Representative brain sections stained for GDNF-biotin with avidine-AlexaFluor®-488. Mice were pretreated with 0.5 mg/kg (i.m.) pyridostigmine, then injected i.v. with vesicles coated with CS groups and DAT ligand with encapsulated GDNF-biotin. After 30 min, animals were sacrificed, perfused with 10 ml PBS, brains removed and striata, cortex and cerebella were dissected out, frozen and cryosectioned. Brain sections from these mice were stained with DAPI (blue) and avidine-AlexaFluor®-488 (green) and observed using confocal microscopy at a magnification of 10×. (Panel A) Stiatum from a mouse treated with PBS; (Panel B) striatum from a mouse injected with GDNF-biotin encapsulated in vesicles; (Panel C) cortex from a mouse injected with PBS; (Panel D) cortex from a mouse injected with GDNF-biotin encapsulated in vesicles; (Panel E) cerebellum from a mouse injected with PBS; (Panel F) cerebellum from a mouse injected with GDNF-biotin encapsulated in vesicles

FIG. 35: Distribution of exogenous GDNF-biotin in the brain after delivering the protein encapsulated in bolavesicles. These micrographs of high magnification, (60×) were taken from brain sections obtained from the mice used in the experiment described in FIG. 29. The nuclei of the cells appear in blue, due to DAPI staining, and the GDNF-biotin appears in green, due to the binding of the avidine-AlexaFluor®-488. (Panel A)) Stiatum from a mouse treated with PBS; (Panel B) striatum from a mouse injected with GDNF-biotin encapsulated in vesicles; (Panel C) cortex from a mouse injected with PBS; (Panel D) cortex from a mouse injected with GDNF-biotin encapsulated in vesicles; (Panel E) cerebellum from a mouse injected with PBS; (Panel F) cerebellum from a mouse injected with GDNF-biotin encapsulated in vesicles.

FIG. 36: Chemical shifts of the chloromethylene (—CH₂Cl) and alkoxymethylene (C(O)—O—CH₂—) groups of comound 4.

FIG. 37: Comparison of the NMR spectrum in CDCl₃ of the dichloroacetate intermediate 4 and the bolaamphiphile 5.

FIG. 38A: TEM micrographs of particles formed from bolaamphiphile GLH-20 and FIG. 38B: TEM micrographs of particles formed from bolaamphiphile GLH-32. Vesicles were prepared by film-hydration-extrusion (FHE) using 200 nm and 100 nm membranes, consecutively

FIG. 39: TEM micrographs of vesicles made from bolaamphiphile GLH-20 (Panel A) and bolaamphiphile GLH-32 (Panel B) formulated with CHOL and CHEMS at a molar ratio of 2:1:1. Vesicles were prepared by FHE using 200 nm and 100 nm membranes, consecutively

FIG. 40A: Vesicle stability determine by changes in vesicle size and FIG. 40B: Vesicle stability determine by changes in percent encapsulation using vesicles made from GLH-20 and GLH-32 with CHOL and CHEMS at a ratio of 2:1:1.

FIG. 41: Hydrolysis of the ACh head group of bioamphiphiles GLH-20 and GLH-32 by AChE. Hydrolysis was measured by determining the pH change after addition of AChE to the incubation medium and was converted to change in the proton concentration.

FIG. 42A: Lineweaver-Burk plots of ATC hydrolysis by AChE in presence of several concentrations of GLH-20 and FIG. 42B: Lineweaver-Burk plots of ATC hydrolysis by AChE in presence of several concentrations of GLH-32

FIG. 43: The effect of AChE on the release of CF from vesicles made from GLH-20 (Panel A) and GLH-32 (Panel B). The released CF was monitored by measuring the fluorescence before and after the addition of X units of AChE dissolved in X μl PBS. The experiment was terminated by the addition of Triton X-100 to disrupt the vesicles and release all the encapsulated CF.

FIG. 44: Percent release of encapsulated CF at different time after exposing bolaamphiphilic vesicles to AChE. Percent release was calculated from the amount of CF that was released at a particular time point versus the total amout of encapsulated CF, which was determined after lysing the vesicles with Triton X100.

FIG. 45: Depicts the ¹³C NMR spectra of the diester diglutarate 3 (Scheme 7), D-mannose and the bola GLH-64a in DMSO-d6.

FIG. 46: Depicts the main fragmentations in ESI-MS (positive mode) of GLH-64a.

FIG. 47: Depicts the HPLC chromatogram of GLH-64a, showing that it was obtained with a high purity.

FIG. 48: Depicts the separation of methyl ricinoleate by liquid-liquid extraction, where H=hexane; M=methanol; MR=methyl ricinoleate; and ME=mixture of methyl esters of castor oil.

FIG. 49: Provides the cryo-TEM images of vesicles prepared from a formulation without GLH-64a (Panel A) in comparison to a formulation that contained 5% GLH-64a (Panel B). The bar represents 100 nm.

FIG. 50: Depicts the ptake of fluorescent vesicles that contain GLH-64a by differentiated and non-differentiated J774 cells, as measured by FACS.

FIG. 51: Depicts the uptake of fluorescent vesicles that contain GLH-64a by differentiated J774 cells in presence and absence of free mannose in the bathing medium, as measured by FACS.

FIG. 52: Depicts the uptake of fluorescent vesicles that contain GLH-64a (Panel A) and GLH-64b (Panel B) by differentiated and non-differentiated J774 cells, as measured by FACS.

FIG. 53: Depicts the uptake of fluorescent vesicles (vesicles with encapsulated siRNA conjugated with AlexaFluor 546) with and without GLH-64a by differentiated J774 cells, as measured by FACS.

FIG. 54: Depicts the uptake of fluorescent vesicles that contain GLH-64d by differentiated and non-differentiated J774 cells, as measured by FACS.

FIG. 55: Depicts the amount of CF encapsulation as a function of time in storage at 4° C. in vesicles made from GLH-19 and GLH-20.

FIG. 56: Depicts the amount of CF encapsulation as a function of time in storage at 4° C. in vesicles made from GLH-19, GLH-20 and GLH-55b.

FIG. 57: Depicts the amount of CF encapsulation as a function of time in storage at 4° C. in vesicles made from GLH-19, GLH-20, GLH-55b and 1% GLH-64a.

FIG. 58: Depicts the amount of CF encapsulation as a function of time in storage at 4° C. in vesicles made from GLH-19, GLH-20, GLH-55b and 5% GLH-64a.

FIG. 59: Depicts the amount of CF encapsulation as a function of time in storage at 4° C. in vesicles made from GLH-19, GLH-20, GLH-55b and 10% GLH-64a.

FIG. 60: Depicts the stability in 4% albumin during storage at 4° C. of vesicles made from GLH-19, GLH-20 and GLH-55b.

FIG. 61: Depicts the stability in 4% albumin during storage at 4° C. of vesicles made from GLH-19, GLH-20, GLH-55b and 1% GLH-64.

FIG. 62: Depicts the stability in 4% albumin during storage at 25° C. of vesicles made from GLH-19, GLH-20 and GLH-55b.

FIG. 63: Depicts the stability in 4% albumin during storage at 25° C. of vesicles made from GLH-19, GLH-20 and GLH-55b and 1% GLH-64a.

FIG. 64: Depicts the effect of GLH-55b and GLH-64a on the release of encapsulated CF from vesicles. Panel A: Vesicles made of GLH-19 and GLH-20, without GLH-55b and GLH-64; Panel B—Vesicles made of GLH-19, GLH-20, GLH-55b and 1% GLH-64a; Panel C—Vesicles made of GLH-19, GLH-20, GLH-55b and 10% GLH-64a.

### DEFINITIONS

### Chemical Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

Compounds described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, NY, 1962); and Wilen, Tables of Resolving Agents and Optical Resolutions p. 268 (E.L. Eliel, Ed., Univ. of Notre Dame Press, Notre Dame, Ind. 1972). The invention additionally encompasses compounds described herein as individual isomers substantially free of other isomers, and alternatively, as mixtures of various isomers.

When a range of values is listed, it is intended to encompass each value and sub-range within the range. For example “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

The following terms are intended to have the meanings presented therewith below and are useful in understanding the description and intended scope of the present invention. When describing the invention, which may include compounds, pharmaceutical compositions containing such compounds and methods of using such compounds and compositions, the following terms, if present, have the following meanings unless otherwise indicated. It should also be understood that when described herein any of the moieties defined forth below may be substituted with a variety of substituents, and that the respective definitions are intended to include such substituted moieties within their scope as set out below. Unless otherwise stated, the term “substituted” is to be defined as set out below. It should be further understood that the terms “groups” and “radicals” can be considered interchangeable when used herein. The articles “a” and “an” may be used herein to refer to one or to more than one (i.e. at least one) of the grammatical objects of the article. By way of example “an analogue” means one analogue or more than one analogue.

“Alkyl” refers to a radical of a straight-chain or branched saturated hydrocarbon group having from 1 to 20 carbon atoms (“C₁₋₂₀ alkyl”). In some embodiments, an alkyl group has 1 to 12 carbon atoms (“C₁₋₁₂ alkyl”). In some embodiments, an alkyl group has 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”, also referred to herein as “lower alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). Additional examples of alkyl groups include n-heptyl (C₇), n-octyl (C₅) and the like. Unless otherwise specified, each instance of an alkyl group is independently optionally substituted, i.e., unsubstituted (an “unsubstituted alkyl”) or substituted (a “substituted alkyl”) with one or more substituents; e.g., for instance from 1 to 5 substituents, 1 to 3 substituents, or 1 substituent. In certain embodiments, the alkyl group is unsubstituted C₁₋₁₀ alkyl (e.g., —CH₃). In certain embodiments, the alkyl group is substituted C₁₋₁₀ alkyl.

“Alkylene” refers to a substituted or unsubstituted alkyl group, as defined above, wherein two hydrogens are removed to provide a divalent radical. Exemplary divalent alkylene groups include, but are not limited to, methylene (—CH₂—), ethylene (—CH₂CH₂—), the propylene isomers (e.g., —CH₂CH₂CH₂— and —CH(CH₃)CH₂—) and the like.

“Alkenyl” refers to a radical of a straight-chain or branched hydrocarbon group having from 2 to 20 carbon atoms, one or more carbon-carbon double bonds, and no triple bonds (“C₂₋₂₀ alkenyl”). In some embodiments, an alkenyl group has 2 to 10 carbon atoms (“C₂₋₁₀ alkenyl”). In some embodiments, an alkenyl group has 2 to 9 carbon atoms (“C₂₋₉ alkenyl”). In some embodiments, an alkenyl group has 2 to 8 carbon atoms (“C₂₋₈ alkenyl”). In some embodiments, an alkenyl group has 2 to 7 carbon atoms (“C₂₋₇ alkenyl”). In some embodiments, an alkenyl group has 2 to 6 carbon atoms (“C₂₋₆ alkenyl”). In some embodiments, an alkenyl group has 2 to 5 carbon atoms (“C₂₋₅ alkenyl”). In some embodiments, an alkenyl group has 2 to 4 carbon atoms (“C₂₋₄ alkenyl”). In some embodiments, an alkenyl group has 2 to 3 carbon atoms (“C₂₋₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₅), octatrienyl (C₅), and the like. Unless otherwise specified, each instance of an alkenyl group is independently optionally substituted, i.e., unsubstituted (an “unsubstituted alkenyl”) or substituted (a “substituted alkenyl”) with one or more substituents e.g., for instance from 1 to 5 substituents, 1 to 3 substituents, or 1 substituent. In certain embodiments, the alkenyl group is unsubstituted C₂₋₁₀ alkenyl. In certain embodiments, the alkenyl group is substituted C₂₋₁₀ alkenyl.

“Alkenylene” refers a substituted or unsubstituted alkenyl group, as defined above, wherein two hydrogens are removed to provide a divalent radical. Exemplary divalent alkenylene groups include, but are not limited to, ethenylene (—CH═CH—), propenylenes (e.g., —CH═CHCH₂— and —C(CH₃)═CH— and —CH═C(CH₃)—) and the like.

“Alkynyl” refers to a radical of a straight-chain or branched hydrocarbon group having from 2 to 20 carbon atoms, one or more carbon-carbon triple bonds, and optionally one or more double bonds (“C₂₋₂₀ alkynyl”). In some embodiments, an alkynyl group has 2 to 10 carbon atoms (“C₂₋₁₀ alkynyl”). In some embodiments, an alkynyl group has 2 to 9 carbon atoms (“C₂₋₉ alkynyl”). In some embodiments, an alkynyl group has 2 to 8 carbon atoms (“C₂₋₈ alkynyl”). In some embodiments, an alkynyl group has 2 to 7 carbon atoms (“C₂₋₇ alkynyl”). In some embodiments, an alkynyl group has 2 to 6 carbon atoms (“C₂₋₆ alkynyl”). In some embodiments, an alkynyl group has 2 to 5 carbon atoms (“C₂₋₅ alkynyl”). In some embodiments, an alkynyl group has 2 to 4 carbon atoms (“C₂₋₄ alkynyl”). In some embodiments, an alkynyl group has 2 to 3 carbon atoms (“C₂₋₃ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include, without limitation, ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₅), and the like. Unless otherwise specified, each instance of an alkynyl group is independently optionally substituted, i.e., unsubstituted (an “unsubstituted alkynyl”) or substituted (a “substituted alkynyl”) with one or more substituents; e.g., for instance from 1 to 5 substituents, 1 to 3 substituents, or 1 substituent. In certain embodiments, the alkynyl group is unsubstituted C₂₋₁₀ alkynyl. In certain embodiments, the alkynyl group is substituted C₂₋₁₀ alkynyl.

“Alkynylene” refers a substituted or unsubstituted alkynyl group, as defined above, wherein two hydrogens are removed to provide a divalent radical. Exemplary divalent alkynylene groups include, but are not limited to, ethynylene, propynylene, and the like.

“Aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆₋₁₄ aryl”). In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl). In some embodiments, an aryl group has fourteen ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Typical aryl groups include, but are not limited to, groups derived from aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, and trinaphthalene. Particularly aryl groups include phenyl, naphthyl, indenyl, and tetrahydronaphthyl. Unless otherwise specified, each instance of an aryl group is independently optionally substituted, i.e., unsubstituted (an “unsubstituted aryl”) or substituted (a “substituted aryl”) with one or more substituents. In certain embodiments, the aryl group is unsubstituted C₆₋₁₄ aryl. In certain embodiments, the aryl group is substituted C₆₋₁₄ aryl.

In certain embodiments, an aryl group substituted with one or more of groups selected from halo, C₁-C₈ alkyl, C₁-C₈ haloalkyl, cyano, hydroxy, C₁-C₈ alkoxy, and amino.

Examples of representative substituted aryls include the following

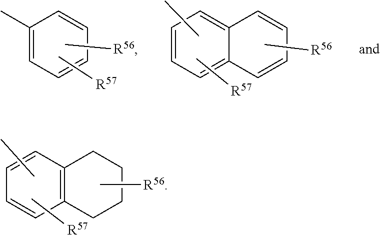

In these formulae one of R⁵⁶ and R⁵⁷ may be hydrogen and at least one of R⁵⁶ and R⁵⁷ is each independently selected from C₁-C₈ alkyl, C₁-C₈ haloalkyl, 4-10 membered heterocyclyl, alkanoyl, C₁-C₈ alkoxy, heteroaryloxy, alkylamino, arylamino, heteroarylamino, NR⁵⁸COR⁵⁹, NR⁵⁸SOR⁵⁹NR⁵⁸SO₂R⁵⁹, COOalkyl, COOaryl, CONR⁵⁸R⁵⁹, CONR⁵⁸OR⁵⁹, NR⁵⁸R⁵⁹, SO₂NR⁵⁸R⁵⁹, S-alkyl, SOalkyl, SO₂alkyl, Saryl, SOaryl, SO₂aryl; or R⁵⁶ and R⁵⁷ may be joined to form a cyclic ring (saturated or unsaturated) from 5 to 8 atoms, optionally containing one or more heteroatoms selected from the group N, O, or S. R⁶⁰ and R⁶¹ are independently hydrogen, C₁-C₈ alkyl, C₁-C₄ haloalkyl, C₃-C₁₀ cycloalkyl, 4-10 membered heterocyclyl, C₆-C₁₀ aryl, substituted C₆-C₁₀ aryl, 5-10 membered heteroaryl, or substituted 5-10 membered heteroaryl.

“Fused aryl” refers to an aryl having two of its ring carbon in common with a second aryl ring or with an aliphatic ring.

“Aralkyl” is a subset of alkyl and aryl, as defined herein, and refers to an optionally substituted alkyl group substituted by an optionally substituted aryl group.

“Heteroaryl” refers to a radical of a 5-10 membered monocyclic or bicyclic 4n+2 aromatic ring system (e.g., having 6 or 10 π electrons shared in a cyclic array) having ring carbon atoms and 1-4 ring heteroatoms provided in the aromatic ring system, wherein each heteroatom is independently selected from nitrogen, oxygen and sulfur (“5-10 membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused (aryl/heteroaryl) ring system. Bicyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl).

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Unless otherwise specified, each instance of a heteroaryl group is independently optionally substituted, i.e., unsubstituted (an “unsubstituted heteroaryl”) or substituted (a “substituted heteroaryl”) with one or more substituents. In certain embodiments, the heteroaryl group is unsubstituted 5-14 membered heteroaryl. In certain embodiments, the heteroaryl group is substituted 5-14 membered heteroaryl.

Exemplary 5-membered heteroaryl groups containing one heteroatom include, without limitation, pyrrolyl, furanyl and thiophenyl. Exemplary 5-membered heteroaryl groups containing two heteroatoms include, without limitation, imidazolyl, pyrazolyl, oxazolyl, isoxazolyl, thiazolyl, and isothiazolyl. Exemplary 5-membered heteroaryl groups containing three heteroatoms include, without limitation, triazolyl, oxadiazolyl, and thiadiazolyl. Exemplary 5-membered heteroaryl groups containing four heteroatoms include, without limitation, tetrazolyl. Exemplary 6-membered heteroaryl groups containing one heteroatom include, without limitation, pyridinyl. Exemplary 6-membered heteroaryl groups containing two heteroatoms include, without limitation, pyridazinyl, pyrimidinyl, and pyrazinyl. Exemplary 6-membered heteroaryl groups containing three or four heteroatoms include, without limitation, triazinyl and tetrazinyl, respectively. Exemplary 7-membered heteroaryl groups containing one heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl.

Examples of representative heteroaryls include the following:

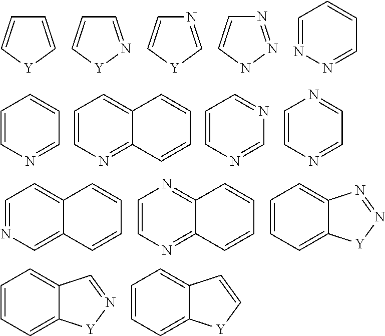

wherein each Y is selected from carbonyl, N, NR⁶⁵, O, and S; and R⁶⁵ is independently hydrogen, C₁-C₈ alkyl, C₃-C₁₀ cycloalkyl, 4-10 membered heterocyclyl, C₆-C₁₀ aryl, and 5-10 membered heteroaryl.

Examples of representative aryl having hetero atoms containing substitution include the following:

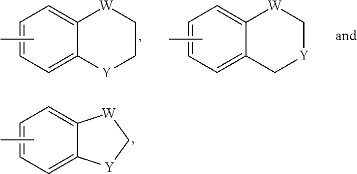

wherein each W is selected from C(R⁶⁶)₂, NR⁶⁶, O, and S; and each Y is selected from carbonyl, NR⁶⁶, O and S; and R⁶⁶ is independently hydrogen, C₁-C₈ alkyl, C₃-C₁₀ cycloalkyl, 4-10 membered heterocyclyl, C₆-C₁₀ aryl, and 5-10 membered heteroaryl.

“Heteroaralkyl” is a subset of alkyl and heteroaryl, as defined herein, and refers to an optionally substituted alkyl group substituted by an optionally substituted heteroaryl group.

“Carbocyclyl” or “carbocyclic” refers to a radical of a non-aromatic cyclic hydrocarbon group having from 3 to 10 ring carbon atoms (“C₃₋₁₀ carbocyclyl”) and zero heteroatoms in the non-aromatic ring system. In some embodiments, a carbocyclyl group has 3 to 8 ring carbon atoms (“C₃₋₈ carbocyclyl”). In some embodiments, a carbocyclyl group has 3 to 6 ring carbon atoms (“C₃₋₆ carbocyclyl”). In some embodiments, a carbocyclyl group has 3 to 6 ring carbon atoms (“C₃₋₆ carbocyclyl”). In some embodiments, a carbocyclyl group has 5 to 10 ring carbon atoms (“C₅₋₁₀ carbocyclyl”). Exemplary C₃₋₆ carbocyclyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include, without limitation, the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₅), cyclooctenyl (C₅), bicyclo[2.2.1]heptanyl (C₇), bicyclo[2.2.2]octanyl (C₅), and the like. Exemplary C₃₋₁₀ carbocyclyl groups include, without limitation, the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. As the foregoing examples illustrate, in certain embodiments, the carbocyclyl group is either monocyclic (“monocyclic carbocyclyl”) or contain a fused, bridged or spiro ring system such as a bicyclic system (“bicyclic carbocyclyl”) and can be saturated or can be partially unsaturated. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently optionally substituted, i.e., unsubstituted (an “unsubstituted carbocyclyl”) or substituted (a “substituted carbocyclyl”) with one or more substituents. In certain embodiments, the carbocyclyl group is unsubstituted C₃₋₁₀ carbocyclyl. In certain embodiments, the carbocyclyl group is a substituted C₃₋₁₀ carbocyclyl.

In some embodiments, “carbocyclyl” is a monocyclic, saturated carbocyclyl group having from 3 to 10 ring carbon atoms (“C₃-10 cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”). Examples of C₅₋₆ cycloalkyl groups include cyclopentyl (C₅) and cyclohexyl (C₅). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₅). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is unsubstituted C₃₋₁₀ cycloalkyl. In certain embodiments, the cycloalkyl group is substituted C₃₋₁₀ cycloalkyl.

“Heterocyclyl” or “heterocyclic” refers to a radical of a 3- to 10-membered non-aromatic ring system having ring carbon atoms and 1 to 4 ring heteroatoms, wherein each heteroatom is independently selected from nitrogen, oxygen, sulfur, boron, phosphorus, and silicon (“3-10 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged or spiro ring system such as a bicyclic system (“bicyclic heterocyclyl”), and can be saturated or can be partially unsaturated. Heterocyclyl bicyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently optionally substituted, i.e., unsubstituted (an “unsubstituted heterocyclyl”) or substituted (a “substituted heterocyclyl”) with one or more substituents. In certain embodiments, the heterocyclyl group is unsubstituted 3-10 membered heterocyclyl. In certain embodiments, the heterocyclyl group is substituted 3-10 membered heterocyclyl.

In some embodiments, a heterocyclyl group is a 5-10 membered non-aromatic ring system having ring carbon atoms and 1-4 ring heteroatoms, wherein each heteroatom is independently selected from nitrogen, oxygen, sulfur, boron, phosphorus, and silicon (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has one ring heteroatom selected from nitrogen, oxygen, and sulfur.

Exemplary 3-membered heterocyclyl groups containing one heteroatom include, without limitation, azirdinyl, oxiranyl, thiorenyl. Exemplary 4-membered heterocyclyl groups containing one heteroatom include, without limitation, azetidinyl, oxetanyl and thietanyl. Exemplary 5-membered heterocyclyl groups containing one heteroatom include, without limitation, tetrahydrofuranyl, dihydrofuranyl, tetrahydrothiophenyl, dihydrothiophenyl, pyrrolidinyl, dihydropyrrolyl and pyrrolyl-2,5-dione. Exemplary 5-membered heterocyclyl groups containing two heteroatoms include, without limitation, dioxolanyl, oxasulfuranyl, disulfuranyl, and oxazolidin-2-one. Exemplary 5-membered heterocyclyl groups containing three heteroatoms include, without limitation, triazolinyl, oxadiazolinyl, and thiadiazolinyl. Exemplary 6-membered heterocyclyl groups containing one heteroatom include, without limitation, piperidinyl, tetrahydropyranyl, dihydropyridinyl, and thianyl. Exemplary 6-membered heterocyclyl groups containing two heteroatoms include, without limitation, piperazinyl, morpholinyl, dithianyl, dioxanyl. Exemplary 6-membered heterocyclyl groups containing two heteroatoms include, without limitation, triazinanyl. Exemplary 7-membered heterocyclyl groups containing one heteroatom include, without limitation, azepanyl, oxepanyl and thiepanyl. Exemplary 8-membered heterocyclyl groups containing one heteroatom include, without limitation, azocanyl, oxecanyl and thiocanyl. Exemplary 5-membered heterocyclyl groups fused to a C₆ aryl ring (also referred to herein as a 5,6-bicyclic heterocyclic ring) include, without limitation, indolinyl, isoindolinyl, dihydrobenzofuranyl, dihydrobenzothienyl, benzoxazolinonyl, and the like. Exemplary 6-membered heterocyclyl groups fused to an aryl ring (also referred to herein as a 6,6-bicyclic heterocyclic ring) include, without limitation, tetrahydroquinolinyl, tetrahydroisoquinolinyl, and the like.

Particular examples of heterocyclyl groups are shown in the following illustrative examples:

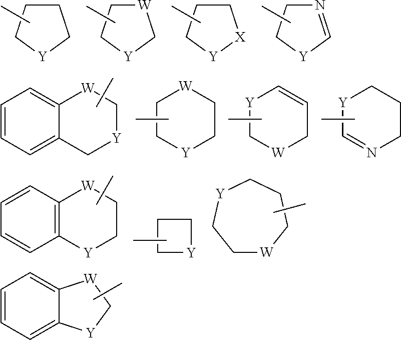

wherein each W is selected from CR⁶⁷, C(R⁶⁷)₂, NR⁶⁷, O, and S; and each Y is selected from NR⁶⁷, O, and S; and R⁶⁷ is independently hydrogen, C₁-C₈ alkyl, C₃-C₁₀ cycloalkyl, 4-10 membered heterocyclyl, C₆-C₁₀ aryl, 5-10 membered heteroaryl. These heterocyclyl rings may be optionally substituted with one or more substituents selected from the group consisting of the group consisting of acyl, acylamino, acyloxy, alkoxy, alkoxycarbonyl, alkoxycarbonylamino, amino, substituted amino, aminocarbonyl (carbamoyl or amido), aminocarbonylamino, aminosulfonyl, sulfonylamino, aryl, aryloxy, azido, carboxyl, cyano, cycloalkyl, halogen, hydroxy, keto, nitro, thiol, —S-alkyl, —S-aryl, —S(O)-alkyl, —S(O)-aryl, —S(O)₂-alkyl, and —S(O)₂-aryl. Substituting groups include carbonyl or thiocarbonyl which provide, for example, lactam and urea derivatives.

“Hetero” when used to describe a compound or a group present on a compound means that one or more carbon atoms in the compound or group have been replaced by a nitrogen, oxygen, or sulfur heteroatom. Hetero may be applied to any of the hydrocarbyl groups described above such as alkyl, e.g., heteroalkyl, cycloalkyl, e.g., heterocyclyl, aryl, e.g,. heteroaryl, cycloalkenyl, e.g,. cycloheteroalkenyl, and the like having from 1 to 5, and particularly from 1 to 3 heteroatoms.

“Acyl” refers to a radical —C(O)R²⁰, where R²⁰ is hydrogen, substituted or unsubstitued alkyl, substituted or unsubstitued alkenyl, substituted or unsubstitued alkynyl, substituted or unsubstitued carbocyclyl, substituted or unsubstituted heterocyclyl, substituted or unsubstituted aryl, or substituted or unsubstitued heteroaryl, as defined herein. “Alkanoyl” is an acyl group wherein R²⁰ is a group other than hydrogen. Representative acyl groups include, but are not limited to, formyl (—CHO), acetyl (—C(═O)CH₃), cyclohexylcarbonyl, cyclohexylmethylcarbonyl, benzoyl (—C(═O)Ph), benzylcarbonyl (—C(═O)CH₂Ph), —C(O)—C₁-C₈ alkyl, —C(O)—(CH₂)ₜ(C₆-C₁₀ aryl), —C(O)—(CH₂)ₜ(5-10 membered heteroaryl), —C(O)—(CH₂)ₜ(C₃-C₁₀ cycloalkyl), and —C(O)—(CH₂)ₜ(4-10 membered heterocyclyl), wherein t is an integer from 0 to 4. In certain embodiments, R²¹ is C₁-C₈ alkyl, substituted with halo or hydroxy; or C₃-C₁₀ cycloalkyl, 4-10 membered heterocyclyl, C₆-C₁₀ aryl, arylalkyl, 5-10 membered heteroaryl or heteroarylalkyl, each of which is substituted with unsubstituted C₁-C₄ alkyl, halo, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxy.

“Acylamino” refers to a radical —NR²²C(O)R²³, where each instance of R²² and R23 is independently hydrogen, substituted or unsubstitued alkyl, substituted or unsubstitued alkenyl, substituted or unsubstitued alkynyl, substituted or unsubstitued carbocyclyl, substituted or unsubstituted heterocyclyl, substituted or unsubstituted aryl, or substituted or unsubstitued heteroaryl—as defined herein, or R²² is an amino protecting group. Exemplary “acylamino” groups include, but are not limited to, formylamino, acetylamino, cyclohexylcarbonylamino, cyclohexylmethyl-carbonylamino, benzoylamino and benzylcarbonylamino. Particular exemplary “acylamino” groups are —NR²⁴C(O)—C₁-C₈ alkyl, —NR²⁴C(O)—(CH₂)ₜ(C₆-C₁₀ aryl), —NR²⁴C(O)—(CH₂)ₜ(5-10 membered heteroaryl), —NR²⁴C(O)—(CH₂)ₜ(C₃-C₁₀ cycloalkyl), and —NR²⁴C(O)—(CH₂)ₜ(4-10 membered heterocyclyl), wherein t is an integer from 0 to 4, and each R²⁴ independently represents H or C₁-C₈ alkyl. In certain embodiments, R²⁵ is H, C₁-C₈ alkyl, substituted with halo or hydroxy; C₃-C₁₀ cycloalkyl, 4-10 membered heterocyclyl, C₆-C₁₀ aryl, arylalkyl, 5-10 membered heteroaryl or heteroarylalkyl, each of which is substituted with unsubstituted C₁-C₄ alkyl, halo, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxy; and R²⁶ is H, C₁-C₈ alkyl, substituted with halo or hydroxy; C₃-C₁₀ cycloalkyl, 4-10 membered heterocyclyl, C₆-C₁₀ aryl, arylalkyl, 5-10 membered heteroaryl or heteroarylalkyl, each of which is substituted with unsubstituted C₁-C₄ alkyl, halo, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxyl; provided that at least one of R²⁵ and R²⁶ is other than H.

“Acyloxy” refers to a radical —OC(O)R²⁷, where R²⁷ is hydrogen, substituted or unsubstitued alkyl, substituted or unsubstitued alkenyl, substituted or unsubstituted alkynyl, substituted or unsubstituted carbocyclyl, substituted or unsubstituted heterocyclyl, substituted or unsubstituted aryl, or substituted or unsubstituted heteroaryl, as defined herein. Representative examples include, but are not limited to, formyl, acetyl, cyclohexylcarbonyl, cyclohexylmethylcarbonyl, benzoyl and benzylcarbonyl. In certain embodiments, R²⁸ is C₁-C₈ alkyl, substituted with halo or hydroxy; C₃-C₁₀ cycloalkyl, 4-10 membered heterocyclyl, C₆-C₁₀ aryl, arylalkyl, 5-10 membered heteroaryl or heteroarylalkyl, each of which is substituted with unsubstituted C₁-C₄ alkyl, halo, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxy.

“Alkoxy” refers to the group —OR²⁹ where R²⁹ is substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl. Particular alkoxy groups are methoxy, ethoxy, n-propoxy, isopropoxy, n-butoxy, tert-butoxy, sec-butoxy, n-pentoxy, n-hexoxy, and 1,2-dimethylbutoxy. Particular alkoxy groups are lower alkoxy, i.e. with between 1 and 6 carbon atoms. Further particular alkoxy groups have between 1 and 4 carbon atoms.

In certain embodiments, R²⁹ is a group that has 1 or more substituents, for instance, from 1 to 5 substituents, and particularly from 1 to 3 substituents, in particular 1 substituent, selected from the group consisting of amino, substituted amino, C₆-C₁₀ aryl, aryloxy, carboxyl, cyano, C₃-C₁₀ cycloalkyl, 4-10 membered heterocyclyl, halogen, 5-10 membered heteroaryl, hydroxyl, nitro, thioalkoxy, thioaryloxy, thiol, alkyl-S(O)—, aryl-S(O)—, alkyl-S(O)₂— and aryl-S(O)₂—. Exemplary ‘substituted alkoxy’ groups include, but are not limited to, —O—(CH₂)ₜ(C₆-C₁₀ aryl), —O—(CH₂)ₜ(5-10 membered heteroaryl), —O—(CH₂)ₜ(C₃-C₁₀ cycloalkyl), and —O—(CH₂)ₜ(4-10 membered heterocyclyl), wherein t is an integer from 0 to 4 and any aryl, heteroaryl, cycloalkyl or heterocyclyl groups present, may themselves be substituted by unsubstituted C₁-C₄ alkyl, halo, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxy. Particular exemplary ‘substituted alkoxy’ groups are —OCF₃, —OCH₂CF₃, —OCH₂Ph, —OCH₂-cyclopropyl, —OCH₂CH₂OH, and —OCH₂CH₂NMe₂.

“Amino” refers to the radical —NH₂.

“Substituted amino” refers to an amino group of the formula —N(R³⁸)₂ wherein R³⁸ is hydrogen, substituted or unsubstituted alkyl, substituted or unsubstitued alkenyl, substituted or unsubstitued alkynyl, substituted or unsubstitued carbocyclyl, substituted or unsubstituted heterocyclyl, substituted or unsubstituted aryl, substituted or unsubstitued heteroaryl, or an amino protecting group, wherein at least one of R³⁸ is not a hydrogen. In certain embodiments,each R³⁸ is independently selected from: hydrogen, C₁-C₈ alkyl, C₃-C₈ alkenyl, C₃-C₈ alkynyl, C₆-C₁₀ aryl, 5-10 membered heteroaryl, 4-10 membered heterocyclyl, or C3-C₁₀ cycloalkyl; or C₁-C₈ alkyl, substituted with halo or hydroxy; C₃-C₈ alkenyl, substituted with halo or hydroxy; C₃-C₈ alkynyl, substituted with halo or hydroxy, or —(CH₂)ₜ(C₆-C₁₀ aryl), —(CH₂)ₜ(5-10 membered heteroaryl), —(CH₂)ₜ(C₃-C₁₀ cycloalkyl), or —(CH₂)ₜ(4-10 membered heterocyclyl), wherein t is an integer between 0 and 8, each of which is substituted by unsubstituted C₁-C₄ alkyl, halo, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxy; or both R³⁸ groups are joined to form an alkylene group.

Exemplary ‘substituted amino’ groups are —NR³⁹—C₁-C₈ alkyl, —NR³⁹—(CH₂)ₜ(C₆-C₁₀ aryl), —NR³⁹—(CH₂)ₜ(5-10 membered heteroaryl), —NR³⁹—(CH₂)ₜ(C₃-C₁₀ cycloalkyl), and —NR³⁹—(CH₂)ₜ(4-10 membered heterocyclyl), wherein t is an integer from 0 to 4, for instance 1 or 2, each R³⁹ independently represents H or C₁-C₈ alkyl, and any alkyl groups present, may themselves be substituted by halo, substituted or unsubstituted amino, or hydroxy; and any aryl, heteroaryl, cycloalkyl, or heterocyclyl groups present, may themselves be substituted by unsubstituted C₁-C₄ alkyl, halo, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxy. For the avoidance of doubt the term ‘substituted amino’ includes the groups alkylamino, substituted alkylamino, alkylarylamino, substituted alkylarylamino, arylamino, substituted arylamino, dialkylamino, and substituted dialkylamino as defined below. Substituted amino encompasses both monosubstituted amino and disubstituted amino groups.

“Azido” refers to the radical —N₃.

“Carbamoyl” or “amido” refers to the radical —C(O)NH₂.

“Substituted carbamoyl” or “substituted amido” refers to the radical —C(O)N(R⁶²)₂ wherein each R⁶² is independently hydrogen, substituted or unsubstituted alkyl, substituted or unsubstitued alkenyl, substituted or unsubstitued alkynyl, substituted or unsubstitued carbocyclyl, substituted or unsubstituted heterocyclyl, substituted or unsubstituted aryl, substituted or unsubstitued heteroaryl, or an amino protecting group, wherein at least one of R⁶² is not a hydrogen. In certain embodiments, R⁶² is selected from H, C₁-C₈ alkyl, C3-C₁₀ cycloalkyl, 4-10 membered heterocyclyl, C₆-C₁₀ aryl, aralkyl, 5-10 membered heteroaryl, and heteroaralkyl; or C₁-C₈ alkyl substituted with halo or hydroxy; or C₃-C₁₀ cycloalkyl, 4-10 membered heterocyclyl, C₆-C₁₀ aryl, aralkyl, 5-10 membered heteroaryl, or heteroaralkyl, each of which is substituted by unsubstituted C₁-C₄ alkyl, halo, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxy; provided that at least one R⁶² is other than H.

Exemplary ‘substituted carbamoyl’ groups include, but are not limited to, —C(O) NR⁶⁴—C₁-C₈ alkyl, —C(O)NR⁶⁴—(CH₂)ₜ(C₆-C₁₀ aryl), —C(O)N⁶⁴—(CH₂)ₜ(5-10 membered heteroaryl), —C(O)NR⁶⁴—(CH₂)ₜ(C₃-C₁₀ cycloalkyl), and —C(O)NR⁶⁴—(CH₂)ₜ(4-10 membered heterocyclyl), wherein t is an integer from 0 to 4, each R⁶⁴ independently represents H or C₁-C₈ alkyl and any aryl, heteroaryl, cycloalkyl or heterocyclyl groups present, may themselves be substituted by unsubstituted C₁-C₄ alkyl, halo, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxy.

‘Carboxy’ refers to the radical —C(O)OH.

“Cyano” refers to the radical —CN.

“Halo” or “halogen” refers to fluoro (F), chloro (Cl), bromo (Br), and iodo (I). In certain embodiments, the halo group is either fluoro or chloro. In further embodiments, the halo group is iodo.

“Hydroxy” refers to the radical —OH.

“Nitro” refers to the radical —NO₂.

“Cycloalkylalkyl” refers to an alkyl radical in which the alkyl group is substituted with a cycloalkyl group. Typical cycloalkylalkyl groups include, but are not limited to, cyclopropylmethyl, cyclobutylmethyl, cyclopentylmethyl, cyclohexylmethyl, cycloheptylmethyl, cyclooctylmethyl, cyclopropylethyl, cyclobutylethyl, cyclopentylethyl, cyclohexylethyl, cycloheptylethyl, and cyclooctylethyl, and the like.

“Heterocyclylalkyl” refers to an alkyl radical in which the alkyl group is substituted with a heterocyclyl group. Typical heterocyclylalkyl groups include, but are not limited to, pyrrolidinylmethyl, piperidinylmethyl, piperazinylmethyl, morpholinylmethyl, pyrrolidinylethyl, piperidinylethyl, piperazinylethyl, morpholinylethyl, and the like.

“Cycloalkenyl” refers to substituted or unsubstituted carbocyclyl group having from 3 to 10 carbon atoms and having a single cyclic ring or multiple condensed rings, including fused and bridged ring systems and having at least one and particularly from 1 to 2 sites of olefinic unsaturation. Such cycloalkenyl groups include, by way of example, single ring structures such as cyclohexenyl, cyclopentenyl, cyclopropenyl, and the like.

“Fused cycloalkenyl” refers to a cycloalkenyl having two of its ring carbon atoms in common with a second aliphatic or aromatic ring and having its olefinic unsaturation located to impart aromaticity to the cycloalkenyl ring.

“Ethenyl” refers to substituted or unsubstituted —(C═C)—.

“Ethylene” refers to substituted or unsubstituted —(C—C)—.

“Ethynyl” refers to —(C≡C)—.

“Nitrogen-containing heterocyclyl” group means a 4- to 7-membered non-aromatic cyclic group containing at least one nitrogen atom, for example, but without limitation, morpholine, piperidine (e.g. 2-piperidinyl, 3-piperidinyl and 4-piperidinyl), pyrrolidine (e.g. 2-pyrrolidinyl and 3-pyrrolidinyl), azetidine, pyrrolidone, imidazoline, imidazolidinone, 2-pyrazoline, pyrazolidine, piperazine, and N-alkyl piperazines such as N-methyl piperazine. Particular examples include azetidine, piperidone and piperazone.

“Thioketo” refers to the group ═S.

Alkyl, alkenyl, alkynyl, carbocyclyl, heterocyclyl, aryl, and heteroaryl groups, as defined herein, are optionally substituted (e.g., “substituted” or “unsubstituted” alkyl, “substituted” or “unsubstituted” alkenyl, “substituted” or “unsubstituted” alkynyl, “substituted” or “unsubstituted” carbocyclyl, “substituted” or “unsubstituted” heterocyclyl, “substituted” or “unsubstituted” aryl or “substituted” or “unsubstituted” heteroaryl group). In general, the term “substituted”, whether preceded by the term “optionally” or not, means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, any of the substituents described herein that results in the formation of a stable compound. The present invention contemplates any and all such combinations in order to arrive at a stable compound. For purposes of this invention, heteroatoms such as nitrogen may have hydrogen substituents and/or any suitable substituent as described herein which satisfy the valencies of the heteroatoms and results in the formation of a stable moiety.

Exemplary carbon atom substituents include, but are not limited to, halogen, —CN, —NO₂, —N₃, —SO₂H, —SO₃H, —OH, —OR^{aa}, —ON(R^{bb})₂, —N(R^{bb})₂, —N(R^{bb})₃⁺X⁻, —N(OR^{cc})R^{bb}, —SH —SR^{aa}, —SSR^{cc}, —C(═O)R^{aa}, —CO₂H, —CHO, —C(OR^{cc})₂, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})₃, —OSi(R^{aa})₃—C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)₂R^{aa}, —OP(═O)₂R^{aa}, —P(═O)(R^{aa})₂, —OP(═O)(R^{aa})₂, —OP(═O)(OR^{cc})₂, —P(═O)₂N(R^{bb})₂, —OP(═O)₂N(R^{bb})₂, —P(═O)(NR^{bb})₂, —OP(═O)(NR^{bb})₂, —NR^{bb}P(═O)(OR^{cc})₂, —NR^{bb}P(═O)(NR^{bb})₂, —P(R^{cc})₂, —P(R^{cc})₃, —OP(R^{cc})₂, —OP(R^{cc})₃, —B(R^{aa})₂, —B(OR^{cc})₂, —BR^{aa}(OR^{cc}), C₁₋₁₀ alkyl, C₁₋₁₀ perhaloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, carbocyclyl, heterocyclyl, aryl, and heteroaryl is independently substituted with 0, 1, 2, 3, 4, or 5 R^{dd} groups;
or two geminal hydrogens on a carbon atom are replaced with the group ═O, ═S, ═NN(R^{bb})₂, ═NNR^{bb}C(═O)R^{aa}, ═NNR^{bb}C(═O)OR^{aa}, ═NNR^{bb}S(═O)₂R^{aa}, ═NR^{bb}, or ═NOR^{cc}; each instance of R^{aa} is, independently, selected from C₁₋₁₀ alkyl, C₁₋₁₀ perhaloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups; each instance of R^{bb} is, independently, selected from hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)₂R^{aa}, —P(═O)(R^{aa})₂, —P(═O)₂N(R^{cc})₂, —P(═O)(NR^{cc})₂, C₁₋₁₀ alkyl, C₁₋₁₀ perhaloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups; each instance of R^{cc} is, independently, selected from hydrogen, C₁₋₁₀ alkyl, C₁₋₁₀ perhaloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups; each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(R^{ff})₃⁺X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R^{ff})₂, —C(=NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)₂R^{ee}, —P(═O)(R^{ee})₂, —OP(═O)(R^{ee})₂, —OP(═O)(OR^{ee})₂, C₁₋₆ alkyl, C₁₋₆ perhaloalkyl, C₂₋₆ alkenyl, C₂₋₆ alkynyl, C₃₋₁₀ carbocyclyl, 3-10 membered heterocyclyl, C₆₋₁₀ aryl, 5-10 membered heteroaryl, wherein each alkyl, alkenyl, alkynyl, carbocyclyl, heterocyclyl, aryl, and heteroaryl is independently substituted with 0, 1, 2, 3, 4, or 5 R^{gg} groups, or two geminal R^{dd} substituents can be joined to form ═O or ═S; each instance of R^{ee} is, independently, selected from C₁₋₆ alkyl, C₁₋₆ perhaloalkyl, C₂₋₆ alkenyl, C₂₋₆ alkynyl, C₃₋₁₀ carbocyclyl, C₆₋₁₀ aryl, 3-10 membered heterocyclyl, and 3-10 membered heteroaryl, wherein each alkyl, alkenyl, alkynyl, carbocyclyl, heterocyclyl, aryl, and heteroaryl is independently substituted with 0, 1, 2, 3, 4, or 5 R^{gg} groups; each instance of R^{ff} is, independently, selected from hydrogen, C₁₋₆ alkyl, C₁₋₆ perhaloalkyl, C₂₋₆ alkenyl, C₂₋₆ alkynyl, C₃₋₁₀ carbocyclyl, 3-10 membered heterocyclyl, C₆₋₁₀ aryl and 5-10 membered heteroaryl, or two R^{ff} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{gg} groups; and each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₆ alkyl)₂⁺X⁻, —NH₂(C₁₋₆ alkyl) ⁺X⁻, —NH₃⁻X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂OC₁₋₆ alkyl, —OSO₂C₁₋₆ alkyl, —SOC₁₋₆ alkyl, —Si(C₁₋₆ alkyl)₃, —OSi(C₁₋₆ alkyl)₃—C(═S)N(C₁₋₆ alkyl)₂, C(═S)NH(C₁₋₆ alkyl), C(═S)NH₂, —C(═O)S(C₁₋₆ alkyl), —C(═S)SC₁₋₆ alkyl, —SC(═S)SC₁₋₆ alkyl, —P(═O)₂(C₁₋₆ alkyl), —P(═O)(C₁₋₆ alkyl)₂, —OP(═O)(C₁₋₆ alkyl)₂, —OP(═O)(OC₁₋₆ alkyl)₂, C₁₋₆ alkyl, C₁₋₆ perhaloalkyl, C₂₋₆ alkenyl, C₂₋₆ alkynyl, C₃₋₁₀ carbocyclyl, C₆₋₁₀ aryl, 3-10 membered heterocyclyl, 5-10 membered heteroaryl; or two geminal Rgg substituents can be joined to form ═O or ═S; wherein X⁻ is a counterion.

A “counterion” or “anionic counterion” is a negatively charged group associated with a cationic quaternary amino group in order to maintain electronic neutrality. Exemplary counterions include halide ions (e.g., F⁻, Cl⁻, Br⁻, I⁻), NO₃⁻, ClO₄⁻, OH⁻, H₂PO₄⁻, HSO₄⁻, sulfonate ions (e.g., methansulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), and carboxylate ions (e.g., acetate, ethanoate, propanoate, benzoate, glycerate, lactate, tartrate, glycolate, and the like).

Nitrogen atoms can be substituted or unsubstituted as valency permits, and include primary, secondary, tertiary, and quarternary nitrogen atoms. Exemplary nitrogen atom substitutents include, but are not limited to, hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{bb})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)₂R^{aa}, —P(═O)(R^{aa})₂, —P(═O)₂N(R^{cc})₂, —P(═O)(NR^{cc})₂, C₁₋₁₀ alkyl, C₁₋₁₀ perhaloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, or two R^{cc} groups attached to a nitrogen atom are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups, and wherein R^{aa}, R^{bb}, R^{cc} and R^{dd} are as defined above.

In certain embodiments, the substituent present on a nitrogen atom is a nitrogen protecting group (also referred to as an amino protecting group). Nitrogen protecting groups include, but are not limited to, —OH, —OR^{aa}, —N(R^{cc})₂, —C(═O)R^{aa}, —C(═O)N(R^{cc})₂, —CO₂R^{aa}, —SO₂R^{aa}, —C(═NR^{cc})R^{aa}, —C(=NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, C₁₋₁₀ alkyl (e.g., aralkyl, heteroaralkyl), C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl groups, wherein each alkyl, alkenyl, alkynyl, carbocyclyl, heterocyclyl, aralkyl, aryl, and heteroaryl is independently substituted with 0, 1, 2, 3, 4, or 5 R^{dd} groups, and wherein R^{aa}, R^{bb}, R^{cc} and R^{dd} are as defined herein. Nitrogen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

For example, nitrogen protecting groups such as amide groups (e.g., —C(═O)R^{aa}) include, but are not limited to, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitophenylacetamide, o-nitrophenoxyacetamide, acetoacetamide, (N′-dithiobenzyloxyacylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivative, o-nitrobenzamide and o-(benzoyloxymethyl)benzamide.

Nitrogen protecting groups such as carbamate groups (e.g., —C(═O)OR^{aa}) include, but are not limited to, methyl carbamate, ethyl carbamante, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, and 2,4,6-trimethylbenzyl carbamate.

Nitrogen protecting groups such as sulfonamide groups (e.g., —S(═O)₂R^{aa}) include, but are not limited to, p-toluenesulfonamide (Ts), benzenesulfonamide, 2,3,6,-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), 0-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

Other nitrogen protecting groups include, but are not limited to, phenothiazinyl-(10)-acyl derivative, N′-p-toluenesulfonylaminoacyl derivative, N′-phenylaminothioacyl derivative, N-benzoylphenylalanyl derivative, N-acetylmethionine derivative, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N-(N′,N′-dimethylaminomethylene)amine, N,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentaacylchromium- or tungsten)acyl]amine, copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, and 3-nitropyridinesulfenamide (Npys).

In certain embodiments, the substituent present on an oxygen atom is an oxygen protecting group (also referred to as a hydroxyl protecting group). Oxygen protecting groups include, but are not limited to, —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃, —P(═O)₂R^{aa}, —P(═O)(R^{aa})₂, —P(═O)(OR^{cc})₂, —P(═O)₂N(R^{bb})₂, and —P(═O)(NR^{bb})₂, wherein R^{aa}, R^{bb}, and R^{cc} are as defined herein. Oxygen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

Exemplary oxygen protecting groups include, but are not limited to, methyl, methoxylmethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl (Bn), p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, a-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyOmethyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl)xanthenyl, 9(9-phenyl-10-oxo)anthryl, 1,3-benzodisulfuran-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), alkyl methyl carbonate, 9-fluorenylmethyl carbonate (Fmoc), alkyl ethyl carbonate, alkyl 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), alkyl isobutyl carbonate, alkyl vinyl carbonate alkyl allyl carbonate, alkyl p-nitrophenyl carbonate, alkyl benzyl carbonate, alkyl p-methoxybenzyl carbonate, alkyl 3,4-dimethoxybenzyl carbonate, alkyl o-nitrobenzyl carbonate, alkyl p-nitrobenzyl carbonate, alkyl S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxyacyl)benzoate, a-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts).

In certain embodiments, the substituent present on an sulfur atom is an sulfur protecting group (also referred to as a thiol protecting group). Sulfur protecting groups include, but are not limited to, —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃, —P(═O)₂R^{aa}), —P(═O)(R^{aa})₂, —P(═O)(OR^{cc})₂, —P(═O)₂N(R^{bb})₂, and —P(═O)(NR^{bb})₂, wherein R^{aa}, R^{bb}, and R^{cc} are as defined herein. Sulfur protecting groups are well known in the art and include those described in detail in Protecting Groups in Organic Synthesis, T. W. Greene and P. G. M. Wuts, 3r^{d} edition, John Wiley & Sons, 1999, incorporated herein by reference.

“Compounds of the present invention”, and equivalent expressions, are meant to embrace the compounds as hereinbefore described, in particular compounds according to any of the Formula herein recited and/or described, which expression includes the prodrugs, the pharmaceutically acceptable salts, and the solvates, e.g., hydrates, where the context so permits. Similarly, reference to intermediates, whether or not they themselves are claimed, is meant to embrace their salts, and solvates, where the context so permits.

These and other exemplary substituents are described in more detail in the Detailed Description, Examples, and claims. The invention is not intended to be limited in any manner by the above exemplary listing of substituents.

### Other Definitions

“Pharmaceutically acceptable” means approved or approvable by a regulatory agency of the Federal or a state government or the corresponding agency in countries other than the United States, or that is listed in the U.S. Pharmacopoeia or other generally recognized pharmacopoeia for use in animals, and more particularly, in humans.

“Pharmaceutically acceptable salt” refers to a salt of a compound of the invention that is pharmaceutically acceptable and that possesses the desired pharmacological activity of the parent compound. In particular, such salts are non-toxic may be inorganic or organic acid addition salts and base addition salts. Specifically, such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, N-methylglucamine and the like. Salts further include, by way of example only, sodium, potassium, calcium, magnesium, ammonium, tetraalkylammonium, and the like; and when the compound contains a basic functionality, salts of non toxic organic or inorganic acids, such as hydrochloride, hydrobromide, tartrate, mesylate, acetate, maleate, oxalate and the like. The term “pharmaceutically acceptable cation” refers to an acceptable cationic counter-ion of an acidic functional group. Such cations are exemplified by sodium, potassium, calcium, magnesium, ammonium, tetraalkylammonium cations, and the like (see, e.g., Berge, et al., J. Pharm. Sci. 66(1): 1-79 (Jan. ''77) .

“Pharmaceutically acceptable vehicle” refers to a diluent, adjuvant, excipient or carrier with which a compound of the invention is administered.

“Pharmaceutically acceptable metabolically cleavable group” refers to a group which is cleaved in vivo to yield the parent molecule of the structural Formula indicated herein. Examples of metabolically cleavable groups include —COR, —COOR, —CONRR and —CH₂OR radicals, where R is selected independently at each occurrence from alkyl, trialkylsilyl, carbocyclic aryl or carbocyclic aryl substituted with one or more of alkyl, halogen, hydroxy or alkoxy. Specific examples of representative metabolically cleavable groups include acetyl, methoxycarbonyl, benzoyl, methoxymethyl and trimethylsilyl groups.

“Prodrugs” refers to compounds, including derivatives of the compounds of the invention,which have cleavable groups and become by solvolysis or under physiological conditions the compounds of the invention that are pharmaceutically active in vivo. Such examples include, but are not limited to, choline ester derivatives and the like, N-alkylmorpholine esters and the like. Other derivatives of the compounds of this invention have activity in both their acid and acid derivative forms, but in the acid sensitive form often offers advantages of solubility, tissue compatibility, or delayed release in the mammalian organism (see, Bundgard, H., Design of Prodrugs, pp. 7-9, 21-24, Elsevier, Amsterdam 1985). Prodrugs include acid derivatives well know to practitioners of the art, such as, for example, esters prepared by reaction of the parent acid with a suitable alcohol, or amides prepared by reaction of the parent acid compound with a substituted or unsubstituted amine, or acid anhydrides, or mixed anhydrides. Simple aliphatic or aromatic esters, amides and anhydrides derived from acidic groups pendant on the compounds of this invention are particular prodrugs. In some cases it is desirable to prepare double ester type prodrugs such as (acyloxy)alkyl esters or ((alkoxycarbonyl)oxy)alkylesters. Particularly the C₁ to C₈ alkyl, C₂-C₈ alkenyl, C₂-C₈ alkynyl, aryl, C₇-C₁₂ substituted aryl, and C₇-C₁₂ arylalkyl esters of the compounds of the invention.

“Solvate” refers to forms of the compound that are associated with a solvent or water (also referred to as “hydrate”), usually by a solvolysis reaction. This physical association includes hydrogen bonding. Conventional solvents include water, ethanol, acetic acid and the like. The compounds of the invention may be prepared e.g. in crystalline form and may be solvated or hydrated. Suitable solvates include pharmaceutically acceptable solvates, such as hydrates, and further include both stoichiometric solvates and non-stoichiometric solvates. In certain instances the solvate will be capable of isolation, for example when one or more solvent molecules are incorporated in the crystal lattice of the crystalline solid. “Solvate” encompasses both solution-phase and isolable solvates. Representative solvates include hydrates, ethanolates and methanolates.

A “subject” to which administration is contemplated includes, but is not limited to, humans (i.e., a male or female of any age group, e.g., a pediatric subject (e.g, infant, child, adolescent) or adult subject (e.g., young adult, middle-aged adult or senior adult)) and/or a non-human animal, e.g., a mammal such as primates (e.g., cynomolgus monkeys, rhesus monkeys), cattle, pigs, horses, sheep, goats, rodents, cats, and/or dogs. In certain embodiments, the subject is a human. In certain embodiments, the subject is a non-human animal. The terms “human”, “patient” and “subject” are used interchangeably herein.

“Therapeutically effective amount” means the amount of a compound that, when administered to a subject for treating a disease, is sufficient to effect such treatment for the disease. The “therapeutically effective amount” can vary depending on the compound, the disease and its severity, and the age, weight, etc., of the subject to be treated.

“Preventing” or “prevention” refers to a reduction in risk of acquiring or developing a disease or disorder (i.e., causing at least one of the clinical symptoms of the disease not to develop in a subject not yet exposed to a disease-causing agent, or predisposed to the disease in advance of disease onset.

The term “prophylaxis” is related to “prevention”, and refers to a measure or procedure the purpose of which is to prevent, rather than to treat or cure a disease. Non-limiting examples of prophylactic measures may include the administration of vaccines; the administration of low molecular weight heparin to hospital patients at risk for thrombosis due, for example, to immobilization; and the administration of an anti-malarial agent such as chloroquine, in advance of a visit to a geographical region where malaria is endemic or the risk of contracting malaria is high.

“Treating” or “treatment” of any disease or disorder refers, in certain embodiments, to ameliorating the disease or disorder (i.e., arresting the disease or reducing the manifestation, extent or severity of at least one of the clinical symptoms thereof). In another embodiment “treating” or “treatment” refers to ameliorating at least one physical parameter, which may not be discernible by the subject. In yet another embodiment, “treating” or “treatment” refers to modulating the disease or disorder, either physically, (e.g., stabilization of a discernible symptom), physiologically, (e.g., stabilization of a physical parameter), or both. In a further embodiment, “treating” or “treatment” relates to slowing the progression of the disease.

As used herein, the term “isotopic variant” refers to a compound that contains unnatural proportions of isotopes at one or more of the atoms that constitute such compound. For example, an “isotopic variant” of a compound can contain one or more non-radioactive isotopes, such as for example, deuterium (²H or D), carbon-13 (¹³C), nitrogen-15 (¹⁵N), or the like. It will be understood that, in a compound where such isotopic substitution is made, the following atoms, where present, may vary, so that for example, any hydrogen may be ²H/D, any carbon may be ¹³C, or any nitrogen may be ¹⁵N, and that the presence and placement of such atoms may be determined within the skill of the art. Likewise, the invention may include the preparation of isotopic variants with radioisotopes, in the instance for example, where the resulting compounds may be used for drug and/or substrate tissue distribution studies. The radioactive isotopes tritium, i.e., ³H, and carbon-14, i.e., ¹⁴C, are particularly useful for this purpose in view of their ease of incorporation and ready means of detection. Further, compounds may be prepared that are substituted with positron emitting isotopes, such as ¹¹C, ¹⁸F, ¹⁵O and ¹³N, and would be useful in Positron Emission Topography (PET) studies for examining substrate receptor occupancy. All isotopic variants of the compounds provided herein, radioactive or not, are intended to be encompassed within the scope of the invention.

It is also to be understood that compounds that have the same molecular formula but differ in the nature or sequence of bonding of their atoms or the arrangement of their atoms in space are termed “isomers”. Isomers that differ in the arrangement of their atoms in space are termed “stereoisomers”.

Stereoisomers that are not mirror images of one another are termed “diastereomers” and those that are non-superimposable mirror images of each other are termed “enantiomers”. When a compound has an asymmetric center, for example, when it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

“Tautomers” refer to compounds that are interchangeable forms of a particular compound structure, and that vary in the displacement of hydrogen atoms and electrons. Thus, two structures may be in equilibrium through the movement of π electrons and an atom (usually H). For example, enols and ketones are tautomers because they are rapidly interconverted by treatment with either acid or base. Another example of tautomerism is the aci- and nitro-forms of phenylnitromethane, which are likewise formed by treatment with acid or base. Tautomeric forms may be relevant to the attainment of the optimal chemical reactivity and biological activity of a compound of interest.

As used herein a pure enantiomeric compound is substantially free from other enantiomers or stereoisomers of the compound (i.e., in enantiomeric excess). In other words, an “S” form of the compound is substantially free from the “R” form of the compound and is, thus, in enantiomeric excess of the “R” form. The term “enantiomerically pure” or “pure enantiomer” denotes that the compound comprises more than 75% by weight, more than 80% by weight, more than 85% by weight, more than 90% by weight, more than 91% by weight, more than 92% by weight, more than 93% by weight, more than 94% by weight, more than 95% by weight, more than 96% by weight, more than 97% by weight, more than 98% by weight, more than 98.5% by weight, more than 99% by weight, more than 99.2% by weight, more than 99.5% by weight, more than 99.6% by weight, more than 99.7% by weight, more than 99.8% by weight or more than 99.9% by weight, of the enantiomer. In certain embodiments, the weights are based upon total weight of all enantiomers or stereoisomers of the compound.

As used herein and unless otherwise indicated, the term “enantiomerically pure R-compound” refers to at least about 80% by weight R-compound and at most about 20% by weight S-compound, at least about 90% by weight R-compound and at most about 10% by weight S-compound, at least about 95% by weight R-compound and at most about 5% by weight S-compound, at least about 99% by weight R-compound and at most about 1% by weight S-compound, at least about 99.9% by weight R-compound or at most about 0.1% by weight S-compound. In certain embodiments, the weights are based upon total weight of compound.

As used herein and unless otherwise indicated, the term “enantiomerically pure 5-compound” or “S-compound” refers to at least about 80% by weight S-compound and at most about 20% by weight R-compound, at least about 90% by weight S-compound and at most about 10% by weight R-compound, at least about 95% by weight S-compound and at most about 5% by weight R-compound, at least about 99% by weight S-compound and at most about 1% by weight R-compound or at least about 99.9% by weight S-compound and at most about 0.1% by weight R-compound. In certain embodiments, the weights are based upon total weight of compound.

In the compositions provided herein, an enantiomerically pure compound or a pharmaceutically acceptable salt, solvate, hydrate or prodrug thereof can be present with other active or inactive ingredients. For example, a pharmaceutical composition comprising enantiomerically pure R-compound can comprise, for example, about 90% excipient and about 10% enantiomerically pure R-compound. In certain embodiments, the enantiomerically pure R-compound in such compositions can, for example, comprise, at least about 95% by weight R-compound and at most about 5% by weight S-compound, by total weight of the compound. For example, a pharmaceutical composition comprising enantiomerically pure S-compound can comprise, for example, about 90% excipient and about 10% enantiomerically pure S-compound. In certain embodiments, the enantiomerically pure S-compound in such compositions can, for example, comprise, at least about 95% by weight S-compound and at most about 5% by weight R-compound, by total weight of the compound. In certain embodiments, the active ingredient can be formulated with little or no excipient or carrier.

The compounds of this invention may possess one or more asymmetric centers; such compounds can therefore be produced as individual (R)- or (S)-stereoisomers or as mixtures thereof

Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art.

One having ordinary skill in the art of organic synthesis will recognize that the maximum number of heteroatoms in a stable, chemically feasible heterocyclic ring, whether it is aromatic or non aromatic, is determined by the size of the ring, the degree of unsaturation and the valence of the heteroatoms. In general, a heterocyclic ring may have one to four heteroatoms so long as the heteroaromatic ring is chemically feasible and stable.

### DETAILED DESCRIPTION OF THE PREFERRED EMBODIMENTS

In certain aspects, provided herein are pharmaceutical compositions comprising of a bolaamp uphile complex.

In further aspects, provided herein are novel nano-sized vesicles comprising of bolaamphiphilic compounds.

In further aspects, provided herein are novel nano-sized vesicles comprising of bolaamphiphilic compounds which are capable of encapsulating NTF, GDNF or NGF.

In still another aspect the vesicles formed from the bolaamphiphiles to encapsulating NTF, GDNF or NGF, contain additives that help to stabilize the vesicles, by stabilizing the vesicle's membranes, such as but not limited to cholesterol derivatives such as cholesterol hemisuccinate and cholesterol itself and combinations such as cholesterol hemisuccinate and cholesterol.

In still another embodiments the vesicles in addition to these components have another addiitves which decorates the outer vesicle memrbanes with groups or pendants that enhance penetration though biological barriers such as the BBB, or groups for targeting to specific sites such as dopaminergic neurons.

In a further embodiment the bolaamphiphile head groups that comprise the vesicles membranes can interact with the neuro active agents such as GDNF or NDF to be delivered in to the brain and brain sites ionic interactions to enhance the % encapsulation via complexation and well as passive encapsulation within the vesicles core. Further the formuation may contain other additives within the veicles membranes to futhjer enhance the degree of encapsulation of neuro active agents such as GDNF or NDF. It is understood by one skilled in the state of art that the pH in which the vesicle formation and encapsulation of the neuro active agent such as GDNF or NDF is such as to maximize the electrostatic or inonic interactions between the said agents and the sai bolaamnphiphiles and or additives to maximize the % encapsulation.

In further aspects, provided herein are novel nano-sized bola vesicles described above that encapsulate GDNF or NGF and are capable of delivering the encapsulated material into the brain.

In further aspects, provided herein are novel nano-sized bola vesicles that encapsulate GDNF or NGF and are capable of delivering, the encapsulated material to the brain, specifically to dopaminergic neurons.

In further aspects, provided herein are novel nano-sized bola vesicles that encapsulate GDNF or NGF and are capable of delivering, the encapsulated material into brain regions affected in neurological disorders. In one particular embodiment, the neurological disorder is Parkinson's disease (PD) or Alzheimer's disease (AD).

in certain aspects, provided herein are novel bolaamphiphile complexes comprising bolaamphiphilic compounds and a compound active against PD. In one embodiment, the compound active against PD is GDNF.

In certain aspects, provided herein are novel bolaamphiphile complexes comprising bolaamphiphilic compounds and a compound active against AD. In one embodiment, the compound active against AD is NGF.

In further aspects, provided herein are novel formulations of GDNF or NGF with bolaamphiphilic compounds or with bolaamhphile vesicles.

In another aspect, provided here are methods of delivering GDNF or NGF agents into animal or human brain. In one embodiment, the method comprises the step of administering to the animal or human a pharmaceutical composition comprising of a bolaamphiphile complex; and wherein the bolaamphiphile complex comprises a bolaamphiphilic compound and GDNF. In another embodiment, the complex comprises bolaamphiphilic compound and NGF.

In one embodiment, the bolaamphiphilic compound consists of two hydrophilic headgroups linked through a long hydrophobic chain. In another embodiment, the hydrophilic headgroup is an amino containing group. In a specific embodiment, the hydrophilic headgroup is a tertiary or quaternary amino containing group.

In one particular embodiment, the bolaamphiphilic compound is a compound according to formula I:

HG²-L¹-HG¹ I

or a pharmaceutically acceptable salt, solvate, hydrate, prodrug, stereoisomer, tautomer, isotopic variant, or N-oxide thereof, or a combination thereof; wherein:

each HG¹ and HG² is independently a hydrophilic head group; and

L¹ is alkylene, alkenyl, heteroalkylene, or heteroalkenyl linker; unsubstituted or substituted with C₁-C₂₀ alkyl, hydroxyl, or oxo.

In one embodiment, the pharmaceutically acceptable salt is a quaternary ammonium salt.

In one embodiment, with respect to the bolaamphiphilic compound of formula I, L¹ is heteroalkylene, or heteroalkenyl linker comprising C, N, and O atoms; unsubstituted or substituted with C₁-C₂₀ alkyl, hydroxyl, or oxo.

In another embodiment, with respect to the bolaamphiphilic compound of formula I, L¹ is

—O-L²—C(O)—O—(CH₂)ₙ₄—O—C(O)—L³-O—, or

—O-L²—C(O)—O—(CH₂)ₙ₅—O—C(O)—(CH₂)ₙ₆—,
and wherein each L² and L³ is C₄-C₂o alkenyl linker; unsubstituted or substituted with C₁-C₈ alkyl or hydroxy;
and n4, n5, and n6 is independently an integer from 4-20.

In one embodiment, each L² and L³ is independently —C(R¹)—C(OH)—CH₂—(CH═CH)—(CH₂)ₙ₇—; R¹ is C₁-C₈ alkyl, and n7 is independently an integer from 4-20.

In another embodiment, with respect to the bolaamphiphilic compound of formula I, L¹ is —O—(CH₂)ₙ₁—O—C(O)—(CH₂)ₙ₂—C(O)—O—(CH₂)ₙ₃—O—.

In another embodiment, with respect to the bolaamphiphilic compound of formula I, L¹ is

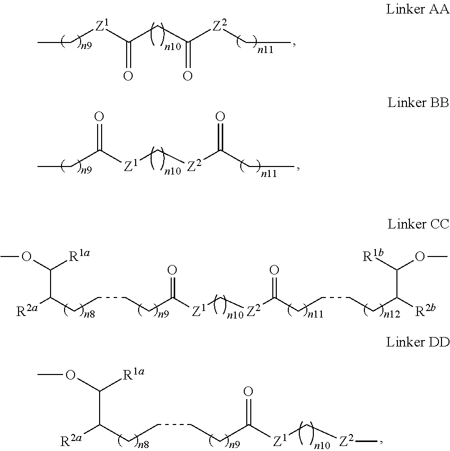

wherein:
each Z¹ and Z² is independently —C(R³)₂—, —N(R³)— or —O—;
each R^{1a}, R^{1b}, R³, and R⁴ is independently H or C₁-C₈ alkyl;
each R^{2a} and R^{2b} is independently H , C₁-C₈ alkyl, OH, or alkoxy;
each n8, n9, n11, and n12 is independently an integer from 1-20;
n10 is an integer from 2-20; and
each dotted bond is independently a single or a double bond.
and wherein each methylene carbon is unsubstituted or substituted with C₁-C₄ alkyl; and each n1, n2, and n3 is independently an integer from 4-20.

In one embodiment, with respect to the bolaamphiphilic compound of formula I, the bolaamphiphilic compound is a compound according to formula II, III, IV, V, or VI:

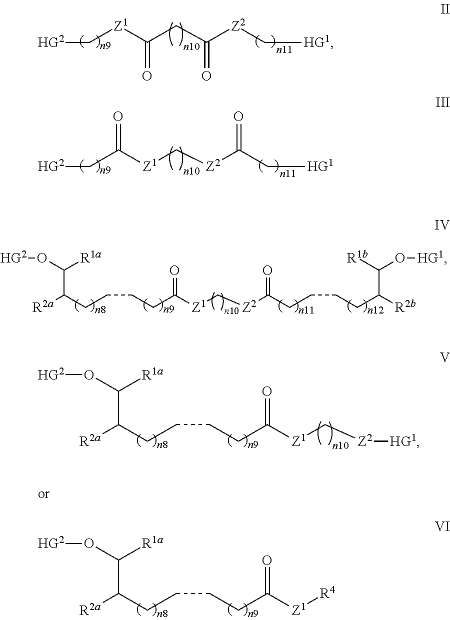

or a pharmaceutically acceptable salt, solvate, hydrate, prodrug, stereoisomer, tautomer, isotopic variant, or N-oxide thereof, or a combination thereof; wherein:

each HG¹ and HG² is independently a hydrophilic head group;

each Z¹ and Z² is independently —C(R³)₂—, —N(R³)— or —O—;

each R^{1a}, R³, and R⁴ is independently H or C₁-C₈ alkyl;

each R^{2a} and R^{2b} is independently H , C₁-C₈ alkyl, OH, alkoxy, or O-HG¹ or O-HG²;

each n8, n9, n11, and n12 is independently an integer from 1-20;

n10 is an integer from 2-20; and

each dotted bond is independently a single or a double bond.

In one embodiment, with respect to the bolaamphiphilic compound of formula II, III, IV, V, or VI, each n9 and n11 is independently an integer from 2-12. In another embodiment, n9 and n11 is independently an integer from 4-8. In a particular embodiment, each n9 and n11 is 7 or 11.

In one embodiment, with respect to the bolaamphiphilic compound of formula II, III, IV, V, or VI, each n8 and n12 is independently 1, 2, 3, or 4. In a particular embodiment, each n8 and n12 is 1.

In one embodiment, with respect to the bolaamphiphilic compound of formula II, III, IV, V, or VI, each R^{2a} and R^{2b} is independently H, OH, or alkoxy. In another embodiment, each R^{2a} and R^{2b} is independently H, OH, or OMe. In another embodiment, each R^{2a} and R^{2b} is independently-O-HG¹ or O-HG². In a particular embodiment, each R^{2a} and R^{2b} is OH.

In one embodiment, with respect to the bolaamphiphilic compound of formula II, III, IV, V, or VI, each R^{1a} and R^{1b} is independently H, Me, Et, n-Pr, i-Pr, n-Bu, i-Bu, sec-Bu, n-pentyl, isopentyl, n-hexyl, n-heptyl, or n-octyl. In a particular embodiment, each R^{1a} and R^{1b} is independently n-pentyl.

In one embodiment, with respect to the bolaamphiphilic compound of formula II, III, IV, V, or VI, each dotted bond is a single bond. In another embodiment, each dotted bond is a double bond.

In one embodiment, with respect to the bolaamphiphilic compound of formula II, III, IV, V, or VI, n10 is an integer from 2-16. In another embodiment, n10 is an integer from 2-12. In a particular embodiment, n10 is 2, 4, 6, 8, 10, 12, or 16.

In one embodiment, with respect to the bolaamphiphilic compound of formula IV, R⁴ is H, Me, Et, n-Pr, i-Pr, n-Bu, i-Bu, sec-Bu, n-pentyl, or isopentyl. In another embodiment, R⁴ is Me, or Et. In a particular embodiment, R⁴ is Me.

In one embodiment, with respect to the bolaamphiphilic compound of formula II, III, IV, V, or VI, each Z¹ and Z² is independently C(R³)₂—, or —N(R³)—. In another embodiment, each Z¹ and Z² is independently C(R³)₂—, or —N(R³)—; and each R³ is independently H, Me, Et, n-Pr, Pr, n-Bu, i-Bu, sec-Bu, n-pentyl, or isopentyl. In a particular embodiment, R³ is H.

In one embodiment, with respect to the bolaamphiphilic compound of formula II, III, IV, V, or VI, each Z¹ and Z² is —O—.

In one embodiment, with respect to the bolaamphiphilic compound of formula I, II, III, or IV, each HG¹ and HG² is independently selected from:

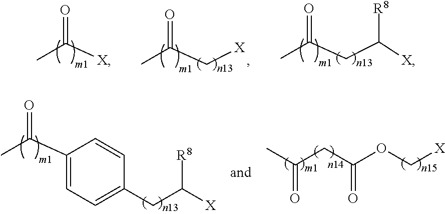

wherein:
X is —NR^{5a}R^{5b}, or —N⁺R^{5a}R^{5b}R^{5c}; each R^{5a}, and R^{5b} is independently H or substituted or unsubstituted C₁-C₂₀ alkyl or R^{5a} and R^{5b} may join together to form an N containing substituted or unsubstituted heteroaryl, or substituted or unsubstituted heterocyclyl; each R^{5c} is independently substituted or unsubstituted C₁-C₂₀ alkyl; each R⁸ is independently H, substituted or unsubstituted C₁-C₂₀ alkyl, alkoxy, or carboxy;
m1 is 0 or 1; and
each n13, n14, and n15 is independently an integer from 1-20.

In one embodiment, with respect to the bolaamphiphilic compound of formula I, II, III, or IV, HG¹ and HG² are as defined above, and each m1 is 0.

In one embodiment, with respect to the bolaamphiphilic compound of formula I, II, III, or IV, HG¹ and HG² are as defined above, and each m1 is 1.

In one embodiment, with respect to the bolaamphiphilic compound of formula I, II, III, or IV, HG¹ and HG² are as defined above, and each n13 is 1 or 2.

In one embodiment, with respect to the bolaamphiphilic compound of formula I, II, III, or IV, HG¹ and HG² are as defined above, and each n14 and n15 is independently 1, 2, 3, 4, or 5. In another embodiment, each n14 and n15 is independently 2 or 3.

In one particular embodiment, the bolaamphiphilic compound is a compound according to formula VIIa, VIIb, VIIc, or VIId:

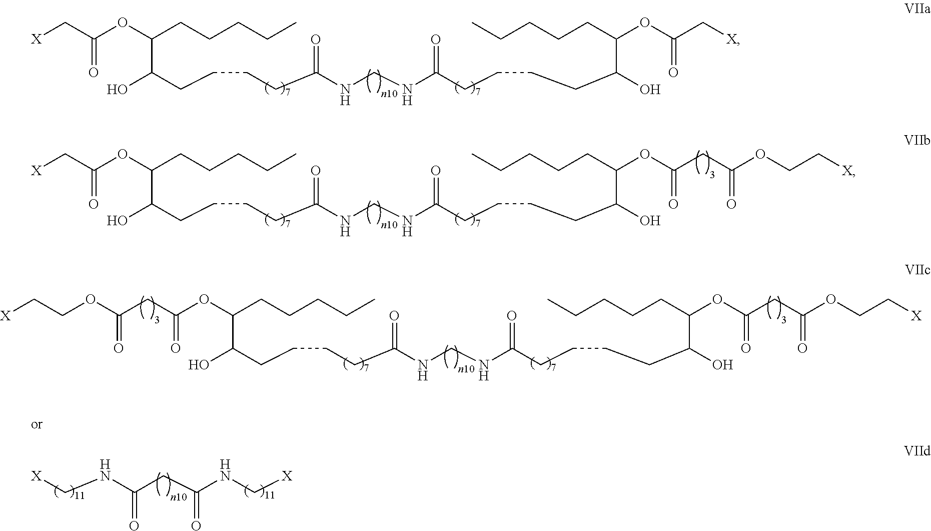

or a pharmaceutically acceptable salt, solvate, hydrate, prodrug, stereoisomer, tautomer, isotopic variant, or N-oxide thereof, or a combination thereof;
wherein:
   each X is —NR^{5a}R^{5b}, or —N⁺R^{5a}R^{5b}R^{5c}; each R^{5a}, and R^{5b} is independently H or substituted or unsubstituted C₁-C₂₀ alkyl or R^{5a} and R^{5b} may join together to form an N containing substituted or unsubstituted heteroaryl, or substituted or unsubstituted heterocyclyl;
   each R^{5c} is independently substituted or unsubstituted C₁-C₂₀ alkyl;
   n10 is an integer from 2-20; and
   each dotted bond is independently a single or a double bond.

In another particular embodiment, the bolaamphiphilic compound is a compound according to formula VIIIa, VIIIb, VIIIc, or VIIId:

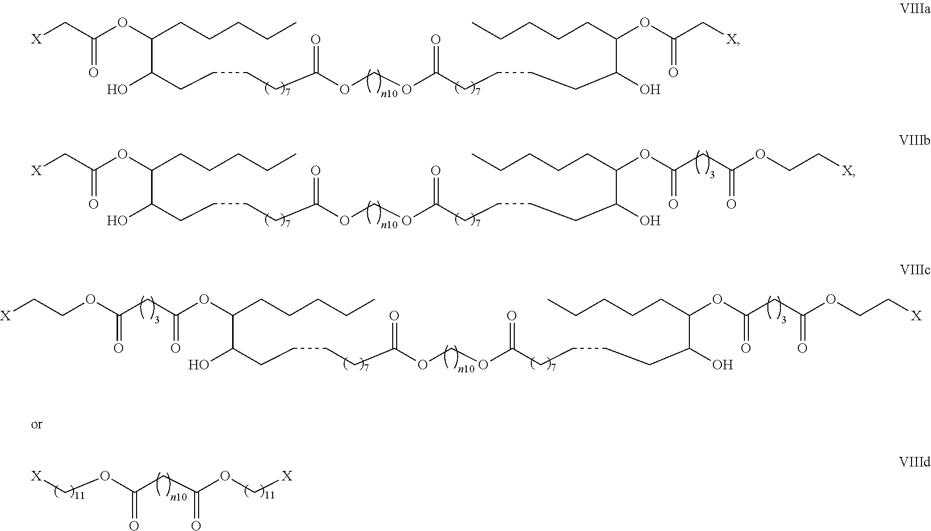

or a pharmaceutically acceptable salt, solvate, hydrate, prodrug, stereoisomer, tautomer, isotopic variant, or N-oxide thereof, or a combination thereof;
wherein:
   each X is —NR^{5a}R^{5b}, or —N⁺R^{5a}R^{5b}R^{5c}; each R^{5a}, and R^{5b} is independently H or substituted or unsubstituted C₁-C₂₀ alkyl or R^{5a} and R^{5b} may join together to form an N containing substituted or unsubstituted heteroaryl, or substituted or unsubstituted heterocyclyl;
      each R^{5c} is independently substituted or unsubstituted C₁-C₂₀ alkyl;;
      n10 is an integer from 2-20; and
      each dotted bond is independently a single or a double bond.

In another particular embodiment, the bolaamphiphilic compound is a compound according to formula IXa, IXb, or IXc:

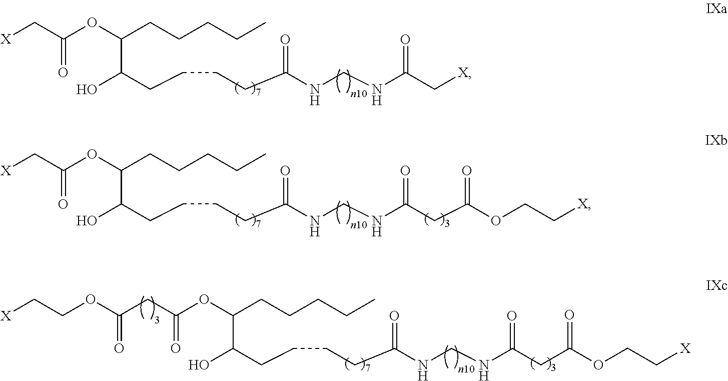

or a pharmaceutically acceptable salt, solvate, hydrate, prodrug, stereoisomer, tautomer, isotopic variant, or N-oxide thereof, or a combination thereof;
wherein:
   each X is —NR^{5a}R^{5b}, or —N⁺R^{5a}R^{5b}R^{5c}; each R^{5a}, and R^{5b} is independently H or substituted or unsubstituted C₁-C₂₀ alkyl or R^{5a} and R^{5b} may join together to form an N containing substituted or unsubstituted heteroaryl, or substituted or unsubstituted heterocyclyl;
      each R^{5c} is independently substituted or unsubstituted C₁-C₂₀ alkyl;;
      n10 is an integer from 2-20; and
      each dotted bond is independently a single or a double bond.

In another particular embodiment, the bolaamphiphilic compound is a compound according to formula Xa, Xb, or Xc:

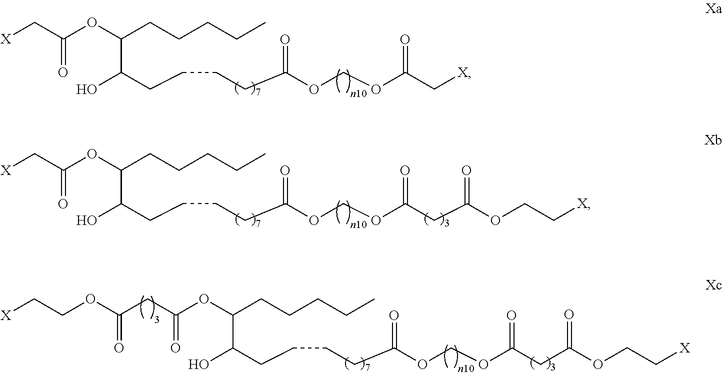

or a pharmaceutically acceptable salt, solvate, hydrate, prodrug, stereoisomer, tautomer, isotopic variant, or N-oxide thereof, or a combination thereof;
wherein:
   each X is —NR^{5a}R^{5b}, or —N⁺R^{5a}R^{5b}R^{5c}; each R^{5a}, and R^{5b} is independently H or substituted or unsubstituted C₁-C₂₀ alkyl or R^{5a} and R^{5b} may join together to form an N containing substituted or unsubstituted heteroaryl, or substituted or unsubstituted heterocyclyl;
      each R⁵′ is independently substituted or unsubstituted C₁-C₂₀ alkyl;;
      n10 is an integer from 2-20; and
      each dotted bond is independently a single or a double bond.

In one embodiment, with respect to the bolaamphiphilic compound of formula VIIa-VIId, VIIIa-VIIId, IXa-IXc, or Xa-Xc, each dotted bond is a single bond. In another embodiment, each dotted bond is a double bond.

In one embodiment, with respect to the bolaamphiphilic compound of formula VIIa-VIId, VIIIa-VIIId, IXa-IXc, or Xa-Xc, n10 is an integer from 2-16.

In one embodiment, with respect to the bolaamphiphilic compound of formula VIIa-VIId, VIIIa-VIIId, IXa-IXc, or Xa-Xc, n10 is an integer from 2-12.

In one embodiment, with respect to the bolaamphiphilic compound of formula VIIa-VIId, VIIIa-VIIId, IXa-IXc, or Xa-Xc, n10 is 2, 4, 6, 8, 10, 12, or 16.

In one embodiment, with respect to the bolaamphiphilic compound of formula VIIa-VIId, VIIIa-VIIId, IXa-IXc, or Xa-Xc, each R^{5a}, R^{5b}, and R^{5c} is independently substituted or unsubstituted C₁-C₂₀ alkyl.

In one embodiment, with respect to the bolaamphiphilic compound of formula VIIa-VIId, VIIIa-VIIId, IXa-IXc, or Xa-Xc, each R^{5a}, R^{5b}, and R^{5c} is independently unsubstituted C₁-C₂₀ alkyl.

In one embodiment, with respect to the bolaamphiphilic compound of formula VIIa-VIId, VIIIa-VIIId, IXa-IXc, or Xa-Xc, one of R^{5a}, R^{5b}, and R^{5c} is C₁-C₂₀ alkyl substituted with —OC(O)R⁶; and R⁶ is C₁-C₂₀ alkyl.

In one embodiment, with respect to the bolaamphiphilic compound of formula VIIa-VIId, VIIIa-VIIId, IXa-IXc, or Xa-Xc, two of R^{5a}, R^{5b}, and R^{5c} are independently C₁-C₂₀ alkyl substituted with —OC(O)R⁶; and R⁶ is C₁-C₂₀ alkyl. In one embodiment, R⁶ is Me, Et, n-Pr, i-Pr, n-Bu, i-Bu, sec-Bu, n-pentyl, isopentyl, n-hexyl, n-heptyl, or n-octyl. In a particular embodiment, R⁶ is Me.

In one embodiment, with respect to the bolaamphiphilic compound of formula VIIa-VIId, VIIIa-VIIId, IXa-IXc, or Xa-Xc, one of R^{5a}, R^{5b}, and R^{5c} is C₁-C₂o alkyl substituted with amino, alkylamino or dialkylamino.

In one embodiment, with respect to the bolaamphiphilic compound of formula VIIa-VIId, VIIIa-VIIId, IXa-IXc, or Xa-Xc, two of R^{5a}, R^{5b}, and R^{5c} are independently C₁-C₂₀ alkyl substituted with amino, alkylamino or dialkylamino.

In one embodiment, with respect to the bolaamphiphilic compound of formula VIIa-VIId, VIIIa-VIIId, IXa-IXc, or Xa-Xc, R^{5a}, and R^{5b} together with the N they are attached to form substituted or unsubstituted heteroaryl.

In one embodiment, with respect to the bolaamphiphilic compound of formula VIIa-VIId, VIIIa-VIIId, IXa-IXc, or Xa-Xc, R^{5a}, and R^{5b} together with the N they are attached to form substituted or unsubstituted pyridyl.

In one embodiment, with respect to the bolaamphiphilic compound of formula VIIa-VIId, VIIIa-VIIId, IXa-IXc, or Xa-Xc, R^{5a}, and R^{5b} together with the N they are attached to form substituted or unsubstituted monocyclic or bicyclic heterocyclyl.

In one embodiment, with respect to the bolaamphiphilic compound of formula VIIa-VIId, VIIIa-VIIId, IXa-IXc, or Xa-Xc, X is substituted or unsubstituted

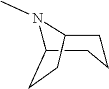

In one embodiment, with respect to the bolaamphiphilic compound of formula VIIa-VIId, VIIIa-VIIId, IXa-IXc, or Xa-Xc, X is

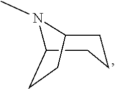

substituted with one or more groups selected from alkoxy, acetyl, and substituted or unsubstituted Ph.

In one embodiment, with respect to the bolaamphiphilic compound of formula VIIa-VIId, VIIIa-VIIId, IXa-IXc, or Xa-Xc, X is

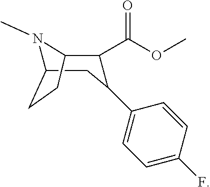 [SMILES: COC(=O)C1C(c2ccc(F)cc2)CC2CCC1N2C]

In one embodiment, with respect to the bolaamphiphilic compound of formula VIIa-VIId, VIIIa-VIIId, IXa-IXc, or Xa-Xc, X is —NMe₂ or —N⁺Me₃.

In one embodiment, with respect to the bolaamphiphilic compound of formula VIIa-VIId, VIIIa-VIIId, IXa-IXc, or Xa-Xc, X is —N(Me)-CH₂CH₂—OAc or —N⁺(Me)₂—CH₂CH₂—OAc.

In one embodiment, with respect to the bolaamphiphilic compound of formula VIIa-VIId, VIIIa-VIIId, IXa-IXc, or Xa-Xc, X is a chitosanyl group; and the chitosanyl group is a poly-(D)glucosaminyl group with MW of 3800 to 20,000 Daltons, and is attached to the core via N.

In one embodiment, the chitosanyl group is

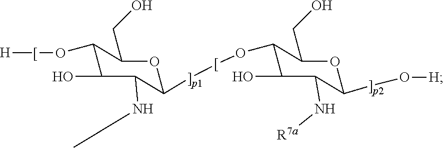

and wherein each p1 and p2 is independently an integer from 1-400; and each R^{7a} is H or acyl.

In one embodiment, with respect to the bolaamphiphilic compound of formula VIIa-VIId, VIIIa-VIIId, IXa-IXc, or Xa-Xc, X is a mannose group.

In one embodiment, with respect to the bolaamphiphilic compound of formula VIIa-VIId, VIIIa-VIIId, IXa-IXc, or Xa-Xc, X is a maleimide group.

In one embodiment, with respect to the bolaamphiphilic compound of formula I, II, III, IV, V, VI, VIIa-VIIc, VIIIa-VIIIc, IXa-IXc and Xa-Xc, the bolaamphiphilic compound is a pharmaceutically acceptable salt.

In one embodiment, with respect to the bolaamphiphilic compound of formula I, II, III, IV, V, VI, VIIa-VIIc, VIIIa-VIIIc, IXa-IXc and Xa-Xc, the bolaamphiphilic compound is in a form of a quaternary salt.

In one embodiment, with respect to the bolaamphiphilic compound of formula I, II, III, IV, V, VI, VIIa-VIIc, VIIIa-VIIIc, IXa-IXc and Xa-Xc, the bolaamphiphilic compound is in a form of a quaternary salt with pharmaceutically acceptable alkyl halide or alkyl tosylate.

In one embodiment, with respect to the bolaamphiphilic compound of formula I, II, III, IV, V, VI, VIIa-VIIc, VIIIa-VIIIc, IXa-IXc and Xa-Xc, the bolaamphiphilic compound is any one of the bolaambphilic compounds listed in Table 1.

In another specific aspect, provided herein are methods for incorporating GDNF in the bolavesicles. In one embodiment, the bolavesicle comporises one or more bolaamphilic compounds described herein.

In another specific aspect, provided herein are methods for brain-targeted drug delivery using the bolavesicles incorporated with GDNF.

In another specific aspect, provided herein are methods for delivering GDNF to the brain.

In another specific aspect, provided herein are nano-particles, comprising one or more bolaamphiphilic compounds and GDNF. In one embodiment, the bolaamphiphilic compounds and GDNF are encapsulated within the nano-particle.

In another specific aspect, provided herein are pharmaceutical compositions, comprising a nano-sized particle comprising one or more bolaamphiphilic compounds and GDNF; and a pharmaceutically acceptable carrier.

In another specific aspect, provided herein are methods for treatment or diagnosis of diseases or disorders selected from PD and related diseases using the nano-particles, pharmaceutical compositions or formulations of the present invention.

In another specific aspect, provided herein are methods for incorporating NGF in the bolavesicles. In one embodiment, the bolavesicle comporises one or more bolaamphilic compounds described herein.

In another specific aspect, provided herein are methods for brain-targeted drug delivery using the bolavesicles incorporated with NGF.

In another specific aspect, provided herein are methods for delivering NGF to the brain.

In another specific aspect, provided herein are nano-particles, comprising one or more bolaamphiphilic compounds and NGF. In one embodiment, the bolaamphiphilic compounds and NGF are encapsulated within the nano-particle.

In another specific aspect, provided herein are pharmaceutical compositions, comprising a nano-sized particle comprising one or more bolaamphiphilic compounds and NGF; and a pharmaceutically acceptable carrier.

In another specific aspect, provided herein are methods for treatment or diagnosis of diseases or disorders selected from AD and related diseases using the nano-particles, pharmaceutical compositions or formulations of the present invention.

In one embodiment, with respect to the bolaamphiphilic compound of formula I, II, III, IV, V, VI, VIIa-VIIc, VIIIa-VIIIc, IXa-IXc and Xa-Xc, the bolaamphiphilic compound is other than Comound ID GLH-16, GLH-19, GLH-20, GLH-26, GLH-29, or GLH-41.

In one embodiment, with respect to the bolaamphiphilic compound of formula I, II, III, IV, V, VI, VIIa-VIIc, VIIIa-VIIIc, IXa-IXc and Xa-Xc, the bolaamphiphilic compound is other than Comound ID GLH-6, GLH-8, GLH-12, GLH-13, GLH-13a, or GLH-49 to GLH-54 (all can be used as intermediates for bolaamphiphiles).

In another specific aspect, provided herein are composition of novel bolaamphiphilic compounds, wherein the bolaamphiphilic compound is selected from the bolaambphilic compounds listed in Table 1. In one embodiment, with respect to the bolaamphiphilic compound, the bolaamphiphilic compound is other than Comound ID GLH-16, GLH-19, GLH-20, GLH-26, GLH-29, or GLH-41. In another embodiment, with respect to the bolaamphiphilic compound, the compound is other than compound with ID GLH-3, GLH-4, GLH-5, or GLH-21.

In one particular embodiment, bolaamphiphilic compound is selected from the bolaambphilic compounds listed in Table 1, and the compound is compound with ID GLH-7, GLH-9, GLH-10, GLH-11, GLH-14, GLH-15, GLH-17, GLH-18, GLH-22, GLH-23, GLH-24, GLH-25, GLH-27, GLH-28, GLH-30 to GLH-48, GLH-55, GLH-56, or GLH-57.

In one embodiment, with respect to the bolaamphiphilic compound of formula I, II, III, IV, V, VI, VIIa-VIIc, VIIIa-VIIIc, IXa-IXc and Xa-Xc, the bolaamphiphilic compound is Comound ID GLH-19, or GLH-20.

In one embodiment, with respect to the bolaamphiphilic compound of formula I, II, III, IV, V, VI, VIIa-VIIc, VIIIa-VIIIc, IXa-IXc and Xa-Xc, the bolaamphiphilic compound is Comound ID GLH-16, GLH-26, GLH-29, or GLH-41.

The Derivatives and Precursors disclosed can be prepared as illustrated in the Schemes provided herein. The syntheses can involve initial construction of, for example, vernonia oil or direct functionalization of natural derivatives by organic synthesis manipulations such as, but not limiting to, epoxide ring opening. In those processes involving oxiranyl ring opening, the epoxy group is opened by the addition of reagents such as carboxylic acids or organic or inorganic nucleophiles. Such ring opening results in a mixture of two products in which the new group is introduced at either of the two carbon atoms of the epoxide moiety. This provides beta substituted alcohols in which the substitution position most remote from the CO group of the main aliphatic chain of the vernonia oil derivative is arbitrarily assigned as position 1, while the neighboring substituted carbon position is designated position 2. For simplicity purposes only, the Derivatives and Precursors shown herein may indicate structures with the hydroxy group always at position 2 but the Derivatives and Precursors wherein the hydroxy is at position 1 are also encompassed by the invention. Thus, a radical of the formula —CH(OH)—CH(R)— refers to the substitution of —OH at either the carbon closer to the CO group, designated position 2 or to the carbon at position 1. Moreover, with respect to the preparation of symmetrical bolaamphiphiles made via introducing the head groups through an epoxy moiety (e.g., as in vernolic acid) or a double bond (—C═C—) as in mono unsaturated fatty acids (e.g., oleic acid) a mixture of three different derivatives will be produced. In certain embodiments, vesicles are prepared using the mixture of unfractionated positional isomers. In one aspect of this embodiment, where one or more bolas are prepared from vernolic acid, and in which a hydroxy group as well as the head group introduced through an epoxy or a fatty acid with the head group introduced through a double bond (—C═C—), the bola used in vesicle preparation can actually be a mixture of three different positional isomers.

In other embodiments, the three different derivatives are isolated. Accordingly, the vesicles disclosed herein can be made from a mixture of the three isomers of each derivative or, in other embodiments, the individual isomers can be isolated and used for preparation of vesicles.

Symmetrical bolaamphiphiles can form relatively stable self aggregate vesicle structures by the use of additives such as cholesterol and cholesterol derivatives (e.g., cholesterol hemisuccinate, cholesterol oleyl ether, anionic and cationic derivatives of cholesterol and the like), or other additives including single headed amphiphiles with one, two or multiple aliphatic chains such as phospholipids, zwitterionic, acidic, or cationic lipids. Examples of zwitterionic lipids are phosphatidylcholines, phosphatidylethanol amines and sphingomyelins. Examples of acidic amphiphilic lipids are phosphatidylglycerols, phosphatidylserines, phosphatidylinositols, and phosphatidic acids. Examples of cationic amphipathic lipids are diacyl trimethylammonium propanes, diacyl dimethylammonium propanes, and stearylamines cationic amphiphiles such as spermine cholesterol carbamates, and the like, in optimum concentrations which fill in the larger spaces on the outer surfaces, and/or add additional hydrophilicity to the particles. Such additives may be added to the reaction mixture during formation of nanoparticles to enhance stability of the nanoparticles by filling in the void volumes of in the upper surface of the vesicle membrane.

Stability of nano vesicles according to the present disclosure can be demonstrated by dynamic light scattering (DLS) and transmission electron microscopy (TEM). For example, suspensions of the vesicles can be left to stand for 1, 5, 10, and 30 days to assess the stability of the nanoparticle solution/suspension and then analyzed by DLS and TEM.

The vesicles disclosed herein may encapsulate within their core the active agent, which in particular embodiments is selected from peptides, proteins, nucleotides and or non-polymeric agents. In certain embodiments, the active agent is also associated via one or more non-covalent interactions to the vesicular membrane on the outer surface and/or the inner surface, optionally as pendant decorating the outer or inner surface, and may further be incorporated into the membrane surrounding the core. In certain aspects, biologically active peptides, proteins, nucleotides or non-polymeric agents that have a net electric charge, may associate ionically with oppositely charged headgroups on the vesicle surface and/or form salt complexes therewith.

In particular aspects of these embodiments, additives which may be bolaamphiphiles or single headed amphiphiles, comprise one or more branching alkyl chains bearing polar or ionic pendants, wherein the aliphatic portions act as anchors into the vesicle's membrane and the pendants (e.g., chitosan derivatives or polyamines or certain peptides) decorate the surface of the vesicle to enhance penetration through various biological barriers such as the intestinal tract and the BBB, and in some instances are also selectively hydrolyzed at a given site or within a given organ. The concentration of these additives is readily adjusted according to experimental determination.

In certain embodiments, the oral formulations of the present disclosure comprise agents that enhance penetration through the membranes of the GI tract and enable passage of intact nanoparticles containing the drug. These agents may be any of the additives mentioned above and, in particular aspects of these embodiment, include chitosan and derivatives thereof, serving as vehicle surface ligands, as decorations or pendants on the vesicles, or the agents may be excipients added to the formulation.

In other embodiments, the nanoparticles and vesicles disclosed herein may comprise the fluorescent marker carboxyfluorescein (CF) encapsulated therein while in particular aspects, the nanoparticle and vesicles of the present disclosure may be decorated with one or more of PEG, e.g. PEG2000-vernonia derivatives as pendants. For example, two kinds of PEG-vernonia derivatives can be used: PEG-ether derivatives, wherein PEG is bound via an ether bond to the oxygen of the opened epoxy ring of, e.g., vernolic acid and PEG-ester derivatives, wherein PEG is bound via an ester bond to the carboxylic group of, e.g., vernolic acid.

In other embodiments, vesicles, made from synthetic amphiphiles, as well as liposomes, made from synthetic or natural phospholipids, substantially (or totally) isolate the therapeutic agent from the environment allowing each vesicle or liposome to deliver many molecules of the therapeutic agent. Moreover, the surface properties of the vesicle or liposome can be modified for biological stability, enhanced penetration through biological barriers and targeting, independent of the physico-chemical properties of the encapsulated drug.

In still other embodiments, the headgroup is selected from: (i) choline or thiocholine, O-alkyl, N-alkyl or ester derivatives thereof; (ii) non-aromatic amino acids with functional side chains such as glutamic acid, aspartic acid, lysine or cysteine, or an aromatic amino acid such as tyrosine, tryptophan, phenylalanine and derivatives thereof such as levodopa (3,4-dihydroxy-phenylalanine) and p-aminophenylalanine; (iii) a peptide or a peptide derivative that is specifically cleaved by an enzyme at a diseased site selected from enkephalin, N-acetyl-ala-ala, a peptide that constitutes a domain recognized by beta and gamma secretases, and a peptide that is recognized by stromelysins; (iv) saccharides such as glucose, mannose and ascorbic acid; and (v) other compounds such as nicotine, cytosine, lobeline, polyethylene glycol, a cannabinoid, or folic acid.

In further embodiments, nano-sized particle and vesicles disclosed herein further comprise at least one additive for one or more of targeting purposes, enhancing permeability and increasing the stability the vesicle or particle. Such additives, in particular aspects, may selected from from: (i) a single headed amphiphilic derivative comprising one, two or multiple aliphatic chains, preferably two aliphatic chains linked to a midsection/spacer region such as —NH—(CH₂)₂—N—(CH₂)₂—N—, or —O—(CH₂)₂—N—(CH₂)₂—O—, and a sole headgroup, which may be a selectively cleavable headgroup or one containing a polar or ionic selectively cleavable group or moiety, attached to the N atom in the middle of said midsection. In other asepcts, the additive can be selected from among cholesterol and cholesterol derivatives such as cholesteryl hemmisuccinate; phospholipids, zwitterionic, acidic, or cationic lipids; chitosan and chitosan derivatives, such as vernolic acid-chitosan conjugate, quaternized chitosan, chitosan-polyethylene glycol (PEG) conjugates, chitosan-polypropylene glycol (PPG) conjugates, chitosan N-conjugated with different amino acids, carboxyalkylated chitosan, sulfonyl chitosan, carbohydrate-branched N-(carboxymethylidene) chitosan and N-(carboxymethyl) chitosan; polyamines such as protamine, polylysine or polyarginine; ligands of specific receptors at a target site of a biological environment such as nicotine, cytisine, lobeline, 1-glutamic acid MK801, morphine, enkephalins, benzodiazepines such as diazepam (valium) and librium, dopamine agonists, dopamine antagonists tricyclic antidepressants, muscarinic agonists, muscarinic antagonists, cannabinoids and arachidonyl ethanol amide; polycationic polymers such as polyethylene amine; peptides that enhance transport through the BBB such as OX 26, transferrins, polybrene, histone, cationic dendrimer, synthetic peptides and polymyxin B nonapeptide (PMBN); monosaccharides such as glucose, mannose, ascorbic acid and derivatives thereof modified proteins or antibodies that undergo absorptive-mediated or receptor-mediated transcytosis through the blood-brain barrier, such as bradykinin B2 agonist RMP-7 or monoclonal antibody to the transferrin receptor; mucoadhesive polymers such as glycerides and steroidal detergents; and Ca²⁺ chelators. The aforementioned head groups on the additives designed for one or more of targeting purposes and enhancing permeability may also be a head group, preferably on an asymmetric bolaamphiphile wherein the other head group is another moiety, or the head group on both sides of a symmetrical bolaamphiphile.

In other embodiments, nano-sized particle and vesicles discloser herein may comprises at least one biologically active agent is selected from: (i) a natural or synthetic peptide or protein such as analgesics peptides from the enkephalin class, insulin, insulin analogs, oxytocin, calcitonin, tyrotropin releasing hormone, follicle stimulating hormone, luteinizing hormone, vasopressin and vasopressin analogs, catalase, interleukin-II, interferon, colony stimulating factor, tumor necrosis factor (TNF), melanocyte-stimulating hormone, superoxide dismutase, glial cell derived neurotrophic factor (GDNF) or the Gly-Leu-Phe (GLF) families; (ii) nucleosides and polynucleotides selected from DNA or RNA molecules such as small interfering RNA (siRNA) or a DNA plasmid; (iii) antiviral and antibacterial; (iv) antineoplastic and chemotherapy agents such as cyclosporin, doxorubicin, epirubicin, bleomycin, cisplatin, carboplatin, vinca alkaloids, e.g. vincristine, Podophyllotoxin, taxanes, e.g. Taxol and Docetaxel, and topoisomerase inhibitors, e.g. irinotecan, topotecan.

Additional embodiments within the scope provided herein are set forth in non-limiting fashion elsewhere herein and in the examples. It should be understood that these examples are for illustrative purposes only and are not to be construed as limiting in any manner.

### Pharmaceutical Compositions

In another aspect, the invention provides a pharmaceutical composition comprising a pharmaceutically acceptable carrier and a pharmaceutically effective amount of a compound of Formula I or a complex thereof.

When employed as pharmaceuticals, the compounds provided herein are typically administered in the form of a pharmaceutical composition. Such compositions can be prepared in a manner well known in the pharmaceutical art and comprise at least one active compound.

In certain embodiments, with respect to the pharmaceutical composition, the carrier is a parenteral carrier, oral or topical carrier.

The present invention also relates to a compound or pharmaceutical composition of compound according to Formula I; or a pharmaceutically acceptable salt or solvate thereof for use as a pharmaceutical or a medicament.

Generally, the compounds provided herein are administered in a therapeutically effective amount. The amount of the compound actually administered will typically be determined by a physician, in the light of the relevant circumstances, including the condition to be treated, the chosen route of administration, the actual compound administered, the age, weight, and response of the individual patient, the severity of the patient's symptoms, and the like.

The pharmaceutical compositions provided herein can be administered by a variety of routes including oral, rectal, transdermal, subcutaneous, intravenous, intramuscular, and intranasal. Depending on the intended route of delivery, the compounds provided herein are preferably formulated as either injectable or oral compositions or as salves, as lotions or as patches all for transdermal administration.

The compositions for oral administration can take the form of bulk liquid solutions or suspensions, or bulk powders. More commonly, however, the compositions are presented in unit dosage forms to facilitate accurate dosing. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Typical unit dosage forms include prefilled, premeasured ampules or syringes of the liquid compositions or pills, tablets, capsules or the like in the case of solid compositions. In such compositions, the compound is usually a minor component (from about 0.1 to about 50% by weight or preferably from about 1 to about 40% by weight) with the remainder being various vehicles or carriers and processing aids helpful for forming the desired dosing form.

Liquid forms suitable for oral administration may include a suitable aqueous or nonaqueous vehicle with buffers, suspending and dispensing agents, colorants, flavors and the like. Solid forms may include, for example, any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate; a glidant such as colloidal silicon dioxide; a sweetening agent such as sucrose or saccharin; or a flavoring agent such as peppermint, methyl salicylate, or orange flavoring.

Injectable compositions are typically based upon injectable sterile saline or phosphate-buffered saline or other injectable carriers known in the art. As before, the active compound in such compositions is typically a minor component, often being from about 0.05 to 10% by weight with the remainder being the injectable carrier and the like.

Transdermal compositions are typically formulated as a topical ointment or cream containing the active ingredient(s), generally in an amount ranging from about 0.01 to about 20% by weight, preferably from about 0.1 to about 20% by weight, preferably from about 0.1 to about 10% by weight, and more preferably from about 0.5 to about 15% by weight. When formulated as a ointment, the active ingredients will typically be combined with either a paraffinic or a water-miscible ointment base. Alternatively, the active ingredients may be formulated in a cream with, for example an oil-in-water cream base. Such transdermal formulations are well-known in the art and generally include additional ingredients to enhance the dermal penetration of stability of the active ingredients or the formulation. All such known transdermal formulations and ingredients are included within the scope provided herein.

The compounds provided herein can also be administered by a transdermal device. Accordingly, transdermal administration can be accomplished using a patch either of the reservoir or porous membrane type, or of a solid matrix variety.

The above-described components for orally administrable, injectable or topically administrable compositions are merely representative. Other materials as well as processing techniques and the like are set forth in Part 8 of Remington's Pharmaceutical Sciences, 17th edition, 1985, Mack Publishing Company, Easton, Pennsylvania, which is incorporated herein by reference.

The above-described components for orally administrable, injectable, or topically administrable compositions are merely representative. Other materials as well as processing techniques and the like are set forth in Part 8 of Remington's The Science and Practice of Pharmacy, 21st edition, 2005, Publisher: Lippincott Williams & Wilkins, which is incorporated herein by reference.

The compounds of this invention can also be administered in sustained release forms or from sustained release drug delivery systems. A description of representative sustained release materials can be found in Remington's Pharmaceutical Sciences.

The present invention also relates to the pharmaceutically acceptable formulations of compounds of Formula I. In certain embodiments, the formulation comprises water. In another embodiment, the formulation comprises a cyclodextrin derivative. In certain embodiments, the formulation comprises hexapropyl-β-cyclodextrin. In a more particular embodiment, the formulation comprises hexapropyl-β-cyclodextrin (10-50% in water).

The present invention also relates to the pharmaceutically acceptable acid addition salts of compounds of Formula I. The acids which are used to prepare the pharmaceutically acceptable salts are those which form non-toxic acid addition salts, i.e. salts containing pharmacologically acceptable aniovs such as the hydrochloride, hydroiodide, hydrobromide, nitrate, sulfate, bisulfate, phosphate, acetate, lactate, citrate, tartrate, succinate, maleate, fumarate, benzoate, para-toluenesulfonate, and the like.

The following formulation examples illustrate representative pharmaceutical compositions that may be prepared in accordance with this invention. The present invention, however, is not limited to the following pharmaceutical compositions.

### Formulation 1—Injection

A compound of the invention may be dissolved or suspended in a buffered sterile saline injectable aqueous medium to a concentration of approximately 5 mg/mL.

### Methods of Treatment

The nano-sized stable vesicles [5,6,7,8,9] can be used to deliver GDNF to the brain. These nano-sized vesicles are made of novel bolaamphiphiles (bolas). The vesicles that these novel bolas form were shown to aggregate into vesicles or nano particleds that cross the BBB and deliver small molecules, peptides and proteins to the brain. Bolas are promising building block candidates for vesicles used as a drug delivery system targeted to the brain, since they can form vesicles with monolayer membranes, which are more stable than liposomes with bilayer membranes, due to the high energy barrier for lipid exchange that characterizes bolas [10]. The high stability of such vesicles allows them to circulate in the blood stream until they reach the brain, and then penetrate the BBB in their intact form. In addition, the monolayer membrane is thinner than a bilayer membrane, thus providing higher inner volume for encapsulation as compared to vesicles of the same size made of an encapsulating bilayer membrane [11]. Moreover, a controlled release mechanism is more likely to be achieved with vesicles made of bolas that form monolayer membranes, as compared to classical liposomes made of bilayer encapsulating membranes, since monolayer membranes are known to rapidly change their morphology from vesicles to fibers and sheets upon small changes in their surface groups [10]. A controlled release mechanism should allow release of the encapsulated material only after the vesicles penetrate into the brain, thus preventing leakage in non relevant tissues. Indeed, the vesicles made from bolas do cross the BBB, transport encapsulated small molecules, peptides and proteins into the brain and release them primarily there. This novel delivery system can be an effective delivery system for GDNF, and has the potential to be used in the treatment of PD, since it can distribute the NTF within a wide brain area and, thus, can positively affect degenerating neurons throughout the brain. The resulting bola-GDNF delivery systems or formulations may be capable of delivering other neurotrophic factors for the treatment of several neurodegenerative diseases, particularly PD.

Thus, various PD active drug molecueles, such as GDNF, can be encapsulated in the bolaamphiphilic vesicles and then delivered to the brain in sufficient concentrations for therapeutic use.

In ceratin embodiments, the active drug molecules, including a neurotrophic factor selected from among Glial cell-derived neurotrophic factor (GDNF), Nerve Growth factor (NGF), Brain-Derived Neurotrophic Factor (BDNF), Neurotrophin-3 (NT-3), Neurotrophin-4/5 (NT-4/5), and combinations of two or more thereof can be encapsulated and delivered to the brain and/or periphperal nervous system in sufficient concentrations for therapeutic use.

The vesicles formed from the bolaamphiphiles to encapsulating NTF, GDNF or NGF, may contain additives that help to stabilize the vesicles, by stabilizing the vesicle's membranes, such as but not limited to cholesterol derivatives such as cholesteryl hemisuccinate and cholesterol itself and combinations such as cholesteryl hemisuccinate and cholesterol. The bola vesicles in addition to these components have another addiitves which decorates the outer vesicle memrbanes with groups or pendants that enhance penetration though biological barriers such as the BBB, or groups for targeting to specific sites such as dopaminergic neurons. In a further embodiment the bolaamphiphile head groups that comprise the vesicles membranes can interact with the neuro active agents such as GDNF or NDF to be delivered in to the brain and brain sites ionic interactions to enhance the % encapsulation via complexation and well as passive encapsulation within the vesicles core. Further the formuation may contain other additives within the veicles membranes to futhjer enhance the degree of encapsulation of neuro active agents such as GDNF or NDF. It is understood by one skilled in the state of art that the pH in which the vesicle formation and encapsulation of the neuro active agent such as GDNF or NDF is such as to maximize the electrostatic or inonic interactions between the said agents and the sai bolaamphiphiles and or additives to maximize the % encapsulation.

The bolaamphiphile vesicles disclosed herein are capable of penetrating the brain blood barrier (BBB) and transporting their encapsulated compounds into the brain. Those encapsulated compounds include small molecules as well as nucleic acids and proteins. Examples of such proteins include trypsinogen (molecular weight ˜24 kDa), the homodimeric protein—GDNF (molecular weight ˜30 kDa), horseradish peroxidase (molecular weight ˜44 kDa) and albumin (molecular weight ˜60 kDa).

Biological activity of these bolaamphiphile-vesicle-encapsulated therapeutic proteins, e.g. in the brain, can be evaluated in SBE transgenic mice. In particular, this animal model system may be used as a tool to measure the activity of Activin A following its delivery to the brain. Activin A, a member of the TGF-β superfamily, is a homodimeric protein with a molecular weight of ˜25 kD.

Preparation of bolaamphiphile-vesicle-encapsulated Activins, including Activin A, is accomplished according to the methods disclosed herein. Similarly, pharmaceutically acceptable formulations for administration of bolaamphiphile-vesicle-encapsulated Activins are also prepared according to the presently-disclosed methods. In particular, bolaamphiphile-vesicle-encapsulated Activin, including bolaamphiphile-vesicle-encapsulated Activin A, may be used for regulation of FSH secretion from the pituitary, neuronal development and spine formation, neurogenesis, late-phase long-term potentiation, and maintenance of long-term memory. Activin receptors are highly expressed in neuronal cells and their activation by Activin A leads to a transduction cascade that involves the phosphorylation of Smad proteins and their translocation into the nucleus where they are used as transcription factors and regulate gene expression. The SBE transgenic mice contain a Smad-responsive luciferase reporter that responds to Smad activation. This Smad-dependent signaling can be assessed non-invasively in the living mouse by bioluminescence imaging. Accordingly, this system is used to determine, in live animals, the activity of Activin A, delivered by the bolaamphiphile vesicles of the present disclosure, in the brain.

Accordingly, in one aspect, the present disclosure is directed to methods for delivery of the protein Activin (in particular Activin A) to the CNS by intravenvous or other non-invasive methods of administration. Activin is a protein complex that can be delivered into the brain by intravenous, oral, intraperitoneal and other noninvasive administration methods using the bolaamphiphilic vesicles of the present disclosure. Activin can be classified as either Activin A, Activin B and Activin AB; Actvin A is a peptide dimer of two βA subunits, Actvin AB is a peptide dimer of a βA and a βB subunit, and Activin B is a dimer of two βB subunits. All these are deliverable into the CNS and other organs by the bolaamphiphilc vesicles of the present disclosure.

Activin can enhance the survival of neural cell and that activin may act in vivo as a neuronal rescue factor. Activin may act in different parts of the CNS, and may be used for the treatment of Huntington's disease; Activin A as well as the other Activins, Activin B and AB may be a useful alternative to NGF in treating those conditions in which NGF therapy has shown promise, including peripheral neuropathy and Alzheimer's disease. Activin A partially reverses the phenotypic degeneration of striatal parvalbumin and NADPH interneurons. Activin A can rescue both striatal interneurons and striatal projection neurons from excitotoxic lesioning with quinolinic acid. Treatment with Activin A may help to prevent the degeneration of vulnerable striatal neuronal populations in Huntington's disease. Together with the localization of activin receptors to certain regions in the brain, specific central roles for activin are now being recognised. One of the first defined roles for activin in the brain was its modulation of oxytocin release and fluid regulation in the neurosecretory cells of the hypothalamus and brain stem. Activin may thus also be useful for mitigating or reversing autism. Given that activin can enhance the survival of neural cell lines and is neuroprotective for cultured midbrain neurons exposed to N-methyl-4-phenylpyridinium (MPP1) 42. Therefore, in one aspect of this embodiment, Activin may be administered (as disclosed herein) in vivo as a neuronal rescue factor and for treatement of diseases and conditions in need thereof

### General Synthetic Procedures

The compounds provided herein can be purchased or prepared from readily available starting materials using the following general methods and procedures. See, e.g., Synthetic Schemes below. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art by routine optimization procedures.

Additionally, as will be apparent to those skilled in the art, conventional protecting groups may be necessary to prevent certain functional groups from undergoing undesired reactions. The choice of a suitable protecting group for a particular functional group as well as suitable conditions for protection and deprotection are well known in the art. For example, numerous protecting groups, and their introduction and removal, are described in T. W. Greene and P. G. M. Wuts, Protecting Groups in Organic Synthesis, Second Edition, Wiley, New York, 1991, and references cited therein.

The compounds provided herein may be isolated and purified by known standard procedures. Such procedures include (but are not limited to) recrystallization, column chromatography or HPLC. The following schemes are presented with details as to the preparation of representative substituted biarylamides that have been listed herein. The compounds provided herein may be prepared from known or commercially available starting materials and reagents by one skilled in the art of organic synthesis.

It is to be understood that the bolaamphiphile compounds may be used as racemic mixtures or mixtures of geometric isomors such as cis or trans, or as mixtures of geometric isomers unless otherwise specified as being enantiomerically pure compounds. The enantiomerically pure compounds thay may be provided herein may be prepared according to any techniques known to those of skill in the art. For instance, they may be prepared by chiral or asymmetric synthesis from a suitable optically pure precursor or obtained from a racemate by any conventional technique, for example, by chromatographic resolution using a chiral column, TLC or by the preparation of diastereoisomers, separation thereof and regeneration of the desired enantiomer. See, e.g., “Enantiomers, Racemates and Resolutions,” by J. Jacques, A. Collet, and S. H. Wilen, (Wiley-Interscience, New York, 1981); S. H. Wilen, A. Collet, and J. Jacques, Tetrahedron, 2725 (1977); E. L. Eliel Stereochemistry of Carbon Compounds (McGraw-Hill, NY, 1962); and S. H. Wilen Tables of Resolving Agents and Optical Resolutions 268 (E. L. Eliel ed., Univ. of Notre Dame Press, Notre Dame, Ind., 1972, Stereochemistry of Organic Compounds, Ernest L. Eliel, Samuel H. Wilen and Lewis N. Manda (1994 John Wiley & Sons, Inc.), and Stereoselective Synthesis A Practical Approach, Mihály Nógrádi (1995 VCH Publishers, Inc., NY, N.Y.).

In certain embodiments, an enantiomerically pure compound of formula (1) may be obtained by reaction of the racemate with a suitable optically active acid or base. Suitable acids or bases include those described in Bighley et al., 1995, Salt Forms of Drugs and Adsorption, in Encyclopedia of Pharmaceutical Technology, vol. 13, Swarbrick & Boylan, eds., Marcel Dekker, New York; ten Hoeve & H. Wynberg, 1985, Journal of Organic Chemistry 50:4508-4514; Dale & Mosher, 1973, J. Am. Chem. Soc. 95:512; and CRC Handbook of Optical Resolution via Diastereomeric Salt Formation, the contents of which are hereby incorporated by reference in their entireties.

Enantiomerically pure compounds can also be recovered either from the crystallized diastereomer or from the mother liquor, depending on the solubility properties of the particular acid resolving agent employed and the particular acid enantiomer used. The identity and optical purity of the particular compound so recovered can be determined by polarimetry or other analytical methods known in the art. The diasteroisomers can then be separated, for example, by chromatography or fractional crystallization, and the desired enantiomer regenerated by treatment with an appropriate base or acid. The other enantiomer may be obtained from the racemate in a similar manner or worked up from the liquors of the first separation.

In certain embodiments, enantiomerically pure compound can be separated from racemic compound by chiral chromatography. Various chiral columns and eluents for use in the separation of the enantiomers are available and suitable conditions for the separation can be empirically determined by methods known to one of skill in the art. Exemplary chiral columns available for use in the separation of the enantiomers provided herein include, but are not limited to CHIRALCEL® OB, CHIRALCEL® OB-H, CHIRALCEL® OD, CHIRALCEL® OD-H, CHIRALCEL® OF, CHIRALCEL® OG, CHIRALCEL® OJ and CHIRALCEL® OK.

Abbreviations

BBB, blood brain barrier

BCECs, brain capillary endothelial cells

CF, carboxyfluorescein

CHEMS, cholesteryl hemisuccinate

CHOL, cholesterol

Cryo-TEM, Cryo-transmission electron microscope

DAPI, 4′,6-diamidino-2-phenylindole

DDS, drug delivery system

DLS, dynamic light scattering

DMPC, 1,2-dimyristoyl-sn-glycero-3-phosphocholine

DMPE, 1,2-dimyristoyl-sn-glycero-3-phosphoethanolamine

DMPG,1,2-dimyristoyl-sn-glycero-3-phospho-(1′-rac-glycerol)

EPR, electron paramagnetic resonance

FACS, fluorescence-activated cell sorting

FCR, fluorescence colorimetric response

GUVs, giant unilamellar vesicles

HPLC, high performance liquid chromatography

IR, infrared

MRI, magnetic resonance imaging

NMR, nuclear magnetic resonance

NPs, nanoparticles

PBS, phosphate buffered saline

PC, phosphatidylcholine

PDA, polydiacetylene.

TMA-DPH, 1-(4 trimethylammoniumphenyl)-6-phenyl-1,3,5-hexatriene

### EXAMPLE 1

### Bolaamphiphile Synthesis

The boloamphiphles or bolaamphiphilic compounds of the invention can be synthesized following the procedures described previously (see below).

Briefly, the carboxylic group of methyl vemolate or vernolic acid was interacted with aliphatic diols to obtain bisvernolesters. Then the epoxy group of the vemolate moiety, located on C12 and C13 of the aliphatic chain of vernolic acid, was used to introduce two ACh headgroups on the two vicinal carbons obtained after the opening of the oxirane ring. For GLH-20 (Table 1), the ACh head group was attached to the vemolate skeleton through the nitrogen atom of the choline moiety. The bolaamphiphile was prepared in a two-stage synthesis: First, opening of the epoxy ring with a haloacetic acid and, second, quaternization with the N,N-dimethylamino ethyl acetate. For GLH-19 (Table 1) that contains an ACh head group attached to the vemolate skeleton through the acetyl group, the bolaamphiphile was prepared in a three-stage synthesis, including opening of the epoxy ring with glutaric acid, then esterification of the free carboxylic group with N,N-dimethyl amino ethanol and the final product was obtained by quaternization of the head group, using methyl iodide followed by exchange of the iodide ion by chloride using an ion exchange resin.

Each bolaamphiphile was characterized by mass spectrometry, NMR and IR spectroscopy. The purity of the two bolaamphiphiles was >97% as determined by HPLC.

Materials. Iron(III) acetylacetonate (Fe(acac)3), diphenyl ether, 1,2-hexadecanediol, oleic acid, oleylamine, and carboxyfluorescein (CF) were purchased from Sigma Aldrich (Rehovot, Israel). Chloroform and ethanol were purchased from Bio-Lab Ltd. Jerusalem, Israel. 1,2-dimyristoyl-sn-glycero-3-phospho-(1′-rac-glycerol) (DMPG), 1,2-dimyristoyl-sn-glycero-3-phosphoethanolamine (DMPE), 1,2-dimyristoyl-sn-glycero-3-phosphocholine (DMPC), cholesterol (CHOL), cholesteryl hemisuccinate (CHEMS) were purchased from Avanti Lipids (Alabaster, Ala., USA), The diacetylenic monomer 10,12-tricosadiynoic acid was purchased from Alfa Aesar (Karlsruhe, Germany), and purified by dissolving the powder in chloroform, filtering the resulting solution through a 0.45 μm nylon filter (Whatman Inc., Clifton, N.J., USA), and evaporation of the solvent. 1-(4 trimethylammoniumphenyl)-6-phenyl-1,3,5-hexatriene (TMA-DPH) was purchased from Molecular Probes Inc. (Eugene, Oreg., USA).

### Synthesis Of Representative Bolaamphiphilic Compounds

The synthesis bolaamphiphilic compounds of this invention can be carried out in accordance with the methods described previously (Chemistry and Physics of Lipids 2008, 153, 85-97; Journal of Liposome Research 2010, 20, 147-59; WO2002/055011; WO2003/047499; or WO2010/128504) and using the appropriate reagents, starting materials, and purification methods known to those skilled in the art. Table 1 lists the representative bolaamphiphilic compounds of the invention.

TABLE 1
Representative Bolaamphiphiles

| # | Structure |
|---|---|
| GLH-3 | 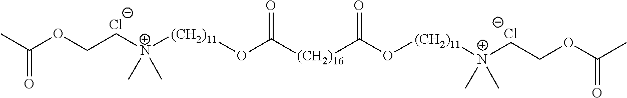 |
| GLH-4 | 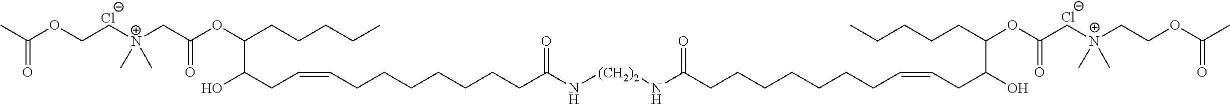 |
| GLH-5 | 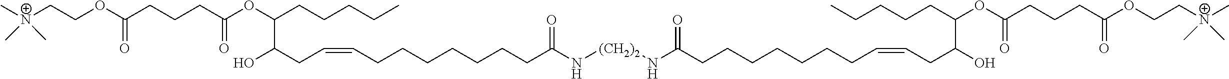 |
| GLH-6 a | 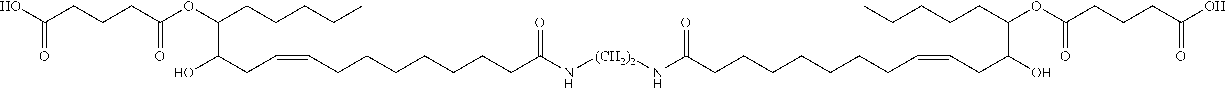 |
| GLH-7 | 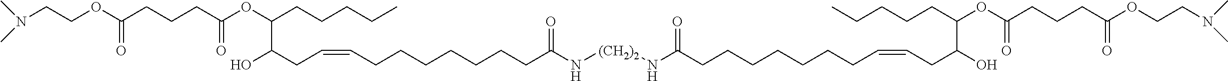 |
| GLH-8 | 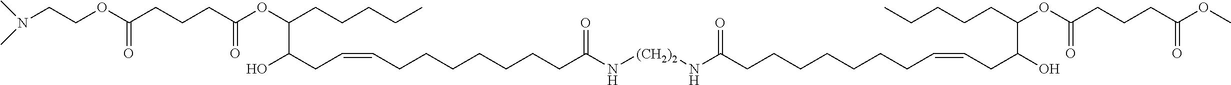 |
| GLH-9 | 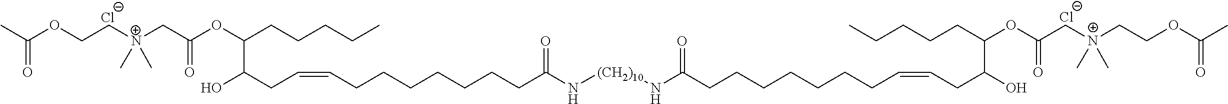 |
| GLH-10 | 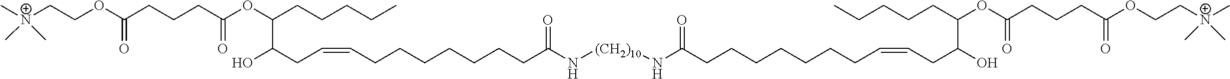 |
| GLH-11 | 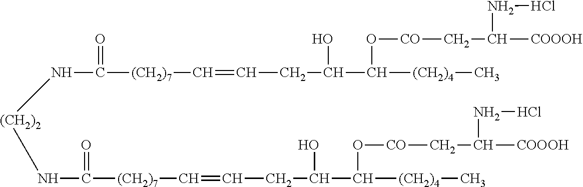 |
| GLH-12 a | 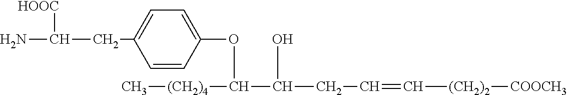 |
| GLH-13 a | 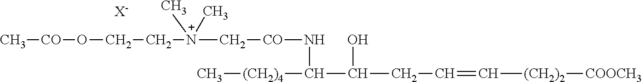 |
| GLH-13 a | 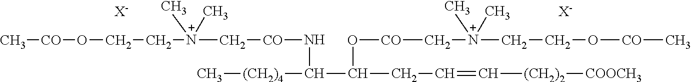 |
| GLH-14 | 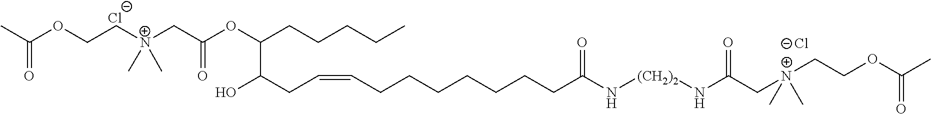 |
| GLH-15 | 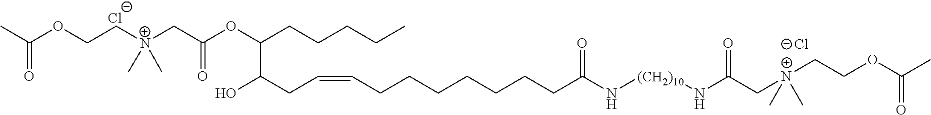 |
| GLH-16 | 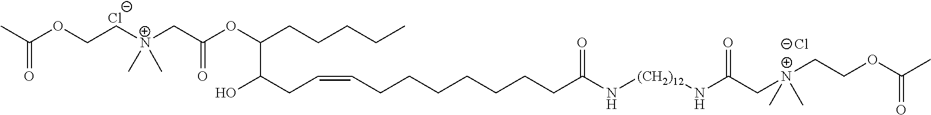 |
| GLH-17 | 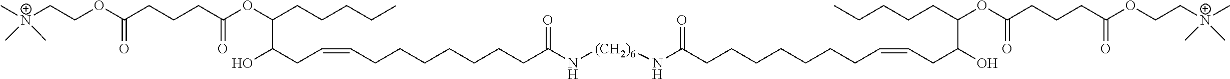 |
| GLH-18 | 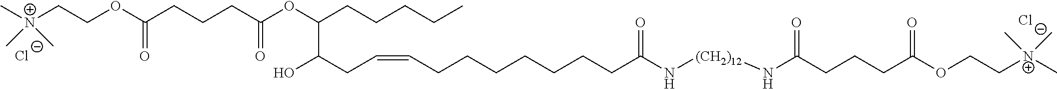 |
| GLH-19 | 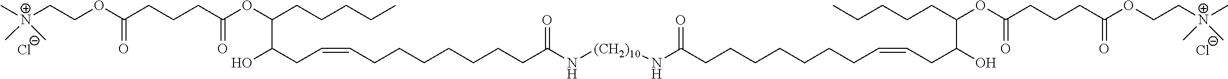 |
| GLH-20 | 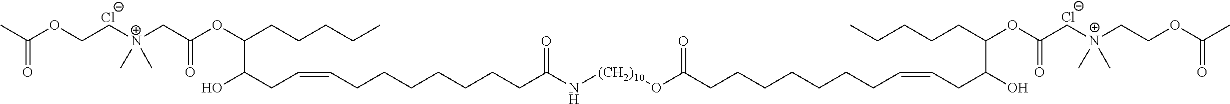 |
| GLH-21 | 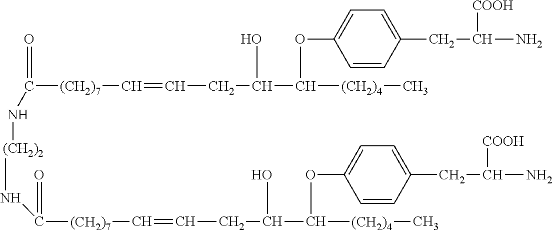 |
| GLH-22 | 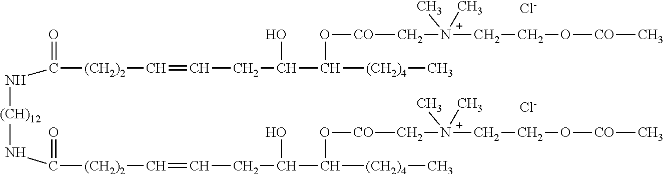 |
| GLH-23 | 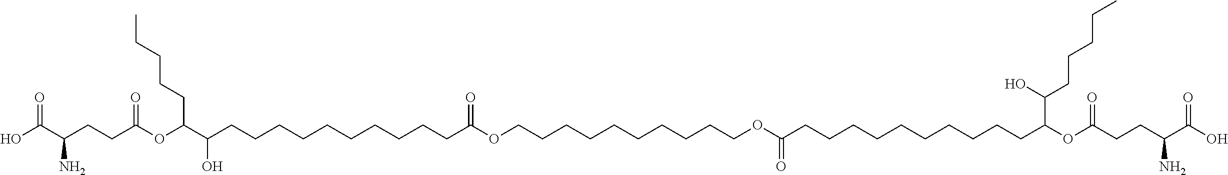 [SMILES: CCCCCC(O)C(CCCCCCCCCCC(=O)OCCCCCCCCCCOC(=O)CCCCCCCCCCC(O)C(CCCCC)OC(=O)CC[C@@H](N)C(=O)O)OC(=O)CC[C@H](N)C(=O)O] |
| GLH-24 | 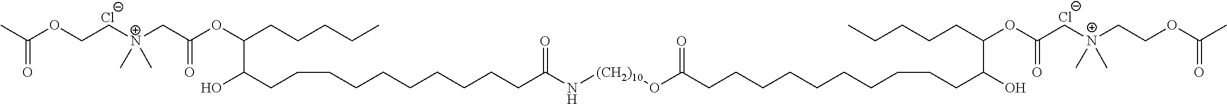 |
| GLH-25 | 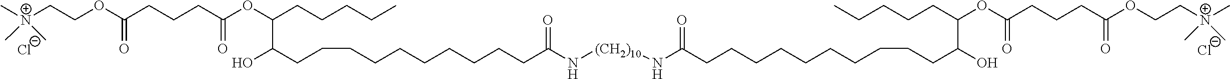 |
| GLH-26 | 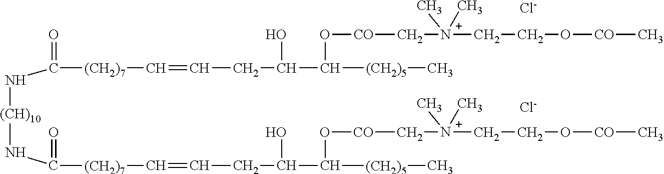 |
| GLH-27 | 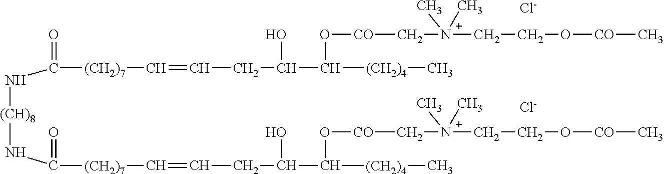 |
| GLH-28 | 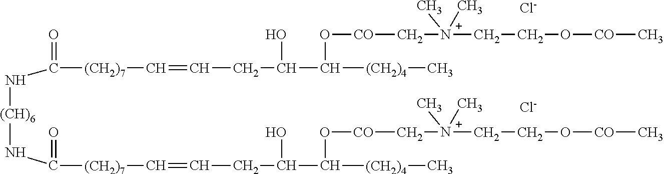 |
| GLH-29 | 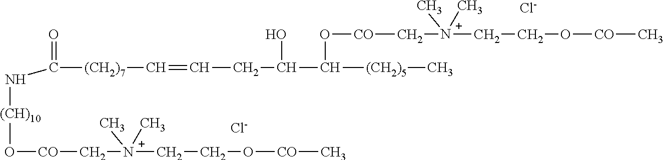 |
| GLH-30 | 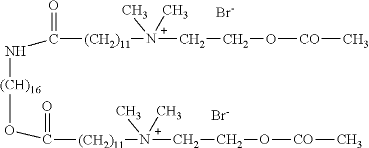 |
| GLH-30 | 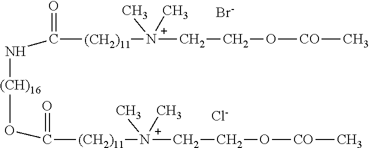 |
| GLH-31 | 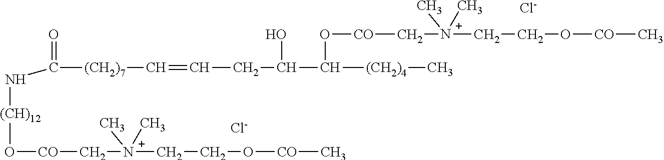 |
| GLH-32 | 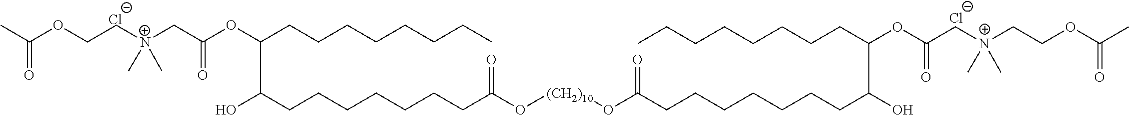 |
| GLH-33 | 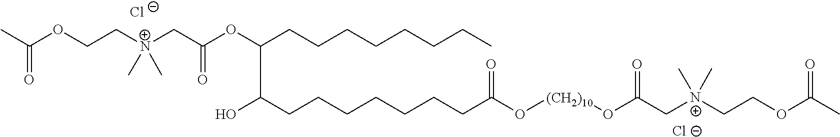 |
| GLH-34 | 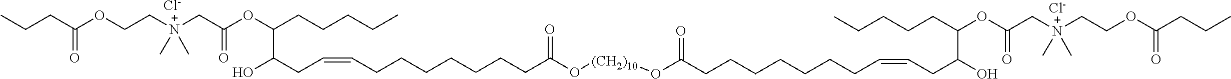 |
| GLH-35 | 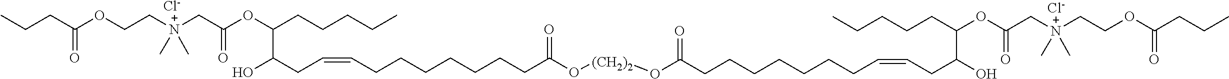 |
| GLH-36 | 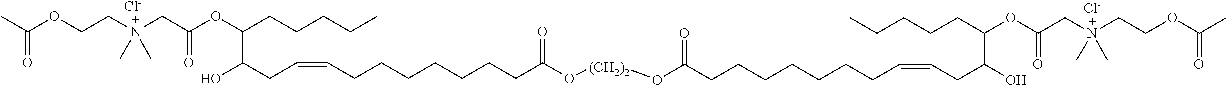 |
| GLH-37 | 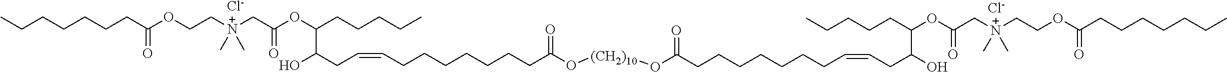 |
| GLH-38 | 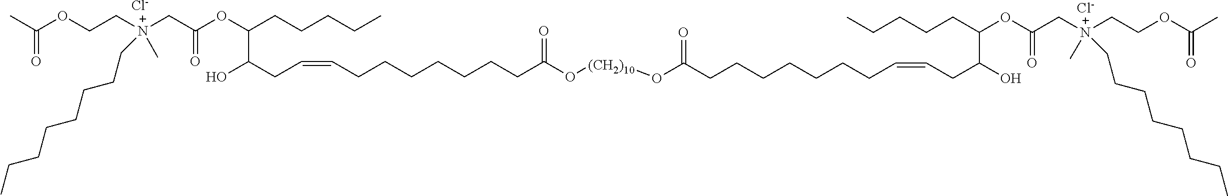 |
| GLH-39a | 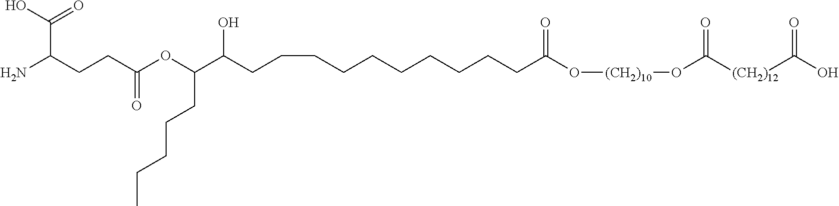 |
| GLH-40 | 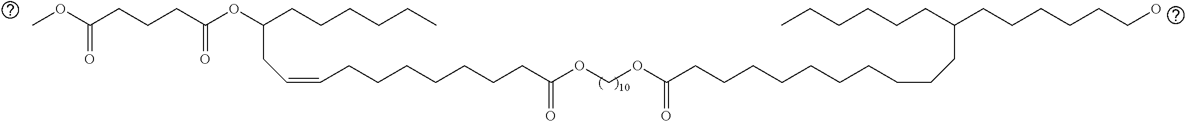 |
| GLH-41 | 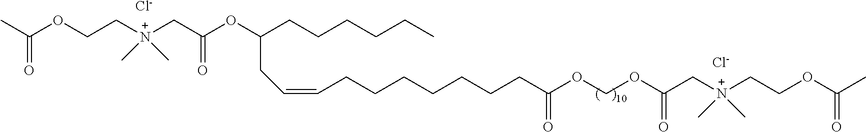 |
| GLH-42 a | 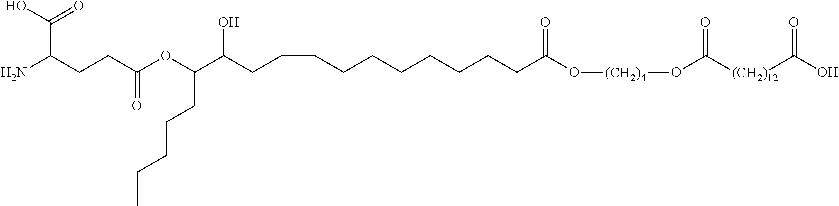 |
| GLH-43 a | 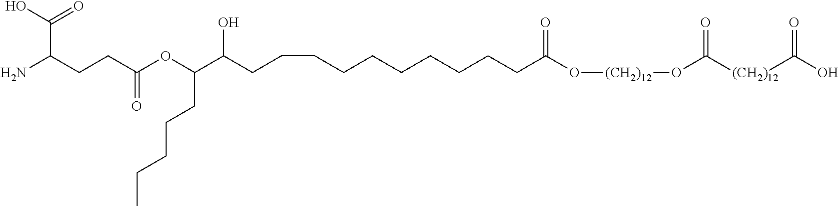 |
| GLH-44 | 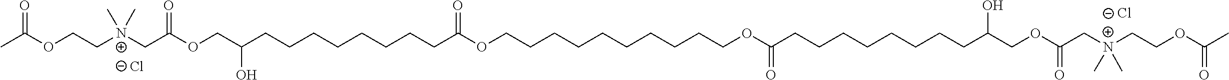 [SMILES: CC(=O)OCC[N+](C)(C)CC(=O)OCC(O)CCCCCCCCC(=O)OCCCCCCCCCCOC(=O)CCCCCCCCC(O)COC(=O)C[N+](C)(C)CCOC(C)=O ; [Cl-] ; [Cl-]] |
| GLH-45 | 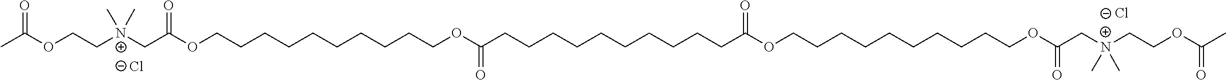 [SMILES: CC(=O)OCC[N+](C)(C)CC(=O)OCCCCCCCCCCOC(=O)CCCCCCCCCCC(=O)OCCCCCCCCCCOC(=O)C[N+](C)(C)CCOC(C)=O ; [Cl-] ; [Cl-]] |
| GLH-46 | 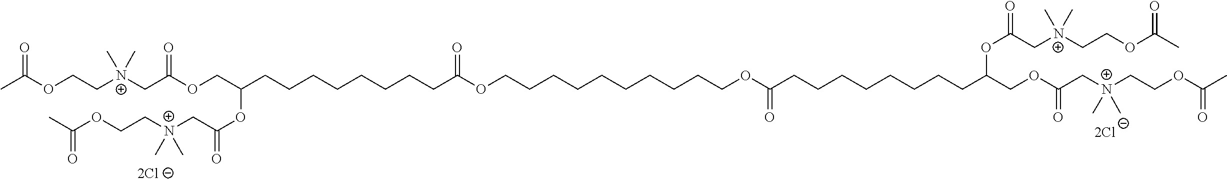 |
| GLH-47 | 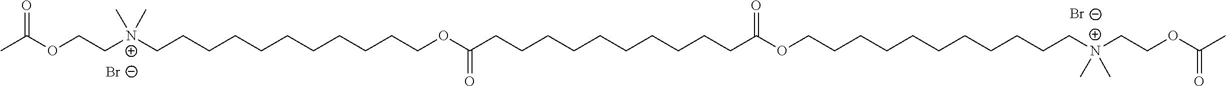 [SMILES: CC(=O)OCC[N+](C)(C)CCCCCCCCCCCOC(=O)CCCCCCCCCCC(=O)OCCCCCCCCCCC[N+](C)(C)CCOC(C)=O ; [Br-] ; [Br-]] |
| GLH-48 | 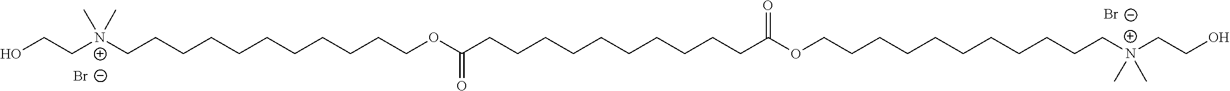 [SMILES: C[N+](C)(CCO)CCCCCCCCCCCOC(=O)CCCCCCCCCCC(=O)OCCCCCCCCCCC[N+](C)(C)CCO ; [Br-] ; [Br-]] |
| GLH-49 a | 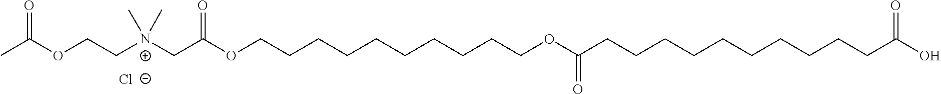 [SMILES: CC(=O)OCC[N+](C)(C)CC(=O)OCCCCCCCCCCOC(=O)CCCCCCCCCCC(=O)O ; [Cl-]] |
| GLH-50a | 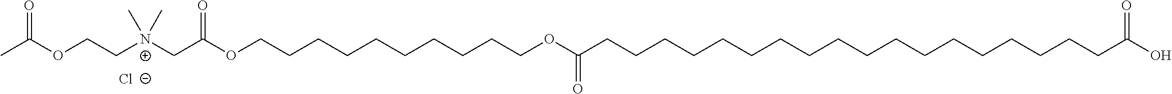 [SMILES: CC(=O)OCC[N+](C)(C)CC(=O)OCCCCCCCCCCOC(=O)CCCCCCCCCCCCCCCCCCC(=O)O ; [Cl-]] |
| GLH-51 a | 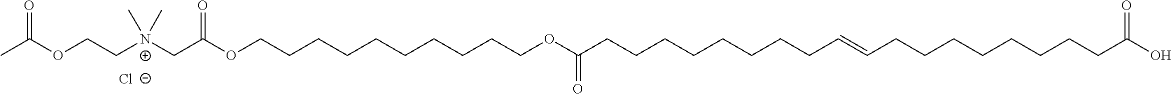 [SMILES: CC(=O)OCC[N+](C)(C)CC(=O)OCCCCCCCCCCOC(=O)CCCCCCCC/C=C/CCCCCCCCC(=O)O ; [Cl-]] |
| GLH-52a | 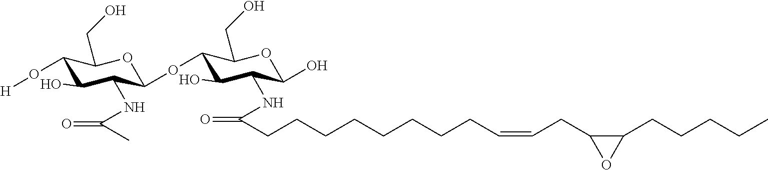 |
| GLH-53 a | 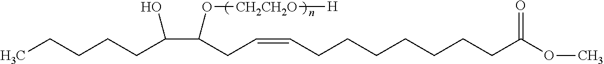 |
| GLH-54 a | 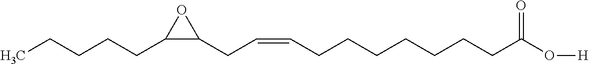 [SMILES: CCCCCC1OC1C/C=C\CCCCCCCC(=O)O] |
| GLH-55 | 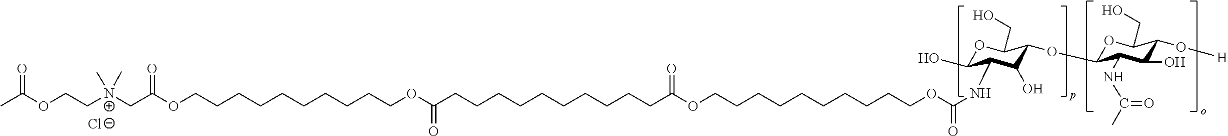 |
| GLH-56 | 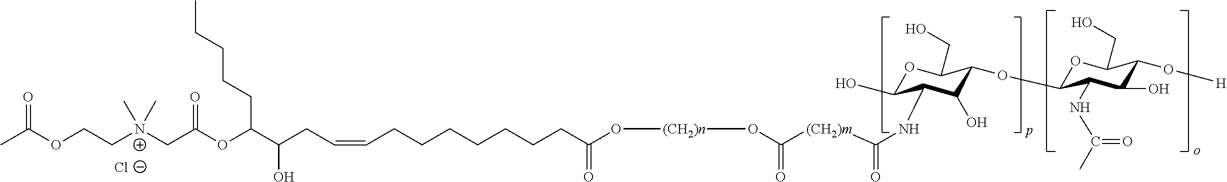 |
| GLH-57 | 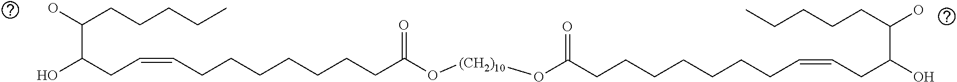 |

^{a}intermediate

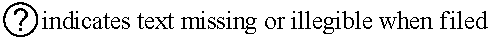

### EXAMPLE 2

### Vesicle Formation and Their Optimization

The vesicles shown to be effective in delivering enkephalin and albumin to the CNS were made from the bola GLH-20, or a mixture of GLH-19 and GLH-20 [Table 1]. Both of these bolas contain acetyl choline (ACh) head groups [8], but only GLH-20 is hydrolyzed by choline esterases (ChE). The mixture of these two bolas enables extended release of the encapsulated material. Stability and release rates can be used as the criteria to get the optimal ratio between GLH-19 and GLH-20. Stability and release rates can be studied using fluorescent measurements of encapsulated CF as described by us previously [7, 8]. Increasing the proportion of GLH-19 (which is not hydrolyzed by AChE) is expected to result in a slower release rate, thus prolonging the duration of action of the encapsulated active compound. Then, vesicles will be prepared by the method of film hydration, followed by sonication [14].

Each of the vesicle formulations can be examined for vesicle size (by dynamic light scattering), morphology (by cryo-transmission electron microscopy), zeta potential (by Zeta Potential Analyzer) and stability (by fluorescence measurements of encapsulated CF at various incubation times before and after exposing the vesicles to AChE and then to a detergent) [5,7,8]. Optimal formulations can be selected based on stability and ability to release encapsulated. material by AChE. Vesicle stability can be tested first in PBS and, then, if stable, in whole serum at 37° C. in the presence and absence of pyridostigmine—an AChE inhibitor.

### EXAMPLE 3

### Bolavesicle Preparation and Characterization

Bolaamphiphiles, cholesterol, and CHMES (2:1:1 mole ratio) are dissolved in chloroform or a suitable solvent. 0.5 mg of the GDNF dispersed in chloroform is added to the mix. The solvents are evaporated under vacuum and the resultant thin films are hydrated in 0.2 mg/mL CF solution in PBS and probe-sonicated (Vibra-Cell VCX130 sonicator, Sonics and Materials Inc., Newtown, CT, USA) with amplitude 20%, pulse on: 15 sec, pulse off: 10 sec to achieve homogenous vesicle dispersions. Vesicle size and zeta potential were determined using a Zetasizer Nano ZS (Malvern Instruments, UK).

### EXAMPLE 4

### Measurement of the Quality and Activity of the Encapsulated GDNF

The encapsulated GDNF can be run on acrylamide gel electrophoresis (after release from vesicles by a detergent) to confirm that it maintained its integrity during the encapsulation process. In addition, the activity of the GDNF affected by the encapsulation process can be tested by measuring the ability of the encapsulated material to induce tyrosine hydroxylase (TH) gene expression in comparison to free GDNF. SK-N-MC cells stably transfected with expression constructs of c-ret and with a luciferase reporter gene driven by 2 kb of the rat TH gene promoter region can be used. In the presence of GDNF, luciferase activity is expected to increase [15].

### Spectral Characterization

### EXAMPLE 5

### Cryogenic Transmission Electron Microscopy (Cryo-TEM)

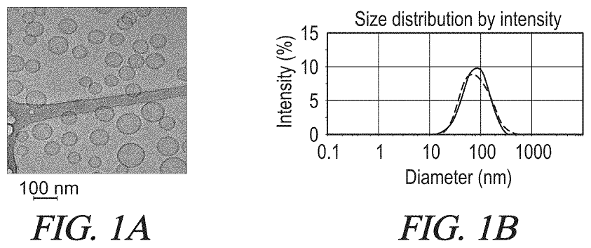

Specimens studied by cryo-TEM were prepared. Sample solutions (4 μL) are deposited on a glow discharged, 300 mesh, lacey carbon copper grids (Ted Pella, Redding, Calif., USA). The excess liquid is blotted and the specimen was vitrified in a Leica EM GP vitrification system in which the temperature and relative humidity are controlled. The samples are examined at −180° C. using a FEI Tecnai 12 G2 TWIN TEM equipped with a Gatan 626 cold stage, and the images are recorded (Gatan model 794 charge-coupled device camera) at 120 kV in low-dose mode. FIG. 1 shows TEM micrograph of vesicles from GLH-20 (A) and their size distribution determined by DLS (B).

### Assays

### EXAMPLE 6

### Lipid/Polydiacetylene (PDA) Assay

Lipid/polydiacetylene (PDA) vesicles (PDA/DMPC 3:2, mole ratio) are prepared by dissolving the lipid components in chloroform/ ethanol and drying together in vacuo. Vesicles are subsequently prepared in DDW by probe-sonication of the aqueous mixture at 70° C. for 3 min. The vesicle samples are then cooled at room temperature for an hour and kept at 4° C. overnight. The vesicles are then polymerized using irradiation at 254 nm for 10-20 s, with the resulting emulsions exhibiting an intense blue appearance. PDA fluorescence is measured in 96-well microplates (Greiner Bio-One GmbH, Frickenhausen, Germany) on a Fluoroscan Ascent fluorescence plate reader (Thermo Vantaa, Finland). All measurements are performed at room temperature at 485 nm excitation and 555 nm emission using LP filters with normal slits. Acquisition of data is automatically performed every 5 min for 60 min. Samples comprised 30 μL of DMPC/PDA vesicles and 54 bolaamphiphilic vesicles assembled with HIV drug, followed by addition of 30 μL 50 mM Tris-base buffer (pH 8.0).

A quantitative value for the increasing of the fluorescence intensity within the PDA/PC-labeled vesicles is given by the fluorescence colorimetric response (% FCR), which is defined as follows²⁷:

$$\% FCR = [ ( {F}_{I} - {F}_{0} ) / {F}_{100} ] \cdot 100. \qquad Eq . 1$$

Where F₁ is the fluorescence emission of the lipid/PDA vesicles after addition of the tested membrane-active compounds, F₀ is the fluorescence of the control sample (without addition of the compounds), and F₁₀₀ is the fluorescence of a sample heated to produce the highest fluorescence emission of the red PDA phase minus the fluorescence of the control sample.

### EXAMPLE 8

### Cell Culture

b.End3 immortalized mouse brain capillary endothelium cells are kindly provided by Prof. Philip Lazarovici (Institute for Drug Research, School of Pharmacy, The Hebrew University of Jerusalem, Israel). The b.End3 cells were cultured in DMEM medium supplemented with 10% fetal bovine serum, 2 mM L-Glutamine, 100 IU/mL penicillin and 100 μg/mL streptomycin (Biological Industries Ltd., Beit Haemek, Israel). The cells are maintained in an incubator at 37° C. in a humidified atmosphere with 5% CO₂.

### EXAMPLE 9

### Internalization of CF by the Cells In Vitro

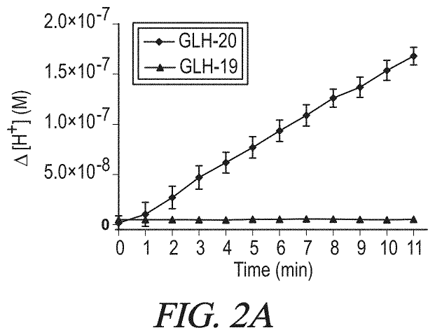

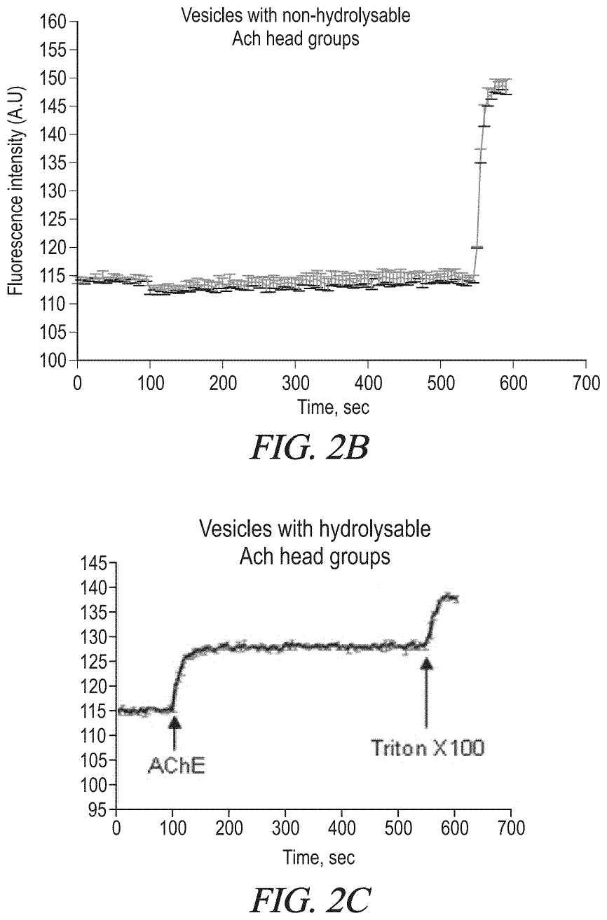

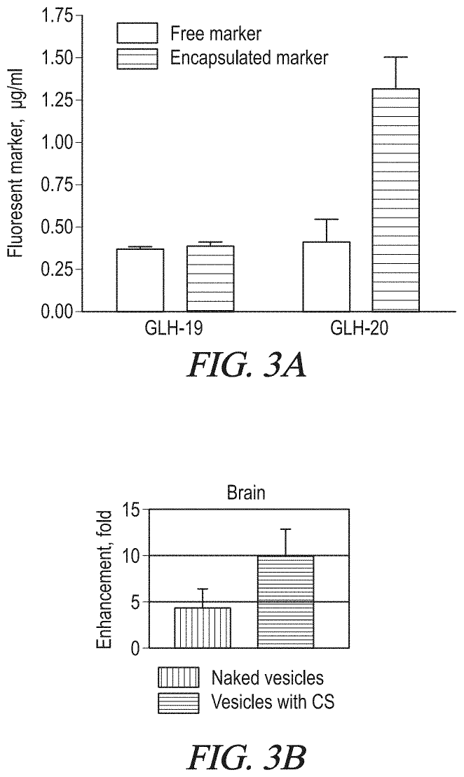

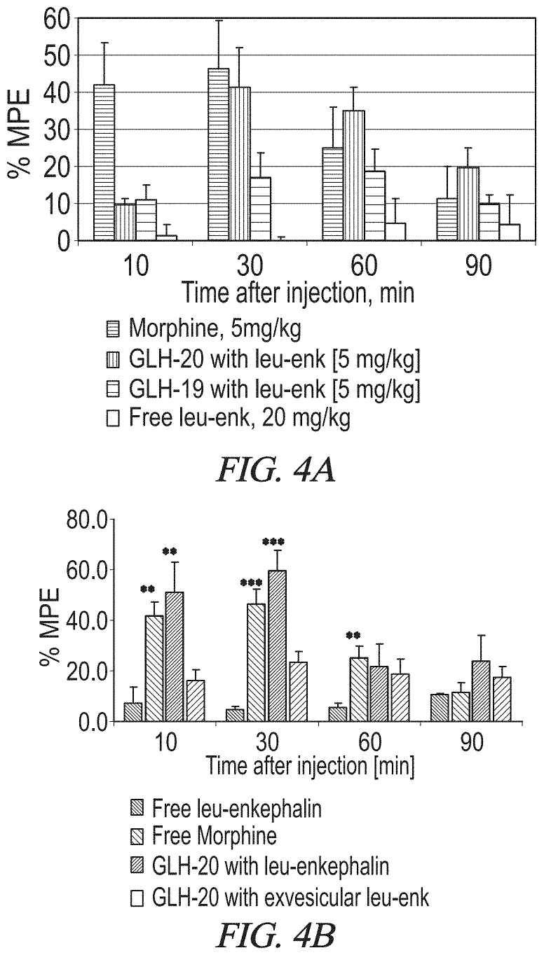

b.End3 cells are grown on 24-well plates or on coverslips (for FACS and fluorescence microscopy analysis, respectively). The medium is replaced with culture medium without serum and CF solution, or tested bolavesicles (equivalent to 0.5 μg/mL CF), or equivalent volume of the medium are added to the cells and incubated for 5 hr at 4° C. or at 37° C. At the end of the incubation, cells are extensively washed with complete medium and with PBS, and are either detached from the plates using trypsin-EDTA solution (Biological Industries Ltd., Beit Haemek, Israel) and analyzed by FACS (FACSCalibur Flow Cytometer, BD Biosciences, USA), or fixed with 2.5% formaldehyde in PBS, washed twice with PBS, mounted on slides using Mowiol-based mounting solution and analyzed using a FV1000-IX81 confocal microscope (Olympus, Tokyo, Japan) equipped with 60× objective. All the images are acquired using the same imaging settings and are not corrected or modified. The FIG. 2 shows head group hydrolysis by AChE (A) of GLH-19 (blue) and GLH-20 (red) and release of CF from GLH-19 vesicles (B) and GLH-20 vesicles (C). AChE causes the release of encapsulated material from GLH-20 vesicles, but not from GLH-19 vesicles (FIG. 2). The vesicles are capable of delivering small molecules, such as carboxyfluorescein (CF), into a mouse brain, but the fluorescent dye accumulates only if it is delivered in vesicles that release their encapsulated CF in presence of AChE, namely, GLH-20 vesicles (FIG. 3A). These results suggest that the release is due to head group hydrolysis by AChE in the brain. Corroboration for this conclusion also comes from an experiment showing that when an analgesic peptide is delivered to the brain by the bola vesicles, analgesia (which is caused when the encapsulated peptide is released in the brain) was observed only with GLH-20 vesicles, but not by GLH-19 vesicles (FIG. 4A).The vesicles do not break the BBB, but rather penetrate it in their intact form, as indicated by the finding that analgesia is obtained only when enkephalin is administered while encapsulated within the vesicles, but not when free enkephalin is administered together with empty vesicles (FIG. 4B).

The ACh head groups also provide the vesicles with cationic surfaces, which promote penetration through the BBB [Lu et al, 2005] and transport of the encapsulated material into the brain. Toxicity studies showed that the dose which induced the first toxic signs was 10-20 times higher than the doses needed to obtain analgesia by encapsulated analgesic peptides.

The addition of chitosan (CS) surface groups, by employing CS-vernolate conjugates, increased BBB permeability of the vesicles (FIG. 4B), probably by increasing transcytosis [Newton, 2006]. However, the CS groups, when added to the vesicles by employing fatty acid-CS conjugate (in this case, vernolic acid), are not stable in circulation as surface groups because of the low energy barrier for lipid exchange of such conjugates. Thus bolaamphiphiles with chitosan head groups were synthesized and used to form vesicles with better penetration into the brain through the BBB as shown in the examples provided below.

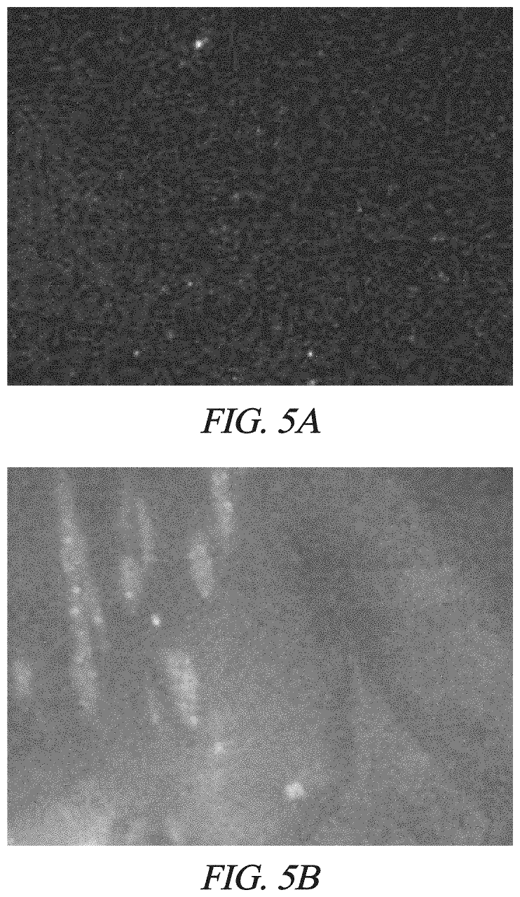

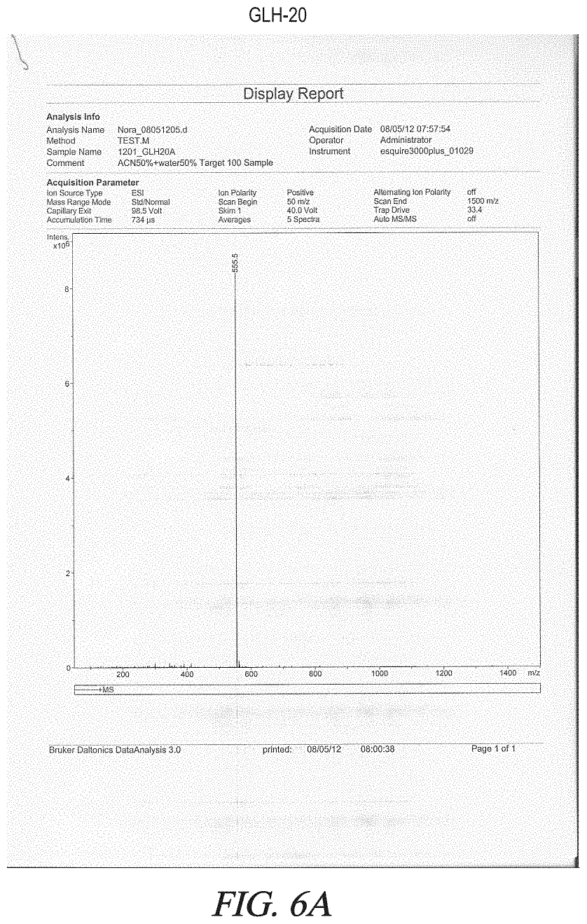

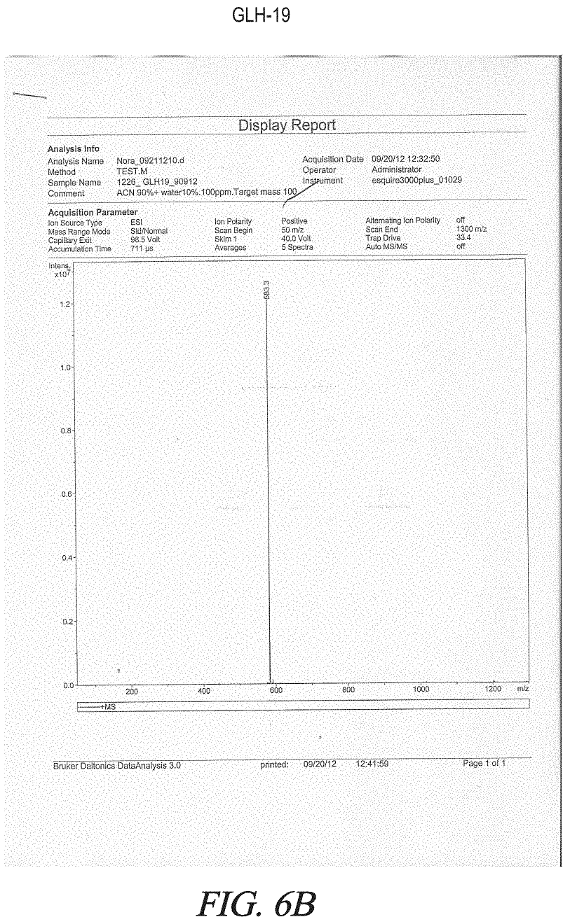

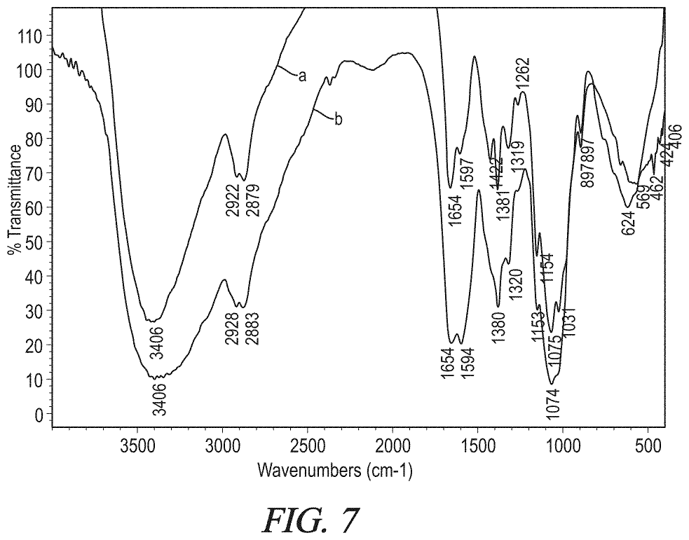

In addition to the peptide leu-enkephalin, and the small molecules: CF, uranyl acetate, kyotorphin and sucrose, the inventors have also successfully encapsulated in these vesicles the proteins albumin and trypsinogen and the polysaccharide Dextran-FITC (MW 9000). Albumin-FITC, encapsulated, was delivered successfully to the brain (FIG. 5B), while un-encapsulated albumin-FITC showed little, if any, brain accumulation (FIG. 5A), indicating that the vesicle transported the protein into the brain through the BBB. These results strongly suggest that the vesicles can be made to encapsulate other molecules, such as agents agains neurodegerative diseases such as GDNF and NGF, and other agents agains other diseases such as anti-retroviral drugs, and deliver them into the brain without harming the BBB.

### EXAMPLE 11

### Statistical Analysis

The data are presented as mean and standard deviations (SD) or standard errors of mean (SEM). Statistical differences between the control and the studied formulations are analyzed using ANOVA followed by Dunnett post-test using InStat 3.0 software (GraphPad Software Inc., La Jolla, Calif., USA). P values of less than 0.05 are defined as statistically significant.

Synthesis and Delivery of Neurotrophin-Containing Nanovesicles to the Brain Introduction

The experiments herein describe the preparation of vesicles of the invention for the delivery of glial cell line-derived neurotrophic factor (GDNF) systemically to the brain and demonstrate the capability of these vesicles to target the delivered GDNF to brain regions affected in Parkinson's disease (PD). Delivering GDNF to brain regions affected in PD, such as the Substantia Nigra pars compacta (SNpc) and the striatum (STR), may be beneficial in slowing down the progression of PD and may even promote neurorestoration, thus improving the status of the PD patient [Kordower J H, Emborg M E, Bloch J, Ma S Y, Chu Y, Leventhal L, McBride J, Chen E Y, Palfi S, Roitberg B Z, Brown W D, Holden J E, Pyzalski R, Taylor M D, Carvey P, Ling Z, Trono D, Hantraye P, Deglon N, Aebischer P. Neurodegeneration prevented by lentiviral vector delivery of GDNF in primate models of Parkinson's disease. Science. 2000; 290(5492):767-773]. However, GDNF does not permeate through the blood-brain barrier (BBB) and, to demonstrate efficacy, it has to be delivered directly into the brain [Slevin J T, Gash D M, Smith C D, Gerhardt G A, Kryscio R, Chebrolu H, Walton A, Wagner R, Young A B. Unilateral intraputamenal glial cell line-derived neurotrophic factor in patients with Parkinson disease: response to 1 year of treatment and 1 year of withdrawal. J Neurosurg. 2007; 106(4):614-620]. Nevertheless, attempts to deliver GDNF directly into the brain by intraputamenal injection, or convection enhanced delivery (CED) were generally not successful, most probably because of poor distribution of the delivered GDNF within the brain, which was restricted to only 2-9% of the application area [Salvatore M F, Ai Y, Fischer B, Zhang A M, Grondin R C, Zhang Z, Gerhardt G A, Gash D M. Point source concentration of GDNF may explain failure of phase II clinical trial. Exp Neurol. 2006; 202(2):497-505; Gash D M, Zhang Z, Ai Y, Grondin R, Coffey R, Gerhardt G A. Trophic factor distribution predicts functional recovery in parkinsonian monkeys. Ann Neurol. 2005; 58(2):224-233]. The limited GDNF diffusion throughout the brain was accounted for by its tight binding to the extracellular matrix [Hamilton J F, Morrison P F, Chen M Y, Harvey-White J, Pernaute R S, Phillips H, Oldfield E, Bankiewicz K S. Heparin coinfusion during convection-enhanced delivery (CED) increases the distribution of the glial-derived neurotrophic factor (GDNF) ligand family in rat striatum and enhances the pharmacological activity of neurturin. Exp Neurol. 2001; 168(1):155-161]. This implies that a delivery system capable of transporting GDNF to a wide area within the brain and targeting it to brain regions which are affected in PD should increase the probability that all affected neurons are exposed to therapeutic concentrations of the neurotrophin and, thus, increase its efficacy in the treatment of PD.

The experiments herein describe the development of nano-sized stable vesicles based on the novel delivery system. These nano-sized vesicles are made of novel bolas that are the building block materials for nanoparticles used as a drug delivery system that can self-assemble into vesicles with monolayer membranes. These nanoparticles are more stable than liposomes made of bilayer membranes, due to the higher energy barrier for lipid exchange that characterizes monolayer membranes made from bolas [Fuhrhop J H, Wang T. Bolaamphiphiles, Chem Rev. 2004; 104:2901-2937]. The high stability of such vesicles allows them to circulate in the blood stream until they reach the brain, and then penetrate the blood brain barrier (BBB) to deliver their cargo into the brain. In addition, the monolayer membrane is thinner than the bilayer membrane, which is an important parmater in nano-sized vesicles, since is provides a higher inner volume for encapsulating drugs and biologically active compounds, as compared to liposomes of the same size that are made of a bilayer membrane. The vesicles described herein may also be characterized as providing a controlled-release mechanism that enables the release of the cargo preferentially in the brain.

The experiments herein describe, for delivery of GDNF to brain regions that are affected in PD, provide vesicles two important components: a) a bola with a chitosan (CS) head group for increasing BBB permeability of the vesicles, and b) a bola with a dopamine transporter (DAT) ligand for targeting the vesicles to dopaminergic cells in the brain.

### Materials And Methods

Chemicals:

Vernonia oil that was used as the starting material for the synthesis of the bolas was purchased from Ver-Tech, Inc., Bethesda, Md., USA. Chitosan-vernolate conjugate was synthesized in our lab. Pyridostigmine(3-(Dimethylamino-carbonyloxy)-1-methylpyridiniumbromide); Carboxyfluorescein; Triton® X-100 (t-Octylphenoxy-polyethoxyethanol); Triton® X-100 —Reduced form; Cholesterol (5-Cholesten-3β-ol); Cholesteryl Hemisuccinate (5-Cholesten-3β-ol 3-hemisuccinate); Sephadex G-50, 50-150 micron; Trizma® Base (Tris{hydroxymethyl} aminomethane) and its hydrochloride salt; Trichloro acetic acid (TCA); trypsinogen from bovine pancreas and chitosan; all were of analytical grade and purchased from Sigma Chemicals. Human GDNF (hGDNF) and hGDNF-sulfo-NHS-LC-biotin (GDNF-biotin) were purchased from Alomone Labs, Jerusalem. Cocaine that was used for the synthesis of the DAT ligand was obtained under license from the Chief Pharmacist of the Regional Health Office, Southern Region, Ministry of Health. AlexaFluor®-488 Protein Labeling Kit (A10235) and AlexaFluor®-488 Microscale Protein Labeling Kit (A30006) were bought from Invitrogen. Other standard chemicals were all purchased from commercial sources. Solutions for inducing anastasia in animals (Ketamine HC1 100 mg/ml and Xylazine 2%) were obtained from the Ben Gurion University (BGU) animal facility.

Synthesis of Bolaamphiphiles (Bolas) and Chitosan-Fatty Acid Conjugate

Vernonia oil was hydrolyzed to obtain vernolic acid or transesterified with methanol to obtain the methyl vernolate, both compounds were used as starting materials for the synthesis of bolaamphiphiles, described below. Vernolic acid has an epoxy group that provides a reactive moiety to which functional groups are conjugated.

Vernolate-Chitosan that was used for comparison to the bola with the chitosan head groups, as described below, was synthesized by attaching a low molecular weight chitosan to N-hydroxy succineimide vernolate.

Analysis of the Synthesized Bolas

Elemental analysis was outsourced to a commercial laboratory. FT-IR analysis was carried out on a Nicolet spectrometer. ¹H and ¹³C NMR (500 MHz) spectra were recorded on a Brucker WP-500 SY spectrometers, respectively, in CDC13 with TMS as the internal standard or d₆ DMSO solutions. HPLC analysis was carried out on a C18RP column with an evaporative light scattering detector (evaporation temperature 46 ° C.; mobile phase methanol:water (9:1, v/v); flow rate 0.5 ml/min). MS analysis was carried out on a Waters Micromass Q-TOF Premier Mass spectrometer (Waters-Micromass, Milford, Mass., USA).

Vesicle Preparation

The basic components of the vesicles were the bolas GLH-19 and GLH-20. In addition to the bolas, the vesicle formulation contained the additives cholesterol and cholesteryl hemisuccinate and as indicated in the text, some formulations included also CS-vernolate conjugate or GLH-55a (a bola with CS head group) and/or GLH-57 (a bola with DAT ligand head groups). Unless otherwise stated, the ratio between GLH-19 and GLH-20 was 2:1. This ratio was found to give stable vesicles that release their content in a controlled manner (see Results). Thus, the different formulations used in this project were: (a) GLH-19+GLH-20 (2:1)/cholesterol/cholesteryl-hemisuccinate (10/1.6/2.1); (b) GLH-19+GLH-20/cholesterol/cholesteryl-hemisuccinate/CS-conjugate (10/1.6/2.1/1); (c) GLH-19+GLH-20/cholesterol/cholesteryl hemisuccinate/GLH-55a (CS-bola) (10/1.6/2.1/1); (d) GLH-19+GLH-20/cholesterol/cholesteryl hemisuccinate/GLH-57 (bola DAT) (10/1.6/2.1/0.8); (e) GLH-19+GLH-20/cholesterol/cholesteryl hemisuccinate/CS conjugate or GLH-55a/GLH-57 (10/1.6/2.1/1/0.8).

Vesicles were prepared from the formulation described above by known methods: (a) Film Hydration followed by extrusion; or (b) Film hydration followed by sonication. Vesicle formation was conducted at room temperature (i.e. 25° C.), which is above the transition point of the bolaamphiphilic compounds used in the present study. When the vesicles were prepared by extrusion, the bolas and the additives were dissolved in an organic solvent (usually chloroform). The solution was then placed in a vial and dried under stream of nitrogen. The film that was formed in the vial was then placed under vacuum overnight to remove residual solvent. Then, the thin film was hydrated by adding an aqueous solution containing the material to be encapsulated in the appropriate buffer solution. Then the solution was vortexed and extruded using a Lipex extruder (Northern Lipids Inc.) via 0.2 and then 0.1 μm Polycarbonate membranes until the solution became transparent (approx. 8-10 times for each membrane). The polycarbonate membranes were manufactured by GE Water & Process Technologies, and purchased from Tamar Laboratory Supplies Ltd., Israel. When vesicles were prepared by film hydration followed by sonication, the first steps of film formation and then hydration are similar to those described for the extrusion method, then, the hydrated film was further sonicated, using a probe sonicator (Vibra Cell Model H540/CV54, Sonics and Materials U.S.A). Probe sonication was carried out at 4° C. for 10-14 min (15 sec pulses and 10 sec rest).

Vesicle Characterization

The vesicles were characterized with respect to morphology (by cryo-transmission electron microscopy—cryoTEM), size and size distribution (by dynamic light scattering—DLS), surface charge (by Zeta potential analyzer) and stability (by fluorescent measurements).

Cryo-Transmission Electron Microscope (Cryo-TEM): Samples of vesicles (about 5-10 μL) were deposited on 300-mesh holey carbon cupper grids (Ted Pella, Inc. Redding, Calif.). A drop of 5 μl was applied to the grid and blotted with a filter paper to form a thin liquid film of the solution. Grids were rapidly plunged into a liquid ethane bath cooled with liquid nitrogen and maintained at a temperature of approximately −170° C. using a cryo-holder. The samples were imaged at −180° C. using a FEI Tecnai 12 G2 TEM, at 120 kV with a Gatan cryo-holder maintained at −180° C. Images were recorded with the Digital Micrograph software package, at low dose conditions, to minimize electron beam radiation damage, at the Ilse Katz Institute for Nanoscale Science and Technology of Ben-Gurion University (“BGU”).

Dynamic light scattering (DLS): Vesicle size and homogeneity was determined by DLS using HPPS-NIBS, light scattering apparatus (ALV-Laser, Langen, Germany) with the laser powered at 3 mW HE-Ne laser line (632.8 nm), at the Ilse Katz Institute for Nanoscale Science and Technology of BGU. Standard vesicle solutions were diluted 1:10 and loaded into a cuvette for light scattering measurements. The measurements were conducted at an angle of 173°, during 30-180 seconds, from different positions of the cell in order to avoid measurements of multiple scattering.

Zeta-potential measurements: Particle size and zeta potential were measured by using zeta potential and Particle size analyzer, ZetaPlus, (Brookhaven Instruments Corporation Ltd, NY, USA), in the range of 10-1000 nm, in the Chemistry Department of BGU. Vesicle solutions were diluted 1:10 in appropriate buffers and loaded into a 4 ml cuvette for light scattering measurements. The measurements were conducted at an angle of 90°, at 10 repeated measurements, and zeta potential was estimated as an average of 5 repeated readings.

Vesicle stability: To determine vesicle stability, samples of carboxyfluorescein (CF)-loaded vesicles (see below method for loading vesicles with CF and determination of percent encapsulation) were incubated in PBS and percent encapsulation was determined at different times. For the measurement of vesicle decapsulation by acetylcholine esterase (AChE), the fluorescence of a sample of intact CF-loaded vesicles was monitored in a quartz cuvette under constant stirring for a few minutes, until a stable fluorescence reading was obtained, and then, AChE (2 μl containing 2 units) was added and the fluorescence measurement continued for additional 5 min. At this point Triton X100 (0.15% final concentration) was added to break the remaining vesicles and to obtain the total fluorescence of the encapsulated CF.

To determine the stability of vesicles with encapsulated protein, vesicle samples were incubated in PBS and the percent encapsulation was determined at different times. Stability was also determined by changes in the vesicle size (by DLS, as was described in previous sections) at various time points after vesicle preparation.

Encapsulation Experiments

Encapsulation was achieved by including the material to be encapsulated in the hydration buffer during the hydration stage of the vesicle preparation (see above). After the vesicles were formed and the material in the hydrating buffer was encapsulated, non encapsulated material was removed over a Sephadex G-50 column (for details see below). Encapsulation efficiency was determined initially with CF as described in Popov et al [10,13]. Briefly, the encapsulation capacity of CF was assessed by measuring the fluorescence intensity (at excitation wavelength of 492 nm and emission wavelength of 517 nm) of CF-loaded vesicular preparation before and after disrupting the vesicular structure by Triton X100 at a final concentration of 0.15%. The released CF is dequenched and emits a fluorescent signal, which is quantified by comparing to a calibration curve. The encapsulation efficiency was calculated according to the following equation:

$$\frac{{R}_{A f} - {R}_{B}}{{R}_{A f}} \times 100 = \% Encapsulation$$

where R_{B} is the initial fluorescence reading and R_{Af} is the fluorescence reading after the addition of Triton X-100.

The encapsulation efficiencies of trypsinogen and GDNF were assessed by first running the sample through a Sephadex G-50 column to remove non-encapsulated protein from the encapsulated protein. The fractions obtained from the column (generally 0.5 ml per fraction) were treated by Triton X-100 reduced form to avoid interference with the absorbance reading of the encapsulated material at 280 nm. The concentration of trypsinogen or GDNF proteins was measured by either UV absorbance at 280 nm, or when the quantities of the proteins were low, the proteins were labeled with AlexaFluor®-488 (see procedure for labeling the proteins below) and their quantities determined by fluorescence spectroscopy. Percent encapsulation was determined by dividing the area under the curve of the vesicle fractions by the total area under the curve, which was the sum of the area under the curve of the vesicle preparation and the area under the curve of the free protein. With the use of a calibration curve the concentrations in each were determined.

To encapsulate biotinylated GDNF (GDNF-biotin) in the vesicles, the following procedure was used for 100 μg GDNF-biotin: The biotinylated GDNF is dissolved in 1 ml distilled water. Empty vesicles are prepared by film hydration followed by sonication, using formulation e, which is described in the section on vesicle preparation above. The GDNF-biotin solution is then added to 1 ml vesicle suspension and the solution is sonicated on ice to form vesicles made of 5 mg/ml of the basic bolaamphiphile with about 70μg of encapsulated GDNF-biotin (the encapsulation efficiency is about 70%).

Labeling of Trypsinogen and GDNF for Detection of Low Quantities

Since GDNF would be used at sub milligram range and its spectroscopic absorption could not be accurately measured at these concentrations, therefore, fluorescent tagging was investigated as a means of increasing the sensitivity of the determination of low quantities of the encapsulated protein. For labeling the protein, we used AlexaFluor®-488, which emits a strong and stable fluorescent signal. To label small quantities of the protein, we used a microscale Protein Labeling Kit (A30006) that was purchased from Invitrogen. For labeling we dissolved 20 μg of the relevant protein (either trypsinogen or GDNF) in sterile 0.1M Na₂CO₃ (total volume of 20 μl to form a concentration of 1000 μg/ml protein). Then, we added the reactive dye, which reacted with the protein to form AlexaFluor®-488-protein conjugate. To purify the labeled protein, we centrifuged the product over a resin provided by the manufacturer and the effluent contained the purified labeled protein, which can be determined quantitatively by fluorescent measurement at an excitation wavelength of 492 nm and an emission wavelength of 517 nm.

Purification of the vesicles from the non-encapsulated material

The vesicles were purified by size exclusion chromatography on Sephadex G50 columns. The eluting buffer for the routine vesicle purification was 16 mM NaCl in phosphate buffer pH 7.3 but other eluting buffers were used as described in the Results Section. The Flow rate used for the elution was 15 ml/hr. Column dimensions were 20cm×0.7 cm (length and diameter, respectively). The volume of each fraction collected from the column was 0.5 ml (equal to 1-2% of the total column volume). Optical density or fluorescence of each fraction was measured to determine the concentration of the eluted material.

Determination of GDNF Activity

To determine the activity of GDNF, we measured the effect of 20 ng/ml hGDNF on the activation of the enzymes AKT and MAPK using SH-SYSY neuroblastoma cells (obtained from the ATTC). For this measurement, we plated the cells in 12 well plates coated with PEI at a density range of 3×10⁵-6×10⁵ cells/well 48 h prior to the bioassay. Cells were deprived of serum for 2.5 h, stimulated by the test material (plain medium as a control or free GDNF or encapsulated GDNF), washed once with ice-cold PBS and then lysed in 120 W/well sample buffer. The lysates were boiled, sonicated and centrifuged and then loaded onto 10% acrylamide gel for SDS-PAGE. After the electrophoresis, the samples were immune-blotted using antibodies against phospho-MAPK44/42 and against phospho-AKT.

Determining the Integrity of GDNF After Encapsulation within the Vesicles

Sodium dodecyl sulfate polyacrylamide gel electrophoresis (SDS-PAGE) was used to examine whether the encapsulation process affects the integrity of the encapsulated GDNF. The test samples were applied on a 5%−/15% SDS-polyacrylamide gel and electrophoresis was performed using a running buffer of 14.4 g glycine and 3 g Tris base per Liter with 1% SDS. The gels were then stained by Coomasie blue using the Bio-Safe Coomassie staining protocol and then destained for 30 min in water.

Determining Targeting of the Vesicles to Cultured Cells that Express the Dopamine Transporter (DAT)

Three cell lines were used to test the ability of the surface DAT ligand to target the vesicles to the dopamine transporter: (a) HeLa cells—human cervical cancer cells that do not express the dopamine transporter and were used as control cells. HeLa cells were grown in DMEM medium supplemented with 5% fetal bovine serum, 2 mM L-Glutamine, 100 IU/mL penicillin and 100 μg/mL streptomycin at 37° C. under humidified atmosphere with 5% CO₂. (b) PC-12 cells—derived from a rat pheochromocytoma and which highly express the dopamine transporter [19]. PC-12 cells were grown in RPMI-1640 medium, supplemented with heat-inactivated horse serum to a final concentration of 10%, fetal bovine serum to a final concentration of 5%, 2 mM L-Glutamine, 100 IU/mL penicillin and 100 μg/mL streptomycin at 37° C. under humidified atmosphere with 5% CO₂. (c) SH-SYSY human neuroblastoma cells that were reported to express the dopamine transporter [20]. SH-SYSY cells were grown in DMEM medium supplemented with 5% fetal bovine serum, 2 mM L-Glutamine, 100 IU/mL penicillin and 100 μg/mL streptomycin at 37° C. under humidified atmosphere with 5% CO₂. Vesicles are taken up by these cells after the vesicles adhere to the cell surface. Vesicles that contain DAT ligand on their surface will bind to cells that express the dopamine transporter. The binding of the vesicles to the cells facilitate the uptake and therefore, the extent of the internalization of the vesicles into the cells may be used as an index for targeting.

To measure uptake of the vesicles into the cells, vesicles were loaded with carboxyfluorescein (CF) and the cells were contacted with these CF-loaded vesicles. Uptake of the vesicles by the cells labeled the cells with the fluorescent marker encapsulated in the vesicles and the fluorescent cells identified by flow cytometry. Thus, to measure uptake of CF-loaded vesicles into the cells, cells were plated in 24 well plates at a density of 200,000 cells/well and after 24 hours, the the medium was replaced with a culture medium without serum and incubated in this medium for 30 min. Encapsulated or non-encapsulated CF were then added to the cells (free 0.1 μg CF or the same amount of CF encapsulated in 5 μg vesicles) and the cells were incubated for 3-5 hours. At the end of the incubation, the cells were washed with PBS and detached from the bottom of the well by trypson-EDTA. The cell suspension was analyzed by flow cytometry (FACS).The fluorescence intensity of the treated cells was done by the FlowJo software.

In Vivo Studies

Animals: Eight-week-old male ICR or 10-week-old male C57BL/6 mice, weighing between 25-30g, were maintained on standard mice chow and tap water ad lib. The mice were kept in 12 hours light/dark cycles at a temperature of 25±3° C. All the animal experiments were performed according to the protocol approved by the Animal Care and Use Committee of BGU, according to an approved protocol (#IL-24-04-2008).

Injection of Test Material

Unless otherwise stated, animals were pretreated 15 min before the injection of the test material by pyridostigmine (o.5 mg/kg, i.m.) to inhibit peripheral ChE and thus, reduce vesicle decapsulation in the blood circulation before they enter the brain. The test material was injected i.v. into the tail vein in a volume of 100-200μ1 per mouse.

Determination of CF Concentrations in Tissues After Injecting CF-Loaded Vesicles into Mice

The test material (either free CF or encapsulated CF) was injected into the tail vein of mice in a volume of up to 200 μl. The quantity of encapsulated CF was always determined prior to the administration and similar amounts of either encapsulated or free CF were injected. At 30 minutes after the injection, mice were anesthetized by Xylazine-Ketamine and blood was withdrawn through cardiac puncture, the mouse was perfused with 10 ml PBS and tissues were dissected out. The specimens were homogenized in PBS, deproteinated by 5% (final concentration) of tricholoro acetic acid (TCA), centrifuged and the supernatants were used for fluorescence measurements.

Tissue Distribution of Trypsinogen AlexaFluor®-488 Conjugate After i.v. Injection

Trypsinogen—AlexaFluor®-488 was injected i.v. in its free form or encapsulated in the bolavesicles. At 30 min after the injection, mice were anesthetized by Xylazine-Ketamine mixture and blood was withdrawn through cardiac puncture, the mouse was perfused with 10 ml PBS and tissues were dissected out. The dissected tissues were attached to labeled paper stripes, frozen in isopentane cooled over liquid nitrogen, and stored at −80° C. The selected tissues were cryosectioned and the fluorescence of the sections was analyzed using confocal fluorescent microscopy. All images were acquired using the same imaging settings and were not corrected or modified. Fluorescence of slices from different organs was quantified by imaging software after subtracting background fluorescence. In effect, for quantifying the fluorescence in the tissue, the average fluorescence obtained from the control mice, which were injected with PBS instead of the fluorescent material was subtracted from the fluorescence values obtained from the same tissue taken from animals that received Trypsinogen-AlexaFluor®-488. At least 4 slices from each organ of each mouse were used for the quantitative analysis and each group contained 4-5 mice.

Distribution of Delivered Biotinylated GDNF (GDNF-Biotin) in the Brain

Mice were injected with the test material that contained either non-encapsulated GDNF-biotin, or encapsulated GDNF-biotin following the procedure that was described above for the labeled trypsinogen, including the procedure for the collection of the tissues. The frozen brains were cryosectioned and then, stained with DAPI and with avidin-AlexaFluor®-488. The details of the staining are as follows: AlexaFluor®-488 was added to the sections and after a few minutes, the sections were washed 3 times with PBS. Then, 20 μl of DAPI solution was placed on each section and incubated at room temperature for 10 min. The sections were then washed twice with PBS and wiped gently around the tissue with a paper towel. At this stage, the sections were left to dry in the air. After the staining procedure, the sections were mounted on slides using Mowiol-based mounting solution and fluorescence of the images (3-4 images per each section) were taken, using the CF and the DAPI channels of the confocal microscope.

Statistical analysis

The Significance of the Differences Between the Experimental Groups was Analyzed Using the Student t-test.

### EXAMPLE 12

### Synthesis of Bolaamphiphiles (Bolas)

Synthesis of Basic Bolas

Bolas GLH-19 and GLH-20 were synthesized and used as the basic building blocks for the preparation of vesicles. Briefly, the carboxylic group of vernolic acid (compound 1 in Scheme 1) was reacted with aliphatic diols to obtain the bisvernolester 2 (Scheme 1, below). This bisvernolester is the skeleton for both bolas GLH-19 and GLH-20. Then, the epoxy group of the vernolate moiety, located on C12-C13 of the aliphatic chain of vernolic acid, was used to introduce two ACh headgroups on each side of the alkyl chain, via one of the two vicinal carbons obtained after opening of the oxirane ring. For GLH-20 (Scheme 2, below), the ACh head group was attached to the vernolate skeleton through the nitrogen atom of the choline moiety. The attachment of the head group was carried out in a two-stage synthesis: First, the epoxy ring of the bisvernolester was opened with an excess of chloroacetic acid in toluene at 85° C. and, in the second stage, we performed quaternization using a threefold excess of the N,N-dimethylamino ethyl acetate. After removing the excess of the amine, the crude product was purified by flash chromatography with acetonitrile:water as the eluent. The mass spectrum, ESI-MS calculated for C₆₂H₁₁₄N₂O₁₄Cl₂: ([M-2Cl⁻]⁺/2=555.5, of the bola GLH-20 is shown in FIG. 1A:

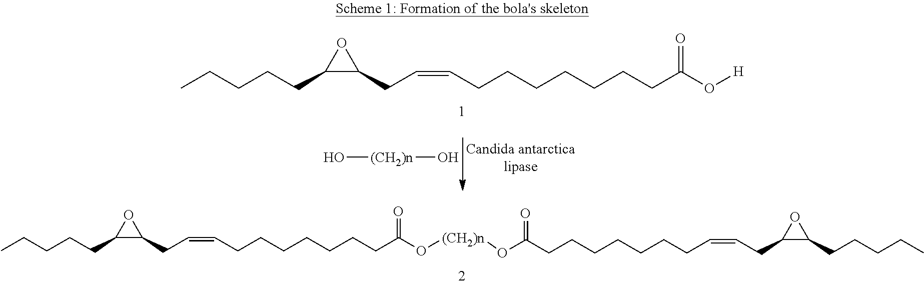

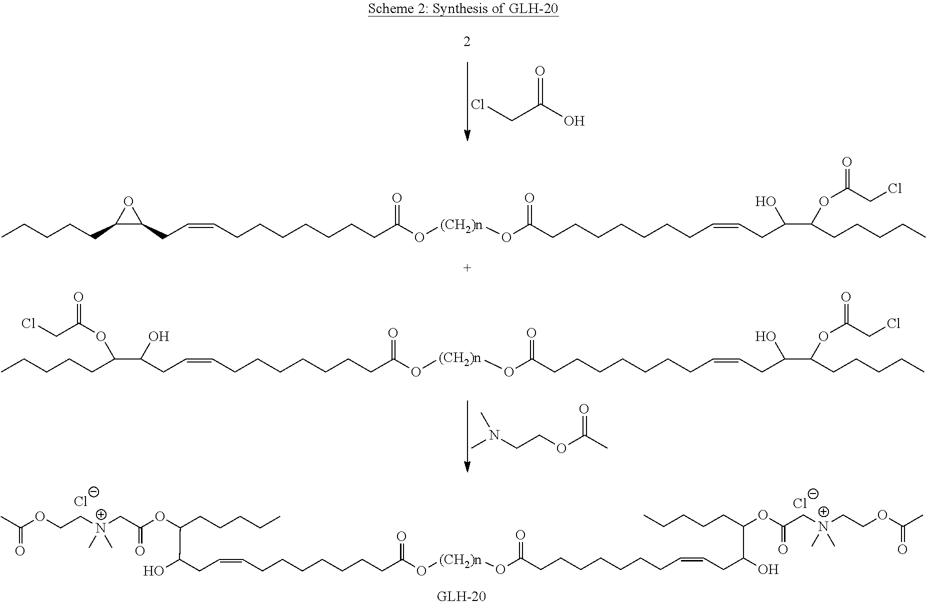

For GLH-19 (Scheme 3, below), a bola with an ACh head group attached to the vernolate skeleton through the acetyl group, the compound was prepared in a three-stage synthesis, including opening of the epoxy ring with glutaric acid, then esterification of the free carboxylic group with N,N-dimethyl amino ethanol and the final product was obtained by quaternization of the tertiary amine, with methyl iodide followed by exchange of the iodide ion by chloride using an ion exchange resin. The mass spectrum for GLH-19 calculated for C₆₆H₁₂₂N₂O₄Cl₂, ESI-MS/MS (positive mode) m/z: [M+]/2=583.6 (z=2) is shown in FIG. 1B.

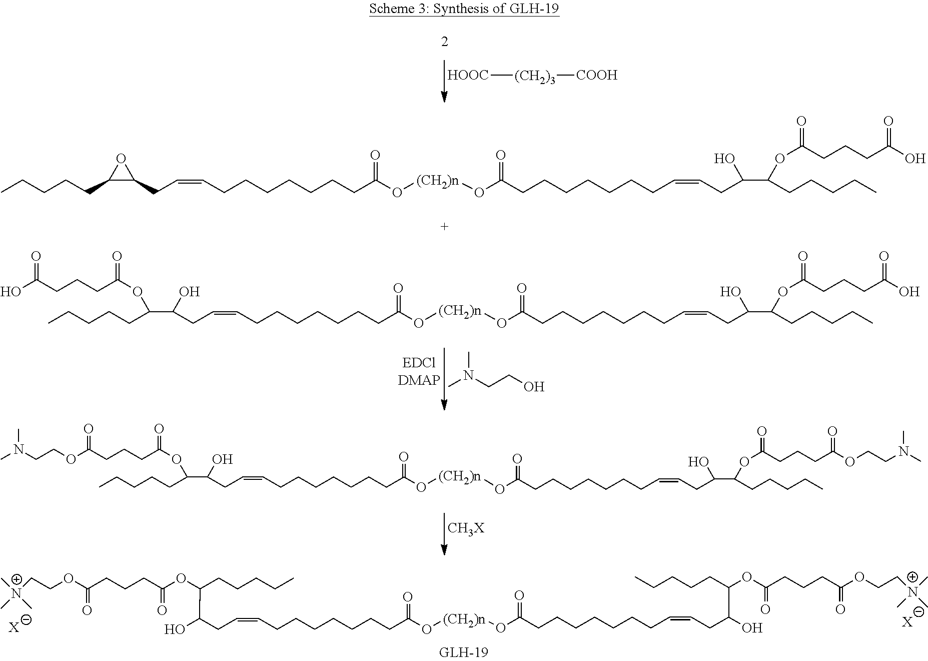

Synthesis of a Bola with Chitosan (CS) Head Group.

Depolymerization of chitosan as a first step for the synthesis of bolaamphiphile with chitosan head group. To enhance penetration of the vesicles into the brain, they were “decorated” on their surface with chitosan (CS) head groups. This was accomplished by incorporating into the vesicle formulation a bola with a novel, chitosan-containing head group.

The synthesis of this new bolaamphiphile included the following: (1) preparation of a low molecular weight chitosan (LMWCS), (2) synthesis of an asymmetric bola skeleton, and (3) binding the head groups to the skeleton. The starting material for the preparation of LMWCS is commercial chitosan, which is of high-molecular weight, and is insoluble in water and organic solvents. The LMWCS, which has improved water solubility, could be obtained by a depolymerization reaction of the commercial high molecular weight chitosan, using hydrogen peroxide (H₂O₂), which is a strong oxidant that produces free radicals, which can attack the β-D-(1-4)-glycosidic bond and depolymerizes chitosan. Oxidative depolymerization of chitosan by heterogeneous treatment of commercial high-molecular chitosan (MW˜50 kDa) with hydrogen peroxide, was accomplished by a dropwise addition of 30% hydrogen peroxide solution to chitosan dispersed in water at 60° C. for 6 h. The filtrate, after separation of insoluble fragments, was evaporated and precipitated by ethanol to obtain LMWCS, providing, e.g., 2 g of the LMWCS using the method described above.

The FT-IR Spectrum of the LMWCS

The FT-IR spectrum of the LMWCS (FIG. 2) exhibited most of the characteristic absorption peaks of the original chitosan with some differences. The vibrational band at 1154 cm⁻¹, which corresponds to the ether bond between the pyranose rings, was weakened, indicating rupture of the β-glucosidic bonds in the molecular chain of chitosan. The band at 1596 cm⁻¹ in LMWCS becomes stronger than that of the original chitosan, suggesting that the content of amino groups and correspondingly, the degree of deacetylation (DD) changed. The decrease in the NH₂ (pH-potentiometric titration of amino groups) content in LMWCS may be explained by the presence of “other groups”. These “other groups” may be titrated together with the amino groups, for example, carboxylic groups. The carboxylic groups of the LMWCS were determined by a direct titration with sodium hydroxide. In fact, LMWCS contained 1.05-1.15 mmol carboxylic groups per gram of chitosan (Table 2), below:

TABLE 2
Chemical analysis of original and LMWCS

| Chitosan | %, C | %, H | %, N | N/C | COOH, mmol/g |
|---|---|---|---|---|---|
| Original | 41.22 | 7.15 | 7.34 | 0.178 | 0 |
| LMWCS | 38.88 | 6.63 | 6.12 | 0.157 | 1.14 |

Table 2 also shows that the depolymerization of the commercial chitosan led to a decrease in nitrogen, carbon and hydrogen contents, suggesting an increase of oxygen content. The mass ratio N/C decreased, confirming the loss of nitrogen as the result of the depolymerization. Thus, the depolymerization of chitosan led not only to the rupture of the I-glucosidic bonds, but also to a change in the chemical structure of the original chitosan and possibly, to the formation of carboxylic groups. This result correlates with data previously presented in the literature [23,24] that show the effect of H₂O₂ treatment on chitosan.

MALDI-TOF Mass Spectrometry of LMWCS

The composition of the LMWCS chitosan was also analyzed by MALDI-TOF mass spectrometry. The analysis of the mixture of oligomers obtained by the depolymerization of the original chitosan was performed in a positive-ion mode. Table 3 (below) shows that the depolymerization of the original chitosan led to the formation of oligomers with a degree of polymerization (DP) between 3 and 8. The peaks correspond to oligomers carrying fragments of deacetylated (GLcN) and acetylated (GLc NAc) chitosan. Deacetylated chitosan (GlcN) contains a repeat unit of C₆H₁₁O₄N, with a MW of 161 Da and the acetylated chitosan contains a repeat unit of C₈H₁₃O₅N, with a MW of 203 Da.

TABLE 3
Assigned ion composition of LMWCS, determined by MAILDI-TOF
analysis (Solvent CH₃CN, H₂O)

| m/z | Types | Ion Composition |
|---|---|---|
| 524.2 | [M + Na]⁺ | (GlcN)₃ |
| 539.6 | [M + K]⁺ | |
| 566.1 | [M + Na]⁺ | (GlcN)₂—(GlcNAc) |
| 582.1 | [M + K]⁺ | |
| 624.1 | [M + K]⁺ | (GlcN)—(GlcNAc)₂ |
| 685.1 | [M + Na]⁺ | (GlcN)₄ |
| 727.3 | [M + Na]⁺ | (GlcN)₃—(GlcNAc) |
| 743.1 | [M + K^{]+} | |
| 769.1 | [M + Na]⁺ | (GlcN)₂—(GlcNAc)₂ |
| 811.1 | [M + Na]⁺ | (GlcN)—(GlcNAc)₃ |
| 846.2 | [M + Na]⁺ | (GlcN)₅ |
| 888.2 | [M + Na]⁺ | (GlcN)₄—(GlcNAc) |
| 930.2 | [M + Na]⁺ | (GlcN)₃—(GlcNAc)₂ |
| 946.2 | [M + K]⁺ | |
| 972.2 | [M + Na]⁺ | (GlcN)₂—(GlcNAc)₃ |
| 988.2 | [M + K]⁺ | |
| 1014.1 | [M + Na]⁺ | (GlcN)—(GlcNAc)₄ |
| 1091.2 | [M + Na]⁺ | (GlcN)₄—(GlcNAc)₂ |
| 1133.2 | [M + Na]⁺ | (GlcN)₃—(GlcNAc)₃ |
| 1175.3 | [M + Na]⁺ | (GlcN)₂—(GlcNAc)₄ |
| 1252.3 | [M + Na]⁺ | (GlcN)₅—(GlcNAc)₂ |
| 1294.3 | [M + Na]⁺ | (GlcN)₄—(GlcNAc)₃ |
| 1455.3 | [M + Na]⁺ | (GlcN)₅—(GlcNAc)₃ |
| 1497.8 | [M + Na]⁺ | (GlcN)₄—(GlcNAc)₄ |
| 1540.0 | [M + Na]⁺ | (GlcN)₃—(GlcNAc)₅ |
| 1658.3 | [M + Na]⁺ | (GlcN)₅—(GlcNAc)₄ |

### EXAMPLE 13

### Synthesis of an Asymmetric Bola with a Chitosan Head Group

This example describes the synthesis of an asymmetric bola with an acetylcholine (ACh) head group on one side of the bola's skeleton and a CS head group on the other side of the bola's skeleton (bola-CS). The rationale behind such a bola comes from packing parameters considerations. The ACh head group on the bola-CS is smaller than the CS head group and is similar to the ACh head groups of the symmetrical bolaamphiphile GLH-20. Thus, during aggregation, the ACh head group of the bola-CS is expected to be situated on the inner membrane surface of the vesicle, together with one of the ACh head groups of GLH-20 and GLH-19 (which also has an ACh head group, but attached in a different way to the hydrophobic skeleton). The CS head group will thus become an outer surface moiety and will be free to interact with the endothelial cells of the BBB, thus enhancing BBB permeability of the vesicles. The synthesis of this asymmetric bola-CS is a multi stage process that is depicted in Scheme 4, below.

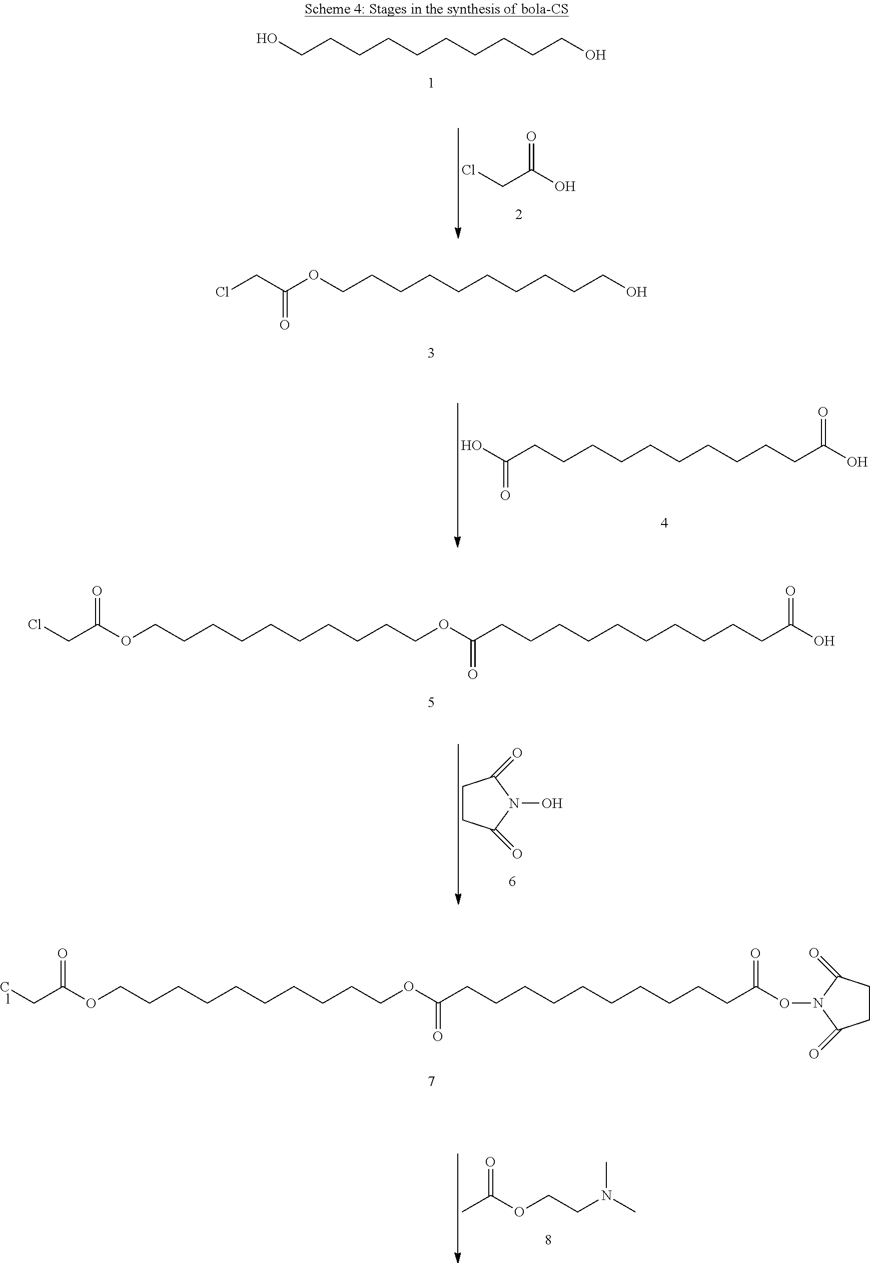

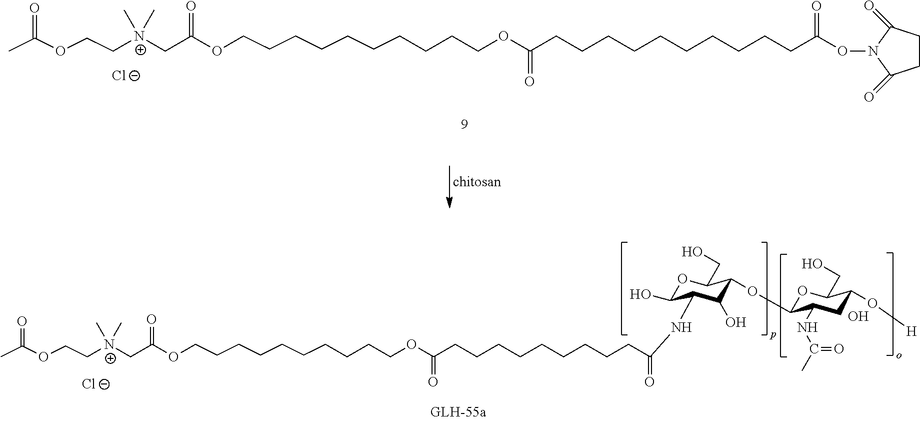

For the synthesis of symmetric bolas, such as GLH-19 and GLH-20, the strategy followed ws to to form the bolaskeleton first and then attach the head groups to both ends of the hydrophobic chain. This strategy was revised for the synthesis of an asymmetric bola as follows:

Stage 1: For the asymmetric bola GLH-55a, the synthesis began with the formation of monochloroacetate of decanediol 3 through the esterification of 1,10-decanediol 1 with chloroacetic acid 2 at a molar ratio of 5:1 respectively (Scheme 4). The reaction was carried out in toluene by azeotropic distillation in the presence of Amberlyst 15 as a heterogeneous acidic catalyst that can be easily removed by filtration at the end of the reaction, avoiding the tedious work needed to neutralize the soluble acidic catalyst. ¹H NMR of the product displayed the characteristic bands attributed to the new chloroacetate moiety: a singlet at 4.05 ppm arising from the chloromethylene protons (CH₂Cl) and a triplet at 4.17 ppm arising from the methylenic protons of the ester group (CH₂—O—CO). The corresponding chemical shifts in ¹³C-NMR spectrum appeared at 40.95 ppm (CH₂Cl) and 66.42 ppm (CH₂—O—CO). The carbonyl signal appeared at 167.45 ppm.

Stage 2: The second step of the synthesis (Scheme 4, above) includes the elongation of the hydrophobic chain. The intermediate 5 was obtained by reacting the monochloroacetate of decanediol 3 with a dicarboxylic acid 4 (1,10-decanedicarboxylic acid) at a molar ratio of 1:5, respectively. The reaction mixture was refluxed in toluene with constant removal of water by azeotropic distillation and was catalyzed by Amberlyst 15. The crude product was purified over a silica gel flash chromatography. The chloroacetate intermediates were then characterized by ¹H and ¹³C NMR spectroscopy (Table 4, below).

TABLE 4
¹H and ¹³C NMR data for derivative 5

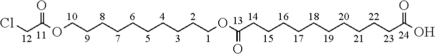

| | δ_{H} | ¹H NMR | ¹³C NMR |
|---|---|---|---|
| Group no. | (ppm) | Multiplicity | δ_{C} (ppm) |
| 1 | 4.05 | t | 64.40 |
| 10 | 4.17 | t | 66.42 |
| 11 | — | — | 167.46 |
| 12 | 4.06^{a} | s | 40.95 |
| 13 | — | — | 174.10 |
| 14 | 2.34 | t | 33.62 |
| 23 | 2.29 | t | 34.40 |
| 24 | — | — | 179.23 |

^{a}overlapping with the triplet at 4.05

Stage 3: The next step was the preparation of an active ester of the carboxylic acid with N-hydroxysuccinimide (NHS). The active N-hydroxy-succinimide of the ester of the carboxylic acid was synthesized by reacting intermediate 5 (Scheme 4) and N-hydroxy-succinimide in the presence of a coupling agent (dicyclohexylcarbo-diimide DCC) at room temperature by the method of [25]. The pure intermediate 7 was isolated by flash chromatography on silica gel with hexane-ether as the eluent. The structure of product was confirmed by FT-IR and NMR spectroscopy. The chemical shift of the protons of the methylene group, CH₂—CO—N, of the NHS in intermediate 7 appeared at 2.83 ppm and the carbon at 25.60 ppm, respectively.

Stage 4: In order to attach the acetylcholine head group, the chloro derivative obtained in the previous stage was reacted with intermediate 7, that will serve as the alkylating agent with a small excess of the tertiary amine, N,N-dimethylaminoethyl acetate. The reaction was carried out in acetone as the solvent at the reflux temperature for about 8 hours. The progress of the reaction was followed by TLC and HPLC. The reaction mixture was washed several times with diethyl ether to remove the excess of the unreacted amine. The degree of quaternization of the amphiphile intermediate 9 was about 95-98%, as determined by argentometric titration. The molecular weight, as determined by electrospray ionization mass spectrometry (ESI -MS), was 655.41 (690-Cl⁻). In the FT-IR spectra, the absorption bands characteristic of the chloroacetate ester group disappeared, and a new absorption band appeared at 1237 cm⁻¹; this is the so-called “acetate band”.

Stage 5: This stage constitutes the conjugation of the low molecular weight chitosan (LMWCS) (prepared as described above) with the bolaskeleton 9 (Scheme 4) containing already the cationic head group at one end, and the N-hydroxysuccinimide ester at the other end. The conjugation was performed by adding the solution of intermediate 9 in DMSO to the solution of LMWCS and triethylamine in DMSO. The molar ratio LMWCS to the activated ester 9 was 10:1. The reaction mixture was stirred for 72 h at RT. The solution was lyophilized. The yellow powder obtained was triturated several times with ether and ethanol, to remove the unreacted intermediate 9, filtered and dried. The obtained product, GLH-55a, is the bolaamphiphile, having the chitosan head group on one side and the acetyl choline head group on the other side of the bolaskeleton, was soluble in DMSO and water.

When the FT-IR spectrum of bola GLH-55a is compared with the spectrum of intermediate 9, the disappearance of the absorption bands at 1784 and 1814 cm⁻¹, characteristic of the N-hydroxysuccinimide group is noticeable. Also noticeable is the appearance of new absorption bands at 1564 cm⁻¹, characteristic of the amide bond, which was formed between the amino group of chitosan and the active ester (NHS ester) of intermediate 9. Additional absorption bands, at 1740 cm⁻¹ for the ester group, and 1247 cm⁻¹ for the acetate group, are also the result of the conjugation.

Table 5 and Table 6 present the chemical shifts of the final bola GLH-55a. As can be seen, the chemical shifts of the original LMWCS could also be found in the modified CS (marked with a star). In ¹³C-NMR spectrum, the new signals at 52.56 ppm [N⁺(CH₃)₂], 58.22 ppm [N⁺—CH₂—CH₂] 62.62 ppm [N⁺—CH₂—CH₂], 61.40 ppm [CO—CH₂−N⁺] indicate the formation of conjugation product.

In the ¹H-NMR spectrum of the asymmetric bolaamphiphile with a chitosan and acetyl choline head group GLH-55a, we could again find the chemical shifts characteristic of the starting LMWCS (marked with a star). The new signals at 3.85 ppm [N⁺(CH₃)₂], 4.40 ppm [N⁺—CH₂—CH₂], indicate the formation of conjugation product.

TABLE 5
¹³C-NMR spectrum of GLH-55a (DMSO)

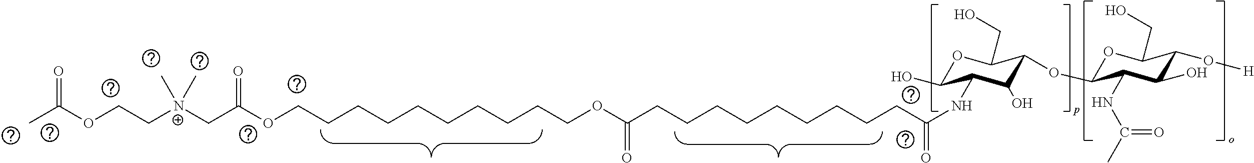

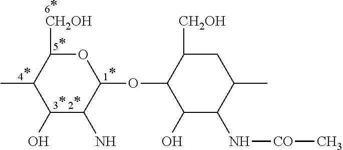

| No | Carbon in the group | δ, ppm |
|---|---|---|
| 1* | Cl | 102.46 |
| 2* | C2 | 57.30 |
| 3* | C3 | 70.45 |
| 4* | C4 | 78.50 |
| 5* | C5 | 75.40 |
| 6* | C6 | 60.91 |
| 7* | CH₃CO | 19.02; 21.03 |
| | | 23.43 |
| 1 | (CH₂)ₙ | 29.30-25.0 |
| 2 | CH₃—CO | 19.78 |
| 3 | NH—CO—CH₂ | — |
| 4 | CH₂—CO—O | 34.10 |
| 6 | N⁺(CH₃)₂ | 52.56 |
| 7 | CH₂—O | 64.52 |
| 8 | CH₂—O | 66.27 |
| 9 | N⁺—CH₂—CH₂ | 62.62 |
| 10 | N⁺—CH₂—CH₂ | 58.22 |
| 11 | CO—CH₂—N⁺ | 61.40 |
| 12 | CH₂—CO—OCH₂ | 174.63; 175.60 |
| 13 | CO—CH₃ | 173.43 |
| | | 173.27 |
| 16 | O—CO—CH₂—N⁺ | 170.13; 169.0 |
| 17 | NH-CO | 169.44 |
| | | 165.41 |

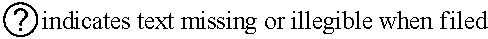

TABLE 6
¹H-NMR spectrum of GLH-55a

| | | δ, ppm | |
|---|---|---|---|
| No | Proton in the group | d₆-DMSO | D₂O |
| 1* | H1 | 4.51 | 4.63; 4.55 |
| 2* | H2 | | |
| 3*, 4*, 5*6* | H3, H4, H5, H6 | | 3.60-3.90 |
| 7* | H7 | | 2.0 |
| 1 | (CH₂)ₙ | 1.60-1.22 | |
| 2 | CH₃—CO | 2.07 | |
| 3 | CO—CH₂ | 2.16 | |
| 4 | CH₂—CO—O—CH₂ | 2.25 | |
| 6 | N⁺(CH₃)₂ | 3.85 | |
| 7 | CH₂—O | 3.98 | |
| 8 | CH₂—O | 4.16 | |
| 9 | N⁺—CH₂—CH₂—O | 4.40 | |

MALDI-TOF Mass Spectrometry of the Bolaamphiphile GLH-55a:

The structure of this bolaamphiphile having the quaternary acetyl choline head group on one side of the hydrophobic skeleton and the chitosan head group on the other side of the hydrophobic skeleton was investigated by MALDI-TOF mass spectrometry. Positive ion MALDI spectrum was acquired in a reflector mode, using DHB (2,5-dihidroxybenzoic acid as matrix). MALDI-TOF-MS was taken over a mass range of 500-2200. The analysis of the MALDI spectrum of this bola showed the appearance of new peaks between 1000-2000 Da, including the polymer unit of the MW 701 Da. This new polymeric unit, which contains the bola with the acetyl choline as one head group and the chitosan as the second head group, has a MW of 736 Da. The repeat unit in the MALDI spectrum was 701 Da (the polymeric unit without the chloride anion has a MW of 701 (736-Cl=701) presented below:

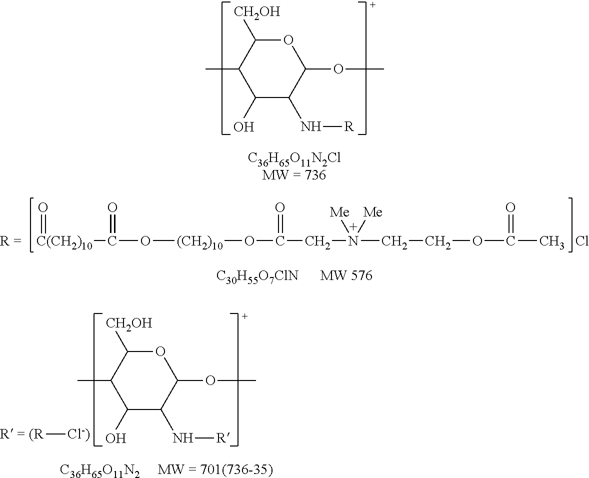

These results are in agreement with the data published in the literature for quaternary ammonium salts with a low molecular weight.

Synthesis of the Bolaamphiphile with DAT Ligand Head Ggroups

These examples describe the at synthesis of a dopamine transporter, β-CFT (3β-(4-fluorophenyl)tropane-2β-carboxylic acid methyl ester), also known as WIN 35, or 428, that is one of the most potent congeners at [³H] cocaine binding sites in striatum [26]. This has been used for imaging in humans as a marker of the nigrostriatal pathway to assess the severity of Parkinson's disease (PD) [27]. This ligand was selected, in part, for its high affinity to dopaminergic cells, as an illustrative surface group for targeting the vesicles to dopaminergic neurons in the Substantia Nigra.

Commercially-available (100 mg) of β-CFT was used as a reference compound while, for the purpose of synthesizing the bola-DAT, synth β-CFT was synthesized as described. The β-CFT ligand was attached to the bolaskeleton described herein by modifiying the β-CFT ligand to perform the alkylation of the amino group of the ligand with the bromoacetate derivative of the bola skeleton. We started the synthesis of the modified β-CFT ligand from cocaine HCl by following the procedures of Clarke et al and Melzer et al. [28,29].

### EXAMPLE 14

### Synthesis of the β-CFT Derivative

Cocaine.HCl was neutralized with ammonium hydroxide, extracted with diethyl ether, and the solvent was removed under reduced pressure. GC-MS m/z [M]⁺=303 and NMR spectroscopy confirmed the structure of cocaine.

Stage 1 encmpasses the three steps ((a)-(c)) described below.

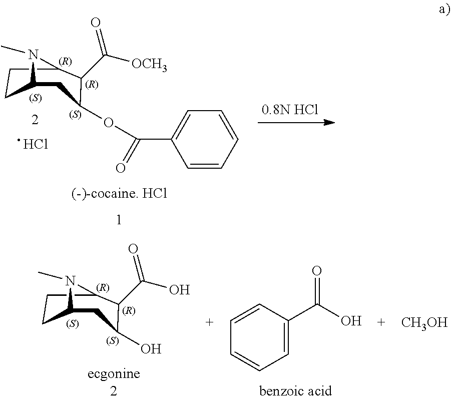

The cocaine hydrochloride 1 was refluxed with dilute hydrochloric acid. The progress of the reaction was followed by TLC. After cooling, the aqueous solution was extracted with ether to remove benzoic acid. The aqueous phase was concentrated to dryness to give ecgonine 2 as a viscose brown oil that gave only one spot on TLC (EtAc:MeOH:H20:25% NH₄OH 85:10:3:1) and was used for the next stage without further purification

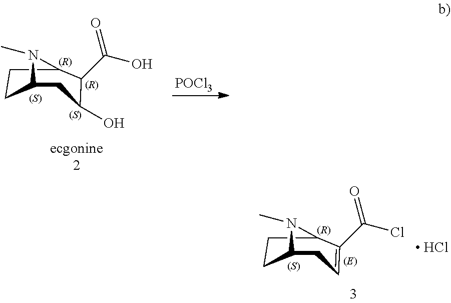

Ecgonine (2) and POC13 were refluxed for lh. The excess of POC13 was removed under reduced pressure. FT-IR spectrum of the residue 3 showed the appearance of the double bond at 3032 cm⁻¹, and the acyl chloride C═O(C₁) absorption peak at 1735 cm¹.

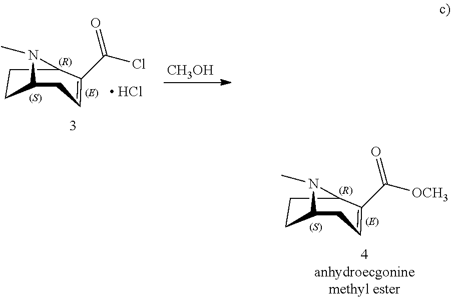

The product (3) was cooled in a dry ice/acetone bath to −73° C. and esterified with methanol,.to obtain the crude anhydroecgonine methyl ester hydrochloride. The procedure was followed by TLC (the same eluent system as mentioned above). The excess of methanol was removed under reduced pressure, the product neutralized with 25% NH4OH and purified by flash chromatography, with a mixture of ethyl acetate: methanol 95:5 as the eluent. The anhydroecgonine methyl ester 4 was obtained in a 55.4% yield (from cocaine hydrochloride). The mass spectrum of the ester 4 calculated for the C₁₀H₁₅NO₂, GC-MS m/z [M]=181.

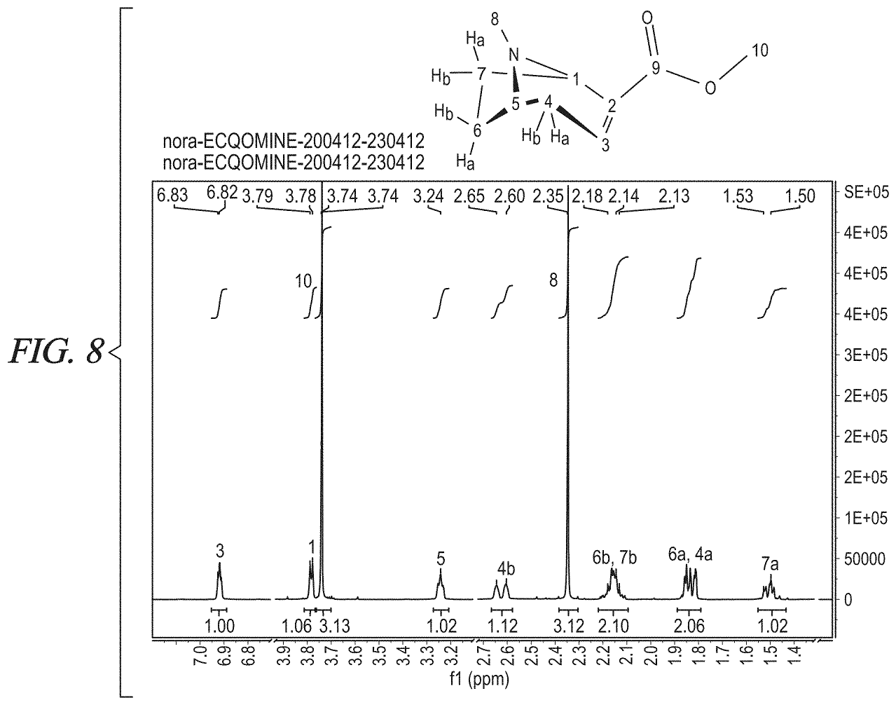

The FT-IR shows the presence of the ester group (COOCH₃) at 1711 cm⁻¹ The NMR spectrum allowed the different protons to be distinguished (FIG. 8).

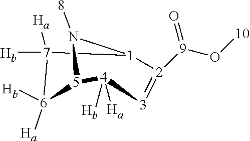

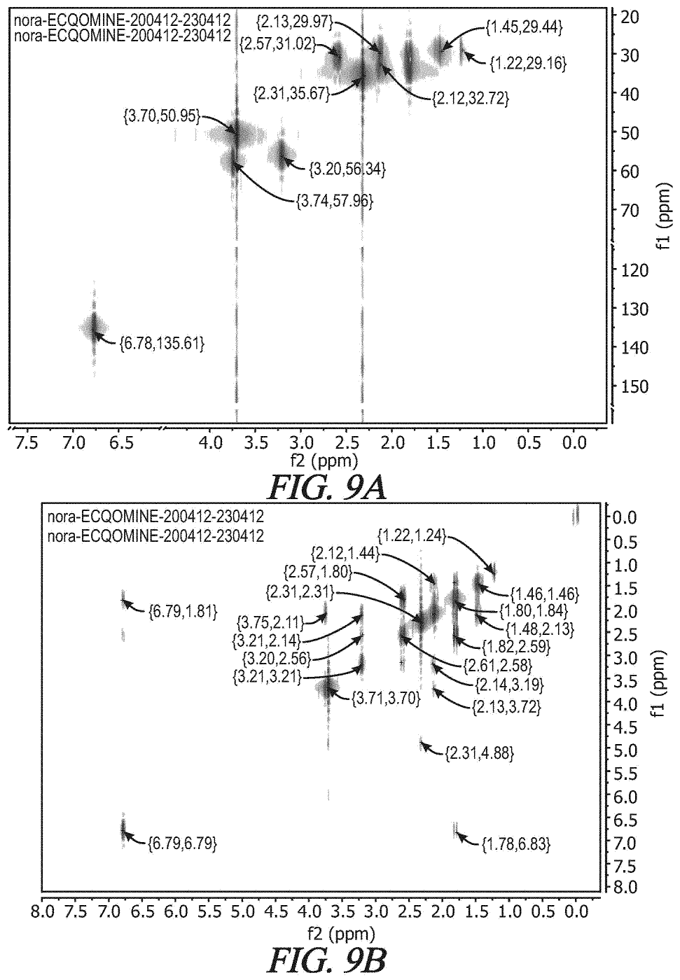

The difference between the protons of the methylene groups 4, 6 and 7 was elucidated with HMQC and ¹H COSY NMR [30,31] (FIG. 9). For example, the H-6a and H-4a overlap at 1.8-1.9 ppm and H-4b appears at 2.6 ppm as a doublet.

Stage 2 encompassed the following reaction:

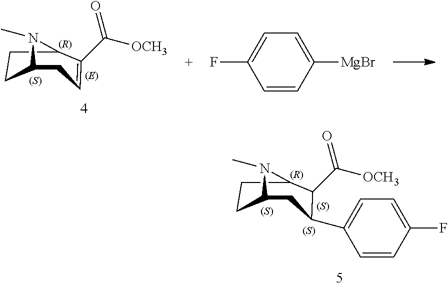

This stage involved the Michael addition of the aromatic Grignard reagent, (p-fluorophenyl) magnesium bromide, to the anhydroecgonine methyl ester 4. The methyl ester 4 in anhydrous ether was added drop wise to a mixture of the Grignard reagent in anhydrous ether at −30° C. under a stream of nitrogen.

The method of quenching can determine the relative distribution of the α- and β-carbomethoxy isomers. Since the a-isomer is biologically inactive, there was a need to optimize the yield of the β-carbomethoxy compound. The improved quenching procedure described herein uses the ethereal solution of hydrochloric acid that is added to the reaction mixture followed by addition of ice. The aqueous layer was basified to pH=10 with ammonium hydroxide and the product extracted with dichloromethane. A total yield of 52.5% of the α-and β-isomer was obtained. When the reaction was performed at −70°, 81.0% yield of α-and β-isomer was obtained. After removing the solvent, the crude mixture was checked by TLC and the GC-MS showed that the mixture contained 9.7% of the α-isomer and 53.8% of the β-isomer. The products were then separated by flash chromatography with a mixture of diethyl ether—triethyl amine as the eluent. The β-isomer (β-CFT) 5 was isolated in a 32.4-36% yield (based on 4) with 87-91% purity (determined by GC).

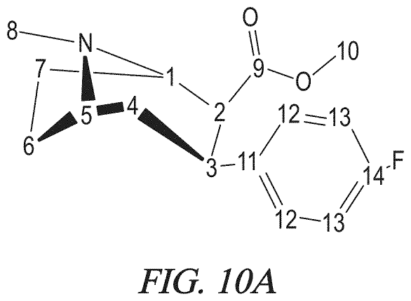

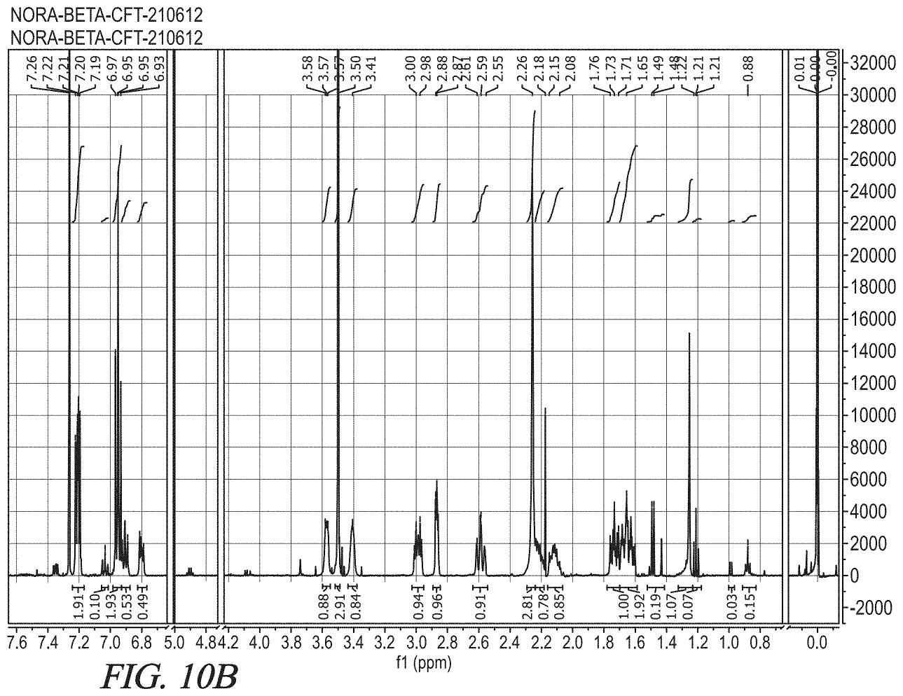

The NMR spectrum of the β-CFT 5 (FIG. 10) shows the disappearance of the double bond proton at 6.79 ppm of the starting methyl ester 4 and the appearance of the aromatic protons at 6.93-7.26 ppm and their corresponding carbons at 114,63, 114.8, 138.44, 138.44, 160.10, 162.12. The carbonyl carbon was shifted from 166.53 in 4 to 172.04 in 5

Stage 3 encompassed the following reaction:

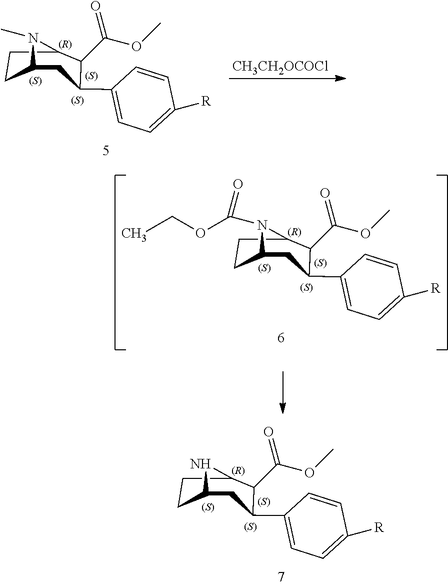

This reaction involves a demethylation reaction providing derivative 7 to be attached to the bolaskeleton. The N-demethylation of the N-methyltropane analog 5 was carried out by using α-chloroethyl chloroformate (ACE-Cl) [32,33]. The reaction of chloroformates with tertiary aliphatic amines provides a convenient method for promoting dealkylation. Compound 5 was reacted with ACE-Cl to provide the a-chloroethyl carbamate intermediate 6. The hydrolysis was then carried out with methanol to obtain the crude compound 7 in a 68.4% yield with a purity of 83% (GC). The mixture still contained 11.8% β-CFT and 4.2% byproducts. Purification by flash chromatography gave fluoro nortropane 7 in about 40% yield with a 98% purity. GC-MS m/z: [M]⁺=263.

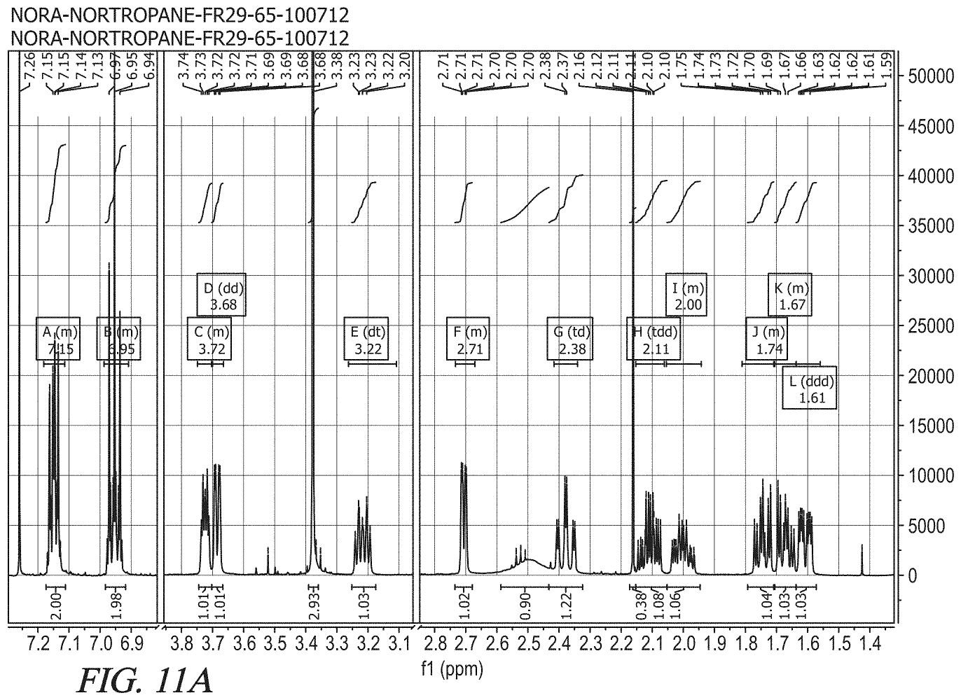

The ¹H (FIG. 11A) and ¹³C-NMR spectra (FIG. 11B) confirmed the structure; the peaks at 2.23 and 51.18 ppm in the proton and respectively, carbon NMR spectra (CH₃N) disappeared.

Synthesis of the Bolaskeleton

Synthesis of the bolaskeletons, GLH-19 and GLH-20 was described above. Attachment of the DAT ligand head group employed the decanedivernolate skeleton in which the opening of the epoxy ring was carried out with bromoacetic acid, as depicted in Scheme 5, below.

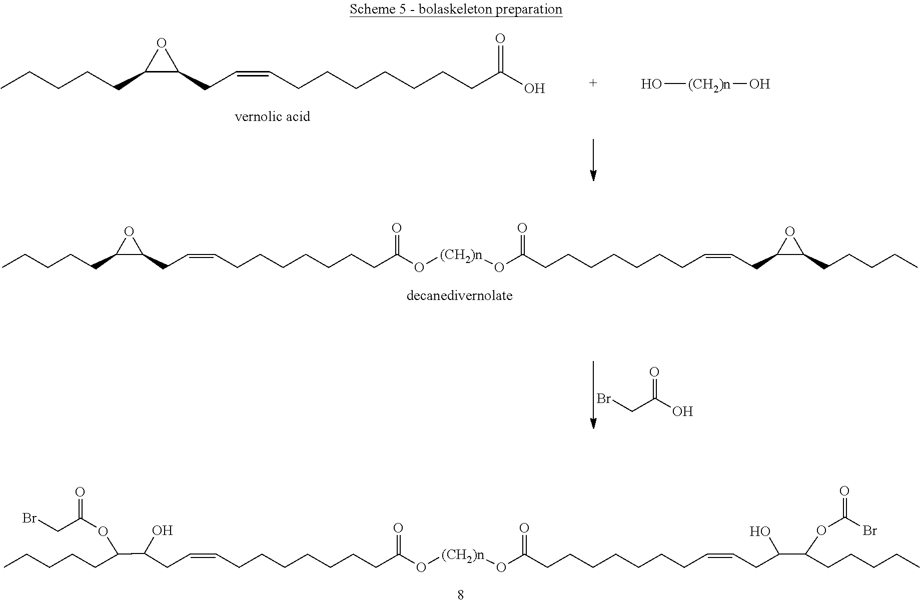

A mixture of decane divernolate with an excess of bromoacetic acid in toluene was heated for 24 h. The reaction mixture was washed several times with a saturated solution of sodium bicarbonate to remove the excess of bromoacetic acid, and the crude product was purified by flash chromatography, using a mixture of dichloroethane:acetone (50:1) to yield 33% of the dibromodiacetate of the divernolester 8, with 96.7% purity (HPLC). In the FT-IR spectrum, we observed a broad absorption peak at 1735 cm⁻¹ characteristic of both ester groups, contrary to the two absorption peaks, one of the original ester and the second one of the chloroacetate group, at 1735 and 1758 cm⁻¹, respectively for the dichlorodiacetate of the divernolester (Scheme 2, above). In the ¹H-NMR spectrum the bromomethylene protons (CH₂—Br) appear at 3.69-3.93 ppm. CH—OH and CH—OC(O) groups obtained after opening the epoxy ring appear at 3.60 and 4.90 ppm, respectively. The presence of structural isomers, I and II,

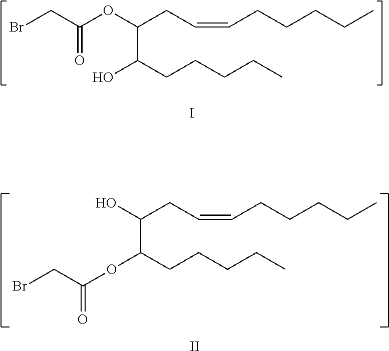

obtained after opening of the epoxy ring, was observed in the ¹H and ¹³C NMR spectra. ¹H-NMR showed two multiplets at 4.94-4.91 ppm and 4.89-4.86 ppm for the CH—O—CO proton and two overlapping triplets at 0.90-0.87 ppm for the terminal methyl group. ¹³C-NMR showed two peaks at 167.11 and 166.93 ppm for the carbonyl adjacent to the bromomethylene group BrCH₂C═O, four peaks at 133.82, 133.60 and 124.08, 123.31 ppm for the double bond and two peaks at 72.03 and 71.88 ppm for the CH—OH carbon

### EXAMPLE 15

### Attachment of the Fluoro Nortropane (7) to the Bola Skeleton

Conjugation of the fluoronortropane 7 to the bolaskeleton dibromodiacetate 8 (Scheme 6), was carried out using the alkylation reaction described by Riss [34] with a short chain alkyl halide.

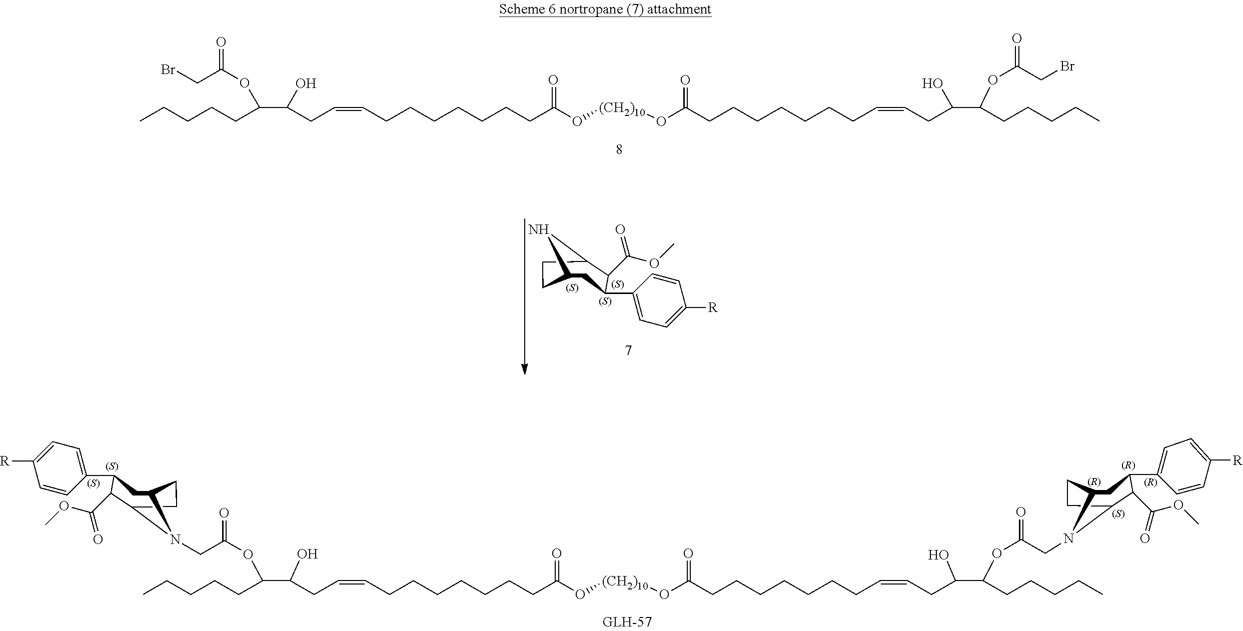

The reaction was performed in acetonitrile as the solvent, in the presence of a strong base, proton sponge or Hünig's base, using a molar ratio of 2:1 of the reagents, respectively. The reaction mixture was refluxed overnight. The progress of the reaction was followed by TLC. The hydrobromide of the base was filtered out, and the solvent removed under reduced pressure. The residue was dissolved in CHC13, and washed with water. The product, GLH-57, from the reaction with the Hünig's base was obtained in a yield of 84.5% and was purer (97% by HPLC) than that obtained with proton sponge base. M/z[M/2]+=687.4504 (MALDI).

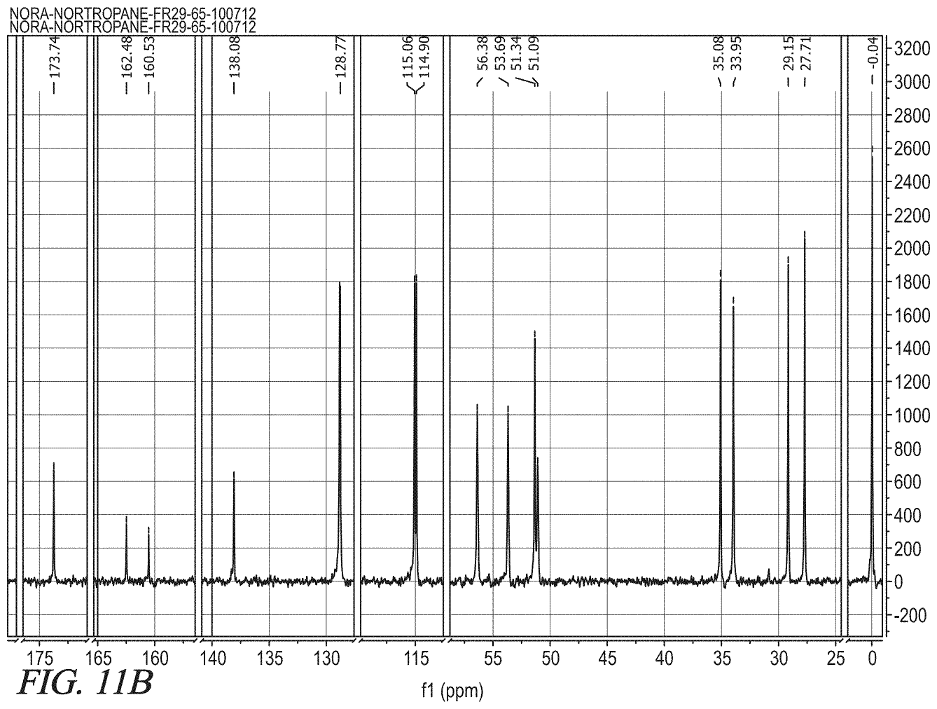

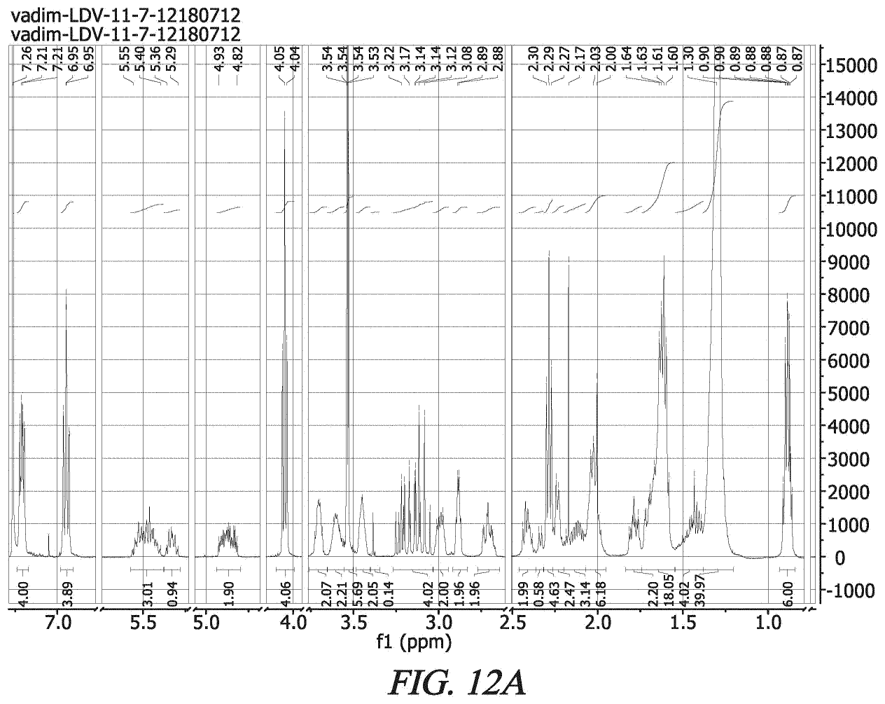

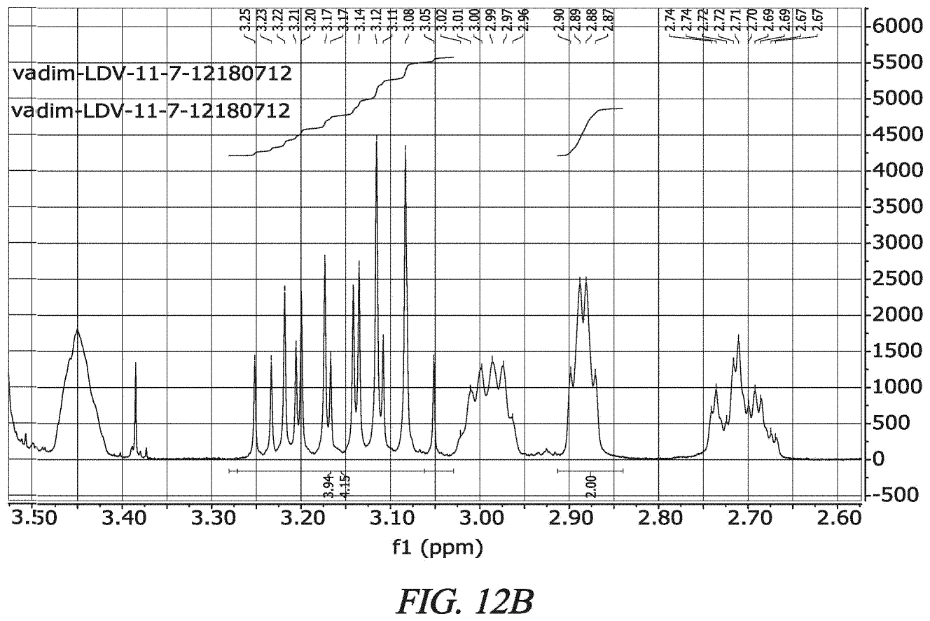

The NMR spectra (FIG. 12) shows the disappearance of the bromomethylene protons CH₂—Br at 3.93 ppm and the appearance of the CH₂—N protons obtained after conjugation at 3.05-3.25 ppm, as well as of the aromatic protons at 6.93-7.21 ppm

The ratio between the structural isomers (where the OH group is once on the 41 carbon, isomer I, and once on the 42 carbon, isomer II)

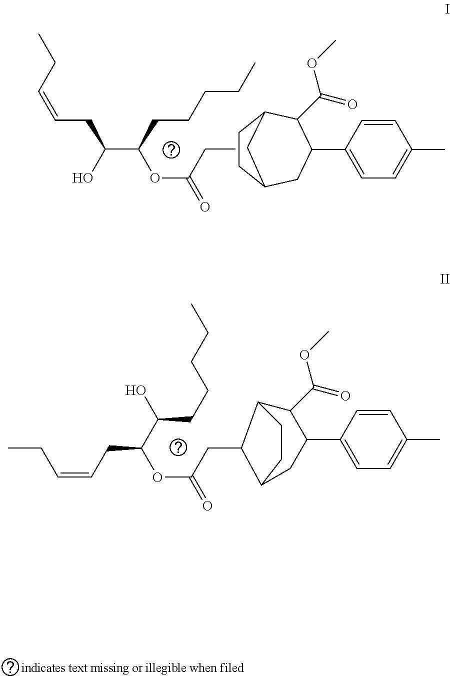

was studied through the double bond protons that appear at 5.35 ppm (one H) and at 5.50 ppm (three H).

The protons at position 38 have a characteristic peak at 5.5 ppm in both I and II configurations. The proton at position 39 of isomer II, shifted from 5.35 ppm to 5.50 ppm compared to isomer I and appears together with the protons at position 38. For a ratio of 1:1 of the isomers I and II (4 protons on the atoms 38 and 39), 3H appeared at 5.5 ppm (2 protons for isomers I and II on 38 atom and 1 proton from isomer I on the atom 39) and 1H on the 39 atom from isomer II appeared at 5.35 ppm.

### EXAMPLE 15

### Vesicle Formation and Characterization

Vesicle Morphology

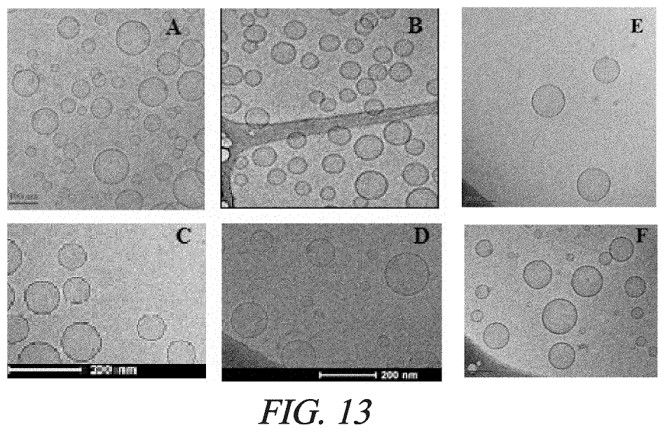

Aggregation of the basic bolas, GLH-19 and GLH-20, nanoparticles was studied using cryo-transmission electron microscopy (cryoTEM). Both bolas, GLH-19 and GLH-20, formed spherical vesicles with a diameter range of 50-120 nm (FIG. 13A and FIG. 13B).

Trypsinogen wasas a model protein for GDNF in initial encapsulation studies, since (a) trypsinogen is considerably less expensive and is available in large quantities for initial exploratory studies, (b) both proteins have similar molecular weights and isoelectric points (molecular weights of 22 KDa and 18 KDa for trypsinogen [35] and glycosylated GDNF [36], respectively, and (c) have similar isoelectric points of 9.25-9.36 and 9.26 for trypsinogen [37] and GDNF [36], respectively. Thus encapsulation, which is mostly affected by molecular weight and isoelectric point, is expected to be almost identical for both proteins. When trypsinogen was encapsulated at a concentration of 2 mg/ml in the vesicles, the spherical morphology of the vesicles remained similar to that of empty vesicles, with a slight increase in vesicles size (FIG. 13 C and FIG. 13D) and with a somewhat wider size distribution, as indicated by the dynamic light scattering data, below. Trypsinogen at concentrations below 1 mg/ml did not affect vesicle morphology at all nor did it affect vesicle size or size distribution, as indicated by the dynamic light scattering data, below.

Vesicle stability and quantitative biodistribution studies used trypsinogen, as well as vesicles loaded with carboxyfluorescein, a self quenched fluorescent dye. As an initial matter, it was determined that CF-loaded vesicles maintained their spherical shape and size, with no apparent effect on any vesicle characteristics or morphology. (FIG. 13E and FIG. 13F).

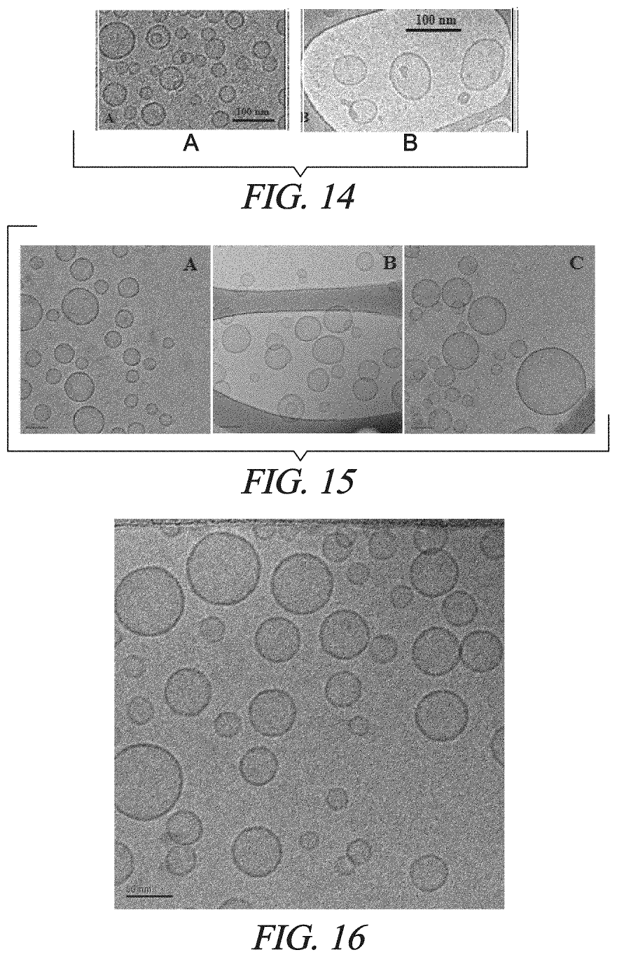

Vesicles were also prepared from a mixture of GLH-19 and GLH-20. The rationale behind such vesicles was an attempt to obtain vesicles with higher stability, lower toxicity (preliminary studies indicate that vesicles from GLH-19 have lower toxicity than vesicles made from GLH-20), and which release their encapsulated content upon exposure to choline esterases (ChE). Notably, GLH-19 vesicles do not release their encapsulated content when exposed to ChE, whereas vesicles made from GLH-20 release their encapsulated content in presence of ChE, yet GLH-19 form more stable vesicles with lower toxicity than GLH-20. Without wishing to be held to this belief, it is our understanding that that in vesicles that are made from a mixture of GLH-19 and GLH-20, the former (GLH-19) will contribute to stability and to a lower toxicity, whereas the latter (GLH-20) will contribute head groups that are sensitive to ChE, thus providing a controlled release mechanism once the vesicles enter into the brain. A formulation of GLH-19 together with GLH-20 and cholesterol and cholesteryl hemisuccinate (the two cholesterol compounds are used in all vesicle formulations as membrane stabilizers) yielded spherical vesicles (FIG. 14A) that were very similar in size and morphology to the vesicles made from either GLH-19 or GLH-20 alone (note that membrane stabilizers were added to all the formulations). Also in vesicles made from a mixture of GLH-19 and GLH-20, the encapsulated trypsinogen did not affect vesicle morphology and only slightly increased their size (FIG. 14B).

The formulations for preparing vesicles, expected to be particularly useful for in vivo studies, further contain small quantities (1 mg/ml) of a bolaamphiphile with a CS head group on one side of the hydrophobic skeleton and an ACh head group on the other side of the hydrophobic skeleton (GLH-55a), and also 0.8 mg /ml of a bolaamphiphile with the DAT ligand on each side of the hydrophobic skeleton (GLH-57). Data obtained indicate that the incorporation of GLH-55a and GLH-57 does not affect vesicle morphology and other vesicle characteristics. As can be seen from FIG. 15, the spherical shape and size of the vesicles was maintained after incorporating into the formulation GLH-55a (FIG. 15A), GLH-57 (FIG. 15B) or both GLH-55a and GLH-57 (FIG. 15C); thus, vesicles that contained GLH-55a and GLH-57 were similar to vesicles made from a mixture of GLH-19 and GLH-20 without the CS and the DAT ligand decoration.

Since trypsinogen at concentrations below 1 mg/ml did not affect vesicle morphology and the concentration of the GDNF was not expected to exceed 1 mg/ml, further studies to determine the effect of the encapsulated protein on vesicle characterization, including vesicle morphology, were done with the “real” protein—GDNF. As can be seen from FIG. 16, encapsulated GDNF had no effect on vesicle morphology and vesicle size

Vesicle Size and Size Distribution Determine by Dynamic Light Scattering

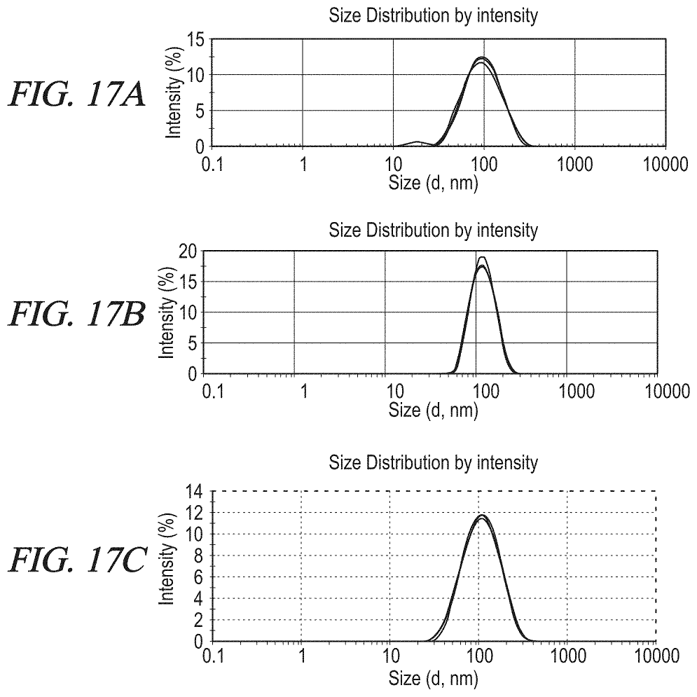

Representative dynamic light scattering profiles are provided in FIG. 17, i.e., for empty vesicles made from GLH-19 (FIG. 17A), GLH-20 (FIG. 17 B), and a mixture of GLH-19 and GLH-20 (FIG. 17C).

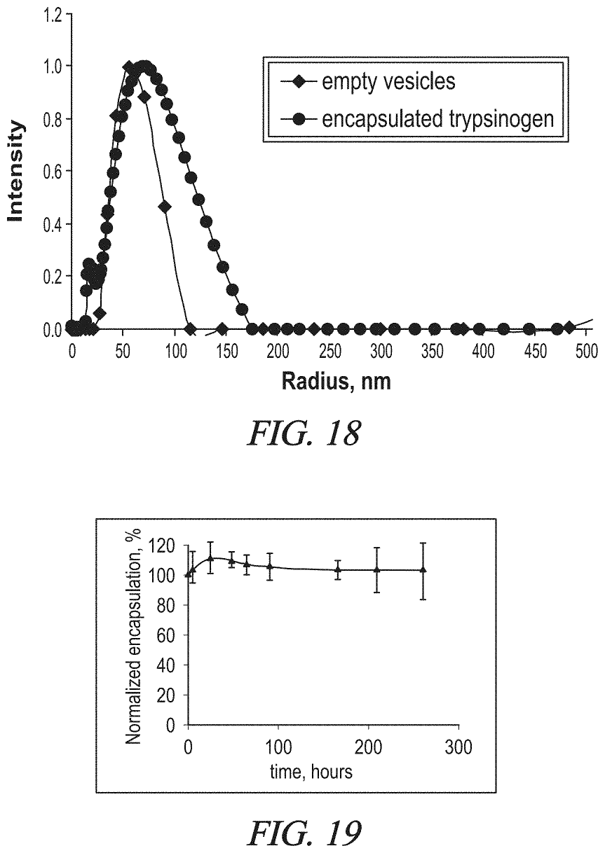

As can be seen, all vesicles preparations were fairly homogeneous, showing one peak in the DLS profile, with a vesicle diameter that averages about 100 nm. The effect of encapsulated trypsinogen on the size of the vesicles and their surface charge (zeta potential) was also tested. In general, it was observed that some increase in vesicle size, and a somewhat wider size distribution than empty vesicles, when we encapsulated trypsinogen in the vesicles. A representative DLS profile of GLH-20 vesicles, with and without encapsulated trypsinogen, is shown in FIG. 18.

The sizes of several types of vesicle preparations and their zeta potentials measured by DLS are summarized in Table 7.

TABLE 7
Vesicle size and zeta potential values measured by DLS

| Type of vesicles and loaded | Diameter in nm | Zeta potential in mV |
|---|---|---|
| protein | (mean ± SEM) | (mean ± SEM) |
| GLH-19 (empty vesicles) | 100.8 ± 0.9 | 28.70 ± 2.52 |
| GLH-19 loaded with 4 mg/ml | 129.2 ± 7.2 | 27.68 ± 2.79 |
| trypsinogen | | |
| GLH-20 (empty vesicles) | 104.7 ± 2.5 | 41.88 ± 1.45 |
| GLH-20 loaded with 4 mg/ml | 165.6 ± 2.0 | 35.50 ± 1.49 |
| trypsinogen | | |
| GLH-19:GLH-20 (2:1) (empty | 113.1 ± 10.6 | 37.30 ± 2.90 |
| vesicles) | | |
| GLH-19:GLH-20 (2:1) loaded | 133.2 ± 6.2 | 33.05 ± 2.69 |
| with 4 mg/ml trypsinogen | | |
| GLH-19:GLH-20 (2:1) with CS | 109.6 ± 0.1 | 33.6 ± 1.23 |
| surface groups (empty vesicles) | | |
| GLH-19:GLH-20 (2:1) with | 123.5 ± 0.5 | 44.6 ± 1.27 |
| DAT ligand surface groups | | |
| (empty vesicles) | | |
| GLH-19:GLH-20 (2:1) with CS | 115.2 ± 1.3 | 34.7 ± 3.96 |
| and DAT ligand surface groups | | |
| (empty vesicles) | | |
| GLH-19:GLH-20 (2:1) with CS | 114.3 ± 1.5 | 36.0 ± 3.16 |
| and DAT ligand surface groups | | |
| (loaded with GDNF) | | |

Vesicles were prepared by film hydration followed by sonication from 10 mg/ml bolas, 2.1 mg/ml cholesteryl hemisuccinate and 1.6 mg/ml cholesterol. Each measurement was done on at least 3 different vesicle preparations and the values are means±SEM.

As can be seen from Table 7, the average diameter of the empty vesicles ranged between 100-123 nm. The empty vesicles are positively charged (cationic vesicles), with GLH-20 vesicles having higher zeta potential than GLH-19 vesicles. Vesicles made from a mixture of GLH-19 and GLH-20 show zeta potential in between those of GLH-19 vesicles and GLH-20 vesicles. Encapsulation of trypsinogen, at a concentration of 4 mg/ml, consistently increased the vesicle size and reduced somewhat the zeta potential in all the vesicle preparations, indicating that some protein binds to the vesicle surface and neutralizing the positively charged groups. Addition of either CS or DAT ligand alone or both of of them together, did not change significantly the vesicle size or zeta potential. Also, encapsulation of GDNF did not change the vesicle size or zeta potential, probably because the concentration of the GDNF was much smaller than the trypsinogen concentration (the GDNF concentration was 40 μg/ml).

Vesicle Stability

Vesicle stability was determined by measuring the amount of encapsulated fluorescent dye (carboxyfluorescein (CF)) as a function of time, during either storage or incubation in whole serum. In general, vesicles made from the different formulations which were shown above, were very stable under storage. Even vesicles from GLH-20, which in whole serum were less stable than vesicles made from GLH-19, were stable in storage (FIG. 19).

Determinatio of vesicle stability in whole serum is complicated by the presence of ChE activity. It had been demonstrated that vesicles from GLH-20 release their encapsulated material when exposed to ChE, whereas vesicles made from GLH-19 were not sensitive to ChE. Vesicles made from a mixture of GLH-19 and GLH-20 are believed (without wishing to be held to that belief) to be potentially more effective for the purpose of delivering GDNF to the brain since the GLH-19 component should reduce toxicity. This inference is based on preliminary studies in mice, that suggested that GLH-19 is less toxic than GLH-20, although the toxicity of either bola is significantly below the dose expected to be used in vesicles that will injected into mice in vivo, as well as on the increased vesicle stability in which the GLH-20 component will contribute the controlled release mechanism. In fact, vesicles made from a mixture of GLH-19 and GLH-20 were more stable in serum than vesicles made from GLH-20 alone and increasing the amount of the GLH-19 component within vesicles made from a mixture of both bolas increased vesicle stability in serum (FIG. 18).

That is, as can be seen from FIG. 18, the half life of vesicles made from a mixture of GLH-19 and GLH-20 is about 4-6 hours (depending on the ratio between GLH-19 and GLH-20), compared with a half life of 2.5 hours for vesicles made from GLH-20 only.

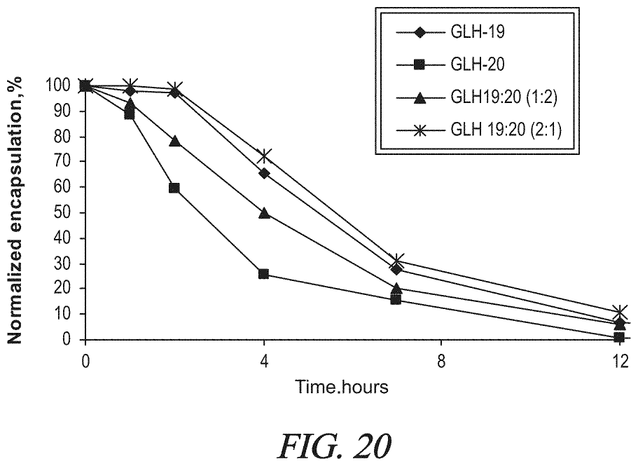

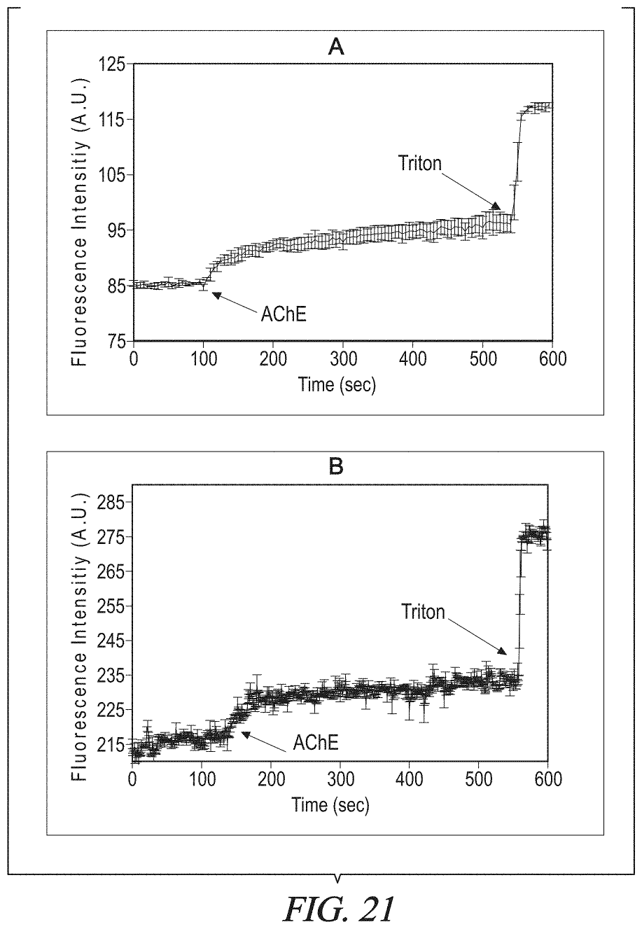

In other experiments, it was observed that vesicles made from GLH-20 decapsulate and release their content in presence of ChE. Since the vesicles are designed to release their content in the brain by the influence of the brain ChE, it was important to confirm that the relatively small amount of GLH-20 in the vesicles made from a mixture of GLH-19 and GLH-20 at a ratio of 2:1, is sufficient to cause decapsulation when exposed to ChE. Accordingly, vesicles were prepared from GLH-20 and from a mixture of GLH-19 and GLH-20, and loaded with CF and exposed to ChE, the release of the fluorescent marker was measured as a function of time after exposing them to the enzyme. The results are shown in FIG. 20.

As can be seen, both vesicle preparations started to release their content after the addition of AChE. However, release from the vesicles made from GLH-20 was somewhat faster than the release from the vesicles made from a mixture of GLH-19 and GLH-20. Thus, 5 min after the addition of AChE, the vesicles made from GLH-20 released 42% of their content whereas at this time point the vesicles made from the mixture of GLH-19 and GLH-20 released 33% of the total CF that was encapsulated. Accordingly, vesicles made from a mixture of GLH-19 and GLH-20 would be predicted to release their content in the brain in response to brain ChE and therefore, these vesicles can be used to deliver compounds to the brain and release their cargo in the brain.

### EXAMPLE 16

### Protein Encapsultion

Optimization of the Encapsulation Using a Model Protein (Trypsinogen)

Initial studies on optimization of the encapsulation, relied on the use of an inexpensive, readily available, model protein (trypsinogen) with similar properties to GDNF. Parameters that may influence encapsulation include the molecular weight of the protein and its isoelectric point. Large proteins may affect vesicle properties in a different way than small proteins. For example, in our preliminary studies we observed that smaller proteins can be used at higher concentrations compared with larger proteins before the vesicles aggregate and form a turbid suspension. The isoelectric point may influence the binding of the protein to the positively charged head groups of the bolaamphiphiles. Trypsinogen, has a molecular weight (22 KDa) comparable to that of GDNF (18 KDa of the glycosylated form), as well as a comparable isoelectric point, with both proteins having an isoelectric point near pH 9.

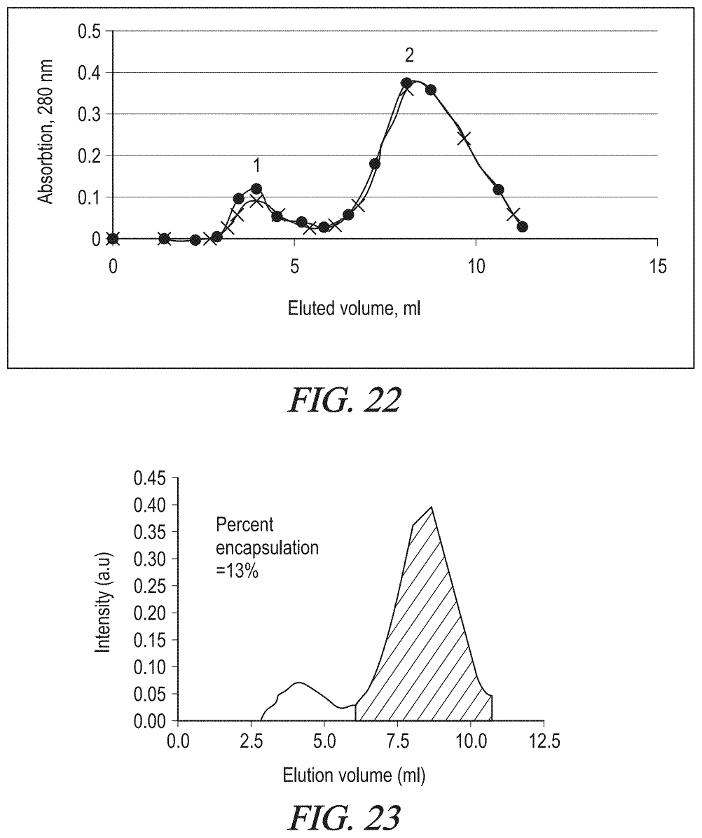

The concentraion of trypsinogen can be measured by UV absorbance at 280 nm. Since the vesicles are prepared in media that contain trypsinogen, encapsulated protein had to be separated from non-encapsulated protein to determine encapsulation efficiency. For example, encapsulated trypsinogen could be separated from free protein by size exclusion chromatography on a Sephadex G50 column. As can be seen from FIG. 22, on a Sephadex G50 column, the vesicles were eluted in the first 3-5 ml of the eluting buffer, while the free trypsinogen was eluted in the next 6-11 ml; thus, the encapsulated protein could be well separated from the non-encapsulated protein.

This method for the separation of the encapsulated trypsinogen from the non-encapsulated protein (free trypsinogen), allowed quantification of the encapsulated protein. As can be seen from FIG. 23, each peak that was eluted from the column could be quantified by determining the area under the curve (AUC), using the Prism Graph Pad software that takes into consideration overlaps between peaks, in case of overlaps.

Based on the above methods for separation of the encapsulated protein and quantifying percent encapsulation, the encapsulation process was optimized to provide maximum encapsulation efficiency. In the first stage of the optimization, several concentrations of trypsinogen were used with a fixed amount of bolaamphiphiles (10 mg/ml of a mixture of GLH-19 and GLH-20 in the ratio of 2:1), using a trypsinogen concentration of 4 mg/ml. This approach facilitated the accurate measurement of the amount of the protein in each fraction collected from the column, without interference by the small light diffraction of the vesicles that were eluted from the column. The parameters varied in the optimization included: a) the ratio between the bolas and the additives—cholesterol and cholesteryl hemisuccinate; b) the pH in which the vesicles were prepared; c) the pH of the eluting buffer; and d) the method for vesicle preparation and encapsulation. The data obtained from these initial optimization studies are summarized in Table 8.

TABLE 8
Encapsualtion Of Trypsinogen In Bolaamphiphilic Vesicles

| Vesicle formulation | | | |
|---|---|---|---|
| Bola (GLH-19:GLH-20): | Preparation | Elution | Percent |
| CHOL:CHEMS | medium | medium | encapsulation |
| 4(2:1):1:1 | PBS | TB (pH = 7.3) | 25 |
| | | AB | 8 |
| 2(2:1):1:1 | PBS | TB (pH = 7.3) | 27 |
| | | AB | 15 |
| 4(2:1):1:1 | TB | TB (pH = 7.3) | 36 |
| | | AB | 13 |
| 2(2:1):1:1 | TB | TB (pH = 7.3) | 30 |
| | | AB | 15 |

The formulations that were used for the vesicle preparations contained 10 mg/ml bolas (the ratio between the bolas was always 2 parts of GLH-19 and 1 part of GLH-20), and the ratios between the bolas and the cholesterol (CHOL) and cholesteryl hemisuccinate (CHEMS) were varided as indicated in the Table 8. Vesicles were prepared in the media as indicated in Table 8. All media contained 4 mg/ml trypsinogen. PBS is phosphate buffered saline, pH=7.4; TB is Tris buffer 10 mM pH=9.5, except for cases when pH=7.3 is indicated; AB is acetate buffer 10 mM, pH=3.5.

As can be seen from Table 8, the highest trypsinogen encapsulation was obtained when the vesicles were prepared in high pH (9.5), possibly due to binding of the protein, at pH above its PI (the PI is about 9), to the positively charged head groups of the bolas. Eluting the vesicles with a buffer of physiological pH (7.3) did not reduce encapsulation even if the vesicles were prepared in high pH, but eluting the vesicles from the Sephadex G50 column with a buffer of a low pH (3.5) significantly reduced encapsulation, probably because at low pH trypsinogen is more positively charged (the PI of trypsinogen is around 9) and its complexation with the cationic head groups of the bolaamphiphiles is weakened. Increasing the ratio of the bolas, in relation to cholesterol and cholesteryl hemisuccinate, did not affect the encapsulation efficiency very significantly.

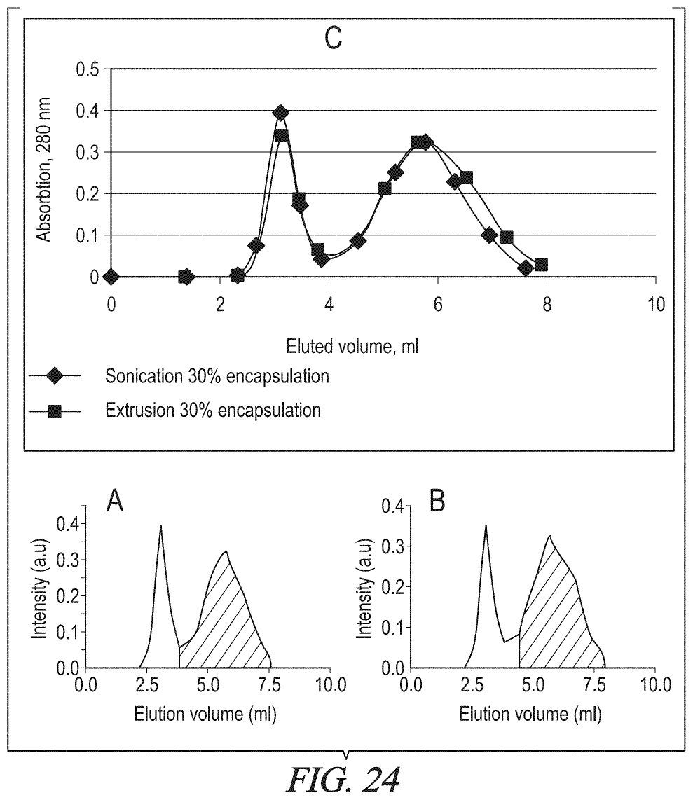

The method of the vesicle preparation on encapsulation was also examined, using two different methods: a) film hydration followed by sonication and b) extrusion via membrane with a pore size of 100 nm. Since sonication may damage the protein, extrusion methods would be viable alternatives, although they require higher working volumes and for scale up, would be advantageous. The date of FIG. 24 demonstrate encapsulation obtained following sonication compared with extrusion.

As can be seen in FIG. 24, similar encapsulation efficiency was achieved by both methods. Accordingly, for small scale studies, film hydration followed by sonication could be used while, for scale-up for larger quantities of vesicles, the extrusion is advantageous.

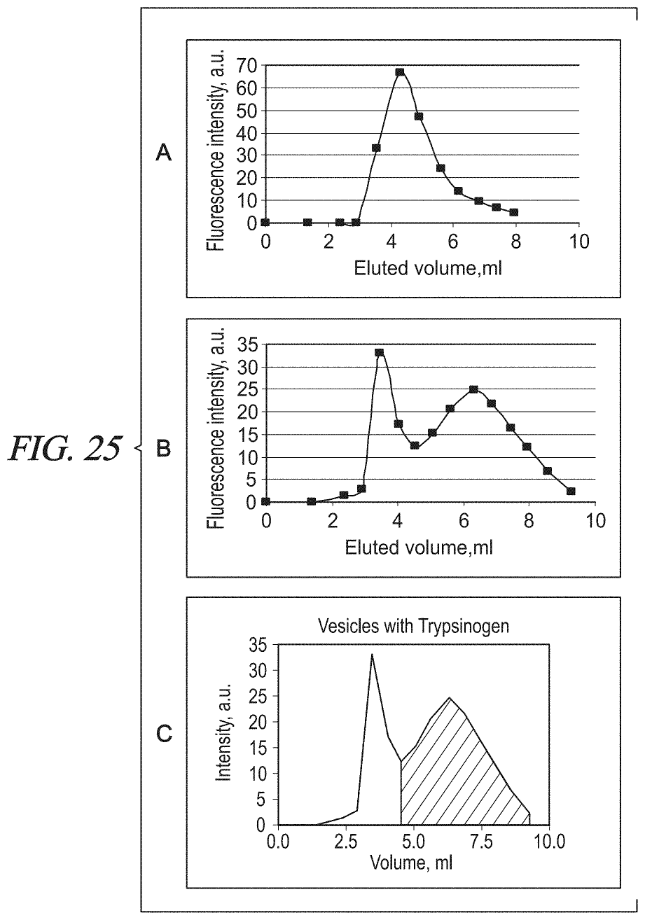

To increase the sensitivity for measurements of encapsulated protein, that material was labled with AlexaFluor®-488 and measured the protein concentration measured by fluorescence. Elution of the vesicles in Tris buffer does not exhbit adequate separation between the encapsulated protein and the free protein (FIG. 25 A). Elution in PBS, however, provided better results and an adequate separation between encapsulated and non-encapsulated trypsinogen (FIG. 25B), with 30% encapsulation obtained under similar conditions to those used for the larger quantities of trypsinogen (FIG. 25 C).

Additional experiments, using several levels of trypsinogen within the range expected for GDNF,were carried out with several amounts of bolas to evaluate encapsulation efficiency. The results are summarized in Table 9.

TABLE 9
Percent Encapsulation Of AlexFluor ® 488 Labled Tyrpsinogen

| | 10 mg/ml GLH-19: | 5 mg/ml GLH-19: | 2.5 mg/ml GLH-19: |
|---|---|---|---|
| Trypsinogen | GLH-20 (2:1) | GLH-20 (2:1) | GLH-20 (2:1) |
| 17.5 μg/ml | 77 | 56 | 59 |
| 35 μg/ml | 60 | 54 | 61 |
| 70 μg/ml | 51 | 28 | 32 |
| 140 μg/ml | 56 | | |

Vesicles were prepared by film hydration followed by sonication in presence of various amounts of AlexaFluor®-488-labeled trypsinogen and various concentrations of the bolas, as indicated. Values are percent encapsulation, calculated by using the amount of the labeled trypsinigen that was present during vesicle preparation as 100%.

As can be seen from the data of Table 9, higher encapsulation efficiencies were obtained with higher bolas/protein ratio.

GDNF Encapsulation

In light of the data obtained for optimized encapsulation of trypsinogen GDNF encapsulation was carried out using the vesicle formulation that gave the highest trypsinogen encapsulation. In this experiment, GDNF was added at a concentration of 12.5 μg/ml for preparation of vesicles for in vivo studies. In this experiment, the percent encapsulation obtained with GDNF was compared to that obtained with trypsinogen under similar conditions.

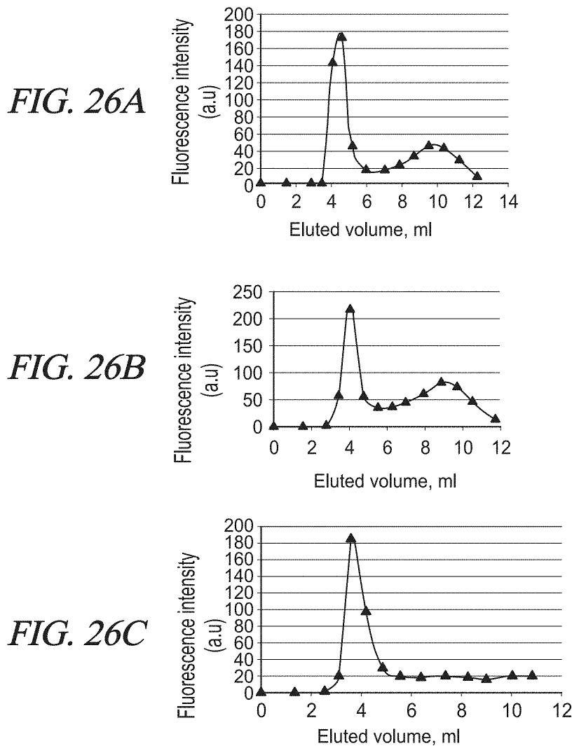

For this experiment, vesicles were prepared by film hydration followed by sonication from a mixture of GLH-19 and GLH-20 at a concentration of 10 mg/ml with 1.6 mg/ml cholesterol and 2.1 mg/ml cholesteryl hemisuccinate. The formulations contained 50 μg/ml trypsinogen (A), 100 μg/ml trypsinogen (B) and 12.5 μg/ml GDNF (C). All proteins were labeled with AlexaFluor®-488. After encapsulation, the vesicles were eluted from a Sephadex G50 column by PBS and the fluorescence of each fraction was determined. The results are shown in FIG. 26.

The percent encapsulation for each vesicle preparation was detrmined and the data presented in FIG. 26. The data reveal 42% and 54% encapsulation for 100 μg/ml and 50 μg/ml trypsinogen (FIG. 26B and FIG. 26A, respectively), and 66% encapsulation for the GDNF (FIG. 26C).

### EXAMPLE 17

Determination of GDNF Integrity and Activity Following Encapsulation

Although sonication may affect the integrity and/or the activity of the GDNF, film hydration followed by sonication were to be employed for initial preparations of vesicles for use with in vivo studies, since this method is economical, does not require a high volume of vesicles compared with the extrusion method, and allows smaller amounts of GDNF. Accordingly, the integrity and the activity of naked GDNF to that of encapsulated GDNF, where encapsulation was achieved by the method of film hydration followed by sonication.

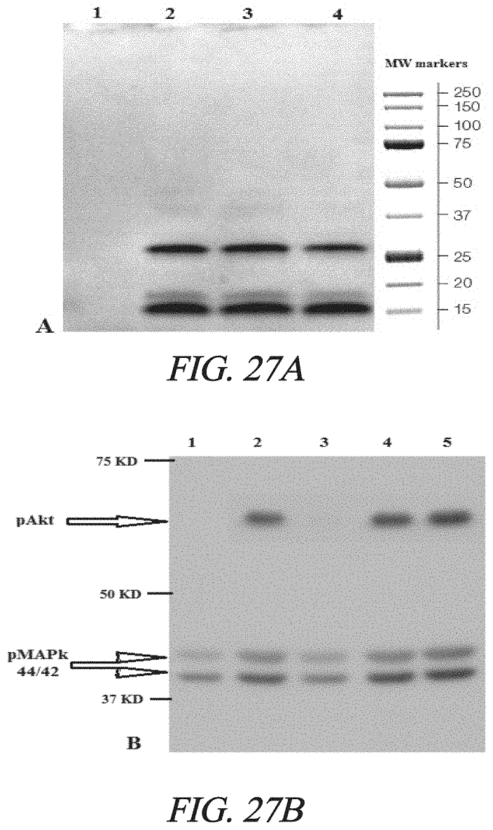

The integrity of GDNF was examined by polyacryl amide gel electrophoresis (PAGE), where we looked for possible changes in molecular weight that may suggest fragmentation due to sonication. The results that are shown in FIG. 27 and clearly suggest that both the monomeric form of the GDNF and its dimeric form were not changed after sonication, and since no additional bands appeared on the gel, indicating that no fragmentation of the GDNF occurred during the encapsulation process.

In particular, FIG. 27 depicts the effect of the encapsulation process on GDNF integrity and activity. (A) Analysis of GDNF on PAGE, where lane 1 is empty vesicles; lane 2 is GDNF encapsulated by the method of film hydration followed by sonication; lane 3 is encapsulated GDNF which was incubated before the PAGE at 40° C. for one hour; and lane 4 is free GDNF. (B) Test of GDNF activity using SH-SYSY neuroblastoma cells where lane 1 is control untreated cells; lane 2 is cells treated with free GDNF; lane 3 is cells treated with empty vesicles; lane 4 is cells treated with free GDNF added to empty vesicles; and lane 5 is cells treated with GDNF encapsulated in bolavesicles by the method of film hydration followed by sonication.

The effect of the encapsulation process on GDNF activity was also examined by treating neuroblastoma cells that respond to GDNF by activation of kinases, particularly AKT and MAPK. Notably, AKT activity regulates cell survival and this activity is particularly relevant to neuroprotection conferred by GDNF and to PD therapy. AKT and MAPK are activated when they are phosphorylated and GDNF induces phosphorylation of these enzymes in SH-SY5Y neuroblastoma cells. If GDNF activity is impaired, it will not phosphorylate AKT and MAPK to pAKT and pMAPK, respectively. pAKT and pMAPK can be detected by specific antibodies for the phosphorylated forms of the enzymes on a Western blot. As can be seen in FIG. 27B, GDNF caused the same degree of phosphorylation in its free form as in its encapsulated state, or after it was added to empty vesicles. These results suggest that neither the bolaamphiphilic vesicles, nor the encapsulation process that included sonication affected GDNF activity.

### EXAMPLE 18

### Targeting of DAT Ligand-Coated Vesicles In Vitro

Vesicles GLH-57, were formed by additiona of bolas with DAT ligand head groups to the vesicle formulation of GLH 19/GLH 20 CHEMS/CHOL. When this bola is included in the formulation, the resulting vesicles are decorated on their surface with the DAT ligand, intended to target cells that express the dopamine transporter, namely, dopaminergic cells. To test if the DAT-ligand-coated vesicles have higher affinity to dopaminergic cells, the vesicles were added to three types of cells: a) PC12 cells that highly express DAT [19]; b) SH-SYSY neuroblastoma cells that are known to express DAT [20]; and c) HeLa cells that do not express DAT.

Vesicles were loaded with CF, and each cell type contacted with added the fluorescently labeled vesicles uptake of the fluorescent dye into the cell measured by flow cytometry. Vesicles were made from 10 mg/ml GLH-19:GLH-20 (2:1) without (uncoated vesicles) and with 0.8 mg/ml GLH-57, a bola that contains DAT ligand as the head group (DAT-vesicles). Cells were incubated for 1 h with the vesicles, and tested by flow cytometry. A shift to the right of the peak indicates fluorescent cells due to uptake of the vesicles.

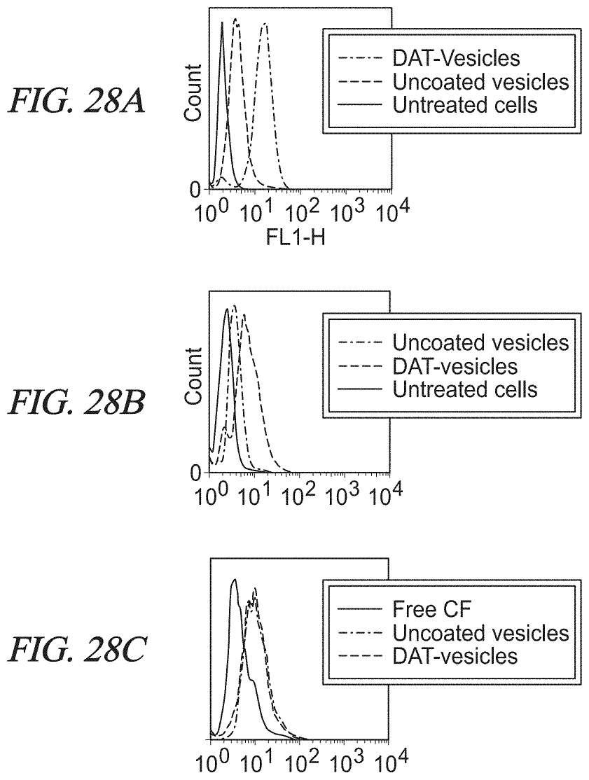

As can be seen from FIG. 28, significantly higher uptake of the fluorescent vesicles was seen when the vesicles were coated with DAT ligand and added to PC12 cells (FIG. 28A) that highly express DAT compared to uncoated vesicles. Also, higher uptake of the DAT ligand-coated vesicles, compared to uncoated vesicles, was observed in SH-SYSY neuroblastoma cells (FIG. 28 B) that also express DAT. Yet, when the fluorescently labeled vesicles were added to HeLa cells that do not express DAT, no difference was observed in the uptake between DAT ligand-coated vesicles and uncoated vesicles (FIG. 28C).

These results strongly suggest that vesicles coated with DAT ligand are targeted to cells that express DAT, and after penetrating the brain, such vesicles will target dopaminergic cells in brain regions such as the striatum and the Sunstantial Nigra pars compacta.

### EXAMPLE 19

### Biodistribution of the Delivered GDNF Withi the Mouse Brain

Effect of CS Surface Groups on BBB Permeability of the Vesicles

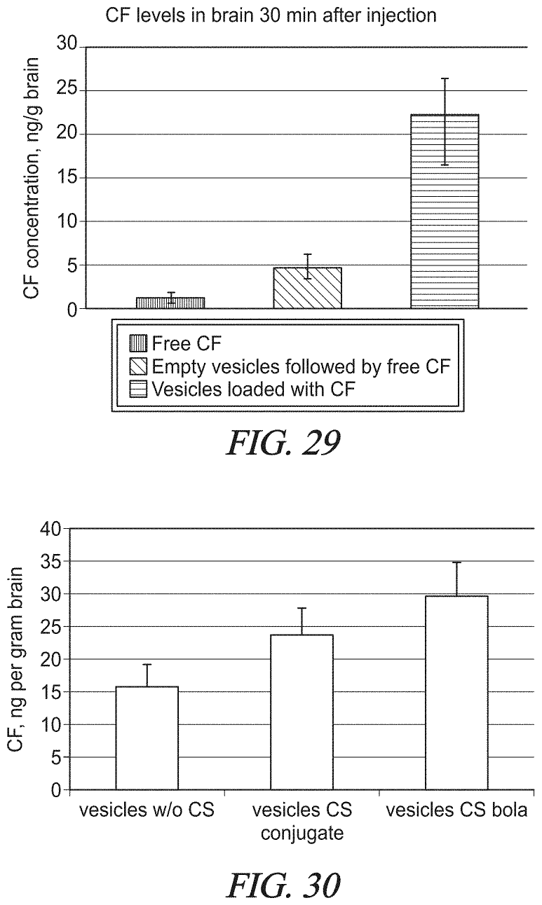

Preliminary studies indicated that vesicles coated with CS surface groups penetrate the BBB better than uncoated vesicles. The CS coating in these preliminary experiments, was achieved by incorporating a CS-fatty acid conjugate into the vesicle formulation. However, the energy barrier for pulling the CS surface group out of the vesicle membrane, which is anchored to the membrane via fatty acid, is low, and therefore, some of the CS surface groups may be lost before the vesicle reaches the BBB. Pulling bolas out of a monolayer membrane is much more difficult since the hydrophilic head group has to pass through the hydrophobic domain of the monolayer membrane, and this takes more energy. Accordingly, bola with CS attached covalently to the bola's skeleton were sued instead of CS-fatty acid conjugate. To verify that the vesicles do not compromise the BBB, but rather crossed the BBB in their intact form, the experiment depicted in FIG. 29 was carried out.

This experiment mesure accumulation of CF in the brain following i.v. administration. Vesicles were made by film hydration followed by sonication from a 10 mg/ml mixture of GLH-19 and GLH-20 (2:1), 1 mg/ml CS-fatty acid (vernolate) conjugate, 2.1 mg/ml cholesteryl hemisuccinate and 1.6 mg/ml cholesterol in absence (empty vesicles) and in presence of 0.2/ml CF (CF-loaded vesicles). Mice were pretreated with 0.5 mg/kg (i.m.) pyridostigmine and 15 min afterward the mice were injected i.v. with either free CF, or empty vesicles and then CF, or CF-loaded vesicles. The total amounts of the CF that were injected in each case were identical (10 mg/kg). 30 min after the injection, the animals were sacrificed, perfused with 10 ml PBS and the brains removed and homogenized, deproteinized by 5% tricholoroacetic acid and fluorescence determined in the supernatants obtained following centrifugation. The data obtained are presented in FIG. 29, where dach bar represents an average value obtained from 5 mice+/−SEM.

As can be seen in FIG. 29, free CF hardly entered the brain and a very little amount of CF was measured in brain homogenate taken from animals that received free CF. By comparison, 15 times more CF was found in the brain after the injection of encapsulated CF. When free CF was injected immediately after the injection of empty vesicles, about 3 times more CF in the brain was obaserved as compared with the amount found after the injection of free CF alone. This increase may be attributed to binding of the negatively charged CF to the positively charged surface groups of the vesicles, before the vesicles entered the brain, thus, some of the dye was carried into the brain while being bound to the vesicles. Similar results were obtained when the CF was injected just before the injection of empty vesicles (not shown). The profound increase in the CF concentration on the brain obtained after the injection CF loaded vesicles compared to that obtained after the injection of empty vesicles and free CF suggest that the vesicles do not compromise the BBB, but rather enter into the brain in their intact form and release the encapsulated drug within the brain after their entry.

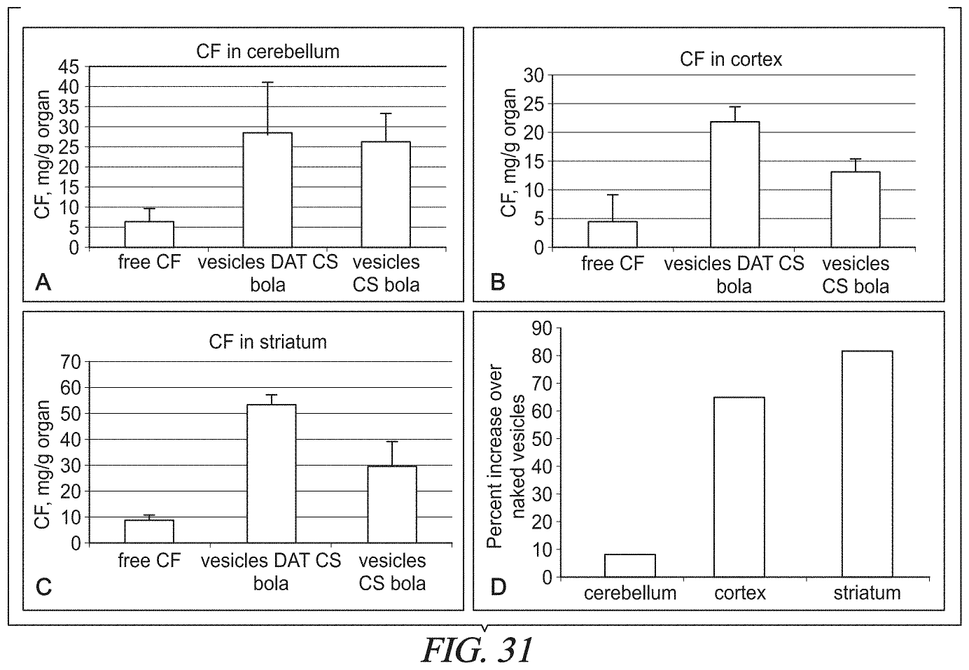

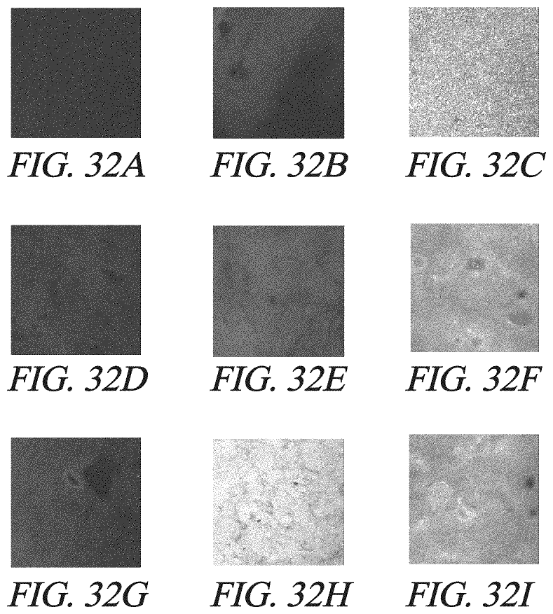

In the experiment shown in FIG. 29, a CS-fatty acid (vernolic acid) conjugate to was used to introduce the CS groups to the surface of the vesicles. BBB permeability of the vesicles with CS surface groups that are an integral part of the membrane structure (using the bola with a CS head group as synthesized herein) to vesicles with CS surface groups that were introduced by the CS-fatty acid conjugate (FIG. 30).

In this experiment, vesicles were prepared as described in FIG. 29, except that in one case 1 mg/ml GLH-55a was used in the vesicle formulation to provide CS surface groups (vesicles with CS-bola), and in the other case, 1 mg/ml CS-fatty acid conjugate was used. Conditions of this experiment were similar to those presented in FIG. 29.

As demonstrated by the data of FIG. 30, the presence of CS surface groups increased the amount of the CF that was measured in the brain. The amount of the CF that was measured in the brain after injecting the dye encapsulated in vesicles, was increased by about 50% when the CF was encapsulated in vesicles to which the CS surface groups were added by using a CS-fatty acid conjugate, compared to naked vesicles. By comparison, the amount of the CF in the brain was increased by about 100% when the CF was encapsulated in vesicles in which the CS surface group was an integral part of the membrane (by using the bola-CS-GLH-55a). These results indicate that bola-CS is better than fatty acid-CS for enhancing the permeability of the vesicles via the BBB.

### EXAMPLE 20

### Targeting of Vesicles Coated with DAT Ligand to the Striatum

As demonstrated above, the vesicles described herein transport their encapsulated content through the BBB into the brain. This experiment was intended to demonstrate that the vesicles that are coated with DAT ligand will be targeted to brain regions that contain dopaminergic neurons. Vescicles were loaded with CF and injected into the tail vein of mice and 30 min after the injection, the mice were sacrificed, the brain removed and dissected into three brain regions: (1) the cortex; (2) the striatum; and (3) the cerebellum. Each of these brain regions was homogenized, deproteinized by trichloroacetic acid, and fluorescence intensity was measured in the supernatant that was obtained after centrifugation.

In particular, vesicles were prepared by film hydration followed by sonication from a 10 mg/ml mixture of GLH-19 and GLH-20 (2:1), 1 mg/ml GLH-55a (a bola with CS head group), 2.1 mg/ml cholesteryl hemisuccinate, 1.6 mg/ml cholesterol, 0.2 mg/ml CF and without (vesicle CS bola) or with GLH-57 (vesicles DAT CS bola). Mice were pretreated with 0.5 mg/kg (i.m.) pyridostigmine (to inhibit peripheral ChE) and 15 min afterward the vesicles were injected i.v. After 30 min the mice were sacrificed, perfused with 10 ml PBS and the brain removed and dissected into cortex, striatum and cerebellum. The tissues were weighed, homogenized and deproteinated by trichloroacetic acid, centrifuged and fluorescence was determined in the homogenates. The amount of the CF in each brain region was calculated from a calibration curve of CF, taking into consideration the weight of the tissue and the dilution done during the homogenization. Each bar represent an average value obtained from 5 mice+/−SEM.

FIG. 31 depicts the results of this experiment. As can be seen from FIG. 31, the highest amount of CF was found in the striatum of animals that were injected with DAT ligand-coated vesicles. The largest difference in CF concentrations between uncoated vesicles and coated vesicles was observed in the striatum, then in the cortex and lastly in the cerebellum, where there was almost no difference between the amounts of the CF that were measured in animals that received uncoated vesicles versus those that received DAT ligand-coated vesicles. Free CF did not penetrate into the brain in significant amounts. These data show that the vesicles penetrate into the brain, and once in the brain, the vesicles that were coated with DAT ligand were targeted to brain regions that are known to have dopaminergic neurons.

### EXAMPLE 21

### Delivery of Labeled Trypsinogen by the Bolavesciles

Prior to delivering GDNF to the brain, pilot experiment, in vivo, studies were carried out with the model protein—trypsinogen, which was labeled for this purpose with AlexaFluor®-488. The experiment was intended to determine whether the labeled protein can be seen in brain sections directly by histofluorescence. Mice were pretreated with pyridostigmine 15 min prior to vesicle injection (to inhibit peripheral ChE) and 30 min after the injection of the vesicles, the mice were sacrificed, perfused with 10 ml PBS and tissues were dissected out, frozen in isopentane that was cooled by liquid nitrogen, sectioned by cryomicrotime and fluorescence was observed by confocal microscopy. The fluorescence that was seen in three different tissues: a) brain; b) liver; c) kidney; is shown in FIG. 32.

FIG. 32, provides representative histofluorescence slides showing AlxaFlour-488-labeld trypsinogen in brain (A-C); liver (D-F) and kidney (G-I) of mice that were injected with the labeled protein encapsulated in CS-coated vesicles or with the free protein. Panels A, D and G are micrographs taken from control untreated mice. Panels B,E and H are micrographs taken from mice injected with 200 μg of free trypsinogen labeled with AlexaFluor®-488 and C,F and I are micrographs taken from mice that were injected with 200 μg of encapsulated trypsinogen labeled with AlexaFluor®-488.

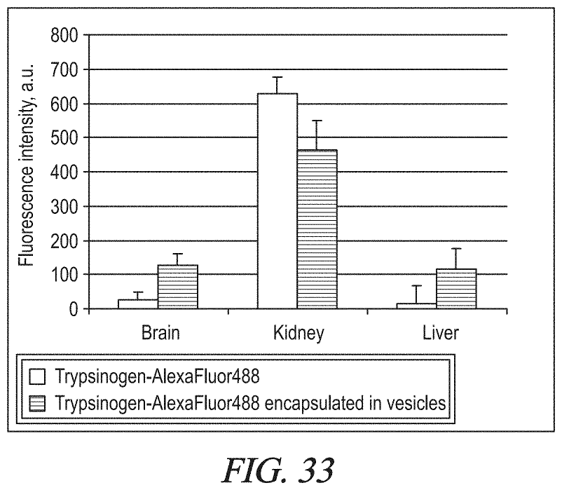

As can be seen in FIG. 32, the labeled trypsinogen is found in the brain only when it was injected encapsulated in the bolavesicles. Also, in the liver, the injection of encapsulated trypsinogen resulted in higher fluorescence than was obtained after injection of the free labeled protein. In the kidney, high fluorescence was observed also after injection of the free labeled protein. Quantification of these results was done by imaging software and is shown in FIG. 33.

More specifically, FIG. 33 depicts that distribution of trypsinogen labeled with AlexaFluor®-488 in brain, kidney and liver after the injection (i.v.) of the labeled protein in its free form or encapsulated in vesicles. This figure presents quantification of the data obtained in the experiment described in FIG. 32. Each bar represent an average value of 5 mice+/−SEM.

The data of FIG. 33 indicate that kidney was highly labeled with the fluorescent protein, due to the high penetration of the free labeled protein into this organ. However, the amount of the delivered protein, as estimated by the fluorescence, is similar in the liver, which is known to take up nanoparticles, and in the brain, to which nanoparticles do not normally enter. These results suggest that the vesicles enter the brain quite efficiently and carry their protein cargo into the brain.

The experiment described above was set to study whether a labeled protein can be detected in the brain, using a relatively high amount of the labeled trypsinogen (200 ₁ig per mouse). These data suggested a more sensitive method would be advantageus for detection and visualization of lower levels of, e.g., GDNF.

### EXAMPLE 22

### Delivery of GDNF to the Brain

In view of the sensitivity issues noted above using the fluorescently labeled protein trypsinogen, this experiment was designed to test the use of GDNF-biotin (Alomone Lab Inc., Jerusalem, Ill.) a derivative protein that maintains all the properties of GDNF, including full GDNF activity. The GDNF-biotin was introduced inot vesicles that were made from a formulation that contained all the components described above, including GLH-55a and GLH-57, bolas that contain CS and DAT ligand head groups, and the GDNF-biotin-loaded vesicles were injected (i.v.) into mice. Based on the above studies with CF and trypsinogen, in which the labeled encapsulated material was seen in the brain 30 min after the injection, this time point was chosen for the initial detection of the GDNF-biotin in the brain. For the detection of the delivered GDNF-biotin in the brain, mice were sacrificed, perfused with 10 ml PBS, to remove the GDNF-biotin from blood vessels, and brains were removed and frozen in isopentane immersed in liquid nitrogen. The frozen brains were cryosectioned and the sections were stained with DAPI (to visualize the nuclei of the cells for orientation purposes). Then, avidine-AlexaFluor®-488 was added to the slides, which were then washed and observed using a confocal microscope. The avidine binds specifically to the GDNF-biotin and only the sites in the brain that contained the delivered GDNF-biotin showed fluoresce. To exclude non specific binding of the GDNF-biotin the avidine-AlexaFluor®-488 was added also to brain sections taken from mice that were injected with PBS.

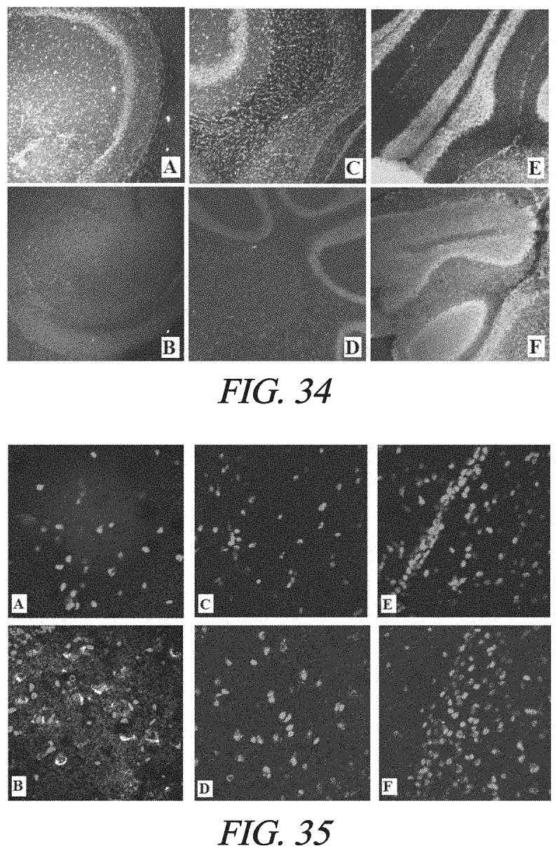

Representative brain sections are shown in FIG. 34. In this experiment, mice were pretreated with 0.5 mg/kg (i.m.) pyridostigmine, then injected i.v. with vesicles coated with CS groups and DAT ligand with encapsulated GDNF-biotin. After 30 min, animals were sacrificed, perfused with 10 ml PBS, brains removed and striata, cortex and cerebella were dissected out, frozen and cryosectioned. Brain sections from these mice were stained with DAPI (blue) and avidine-AlexaFluor®-488 (green) and observed using confocal microscopy at a magnification of 10×. (A) Stiatum from a mouse treated with PBS; (B) striatum from a mouse injected with GDNF-biotin encapsulated in vesicles; (C) cortex from a mouse injected with PBS; (D) cortex from a mouse injected with GDNF-biotin encapsulated in vesicles; (E) cerebellum from a mouse injected with PBS; (F) cerebellum from a mouse injected with GDNF-biotin encapsulated in vesicles.

As can be seen in FIG. 34, sections of the striatum from animals that were injected with vesicles with encapsulated GDNF-biotin, show a sharp focused fluorescence arranged in a circular shape around and within the striatum, whereas less fluorescence was seen in the cortex and even less fluorescence was seen in the cerebellum. The small amount of the fluorescence seen in the cortex was diffused and as focused as in the striatum. No fluorescence was seen in the control mice, indicating that the fluorescence which is seen in the brain section is specific for GDNF-biotin. Localization of the fluorescence in the brain section were also examined under higher magnification, and these results are shown in FIG. 35. It is clear from FIG. 35 that the GDNF-biotin is concentrated around many cells in the striatum and is found to a lesser extent in the cortex and the cerebellum. In particular, the the micrographs of high magnification, (60×) of FIG. 35 were taken from brain sections obtained from the mice used in the experiment described in FIG. 34. The nuclei of the cells appear in blue, due to DAPI staining, and the GDNF-biotin appears in green, due to the binding of the avidine-AlexaFluor®-488. (A)). Stiatum from a mouse treated with PBS; (B) striatum from a mouse injected with GDNF-biotin encapsulated in vesicles; (C) cortex from a mouse injected with PBS; (D) cortex from a mouse injected with GDNF-biotin encapsulated in vesicles; (E) cerebellum from a mouse injected with PBS; (F) cerebellum from a mouse injected with GDNF-biotin encapsulated in vesicles. Whether all the cells which stained for GDNF-biotin are dopaminegic neurons will be answered from co-localization studies that performed using antibodies against tyrosine hydroxylase (TH) to stain specifically the TH expressing cells.

In view of all of the above, it is apparent that vesicles have been prepared from bolas and coated with CS groups and DAT ligands. It is also apparent that these vesicles are capable of delivering GDNF to brain regions affected in Parkinson's disease. In these studies, building blocks (bolas) were designed and synethesized, and vesicles that were made from these building blocks were characterized. Further, GDNF encapsulation in these vesicles was optimized, and it has been demonstrated that they have a controlled release mechanism enabling the vesicles to release their content via the hydrolysis of the ACh head groups by brain ChE. It has also been demonstrated that the vesicles are targeted to dopamine transporter expressing cells, but not to cells that do not express the dopamine transporter. In particular, these experiments had demonstrated that the vesicles described herein are capable of transporting GDNF to the brain following systemic administration, and targeting the neurotrophin to brain regions that are affected in PD.

### EXAMPLE 23

### Controlling the Rate of Drug Release from Bolaamphiphilic Vesicles

The present disclosure further provides a method for controlling the rate of drug release from bolaamphiphilic vesicles with acetylcholine head groups by changing the length of an alkyl chain adjacent to the head group. Bolaamphiphilic compounds with acetyl choline (ACh) head groups with two different alkyl chains adjacent to the head groups were investigated for their ability to form vesicles that release the encapsulated material upon the introduction of a triggering event. One of these bolaamphiphiles, which was synthesized from vernolic acid, has an alkyl chain with 5 methylene groups adjacent to the ACh head group and the other, which was synthesized from oleic acid, has an alkyl chain with 8 methylene groups adjacent to the ACh head group. Both bolaamphiphiles formed stable vesicles with a diameter of about 100 nm. The ACh head groups of both bolaamphiphiles were hydrolyzed by acetylcholine esterase (AChE), however, the hydrolysis rate was significantly faster for the bolaamphiphile with the shorter aliphatic chain pendant. Likewise, when vesicles made from these bolaamphiphiles were subjected to AChE, those made from the bolaamphiphile with the shorter alkyl chain near the ACh head groups, released their encapsulated content faster than vesicles made from the bolaamphiphile with the longer alkyl chain pendant. That is, the rate of drug release from bolaamphiphilic vesicles with acetylcholine head groups can be controlled by varying the length of the alkyl chain adjacent to the ACh head, and, therefore, this approach can be used to design vesicles that with different varied rates of drug release.

Synthesis of Bolamphiphiles

The starting materials for bolaamphiphile synthesis are functional vegetable oils and their corresponding fatty acids. Vemolic acid, a naturally epoxidized fatty acid (cis-12,13 epoxy, cis-9 octadecenoic acid) that constitutes the main constituent of vernonia oil was used for the synthesis of the bolaamphile GLH-20, noted above, which has a head group hydrolyzed by AChE. In order to compare the rate of the hydrolysis of a similar ACh head group that contains an adjacent longer alkyl chain, a second bolaamphiphile (GLH-32) was prepared from oleic acid.

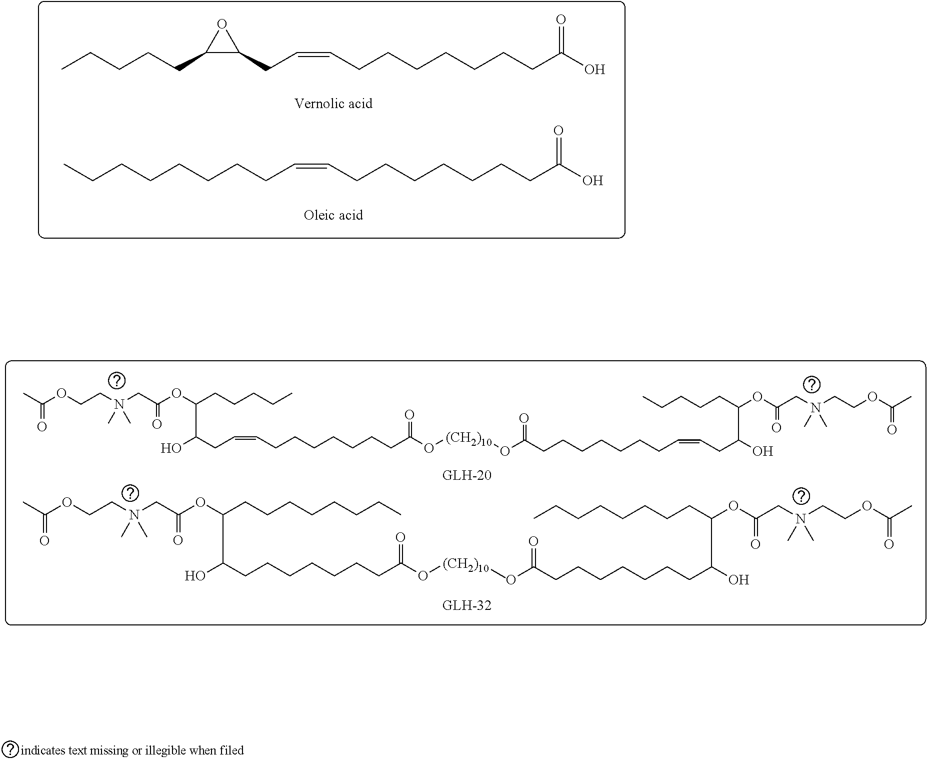

For this synthesis the oleic acid was first epoxidized by a novel approach that yielded the corresponding C₉-C₁₀ epoxy stearic acid. The synthetic strategy included two main steps: (a) synthesis of the bolaamphiphile's skeleton by elongation of the corresponding fatty acid through its carboxylic group in an esterification reaction and (b) incorporation of the head groups through the functional groups on the fatty acid aliphatic chain, as depicted in Scheme 7, below.

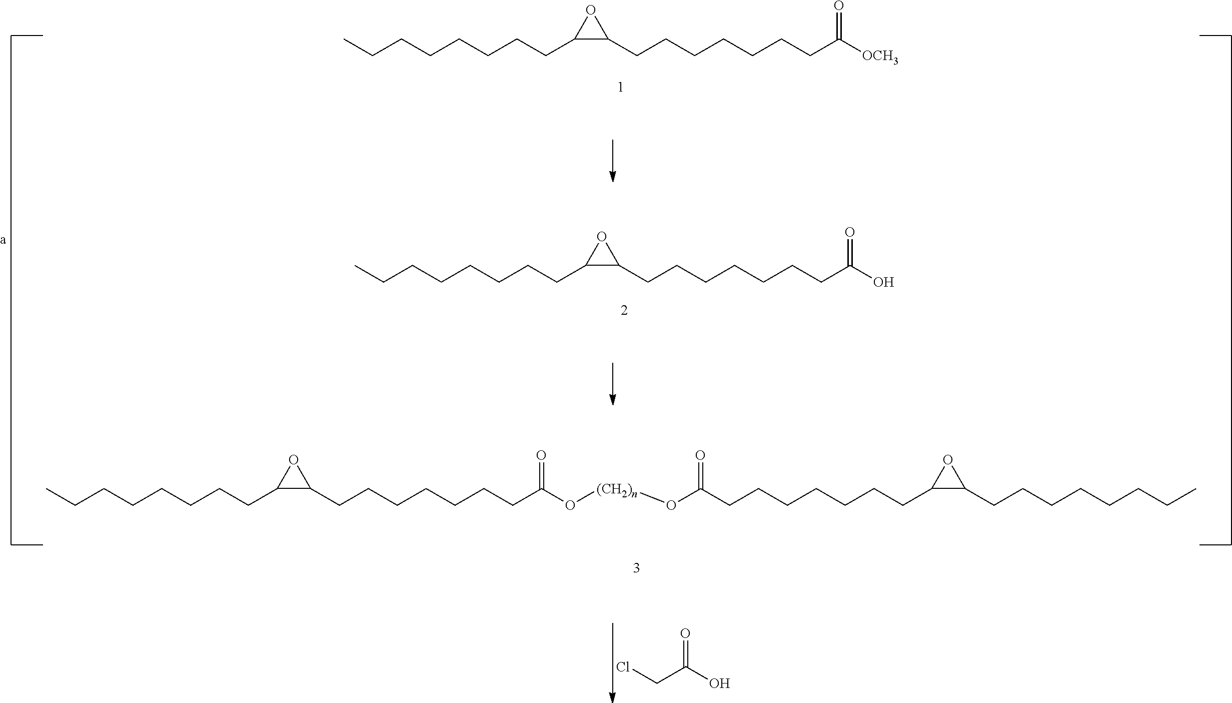

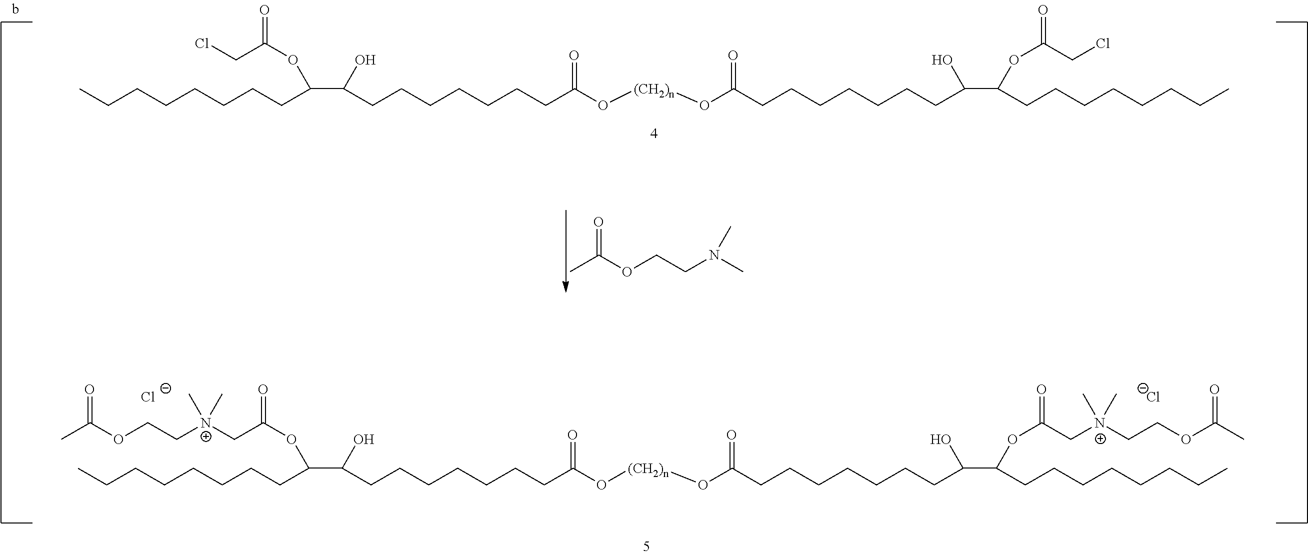

Bolaskeleton Formation

To synthesize the skeleton 3 (Scheme 7a) of the bolaamphiphile, GLH-32, the methyl monoepoxy stearate 1 was used that was obtained by the epoxidation of methyl oleate in the presence of grafted titanium-containing silica materials as the catalyst. The methyl monoepoxy stearate 1 was hydrolyzed to obtain the monoepoxy stearic acid 2. MS at the negative mode showed m/z=296.8 [M−1]⁺. A peak at 1700 cm⁻¹ appeared in the IR spectra, indicating the presence of the carbonylic carboxyl group. NMR spectra showed that the epoxy group remained untouched (2.93-2.91 ppm), and CH₂—COOH was shifted at 2.37-2.34 ppm. The monoepoxy stearic acid 2 was reacted, using a chemo enzymatic reaction, with an aliphatic diol, 1,10-decane diol, in toluene, in stoichiometric amounts, in the presence of immobilized Candida antarctica lipase as the catalyst. The product, diepoxy distearate 3 is the skeleton of the bolaamphiphilic compound.

The FT-IR spectrum of the diester 3 showed the disappearance of the absorption band at 1700 cm-1, which is related to the carboxylic acid group, and the appearance of the absorption band at 1727 cm-1, characteristic of the new ester group. The new alkoxy methylene group CH₂—O—C(O)— appeared at 4.04 ppm in the 1H-NMR spectrum and at 64 ppm in the 13C-NMR. The epoxy group remained unchanged

Attachment of the Head Group

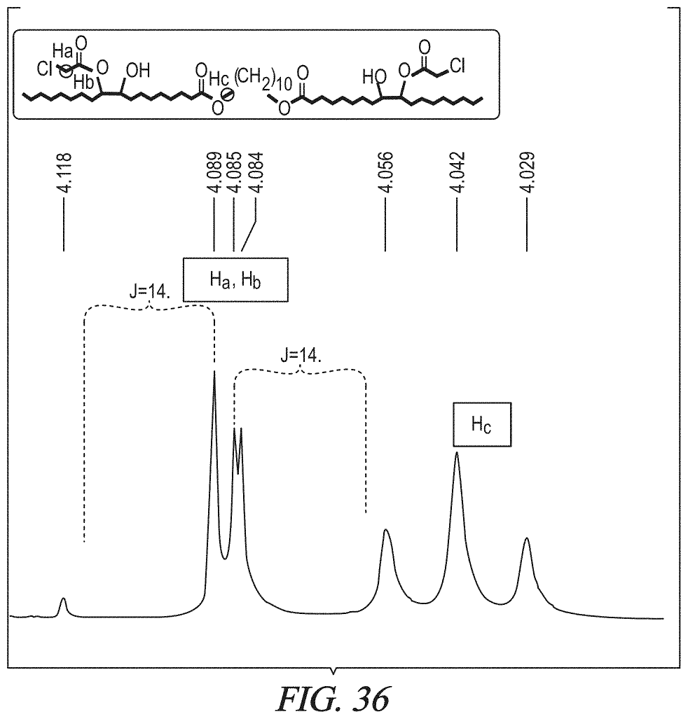

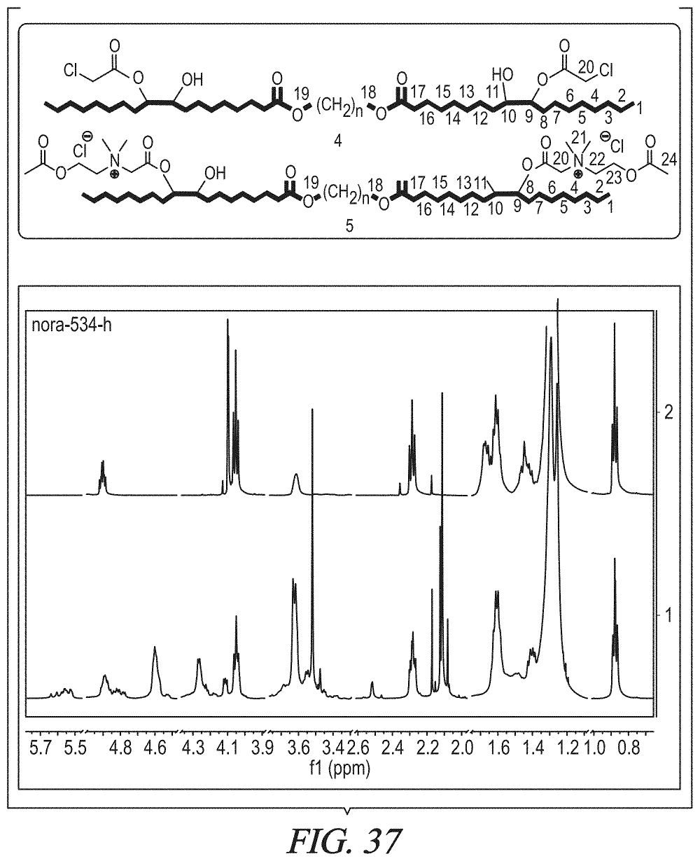

After obtaining the decane diepoxy distearate as the bolaamphiphile's skeleton, the head groups were attached in a two-stage reaction (Scheme 7b), involving (1) opening the epoxy ring with chloroacetic acid to give the dichloroacetate, derivative 4, and (2) quaternization stage of N,N-dimethylaminoethyl acetate with 4 to give the bolaamphiphile 5 with two acetylcholine head groups bound to the hydrophobic chain through the nitrogen of the choline moiety. The diepoxy distearate 3 was reacted with an excess of chloroacetic acid in dry toluene at 85° C. for 48 h. The progress of the reaction was followed by TLC and HPLC. In order to remove the excess of chloroacetic acid, the reaction mixture was washed with a concentrated solutionconcentrated solution of NaHCO₃, and the product was purified by column chromatography. The FT-IR spectrum of the dichloroacetate derivative 4 showed a new absorption band of the chloroacetate group at 1758 cm-1 and carboxylic ester absorption band at 1732 of the starting diester. In the ¹H NMR spectrum the following new signals appeared: a peak at 4.09 ppm of the methylene protons of the chloroacetate group (—CH₂Cl), a peak at 4.87-4.91 ppm of the proton of the new ester group (CH—O—C(O)), and a peak at 3.60 ppm of the proton near the hydroxyl group —CH—OH group (FIG. 36). The corresponding chemical shifts in the 13C NMR spectrum appeared at 40.96 ppm (—CH₂Cl) for the chloroacetate group, at 72.32 ppm for the carbon near the hydroxyl group (—CH-OH), at 78.81 and 78.89 ppm for the carbon adjacent to the new ester group (—CH—O—CO—CH₂Cl) and at 167.2 ppm for the carbonyl carbon of this new ester group. The formation of structural isomers in 4 was expressed in the appearance of the multiplet of the methylene protons of chloroacetate group at 4.09 ppm in the ¹H NMR spectrum and two peaks at 71.70 and 71.83 ppm, of the carbon atom adjacent to the OH group (CH-OH) and 78.81, 78.89 for CH—OC(O) in the ¹³C NMR spectrum, confirming previously reported data regarding the presence of structural isomers for the chloroacetate of methyl vernolate. The presence of structural isomers, can also be followed from the terminal methyl group at 0.87 ppm and the a-carbonyl group at 2.27 ppm; both appear as two triplets.

In FIG. 36 it can be seen that due to the proximity of the chiral carbon, the two protons of the chloromethylene group, are diastereotopic hydrogens, and they split each other. Two doublets were obtained, one for the Ha proton and the second one for the Hb proton, one of the signals overlap with the triplet of the alkoxy methylene group of the original ester. The different intensities of the peaks at 4.056 and at 4.028 ppm are due to this secondary phenomena.

The last stage of the synthesis is the quaternization reaction of N,N-dimethylamino ethyl acetate with the dicholoro acetate 4 (scheme 7B) that yields the final bolaamphiphile 5 with two acetyl choline head groups. The reaction was carried out with a large excess of the amine at 45° C. for 6 h followed up by repeated washings with ether to remove the excess of the tertiary amine and the desired bolaamphiphile was obtained as a yellow viscous product.

The ¹H-NMR of the bolaamphiphilic compound (FIG. 4) can distinguish the new peaks of the ACh head group. The methyl (24) of the acetate CH₃—C(O)—O appear as a singlet at 2.12 ppm. The methylene group (22) N⁺—CH₂—CH₂—O— near the quaternary nitrogen appeared at 4.25 ppm and the methylene group (23) N⁺—CH₂—CH₂—O—C(O)—near the oxygen appeared at 4.60 ppm. The two methyl groups (21) of the quaternary nitrogen appeared as two singlets at 3.61 and 3.62 ppm, while the two different protons of the methylene group (20) between the quaternary nitrogen and the carbonyl —O—C(O)—CH₂—N⁺— appeared each one as a multiplet at 4.79 and 5.46 ppm.

Vesicle Formation and Characterization

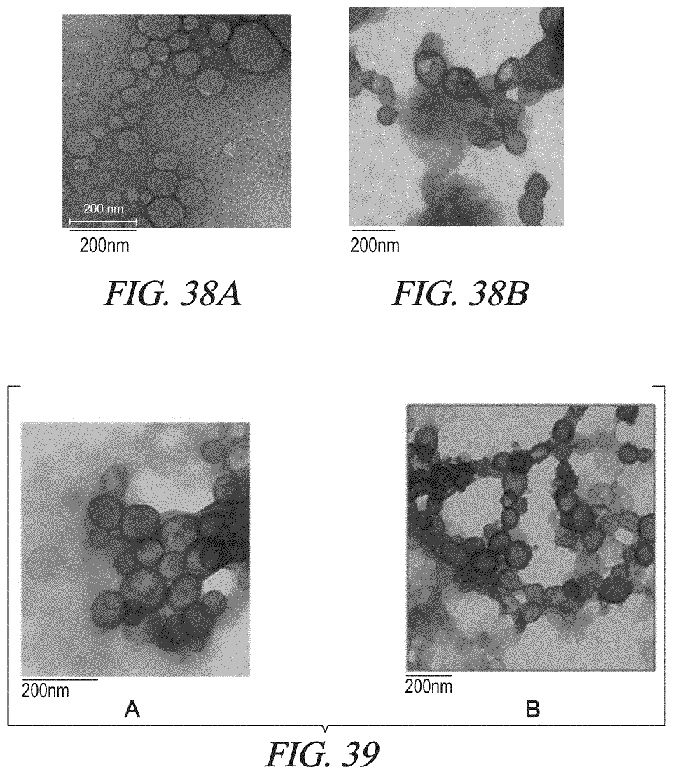

Amphiphiles in general, and specifically bolaamphiphiles, can form micelles, multilayered sheets, vesicles, rings, or a variety of microstructures with cylindrical geometry, such as rods, tubules, ribbons, and helices. The morphology of the self-aggregate structure is a function of the molecular parameters of the specific bolaamphiphile. The morphology of aggregate structures formed by film formation-hydration and sonication of the tested bolaamphiphiles was studied by transmission electron microscopy (TEM), and showed spherical aggregate nanostructures for both bolaamphiphilic compounds (FIG. 38).

As can be seen from FIG. 38, the vesicles were fairly heterogeneous in size with diameters ranging between 50-300 nm. The size distribution was determined by dynamic light scattering (DLS) and the data are shown in Table 11. The average diameter of the vesicles that were made from GLH-20 was 368 nm, whereas the average diameter of vesicles made from GLH-32 was 345. However, vesicles made from GLH-32 were more heterogeneous in size as only 68% of the main peak was within the range of the average diameter. The hydrodynamic diameter of the vesicles, as determined by DLS, was higher than the size seen in the TEM because the DLS measurements are size average dependent and also measure the size of the hydrated particles, whereas the hydration layer is not seen in the TEM micrographs.

TABLE 11
DLS measurements of vesicles from GLH-20 and GLH-32

| Bolaamphiphile | Diameter (nm) | Weight of Main peak (%) |
|---|---|---|
| GLH-20 | 368 | 97% |
| GLH-32 | 345 | 68% |

GLH-20 and GLH-32 are symmetrical bolaamphiphiles forming monolayer membranes. Due to differences in the void spaces between the bolaamphiphiles at the inner versus the outer surfaces of the vesicle's membrane, the aggregation of the bolaamphiphiles into a stable vesicle structure requires relatively large diameter vesicles to reduce the relative large differences in surface areas the inner and the outer surfaces. One way of stabilizing smaller vesicles made of symmetrical bolaamphiphiles is by incorporating additives that act as membrane stabilizers that will be situated among the outer parts of the bolaamphiphiles and thus, will be used as spacers that stabilize a higher curvature between the bolaamphiphiles. Membrane stabilizers, such as cholesterol (CHOL) and cholesteryl hemysuccinate (CHEMS), may be used for this purpose. In addition to serving as spacers, such compounds raise the order-disorder transition temperature and make the membrane more stable at higher temperatures. Upon incorporating CHOL and CHEMS, together with the symmetrical bolaamphiphiles, into the vesicle formulation, we observed that the bolaamphiphiles aggregated into smaller vesicles (FIG. 39), which were also more homogeneous in size as compared to vesicles that were made from the bolaamphiphiles without the additives (Table 12).

TABLE 12
DLS measurement of vesicles made from GLH-20 and GLH32 formulated
with CHOL and CHEMS at a ratio of 2:1:1.

| | Diameter | Weight (%) |
|---|---|---|
| Bolaamphiphilic compond | (nm) | main peak |
| GLH-20 | 120 | 100 |
| GLH-32 | 134 | 100 |

Vesicle Stability

Vesicle stability was evaluated by measuring both changes in the concentration of encapsulated carboxyfluorescein (CF) and vesicles size as a function of time when incubated in PBS at room temperature.

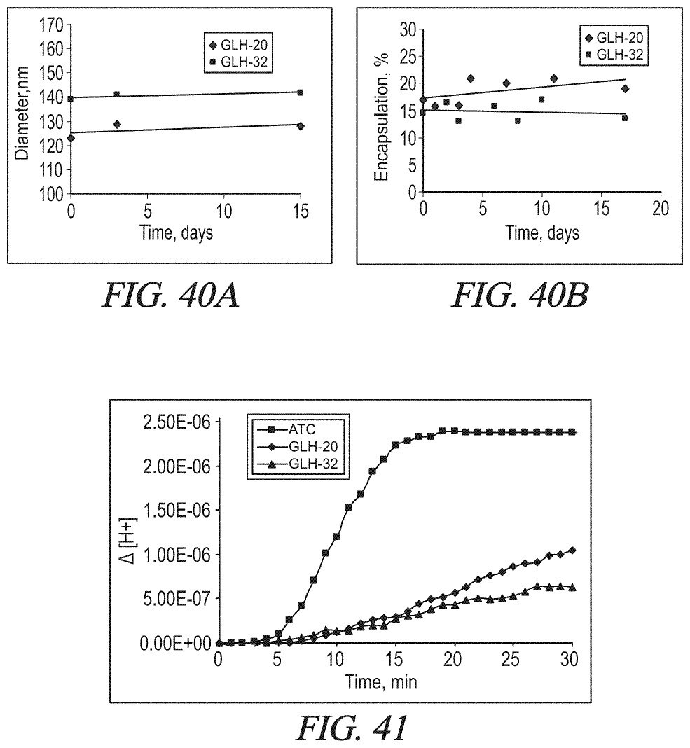

Preliminary studies showed that vesicles that were made from the bolaamphiphiles without CHOL and CHEMS tended to aggregate during time and form large particles. Therefore, the stability studies were performed with the vesicles that contained CHOL and CHEMS. Vesicles that were formulated with CHOL and CHEMS remained stable for at least 16 days (the last time point measured), without changing their size (FIG. 40A) or the amount of CF encapsulation (FIG. 40B).

Enzymatic Cleavage of the Head Group by AChE and Release of Encapsulated CF from the Vesicles Upon their Exposure to the Enzyme

When ACh head groups are covalently attached to the skeleton of bolaamphiphiles via the nitrogen atom of the choline moiety, the head groups are hydrolyzed by AChE (data not shown). For this study, it was hypothesized that the length of the alkyl chain which is adjacent to the ACh head group may affect head group's hydrolysis rate by by affecting how the ACh head group sterically fits into the the enzyme's hydrolytic site. FIG. 41 shows that indeed, the head groups of both bolaamphiphiles are hydrolyzed by AChE, but the rate of the hydrolysis of GLH-20's head group is significantly faster than that of GLH-32's head group, suggesting that a longer alkyl chain near the ACh moiety retards the hydrolysis rate. By comparison, the hydrolysis of free acetylthiocholine (ATC—an analogue of ACh) is much faster than that of the ACh head groups of both bolaamphiphiles, corroborating our conclusion that an alkyl chain adjacent to the ACh head group affects the rate of the hydrolysis.

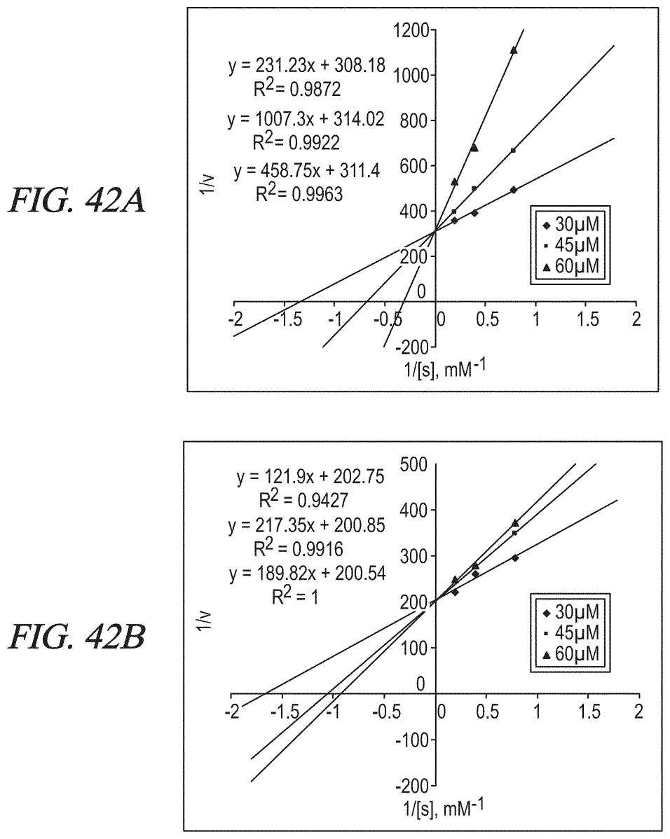

The finding that the head groups of both GLH-20 and GLH-32 are hydrolyzed by AChE suggests that both bolaamphiphiles bind to the enzyme and therefore, may compete with ATC for binding to the enzyme and inhibit its hydrolysis. In order to compare between the inhibitory potentials of the two bolaamphiphiles, the rate of ATC hydrolysis was measured in presence of three concentrations of the GLH compounds and assessed the results by a Lineweaver-Burk analysis. From FIG. 42 it can be seen that both GLH-20 and GLH-32 acted as competitive inhibitors, as increasing their concentrations affected the Kₘ but not the Vₘₐₓ. Yet, the tested concentrations of GLH-20 (FIG. 42A) affected the Km significantly more than the same concentrations of GLH-32 (FIG. 42B), suggesting that the affinity of GLH-20's head group to the enzyme is higher than those of GLH-32. This finding suggest that GLH-20 is a better substrate for AChE than GLH-32, explaining why the rate of the hydrolysis of the GLH-20's head groups is faster than the rate of hydrolysis of GLH-32's head groups.

The hydrolysis of the surface groups on bolaamphiphilic vesicles results in the destabilization of the vesicular structure and the release of the encapsulated material (data not shown). The hydrolysis of the bolaamphiphile's head group was tested to see if there were a correlation between that rate and the rate of release of CF from vesicles after exposing them to AChE. Release of the encapsulated CF was measured by an increase in fluorescence that occurs when the released CF is diluted in the medium in which the vesicles are incubated. The encapsulated CF in the vesicles is quenched and when it is released into the medium it is diluted and dequenched, emitting a fluorescence signal.

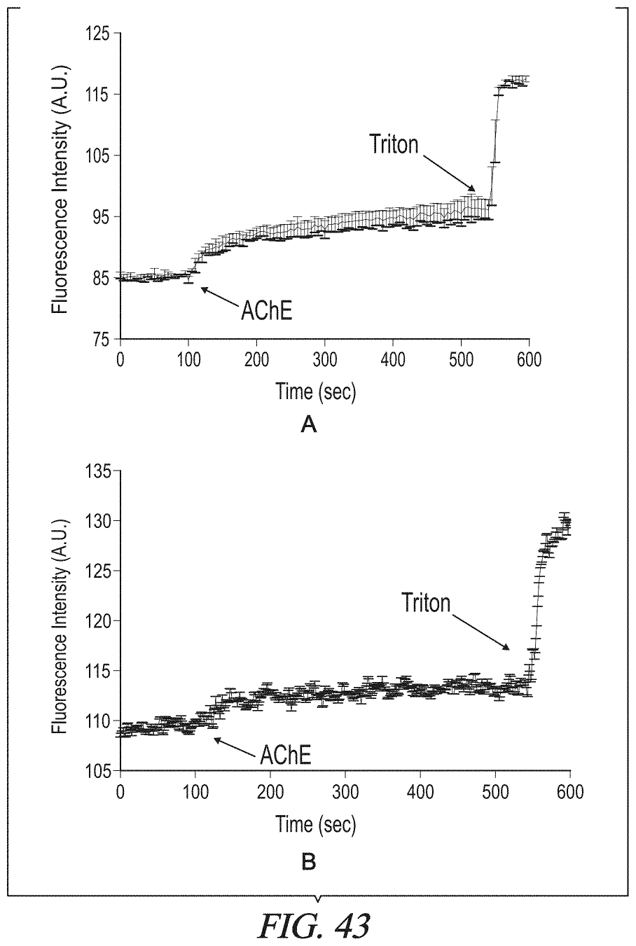

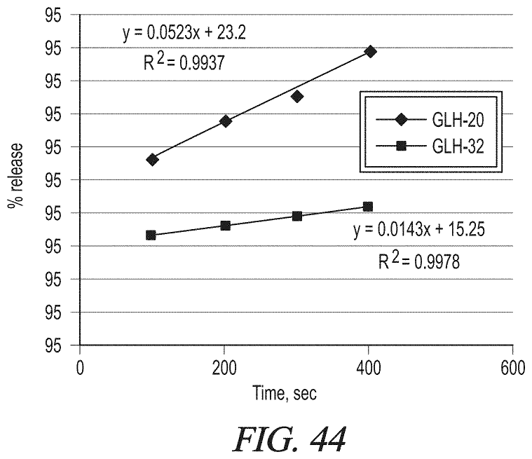

As can be seen from FIG. 43, both vesicles started to release their encapsulated material immediately after the addition of AChE to the vesicle suspension. The release rate was biphasic with a more rapid release rate seen immediately after the addition of the enzyme and then, after about 20-50 seconds, the release stabilized at a slower but constant rate. From FIG. 43 it can be seen that for both phases, the rate of release from GLH-20 vesicles was more rapid than the release rate from GLH-32 vesicles. To quantify the differences in the release rates from both vesicle types, the percent release from each vesicle preparation was calculated at 4 times after the addition of AChE, which were taken during the second phase. The results of this analysis (FIG. 44), show that the slope of the curve that represents the release as a function of time is significantly greater for GLH-20 vesicles, compared to GLH-32 vesicles.

The greater slope represents a faster release rate; indeed, at 400 seconds after the exposure of the vesicles to the enzyme, GLH-20 vesicles released about 44% of their content, whereas GLH-32 vesicles released only about 20% of their content (FIG. 44).

Altogether, these results demonstrate that although both GLH-20 and GLH-32 form similar vesicles that release their content upon exposure to AChE but release their encapsulated material at a different rate.

### EXAMPLE 24

### Compositions and Methods for Treatment of ALS

In another embodiment, the present disclosure is directed to compounds, compositions, and method of the treatement of neurological diseases including, for illustrative purposes amyotrophic lateral sclerosis (ALS). In one aspect of this embodiment, the present disclosure is directed to testing demonstrate in a mouse model of ALS beneficial effects of systemically administered GDNF, encapsulated in novel nano-sized vesicles provided herein.

The present disclosure provides vesicles that will be designed to target sites in the CNS where motor neurons degenerate and the encapsulated GDNF will be released at these sites, where the neurotrophin, upon its release, will induce its neuroprotective effect and may also cause neuronal regeneration. Targeting of the vesicles to sites in the CNS where motor neurons degenerate will be achieved by coating the vesicles with manose pendants that will direct the vesicles to activated microglia, which over express manose receptors. Selective release of the encapsulated GDNF is achieved by enzymatic hydrolysis of the head groups at the sites where the vesicles accumulate; in the case of the proposed vesicles—in regions of the CNS where activated microglia accumulate due to motor neurons degeneration in the ALS mouse. Specific elements of this approach include: 1) synthesis of bolaamphiphiles (bola)—the vesicle's building blocks; 2) formation of vesicles coated with manose pendants and encapsulation of GDNF in these vesicles; 3) testing the nano-sized vesicles for brain delivery and for targeting to activated microglia pharmacokinetic (PK) studies); and 4) demonstrating the beneficial effects of the delivered GDNF in an ALS mouse model. The above was repeated using neurotrophins such as insulin-like growth factor 1 (IGF-1) instead of GDNF, demonstrating significant beneficial effects in an ALS mouse model.

The GDNF-loaded vesicle system disclosed herein may be a breakthrough in the treatment of ALS for which there is no effective treatment. Moreover, developing the presently-disclosed nanovesicle platform for GDNF has wider implications for additional neurotrophic factors with the potential for ALS therapy as well as for other neurodegenerative diseases that may benefit from neurotrophic factors.

As demonstrated above, the present disclosure provides nano-sized vesicles made from bolaamphiphiles (bolas) that were designed drug delivery and were synthesized as described herein along with vesicles made of monolayer membrane that provides stability (due to high energy barrier for lipid exchange); high encapsulation capacity (due to their thin membrane that makes vesicles with a large inner volume), good brain penetrability (due to surface pendants that induce transcytosis via the brain microvessels endothelial cells) and an efficient controlled release mechanism (due to specific hydrolysis of the head groups at the target site). These vesicles have been used to deliver a variety of compounds into the brain, including small molecules, peptides, nucleic acids; proteins; and, as demonstrated above, e.g., GDNF.

Similar vesicles can be coated with manose pendants that will direct the vesicles to sites in the brain where motor neurons degenerate, for use in the treatment of ALS. The targeting concept is based on the notion and findings that in brain regions in which motor neurons degenerate, there is an accumulation of activated microglia [Xiao et al, 2007; Corcia et al, 2012; Liao et al, 2012; Hoyden et al, 2013] that overexpress manose receptors [Galea et al, 2005]. Our success in targeting vesicles coated with dopamine transporter ligand to dopaminergic neurons, suggest that there is a high probability that vesicles coated with manose pendants will be targeted to activated microglia, which are abundant in regions of degenerating motor neurons and over express manose receptors.

For ALS treatment, GDNF will be encapsulated in the vesicles described herein. As demonstrated herein, conditions for encapsulation of GDNF in vesicles have been worked out. In one approach will be to coat the vesicles with manose pendants followed by demonstrating that the encapsulated GDNF is beneficial in the treatment of ALS in an animal model. This will allowd encapsulation of additional neurotrophins with the aim of obtaining synergism and will also provide a strong rationale for performing clinical trials in human subjects. In another embodiment, ALS can be treated by administration of IGF-1 encapsulated in vesicles of the present disclosure, including, in one aspect vesicles coated with mannose pendants prepared according to the present disclosure. Therefore, in certain embodiments of the present disclosure ALS is treated by delivery of neurotropic factors including but not limited to GDNF, IGF-1, and combinations thereof, in vesicles of the disclosure, including those that comprise mannose surface groups.

Synthesis of bolaamphiphiles and the preparation and characterization of vescicles of the disclosure have been described above. In the present instance, vesicles will be prepared that are coated with mannose pendants will include encapsulated GDNF and or other neurotrophins such as insulin-like growth factor 1 (IGF-1). That is, a bola for the in vivo studies, i.e., a bola with the manose head groups, will be added to the vesicle formulation to coat the vesicles with manose pendants for targeting to activated microglia. The synthesis of this bola will be based on the methods used with many other bolas described herein. In various approaches, at lest two different vegetable oils can be used as the starting material (see below), including castor oil and vernonia oil.

### Formation of Vesicles Coated with Manose Pendants and Encapsulate GDNF and or Other Neurotrophins in these Vesicles.

In view of the data provided above, the manose head groups are expected not to affect vesicle properties except for targeting them to cells that express manose receptors. However, the proportions between the bolas within the vesicle formulation that will yield stable vesicles that are targeted to cells that express manose receptors will be determined before initiation of the in vivo studies.

In particular, vesicles made from GLH-19 are quite stable in whole serum (GLH-19 vesicles are not disrupted by choline esterase), whereas vesicles made of GLH-20 release their content in whole serum relatively quickly. In one approach, the ratio between GLH-19 and GLH-20 will be gradually adjusted to provide the most stable vesicles that still release their content upon exposure to choline esterase and this basic formulation will be used for all future studies. With the optimal formulation of GLH-19 and GLH-20 determined, the bola GLH-55B (an asymmetric bola with a CS head group on one side and acetylcholine head group on the other side) can be we incorporated into the formulation at the highest proportion that will not change vesicle stability. The last stage of optimizing the vesicle formulation will be an introduction of the bola with manose head groups into the vesicle formulation and test the proportion of this bola that does not affect vesicle properties. As a parameter for the targeting efficiency, endocytosis of vesicles with and without manose pendant into macrophage cell line that express manose receptors will be tested.

### Testing the Nano-Sized Vesicles for Brain Delivery and for Targeting to Activated Microglia Pharmacokinetic (PK) Studies)

Since GDNF is rather expensive, for testing targeting in vivo and for the PK studies, vesicles loaded with a fluorescent marker (carboxyfluorescein or FITC-dextran) will be used as a model system for initial experiments and the initial PK studies will be carried out with control mice. Mice will be injected with vesicles loaded with a fluorescent marker (vesicles with and without manose pendants) and the amount of the fluorescent dye in the brain will be measured. The proportion of the manose-bola trying will be varied (along with other parameters) optimize targeting efficiency without losing penetration into the brain in normal mice. In normal mice biodistribution of the encapsulated fluorescent dye in various tissues will also be tested. Upon finalization of an optimal composition, PK studies will be carried with ALS mice, using vesicles loaded with GDNF. Mice will be injected with GDNF-loaded vesicles with and without manose pendant and the distribution and the quantities of the GDNF in the brain will be determined using ELISA and immunohistochemical techniques (for details see method section).

### Demonstrating Beneficial Effects of the Delivered GDNF in an ALS Mouse Model

ALS mice were injected with the optimal vesicle formulation and efficacy parameters were assessed. The following experimental groups were used (10 animals per group): 1) Mice injected with empty vesicles as control; 2) Mice injected with optimal vesicles containing encapsulated GDNF; 3) Mice injected with free GDNF as a negative control. The mice received multiple injections of the test material during 45 days and the intervals between injections were determined in the PK studies (see above), whereas the criteria for the intervals were the time period that takes for the clearance of the GDNF from the brain. During the treatment period changes in body weight, test motor behavior, and performance of electromyographical analysis (see below) were measured. Life span of the treated mice was determined and used as one criterion for efficacy. In case of a life span shorter than the planned duration of the experiment, the treatment period was shortened accordingly and the mice were sacrificed at the end of the treatment period to test the effect of the treatment on motor neurons (see below).

Synthesis of bolas GLH-19, GLH-20, and GLH-55b are described above. Synthesis of the bolaamphiphile with the mannose head groups is provided in Scheme 7 (below).

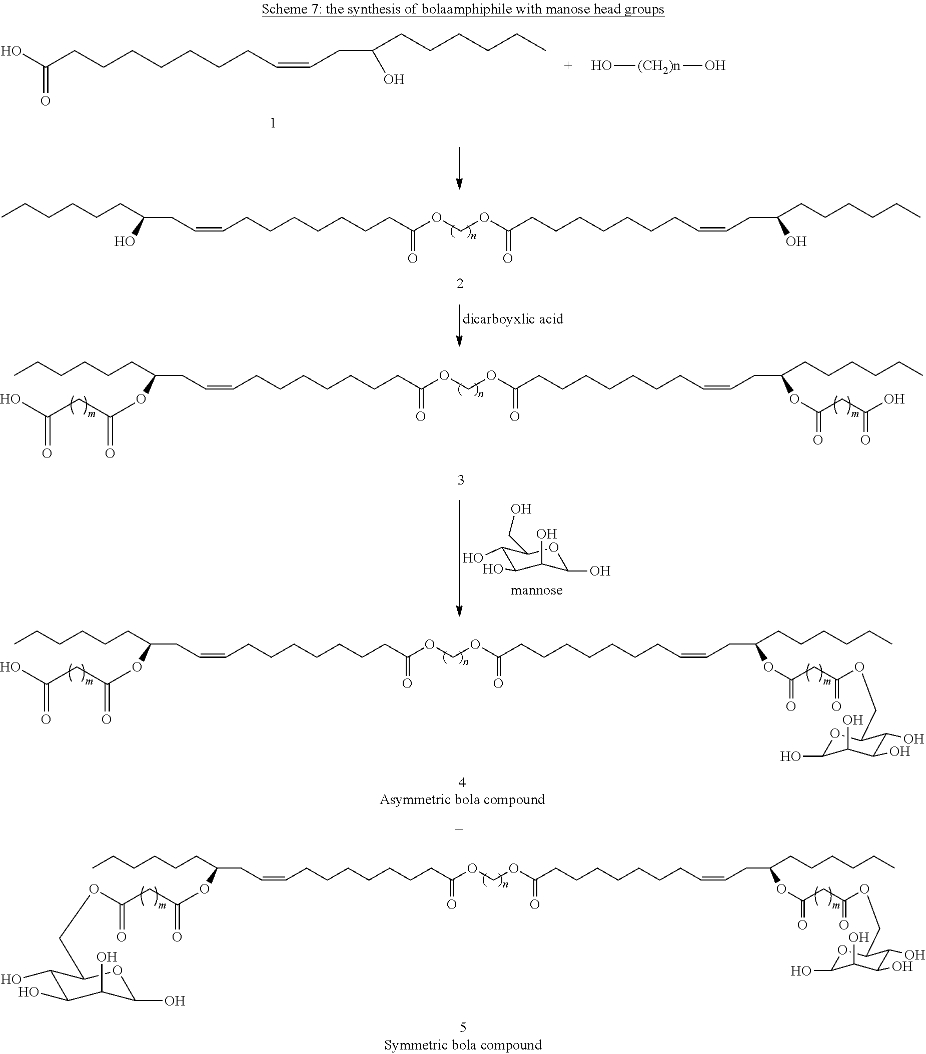

As indicated in this Scheme, ricinoleic acid (the main component of castor oil, >97%) was used as the starting material to form the hydrophobic skeleton of a symmetric bolaamphiphile. The diester 3 (see Scheme 7) was synthesized by the extension of the ricinoleic moiety in a chemoenzymatic esterification or transesterification reaction of ricinoleic acid (1, R=H) or methyl ricinoleate (1, R═CH₃) with aliphatic diols of different chain lengths using Candida antarctica lipase as the catalyst.

The second stage was the esterification of the secondary hydroxyl groups of the ricinoleic moiety of the diricinoleate 3 with a dicarboxylic acid in the presence of an acidic catalyst under azeotropic conditions.

The attachment of the mannose head group was performed by a chemoenzymatic esterification, in order to obtain selective binding to the primary hydroxylic position. This is a consecutive nucleophilic substitution reaction, which yields a mixture of monoester 4 and diesters 5, allowing the formation of a symmetric and asymmetric bolacompounds that were further separated by flash chromatography and their effect on vesicle formation, vesicle stability and targeting was investigated.

An alternative approach to this synthesis of a bola with the manose head groups was to use vernodiester 6 as the starting material similar to GLH-19 and GLH-20. The synthesis started by opening the epoxy group with a dicarboxylic acid. The attachment of D-mannose to the intermediate diester dicarboxylate was done by enzymatic esterification with Candida antarctica lipase.

Characterizatinn of the synthesized bolas, vesicle formation and characterization, GDNF encapsulation, determination of GDNF activity, determination of vesicle stability, and determination of GDNF release in vitro were carried out as described herein.

### Investigation of Targeting to Manose Receptors In Vitro

Macrophages that express manose receptors on their surface were grown in 24-well plates the medium was replaced with culture medium without serum and samples of carboxyfluorescein-loaded vesicles with and without manose pendants or free (non-encapsulated) carboxyfluorescein (equivalent to the encapsulated carboxyfluorescein) were added to the cells and incubated for 1-5 h at 4° C. or at 37° C. At the end of the incubation, cells were washed and either detached from the plates using cell detachment medium and analyzed by FACS (FACSCalibur Flow Cytometer, BD Biosciences, USA). The presence of manose pendants on the surface of the vesicles increased the uptake of the vesicles. Higher uptake was expressed in a shift of the peak in the flow cytometry profile and was indicative for targeting in vitro. This technique to measure targeting of vesicles that were coated with dopamine transporter ligand to cells that express dopamine transporter has been verified (data not shown).

### Pharmacokinetic (PK) Studies

Since GDNF is expensive, until the vesicle formulation is fully optimized encapsulated fluorescent marker were used to follow the PK properties of the vesicles. This approach was employed successfully when the biodistribution of vesicles that coated with dopamine transporter were studied and were targeted in vivo to dopaminergic cells in the brain (see above). Initially vesicles with encapsulated carboxyfluorescein were injected into normal mice and fluorescence in various tissues (blood, liver, kidney, lung, spleen, spinal cord and brain) was measured at various times after the injection. Mice (5 per group) were pretreated 15 min prior to the injection of the vesicles with 0.5 mg/Kg pyridostigmine to inhibit peripheral choline esterase. For comparison, in parallel to the pyridostigmine-treated animals, similar experiment was performed with animals that did not receive pretreatment with pyridostigmine. Tissues were removed at various times after the injection of the vesicles (1, 2, 4, 8, 12, 24, 48 hours), homogenized and deproteinized by trichloroacetic acid or perchloric acid. Fluorescence intensities were determined in the supernatant of the tissue extracts after centrifugation.

The next stage of the PK studies was done with SOD1 transgenic mice as an animal model for ALS. In this experiment vesicles coated with a targeting ligand (manose) were compared to vesicles without targeting ligand (naked vesicles). For this experiment GDNF-loaded vesicles were used and concentrations of GDNF in several regions of the CNS (spinal cord, cortex and cerebellum) were determined by ELISA, e.g., using commercially-available kits (e.g. Promega, Fitchburg WI). This testing was carried out using, e.g., 5 animals per group and the time points were selected according to the results obtained in the first stage of the PK studies (see above).

Another set of PK studies with SOD1 transgenic mice was done with vesicles loaded with GDNF-biotin. In this set of experiments the distribution of the injected GDNF in the CNS was determined by histofluorescence technique. Vesicles with and without targeting ligand were loaded with GDNF-biotin and were injected to ALS mice (3-4 animals per group). At various times after the injection, the animals were deeply anasthetized, perfused with PBS, brains and spinal cords removed and frozen sections were prepared from these tissues. The sections were stained with DAPI to visualize the nuclei and identify the region of the CNS according to the morphology. Co-staining with avidine-AlexaFlour was performed on the same sections to visualize the GDNF-biotin and histochemical staining of nucleoside-diphosphatase, an enzyme specific to microglia were performed in order to see if the GDNF was concentrated around the microglia.

### Efficacy Studies

ALS mice were divided into three groups (10 animals per group) as follows: 1) Mice that were treated with empty vesicles; 2) Mice that were treated with optimal vesicles loaded with encapsulated GDNF; 3) Mice that were treated with free GDNF. The treatment regimen was determined according to the results from the PK studies and continued for up to 45 days. The following parameters were used for the assessment of the efficacy of the treatment: A) Measurements of changes in body weight; B) Motor behavior: Several motor tests were performed in order to assess the condition of the ALS mice during the treatment period. The various tests were performed at different days. These tests were (1) Open field test: The animal was placed in the center of the open field apparatus and allowed to move freely for 10 min. The total distance moved, the frequency and duration of rearing (standing on the hind legs) and the time spent in the center area were recorded. The total distance moved was evaluated as an index for locomotor behavior and rearing behavior and time spent in the center was evaluated as an index for exploratory behavior. This test was performed every 4 days from the beginning of the treatmen; (2) Rotarod test: Mice were tested on the rotarod for the assessment of their motor function. The rotarod consists of five textured drums of 1.25 cm diameter. Total time that the mouse was able to remain on the rotating drum was recorded. Training consisted of habituation during which the mice were acclimatized to the rotarod at 5 rpm for 180 seconds and training during which they were allowed to remain on the rotarod at 10 and 15 rpm for 180 sec. On the test day, all mice were tested at 15, 20, 25 and 30 rpm for 180 sec and 10 min rest period were allowed between each trial. This test was performed one day befor the treatment and again before sacrificing the mice; (3) Swimming tank test. To assess progression of motor deficit, swimming movements were monitored in a water-filled tank. The device consisted of a 90-cm long, 6-cm wide and 40-cm high tank filled to a depth of 20 cm with water at a temperature of 24° C. A visible escape platform was positioned at the end of the tank. As a starting point for recording swimming performance, a vertical black line was drawn on one side of the tank, marking a horizontal 70-cm distance to reach the platform. A high-resolution web camera for video recording of limb kicks during swimming was used. As a training procedure, the mouse was allowed to swim from the starting line to the platform for 2 consecutive days with five trials per day. The mice were given five consecutive trials on a 10-d basis. To avoid artifacts and to always obtain the fastest swimming performance for each animal, analysis of the swimming latency was based on the mean scores of the three shortest latencies. The number of hindlimb kicks will be video recorded once for each animal and for each session. For mice with late-stage disease that may not be able to climb onto the platform anymore, the timer was stopped once the forepaws touched the platform. The maximum swimming latency was set at 20 sec.

C) Electromyographical analysis. Evoked CMAP amplitudes and spontaneous fibrillation potentials (SFPs) were evaluated with an electromyogram apparatus. Measurements were repeated every 7 days. Mice were anesthetized with isoflurane and the sciatic nerve was stimulated at a paraspinal site by a single 0.1-ms, 1-Hz supramaximal pulse through an unipolar needle electrode and recorded CMAPs from the medial part of the gastrocnemius with the same type of electrode. SFPs were recorded through a concentric needle electrode and only SFPs with an amplitude ranging from 20 to 300 μV were considered.

D) Histological analysis: At the end of the treatment session, mice were anesthetized and transcardially perfused with 50 ml of 4% paraformaldehyde in phosphatebuffered saline (PBS). Brain and spinal cords were post-fixed in the same fixative for 4 h and processed for either paraffin or cryoprotective embedding. For immunohistochemistry the tissue was stained using antibodies against EGFP, nonphosphorylated neurofilament and NF-L. Cryostat sections of 16-μm thickness were incubated with primary antibodies diluted in 4% bovine serum albumin, 5% donkey serum in PBS containing 0.1% Triton-X100. Immunoreactivity was visualized with AlexaFlour-conjugated secondary antibodies diluted in the same solution. For histopathological analysis, 8-μm deparaffinized sections were stained with cresyl violet and motor neurons were ccounted every five sections.

### EXAMPLE 25

### Compositions and Methods Comprising Bolaamphiphiles with Mannose Head Groups for Specific Targeting of Vesicles to ALS Sites in the CNS

Disclosed herein is a novel treatment for amyotrophic lateral sclerosis (ALS) that is based upon the use of bolaamphiphile vesicles capable of targeting degenerating motor neurons in the central nervous system (CNS) of ALS patients and release encapsulated GDNF near the degenerating motor neurons, where the released GDNF will provide its neurotrophic activity, namely, promoting neuroprotection and neuroregeneration. The targeting of these vesicles is achieved by surface groups that have high affinity to mannose receptors highly expressed in activated microglia that accumulate near degenerating motor neurons. The present disclosure provides vesicles with surface targeting ligands and that have been tested to demonstrate the vesicles' capability to target cells that highly express mannose receptors.

Described are vesicle compositions comprising the following components 1) the bolaamphiphiles (bolas) GLH-19 and GLH-20, which contain acetyl choline (ACh) head groups for controlled release of encapsulated GDNF; 2) the bola GLH-55b, which contains a chitosan (CS) head group to enhance penetration of the vesicles via the blood-brain barrier (BBB); 3) several types of GLH-64 (GLH-64a-e), a bola family with mannose head groups that target the vesicles to mannose receptors.

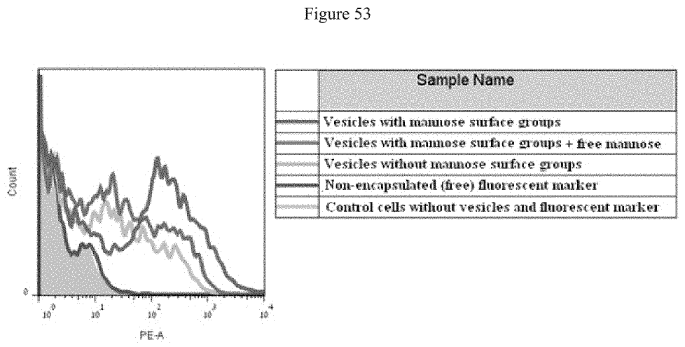

As demonstrated herein, vesicles that contain mannose moieties on their surface (mannose surface groups were introduced by inclusion in the vesicle formulation one of the GLH-64 bola's family, particularly GLH-64a), provide efficient targeting of GLH 64 with the mannose head group. As can be seen from FIG. 53 (below), the vesicles that contained mannose surface groups were taken up about 10 times more than vesicles that did not contain mannose groups on their surface. Inclusion of free mannose in the bathing medium (10 mM) completely abolished the effect of the mannose surface groups since it competed with the mannose surface groups for binding to the mannose receptors that were expressed on the membrane of the differentiated cells. Free (non-encapsulated) fluorescent probe (siRNA conjugated with alexaFluor 546) was not taken up by the cells at all and the peak of the cells that were exposed to the free fluorescent probe was identical to the peak of the control cells that were not exposed to neither vesicles and fluorescent probe. The data below show that the vesicles with their encapsulated fluorescent probe, and not the free fluorescent probe, were taken up by the cells and that vesicles with mannose surface groups were taken up by the cells much more than vesicles without the targeting ligand on their surface. Altogether, these results indicate that vesicles with mannose surface groups target cells that express mannose receptors.

Theresults obtained with GLH-64a are conclusive and show that a bola which is bound to the mannose moiety via the primary hydroxyl which is situated on carbon 6 is capable of providing efficient targeting.

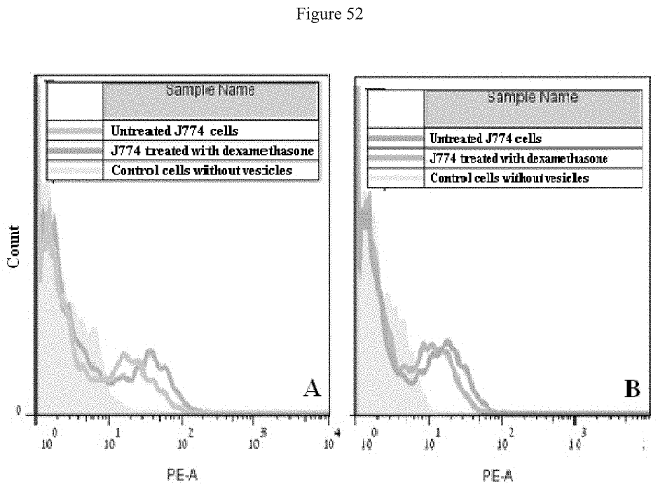

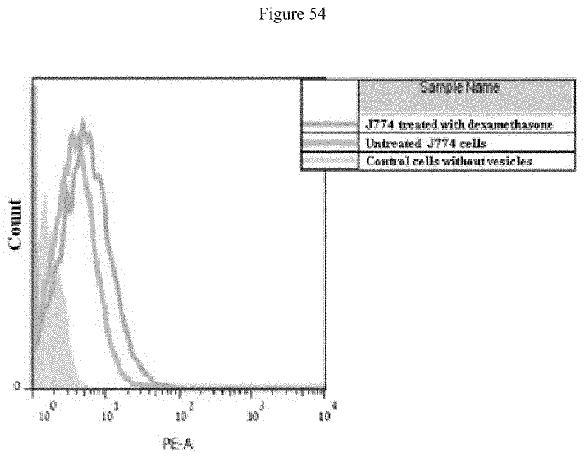

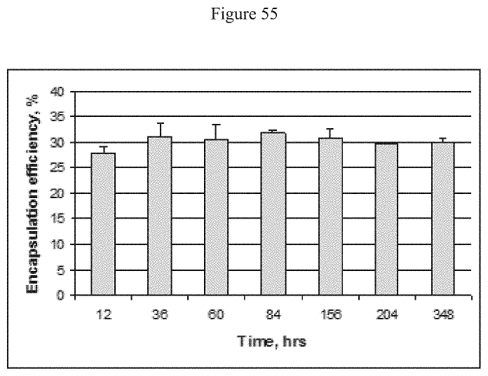

The results obtained with GLH-64b showed that targeting can be achieved with this bola as well, although the uptake of the vesicles that contain GLH-64b (uptake indicates targeting) was somewhat smaller than that obtained with GLH-64a (see FIG. 52). GLH-64b contains a mixture of bolas where the mannose is bound to the bola skeleton either via the primary or the secondary hydroxyls. Therefore, it was interesting to see whether a bola in which the mannose moiety is bound only via the secondary hydroxyl is capable of providing good targeting. GLH-64d is such a bola in which the mannose moiety is bound via the secondary hydroxyl, which is situated on carbon number 1 of the mannose. The results of the targeting experiment with GLH-64d are described in FIG. 54 (below) As can be seen from Figure54, vesicles that contain GLH-64d were taken up somewhat better by differentiated cells than by non-differentiated cells, but the shift was much smaller than that obtained with GLH-64a. Based upon all results of the targeting experiments described herein, targeting can be achieved with vesicles that contain mannose surface moieties, particularly when the mannose is bound to the bola's skeleton via the primary hydroxyl situated on carbon 6 of the mannose.

### Synthesis of GLH 64a-e, a Bola Family with Mannose Head Groups

This bola family contains D-mannose head groups for targeting of the vesicles to mannose receptors. When such bolas are included in vesicle formulation, the mannose head group is positioned on the vesicle's surface and provides a targeting moiety that is expected to be recognized by and bind to the mannose receptor. Binding of the mannose moiety to the bola's skeleton can be done via each of the hydroxyl groups of the mannose, Since it was not clear which hydroxyl group or groups are important for the recognition and binding to the mannose receptors, several species of GLH-64 (GLH-64a-e) were prepared that contain both alpha and beta configurations of the sugar and the binding was done via either the primary hydroxyl, or via one of the secondary hydroxyl groups of the mannose. The synthesis of these bolas is described herein.

### Synthesis of GLH-64a, a Bola with Mannose Moiety Bound to the Bola'S Skeleton Via the Primary Hydroxyl of the Mannose

The starting material for the synthesis of this first species of GLH-64 was ricinoleic acid (the main component of castor oil, >97%), which was obtained by the hydrolysis of castor oil. The synthetic steps are shown in Scheme 7 (above). Ricinoleic acid (compound 1 in Scheme 7) was reacted with aliphatic diol to achieve extension of the ricinoleic moiety by a chemoenzymatic esterification of ricinoleic acid (1, R═H), using Candida antarctica lipase as the catalyst, providing compound 2 of Scheme 7.

The second stage was the synthesis of the bola's skeleton (compound 3 in Scheme 7) by esterification of the secondary hydroxyl groups of compound 2 (Scheme 7) with a dicarboxylic acid in the presence of an acidic catalyst under azeotropic conditions. The attachment of the mannose head group to the bola's skeleton was achieved by a chemoenzymatic esterification, in order to obtain selective binding to the primary hydroxylic position. This constituted a consecutive nucleophilic substitution reaction, which yielded a mixture of the monoester 4 and the diester 5 (GLH 64a). The mixture of the monoester D-mannose bolaamphiphile (compound 4, 36.4%) and the diester D-mannose bolaamphiphile GLH-64a (2.3%) were separated by flash column chromatography on silica gel, using detection by common spectroscopic methods.

### Analysis of Reaction Products by FT-IR, ¹H and ¹³C NMR as Well as by ESI-MS.

FT-IR analysis showed the appearance of the broad absorption band of O—H for GLH-64a and compound 4 and the disappearance of the carbonyl band of the carboxylic acid (1712 cm⁻¹) for GLH-64a.

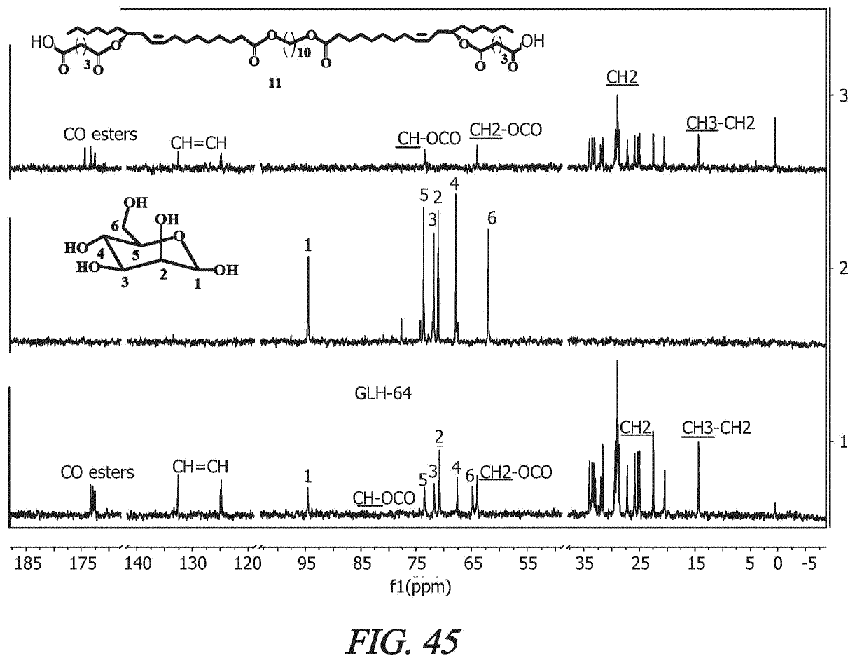

¹H and ¹³C NMR spectra were performed in DMSO-d6 and it appeared that both compound 4 and GLH-64a showed the same ¹H NMR signals, differentiated only by the number of their protons in the integration curves. In the ¹H NMR spectrum of GLH-64a, in addition to the signals of the D-mannose moieties, we observed the presence of two multiplets of the protons from CH═CH groups (5.42 and 5.28 ppm), multiplet of the methine protons of the ricinoleic moieties (4.76 mppm), as well as the methylene protons of the glutarate unit near the carbonyl esters at 2.31 and 2.29 mppm. The chemical shifts of the methine and methylene protons of D-mannose groups were assigned by analyzing the two dimensions HMQC spectrum of GLH-64a in comparison with the HMQC spectrum of D-mannose in the same solvent. This spectrum showed the following direct correlations: C1-H (94.49 and 4.86 ppm), C2-H (71.75 and 3.54 ppm), C3-H (70.73 and 3.71 ppm), C4-H (67.47 and 3.37ppm), C5-H (73.36 and 4.78 ppm) and C6-H2 (64.73 & 4.31). However, it is worth mentioning that the signals of the methylene group of the primary alcohol (CH2-OH) appearing in the D-mannose HMQC spectrum at 62.01 ppm and 3.61and 3.42 ppm were deshielded and shifted to lower magnetic fields for GLH-64a (64.73 and 4.31 ppm). FIG. 45 provides the 13C NMR spectra of the diester diglutarate 3 (Scheme 7), D-mannose and the bola GLH-64-a in DMSO-d6. Contrary to the ¹³C NMR spectrum of GLH-64a (FIG. 45) that shows the disappearance of the peak of the carbonyl of COOH groups of the glutaric acid moieties, the ¹³C NMR spectrum of compound 4, showed the presence of the carbonyl of the carboxylic acid at 174.43 ppm and the appearance of a new peak of carbonyl of ester at 172.60 ppm (COO-mannose) in addition to the two other peaks of carbonyl of ester groups of the molecule. We also saw in the spectrum of compound 4 the presence of the peak at 33.11 ppm for the methylene carbon of the glutaric acid unit adjacent to the carboxylic acid moiety (CH2-COOH), the peak at 33.41 ppm for the methylene carbon of the glutaric acid moiety attached to the mannose ester unit (CH2-COO-mannose) and the signals of the D-mannose carbons at 94.54, 73.53, 71.75, 70.81, 67.61 and 64.87 ppm.

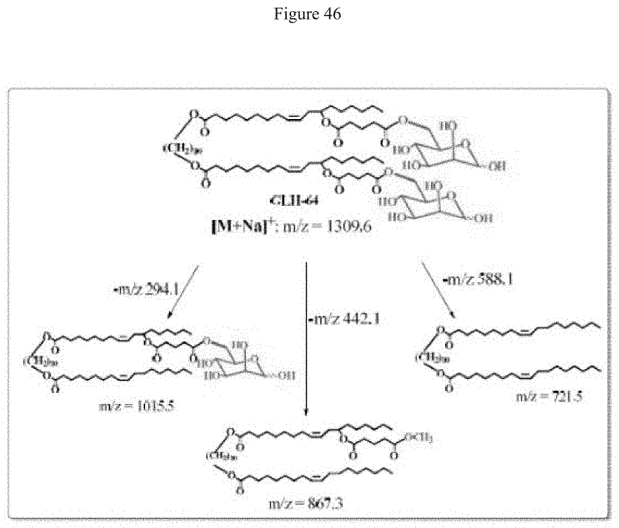

The formation of bolaamphiphiles GLH-64a and compound 4 was also confirmed by ESI-MS analyses. In a negative mode, the mass spectrum of compound 4 (containing one mannose head group and one carboxylic acid head group) showed the peak of the molecular ion at m/z 1124.3, matching with the molecular weight of its formula C₆₂H₁₁₈O₁₇. The mass spectrum of bola GLH-64a showed in a positive mode the peak of the molecular ion at m/z 1286.6, corresponding to the molecular weight of the formula C68H118O22, which is GLH-64a. The fragmentations of the molecular ions [M+Na]⁺ in positive mode of both compounds showed the presence of peaks at m/z 721.4 and 1015.4. The signal at m/z 721.4 corresponds to the fragment of their molecular ions without all the glutaric acid and D-mannose moieties. The peak at 1015.4 in the case of compound 4, is the fragment of the molecular ion without one glutaric acid moiety and for GLH-64a, this signal represents the fragment of the molecular ion without one glutaric acid and unit of D-mannose, as shown in FIG. 46 which presents the main fragmentations of GLH-64a in ESI-MS (positive mode).

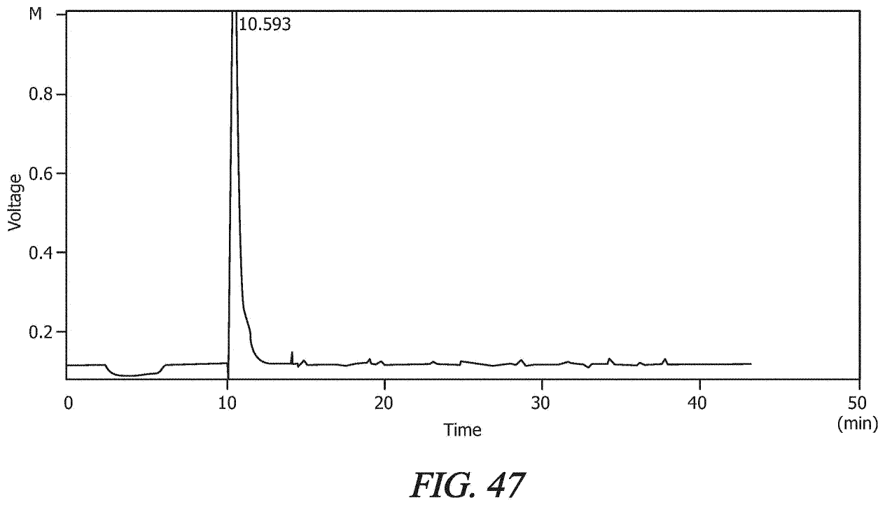

Initial experiments provided about 50 mg of GLH-64a with high purity as can be seen from the HPLC chromatogram presented in FIG. 47. This quantity was sufficient for product characterization and to perform initial studies with vesicles that contain GLH-64a. Described below is an alterntive, improved process for the synthesis of GLH-64a.

### Experiment 26

### Alternative Proess for GLH-64a Synthesis

Improvements in the synthesis of GLH-64a were achieved by varying several parameters as follows:

A) Previously, to obtain GLH-64a the starting material ricinoleic acid or methyl ricinoleate (see Scheme 7) was that obtained by hydrolysis of triricinolein, or as a product of transesterification of castor oil (Scheme 8).

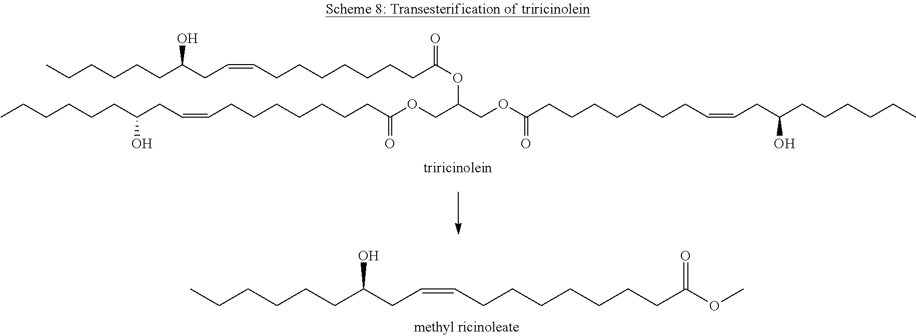

Castor oil contains, besides the triricinolein, other triglycerides (about 10%) and the metyl ricinoleate that was obtained from the transesterification of triricinolein needed to be purified, e.g., using flash chromatography to separate the methyl ricinoleate as in the procedures above, a procedure requiring expensive silica gel and substantial amounts of a hexane-diethyl ether (7%) mixture. It also involved the collection and analysis of many fractions, a method better suited for milligram to gram scale.

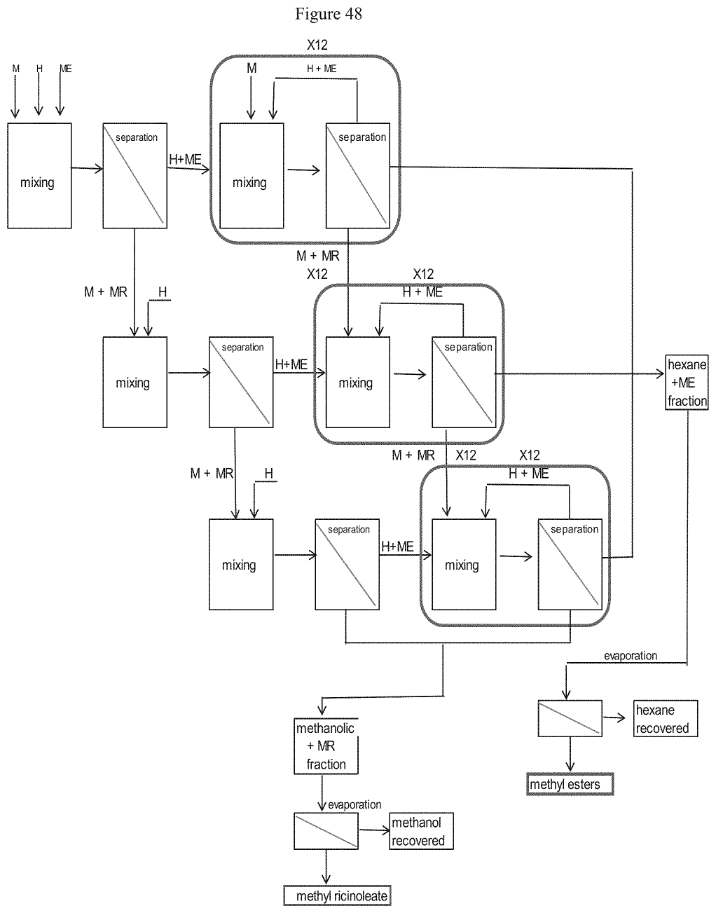

To improve the synthesis of GLH-64a, particularly at a larger scale, a liquid-liquid extraction of methyl ricinoleate was employed, without using the chromatography separation according to procedures described in the literature [Bordeaux et al., JAOCS 1997; 74 (8):1011]. This liquid-liquid extraction procedure which enabled purification of large amounts of methyl ricinoleate, is presented schematically in FIG. 48, and is described below:

Crude methyl ricinoleate (20 g) was shaken with 120 mL of hexane and 60 mL of 90% aq. methanol in the first separating funnel. The layers were separated, and the methanolic phase was removed. The hexane phase was extracted with another 12 portions of 60 mL of 90% aq. methanol consequently one after another to yield 13 portions of methanolic solution. Each methanolic solution was sequentially passed through two more separating funnels, each containing 120 mL of hexane. Each methanolic solution was examined by thin layer chromatography (TLC) using hexane:ether (1:1) to obtain pure methyl ricinoleate. The solvent was removed under reduced pressure. 35.8 g of the mixture methyl ricinoleate and was used without further purification.

The procedure was repeated using 76 g of crude methyl ricinoleate. The same proportions of hexane and methanol were used. 702.6 g of hexane and 2065 g of methanol were recovered. The hexane residue was examined and no methyl ricinoleate was found in this fraction, but it contained about 10 g (about 13% from total esters) of other methyl esters that were separated from the methyl ricinoleate.

B) In initial experiments for the synthesis of GLH-64a p-toluene sulfonic acid was used as a catalyst in the reaction of esterification of diester diricinoleate with glutaric acid (scheme 9)

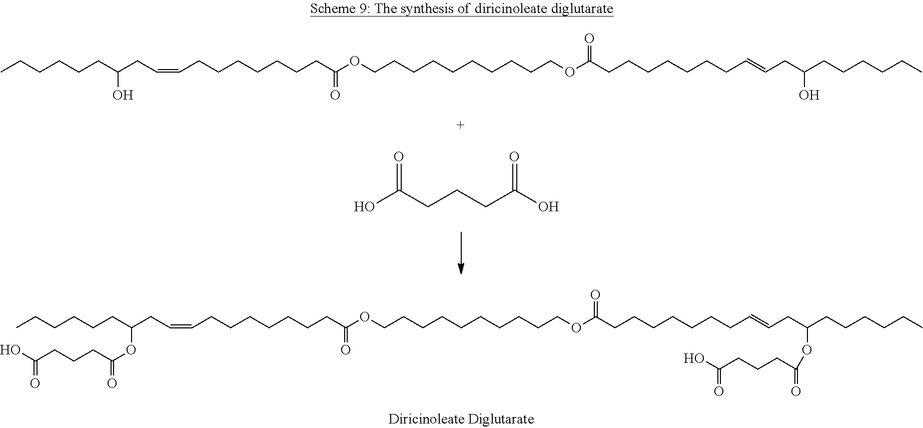

Initial experiments required the presence of a soluble catalyst that required multiple washings with water at the end of the reaction. Then the solution that contained the product had to be dried out with MgSO₄. In this alternative, improved approach, Amberlyst 15 was used as an acidic solid catalyst. Since it is a solid, it is easy to remove this catalyst by filtration and the washings was saved.

C) In initial experiments, binding of the mannose moiety to diricinoleate diglutarate (scheme 10), provided only a low product yield, apparently because only a small amount of mannose could have been dissolved in most of the organic solvents used in esterification reaction. Accordingly, solvents that dissolve higher amounts of mannose, such as DMSO or pyridine, were tested but the enzyme lipase that was used in this reaction was not active in these solvents. Therefore, the reaction was performed in heterogeneous conditions using t-butanol as a solvent, but the yield was low. In an attempt to overcome this problem, a super-saturated solution of mannose in ionic liquid (1-butyl-1-methylpyrolidinium trifluoromethanesulfonate) was used, but it was difficult to remove the ionic liquid. In an attempted alternative approach, involved binding the mannose without using the lipase.

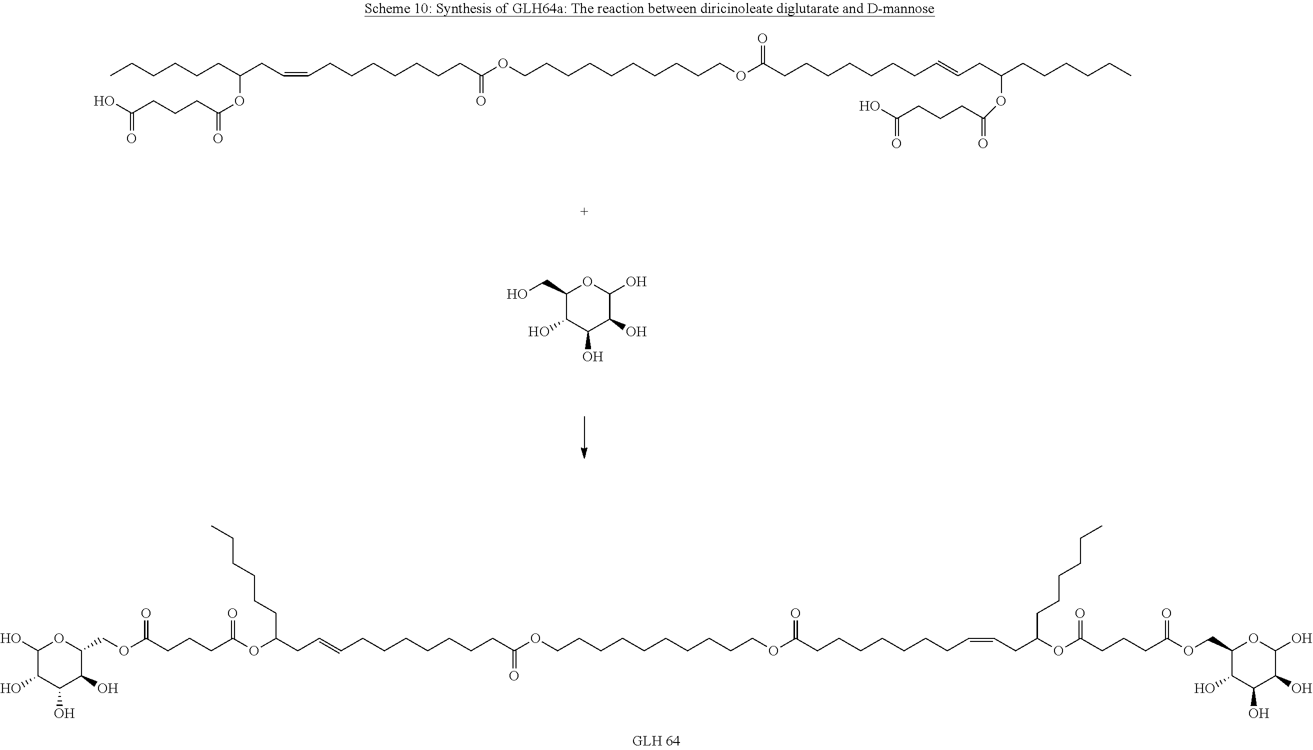 [SMILES: CCCCCCC(C/C=C\CCCCCCCC(=O)OCCCCCCCCCCOC(=O)CCCCCCC/C=C/CC(CCCCCC)OC(=O)CCCC(=O)O)OC(=O)CCCC(=O)O ; OC[C@H]1OC(O)[C@@H](O)[C@@H](O)[C@@H]1O ; CCCCCCC(C/C=C\CCCCCCCC(=O)OCCCCCCCCCCOC(=O)CCCCCCC/C=C/CC(CCCCCC)OC(=O)CCCC(=O)OC[C@H]1OC(O)[C@@H](O)[C@@H](O)[C@@H]1O)OC(=O)CCCC(=O)OC[C@H]1OC(O)[C@@H](O)[C@@H](O)[C@@H]1O]

This reaction was performed in pyridine (in which mannose is soluble in relatively high quantities) as a solvent and EDCI*HCl was used as a water scavenger. A solution of EDCI*HCl in dry CHCl₃ was added drop-wise to a solution of diglutarate diricinoleate in dry CHCl₃ at −5° C. (over ice with NaCl). Then the reaction was stirred overnight at room temperature. After the removal of the solvent under reduced pressure, the product was purified by flash chromatography using CHCl₃—CH₃OH (7 -8%) as an eluent. Although the product was obtained in relatively high yield, it contained a mixture of bolas with the mannose bound via both the primary and secondary hydroxyls of the mannose (note that the lipase binds the mannose selectively via the primary hydroxyl and in the absence of the lipase the binding was not selective). Since the product was a mixture of bolas in which the mannose moiety is bound via both the primary and the secondary hydroxyls, it was essentially different than GLH-64a and therefore, it was named GLH-64b.

### EXAMPLE 27

### Synthesis of Additional GLH-64 Species

To facilitate the synthesis of additional GLH-64 species (GLH-64 c-e) with the mannose bound specifically via the primary or the secondary hydroxyls of the mannose, selective binding of the mannose to the bola's skeleton via either the primary or one of the secondary hydroxyls of the sugar, was provided by the use of protected mannose compounds as reagents.

A) The use of monoprotected mannose-alfa D-1-benzyl-mannopyranose to prepare GLH-64c: The compound alfa D-1-benzyl-mannopyranose (a derivative of mannose with protection on the hydroxyl group of carbon number 1) is soluble in tert-butanol, but not in chloroform. Therefore, the reaction was performed in tert-butanol in the presence of lipase Novozym 435 as a catalyst (Scheme 11).

The reaction was performed at room temperature and at 60° C. The products contained a mixture of many materials. The target product (GLH-64c) was obtained in less than 5% yield by chromatography when the reaction was carried out at 60° C. The reaction at room tem perature gave almost no target product.

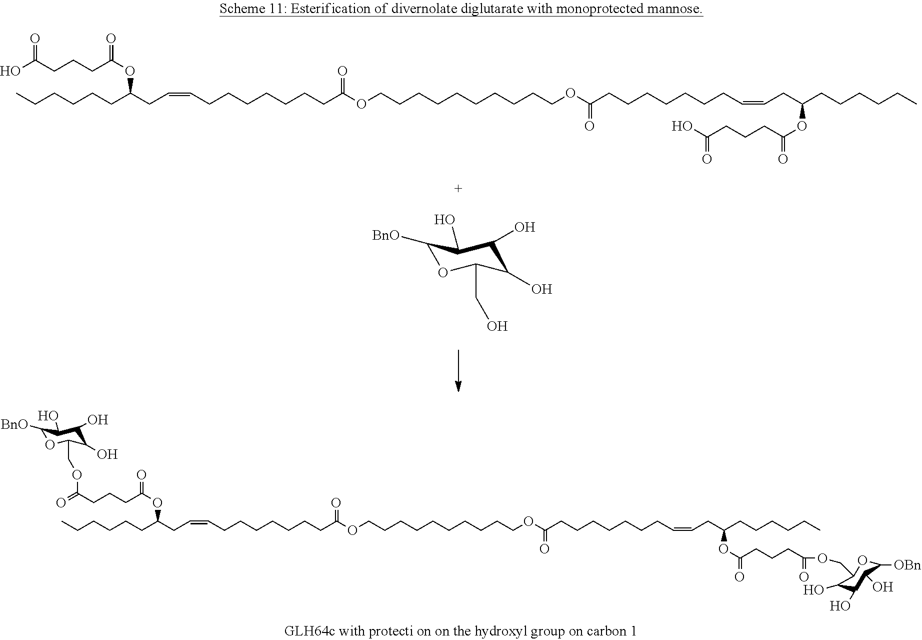

B) Completely protected mannose was used to prepare other GLH-64 species (GLH-64d-e): The protected mannose is soluble in organic solvents and the reaction should be selective. In this case specific binding of the mannose to the bola's skeleton was obtained without the use of lipase.

The protected mannose was synthesized using benzyl bromide [Lu et al. Carbohydrate Research 2005; 340:123] (Scheme 12)

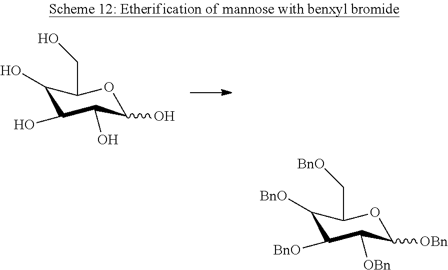

Mannose was added to a suspension of powdered KOH in DMSO. The suspension was cooled in an ice bath and stirred with mechanical stirrer. Benzyl bromide was added drop wise. The temperature was allowed to reach room temperature and the reaction was stirred overnight. The product was extracted with diethyl ether, washed with water and saturated NaCl solution, dried with MgSO₄, and the solvent was removed under reduced pressure. A mixture of hexane and ethyl acetate (8% ethyl acetate) was added and the solution was kept in a freezer overnight. Decantation and recrystallization from the hexane ethyl acetate mixture were performed. The precipitate was filtered out and TLC (hexane:ethyl acetate 8:2 was used as the running solvent) showed one spot. HPLC (CH₃CN 100%, 97.6% purity), Mass Spectrometry (MS) m/z [M+23]⁺=654 and NMR spectra of the product confirmed the identity of the obtained compound.

The next step was to replace the benzyl group on C6 of protected mannose with the acetate group (selective transesterification) , as depicted in Scheme 13:

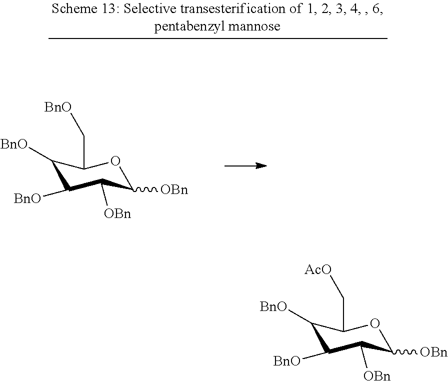

This reaction was performed in two ways: The freshly prepared solution of 1:1 TMSOTf-CH₂Cl₂ was added drop-wise to a solution of carbohydrate in freshly distilled acetic anhydride at −78° C. (cooled in acetone bath with dry ice). Then, the reaction was stirred at −78° C. for lh under nitrogen. The cold bath was removed and a saturated solution of NaHCO₃ and CH₂Cl₂ was added. The mixture was stirred for 0.5 h. The organic layer was separated. The aqueous layer was extracted with CH₂Cl₂ and the organic layers were combined, washed with water, dried with Na₂SO₄ and the solvent was removed under reduced pressure. Acetic anhydride was still present in the mixture and MS showed the presence of the mixture of products containing 1, 2, and 3 acetic groups.

The other way of selective 6-O-debenzylation was to use ZnCl₂. ZnCl₂ was melted at 340° C. for lh, cooled and a solution of 1:5 HOAc : Ac₂O was added. The carbohydrate was added drop wise at −5° C. for about lh. Then ice water was added. The aqueous phase was extracted with CHCl₃ and the combined organic phases were washed with NaHCO₃sat., H₂O, NaCl sat., dried over MgSO₄ and the solvent was removed under reduced pressure. MS showed the presence of a mixture of products containing 1, 2, and 3 acetic groups, but the main product was the target compound with one acetate group, yet, acetic anhydride was still present. Nevertheless, this method provided advantages over that described above, in that there was no need to use very low temperatures (−78° C.) and no need to use expensive reagent (TMSOTf). The results were also better in terms of yield.

The next step was the hydrolysis of the acetate 6-O mannose group, as depicted in Scheme 14.

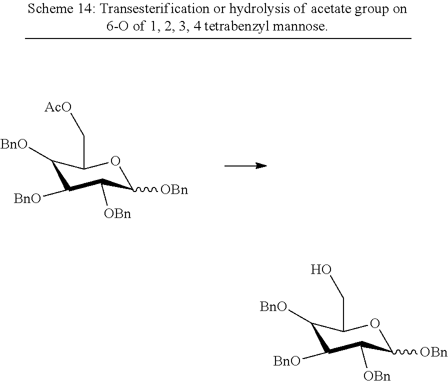

Initially, the reaction was performed according the procedure described in Lu et al. Carbohydrate Research 2005; 340:123. A solution of NaOMe in methanol was added to a suspension of crude material from the previous reaction in dry methanol. The reaction was stirred for 6h. No target product was observed. NaOH and water were then added and the mixture was stirred for 0.5h. Methanol was removed under reduced pressure. Water and diethyl ether were added, the phases were separated. The organic phase was washed with water, NaClₛₐₜ, dried over MgSO₄ and the solvent was removed under reduced pressure. The purification of the product was performed using flash chromatography with hexane:ethyl acetate 8:2 as an eluent. TLC (hexane:ethyl acetate 7:3), MS m/z [M+18]⁺=558.07 and HPLC (CH₃CN 100%) confirmed the identity of the product.

C) Binding protected mannose to diglutarate diricinoleate through secondary hydroxyl on C-1 to obtain GLH-64d was carried out using commercially available 1-OH-2,3,4,6, tetrabenzyl mannose that was reacted with diglutarate diricinoleate by the addition of the solution of EDCI*HCl in dry CHCl₃ to a solution of diglutatate diricinoleate, tetrabenzyl mannose and DMAP in dry CHCl₃ and cooling with ice+NaCl. The reaction mixture was allowed to reach room temperature and was stirred overnight. TLC (hexane:ethyl acetate 7:3) showed new spots and no diglutarate was observed. Water and more chloroform were added. The phases were separated and the organic phase was washed with 2M HCl, NaCl_{sat.}, dried over MgSO₄ and the solvent was removed under reduced pressure. The reaction is described in Scheme 15.

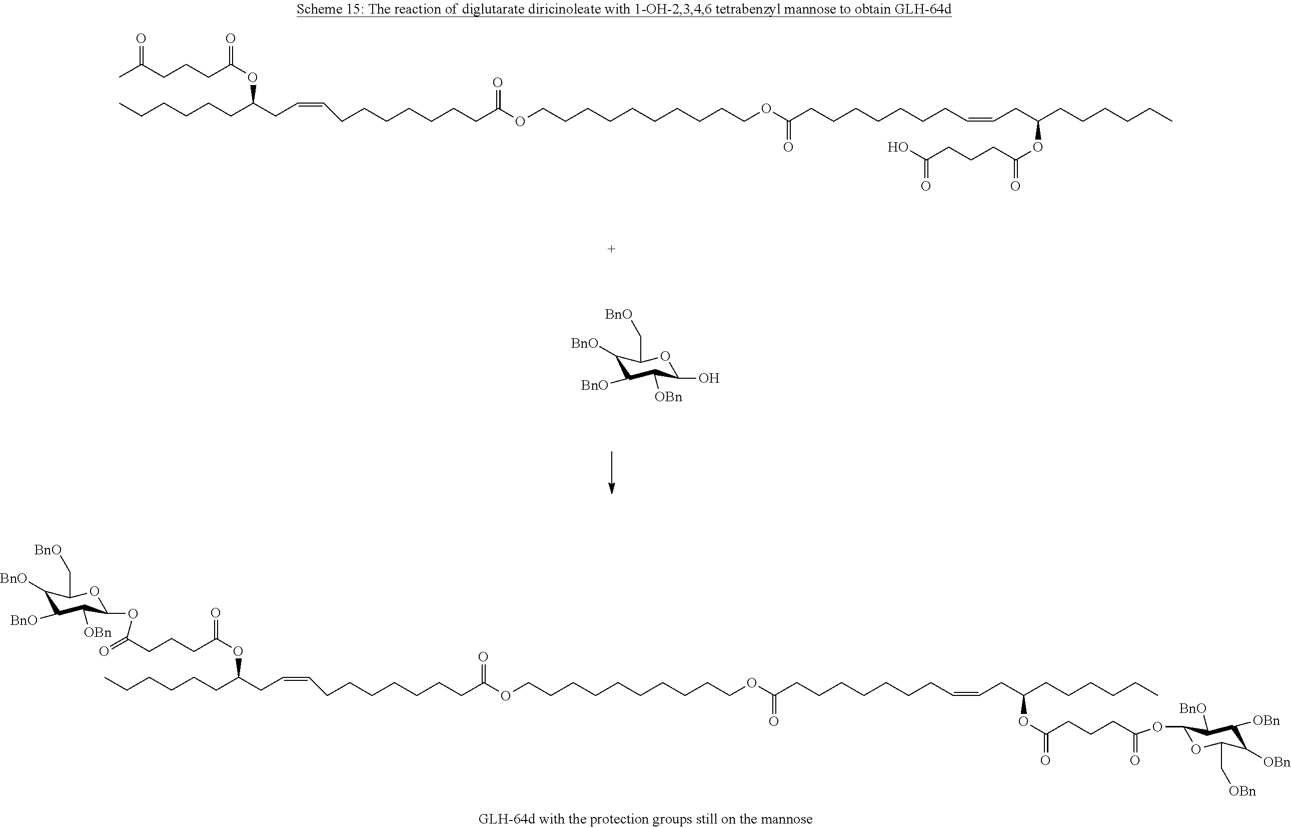

Purification of the product was performed using flash chromatography with hexane:ethyl acetate 8:2 as an eluent. HPLC (CH₃CN 100%) showed two main fractions that contained the target product that was characterized by MALDI m/z [M+Na]=2030.

D) Binding of the protected mannose to diglutarate diricioleate via the primary hydroxyl on C-6 to obtain GLH-64e, was carried out using a reaction performed similarly to the reaction described above and is described in Scheme 16. MALDI m/z [M+Na]+=2030 confirmed the identity of the product.

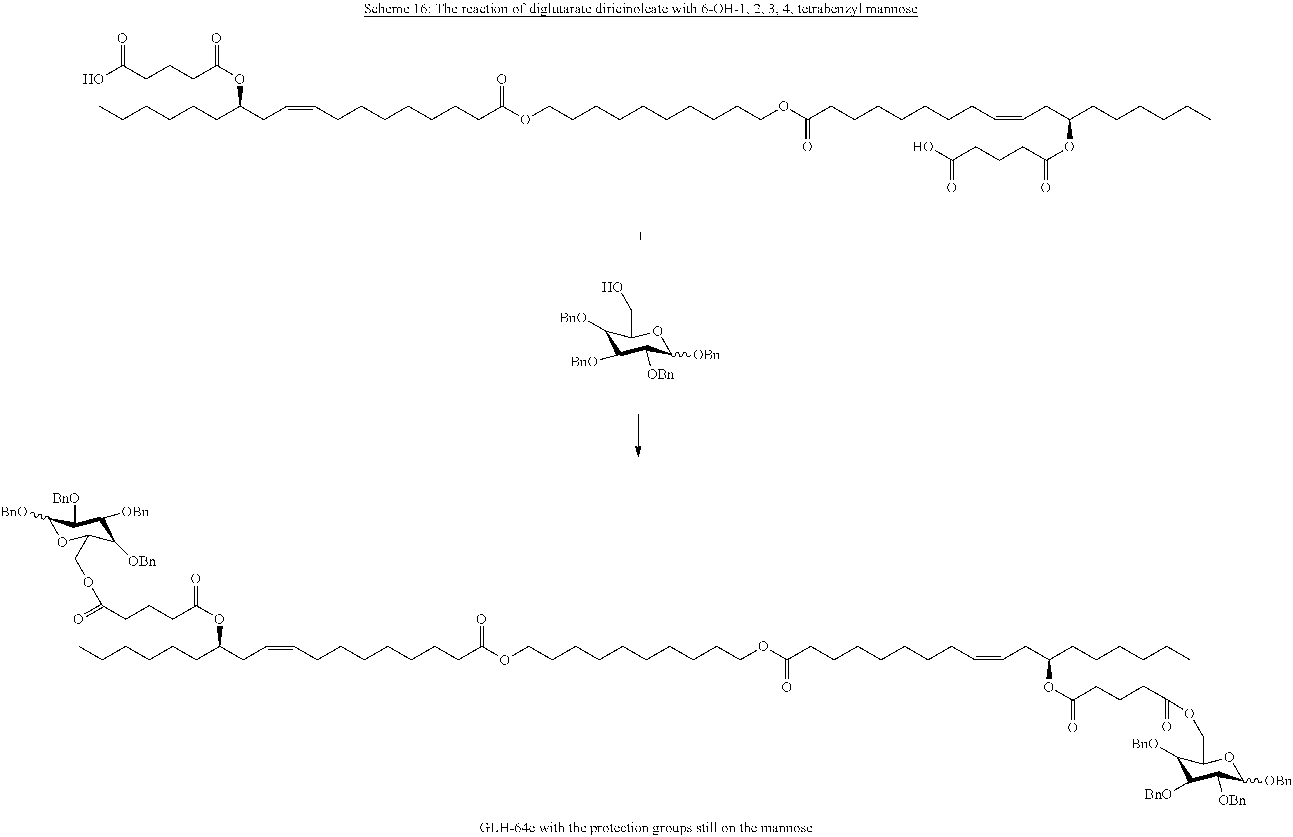

Removal of the protection groups to obtain GLH-64a with unprotected mannose bound to its skeleton, required removal of the protection from the mannose moiety. Removal of benzyl groups from the products of bola-protected mannose was performed in the ethyl acetate:methanol mixture 1:3 with 10% Pd/C as a catalyst as described in Scheme 17.

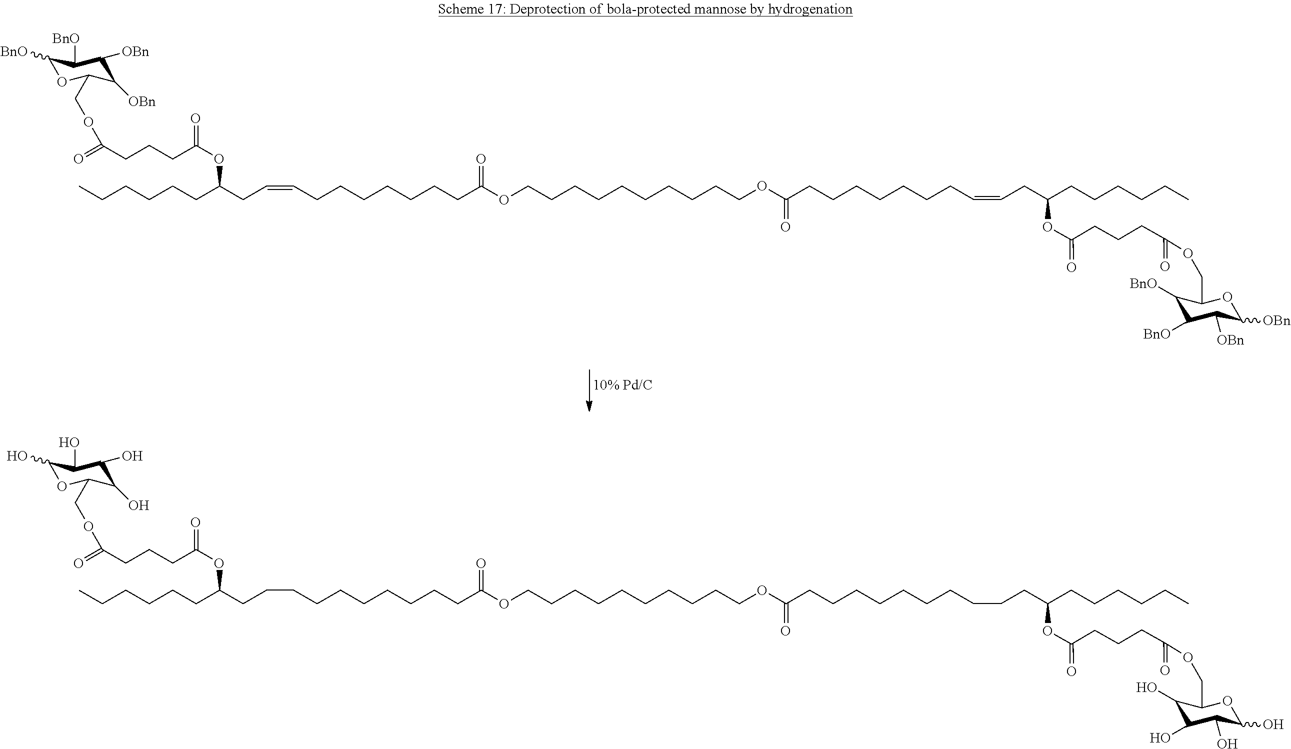

### EXAMPLE 28

### Studies with the Synthesized Bolas

Toxicity of the newly-prepared GLH-64 bolas was examined to determine the levels to be used in vesicle formulation and testing. The newly-synthesized bolas were also used for vesicle formation and characterized the resulting vesicles. In addition, a model protein was encapsulated and the efficiency of that process determined. The model protein used, trypsinogen, resembles GDNF in both molecular weight and pI point (characteristics relevant for encapsulation). In particular, these studies were to determine the effect of inclusion of GLH64 bola family members on vesicle formulation, encapsulation efficiency, as well as other properties of the vesicles that are needed for drug delivery, including but not limited to stability and controlled release.

The customized, stable vesicles obtained, which ere capable of encapsulating a protein similar to GDNF, were tested for their ability to target cultured cells that express mannose receptors. For these purposes siRNA conjugated with AlexaFluor 546 was encapsulated since this fluorescent probe provided the strongest signal in the FACS used to separate fluorescent cells from non-fluorescent cells (the cells become fluorescent after taking up vesicles with encapsulated fluorescent probe and targeting of the vesicles to mannose receptors causes more vesicle uptake and therefore, more cells became fluorescent). The results obtained are described below:

Toxicity studies: a suspension of GLH-64 was injected intravenously into the tail vein of male mice, starting with a dose of 100 mg/kg. This dose was selected as the initial dose for the toxicity studies based on the preliminary estimation that with the vesicle formulation would include no more than 10% of a GLH-64 species and previous studies showed that the maximal tolerated dose of a mixture of GLH-19, GLH-20 and GLH-55b (at a ratio of 2:1:0.1) was about 100 mg/kg. Therefore, if a GLH-64 species were to be found to be non-toxic at 100 mg/kg, the use of this bola in the vesicle formulation is safe at any potential vesicle formulation comprising a mixture of GLH-19, GLH-20, GLH-55b and a GLH-64 species. Three male mice were injected with 100 mg/kg GLH-64a and 3 other male mice with a mixture of bolas GLH-19, GLH-20, GLH-55b and GLH-64a at a ratio of 2:1:0.1:0.1, respectively. No signs of toxicity were observed at a dose of 100 mg/kg for either GLH-64a alone, or with the mixture of the bolas as described above. Thus, GLH-64a does not seem to be toxic at the dose range that will be used for the PK and efficacy studies.

Vesicle formation and characterization: The bolaamphiphiles that were synthesized for this project were used to form vesicles (V-Smart™ vesicles) customized for targeting to cells that express mannose receptors and release their encapsulated compounds there. The following sections describe the studies that were done with these vesicles (V-Smart™ vesicles).

The effect of GLH-64a on vesicle shape, size and surface charge (zeta potential): In previous studies, spherical vesicles were routinely obtained with diameters ranging between 50-150 nm with a net positive surface charge (Zeta potential in the range of 30-50 mV). Vesicles with this range of size and surface charge showed good drug delivery properties. To assess the effect of GLH-64a on these vesicles' properties, we prepared vesicles with different amounts of GLH-64a in the vesicle formulation and investigated their properties in term of shape (cryo-TEM), size distribution (DLS) and zeta potential.

Vesicles were prepared by film hydration followed by sonication as described above. The tested formulations included: a) GLH-19 and GLH-20 at a molar ratio of 2:1, respectively. This formulation contains only the basic bolas that make up the membrane matrix of the vesicles; b) GLH-19:GLH-20:GLH-55b at molar ratios between the bolas of 2:1:0.1, respectively. This formulation contains also a bola with CS (chitosan) head groups and it represents our standard formulation that showed, in previous experiments, the capability of delivering small molecules [Popov et al. (2012) Site-directed decapsulation of bolaamphiphilic vesicles with enzymatic cleavable surface groups. J Controlled Release, June 10; 160(2):306-14], peptides [Popov et al. Delivery of analgesic peptides to the brain by nano-sized bolaamphiphilic vesicles made of monolayer membranes. Eur J Pharm Biopharm. 2013 November; 85 (3 Pt A): 381-9] and proteins [Dakwar et al. (2012) Delivery of proteins to the brain by bolaamphiphilic nano-sized vesicles. J. Controlled Release, June 10; 160(2):315-21] into a mouse brain, thus this formulation was used as a control for comparison purposes; c) GLH-19:GLH-20:GLH-55b:GLH-64a at molar ratios of 2:1:0.1:0.01, respectively; d) GLH-19:GLH-20;GLH-55b:GLH-64a at molar ratios of 2:1:0.1:0.05, respectively; e) GLH-19:GLH-20:GLH-55b:GLH64a at molar ratios of 2:1:0.1:0.1, respectively. All the formulations contained also CHOL (cholesterol) and CHEMS (cholesterol hemisuccinate) at a molar ratio of 2:1:1 (bolas:CHOL:CHEMS, respectively).

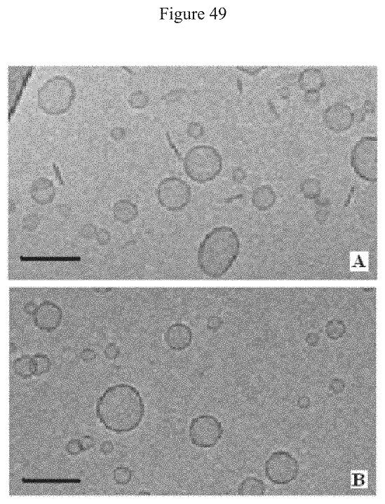

Cryo-TEM of vesicles with and without the bola that contains mannose head groups (GLH-64a): Images of vesicles with and without mannose surface pendants are shown in FIG. 49. As can be seen, spherical vesicles were obtained from formulation without GLH-64a (FIG. 10, Panel A) and formulation with 5% GLH-64a (FIG. 10, Panel B). The size of the vesicles ranged from about 50 nm to about 120 nm. A more quantitative analysis of size distribution was obtained by dynamic light scattering (DLS) measurements

Size distribution and zeta potential of vesicles without and with GLH-64a bolas; i.e., the effect of the bola with the mannose head groups (GLH-64a) on vesicle size and surface charge is shown in Table 14.

TABLE 14
Effect of GLH-64a bolas with mannose head groups on vesicle size
and charge.

| | Without (−) or | | |
|---|---|---|---|
| | with (+) CS | Vesicle size | |
| Percent of GLH-64a | (GLH-55b) in | determined by | Zeta |
| in the vesicle | the | DLS | potrential |
| formulation | formulation | (nM) | (mV) |
| 0 | − | 148.7 ± 0.7 | 50.2 ± 0.9 |
| 0 | + | 136.5 ± 5.8 | 47.6 ± 0.7 |
| 1 | + | 137.5 ± 7.9 | 42.4 ± 0.6 |
| 5 | + | 107.3 ± 0.9 | 48.4 ± 1.1 |
| 10 | + | 87.6 ± 5.9 | 41.3 ± 0.1 |

As demonstrated in Table 14, increasing the amount of GLH-64a in the vesicle formulation caused a gradual decrease of the vesicle size from a diameter of 136.5 nm, which was measured for vesicles without GLH-64, to a diameter of 87.6 nm measured for vesicles with 10% GLH-64a. Applicants believe, without wishing to be held to that belief, that smaller vesicles may accumulate more selectively in the brain due to less filtration in the lung and better permeability through the BBB.

Vesicle stability in storage: To study the effect of vesicle composition on stability, vesicleswith encapsulated CF (5,6-carboxyfluorescein) were prepared and the percent CF still encapsulated was measured as a function of time in storage. Vesicles of the following formulations were prepared: a) A mixture of GLH-19 and GLH-20 (2:1) was used as a control; and b) A formulation similar to that described in ‘a’ above with the addition of different amounts of GLH 64a (1%, 5% and 10% of the total bolas in the vesicle formulation). Each of the formulations described in ‘a’ and ‘b’ above, was prepared with or without the bola that contains the chitosan head group (GLH-55b). All formulations contained also CHOL and CHEMS at a molar ratio of 1:1 as described.

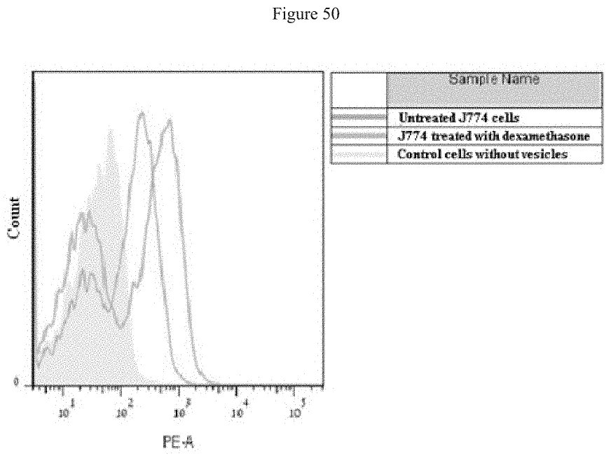

Targeting of (V-Smart™) vesicles to cultured macrophages that express mannose receptors: The ability of the customized vesicles (V-Smart™ vesicles) (vesicles that were customized to target to mannose receptors) to target cells that express mannose receptors was examined using J774 macrophage cell line. This cell line does not normally express mannose receptors, but it can be differentiated by dexamethasone to express significant number of mannose receptor [Fiani et al. Regulation of mannose receptor synthesis and turnover in mouse J774 macrophages. J Leukoc Biol. 1998; 64(1):85-91]. Vesicles with the capability of binding to mannose receptors are expected to bind preferentially to differentiated J774 cells, but not to non-differentiated J774 cells [Dubey et al. Surface structured liposomes for site specific delivery of an antiviral agent-indinavir. J Drug Target. 2011; 19(4):258-69]. The vesicles described herein are designedt to target microglia that accumulate in the CNS near degenerating motor neurons in ALS. These activated microglia express mannose receptors. Accordingly, the mannose-receptor-expressing J774 macrophage cell line can be used a s model cells to study targeting. Binding of the vesicles to the cell surface results in the uptake of the vesicles into the cells by means of endocytosis [Dakwar et al. (2012) Delivery of proteins to the brain by bolaamphiphilic nano-sized vesicles. J. Controlled Release, June 10; 160(2):315-21]. When more vesicles bind to the cell's surface, more vesicle uptake will occur, therefore, targeting of the vesicles to cells will increase their uptake into the cells. To assess the amount of the uptake of the vesicles by J774 cells, vesicles loaded with a fluorescent probe were used that when are taken up by the cells make them fluorescent. Fluorescent cells are separated from non-fluorescent cells by FACS, where the peak of the fluorescent cells is shifted to the right (see, e.g. FIGS. 50-54) as compared to non-fluorescent cells. The degree of the shift indicates the degree of the binding and is related to targeting.

For the assessment of the targeting, binding of the customized vesicles (vesicles that contain mannose on their surface) was compared to that of non-differentiated and differentiated J774 cells. Vsicles that contain mannose surface groups with vesicles that do not contain mannose surface groups were also compared for their ability to bind differentiated J774 cells. To investigate the specificity of the targeting, the binding to differentiated J774 cells of vesicles with mannose surface groups in presence and absence of free glucose in the bathing medium was also mesured. That is, if the binding of the mannose surface groups to the mannose receptor is specific, then free mannose will compete with the bound mannose (the mannose on the vesicle surface) and will reduce binding (thus will reduce uptake of the encapsulated fluorescent materials).

FIG. 50 shows results from flow cytometry (FACS) obtained with customized vesicles carrying surface mannose groups (V-Smart™), demonstrating the uptake of fluorescent vesicles that contain GLH-64a by differentiated and non-differentiated J774 cells. Cells were differentiated by exposing them to 1 μg/mL dexamethasone for 24 hours. Untreated cells were grown in parallel to the differentiated cells, but without dexamethasone. Cells were incubated with fluorescent vesicles that contained mannose moieties on their surface (by the including 5% GLH-64a in the vesicles formulation) for 4 hours and were examined by FACS. A shift of the peak to the right indicates higher uptake (namely higher binding)Binding of the vesicles was measured to non-differentiated cells (cells that do not express mannose receptors) in comparison to differentiated cells (cells that highly express mannose receptors). As can be seen, the peak of the fluorescent cells was shifted more to the right (about 8 times more) for differentiated cells compared to non-differentiated cells. These results indicate that the customized vescicles bind 8 times more to cells that express mannose receptors compared to cells that do not express mannose receptors.

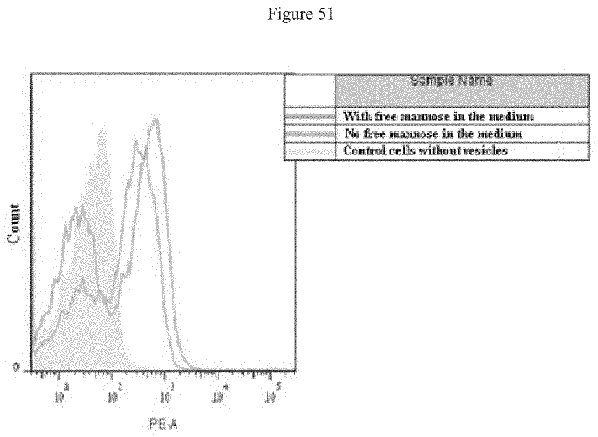

The specificity of the binding is demonstrated in FIG. 51, which depicts a comparison between the bindings of the customized vesicles carrying surface mannose groups described herein to differentiated cells that was done in presence and absence of free mannose in the bathing medium. More specifically, FIG. 51 depicts that uptake of fluorescent vesicles formulated with GLH-64a by differentiated J774 cells in presence and absence of free mannose in the bathing medium. Cells were differentiated by exposing them to 1 μg/mL dexamethasone for 24 hours. Cells were incubated with fluorescent vesicles that contained mannose moieties on their surface (by the including 5% GLH-64a in the vesicles formulation) for 4 hours and were examined by FACS. A shift of the peak to the right indicates higher uptake (namely higher binding. As can be seen, the presence of free mannose in the bathing medium decreased the uptake of the fluorescent vesicles, indicating that the free mannose competed with the mannose surface groups of the vesicles and interfered with the binding of the vesicles to the cells.

Additional targeting experiments were done with vesicles that contain higher proportion of GLH-64a, to determin if increasing the surface density of mannose groups would improve targeting. In this experiment, the GLH-64a level in the formulation was increased to 10% and the contributions of GLH-64a and GLH-64b in the formulation were compared. The mannose moiety in GLH-64a is bound via the primary hydroxyl on carbon number 6 of the mannose while GLH-64b is a mixture of bolas in which the mannose is bound via either the primary or any one of the secondary hydroxyls of the mannose as the reaction was done without lipase and the binding of the mannose was not site specific, as described above. The advantage of GLH-64b is that it can be obtained in high yield and its synthesis is simpler than that of GLH-64a. The results of the targeting experiment with vesicle formulations that contained 10% GLH-64a and GLH-64b in comparison are shown in FIG. 52, which presents the uptake of fluorescent vesicles that contain GLH-64a (Panel A) and GLH-64b (Panel B) by differentiated and non-differentiated J774 cells. Cells were differentiated by exposing them to 1 μg/mL dexamethasone for 24 hours. Untreated cells were grown in parallel to the differentiated cells, but without dexamethasone. Cells were incubated with fluorescent vesicles that contained mannose moieties on their surface (by the including 10% GLH-64a (Panel A) or 10% GLH-64b (Panel B) in the vesicles formulation) for 4 hours and were examined by FACS. A shift of the peak to the right indicates higher uptake (namely higher binding).

As can be seen from FIG. 52, vesicles that contained both GLH-64a and GLH-64b were taken up more by differentiated cells that express mannose receptors than by non-differentiated cells that do not express mannose receptors. However, the signal of the fluorescent cells was smaller than the signal obtained in the first experiment (compare the data of FIGS. 50 and 51 to that of FIG. 52). Although these results migh reflect a different amount of the fluorescent probe that was used in the second experiment, or less efficient differentiation of the cells that resulted in lower expression of mannose receptors by the differentiated cells, the shifts of the peaks of the differentiated cells that were exposed to the customized vesicles are clear and significant. These data therefore indicate that the vesicles with the mannose surface group target cells that express mannose receptors. To validate the conclusion that only the vesicles that contain mannose surface group target cells that express mannose receptors, binding of vesicles without mannose head groups (vesicles that were prepared from formulations that did not contain GLH-64) was compared to the binding of vesicles that contain mannose surface groups (a formulation with GLH-64a), using differentiated cells that express mannose receptors. In this experiment for the differentiation of the cells was obtained by contact with 10 μg/mL dexamethasone (instead of 1 μg/mL that was used in earlier experiments) to assure efficient differentiation. The results of the experiment in which vesicles without mannose surface groups were compared to vesicles with mannose surface groups are shown in FIG. 53, which depicts uptake of fluorescent vesicles with and without GLH-64a by differentiated J774 cells. Cells were differentiated by exposing them to 10 μg/mL dexamethasone for 24 hours. Cells were incubated with fluorescent vesicles (vesicles with encapsulated siRNA conjugated with AlexaFluor 546) with mannose moieties on their surface (mannose surface groups were introduced by the including 5% GLH-64a in the vesicles formulation) or with fluorescent vesicles without mannose surface groups (without GLH-64 in the vesicle formulation) for 4 hours and were examined by FACS. Cells were also incubated with non-encapsulated (free) siRNA conjugated with AlexaFluor 546, which was used as the fluorescent probe. A shift of the peak to the right indicates higher uptake (namely higher binding).

As can be seen from FIG. 53, the vesicles that contained mannose surface groups were taken up about 10 times more than vesicles that did not contain mannose groups on their surface. Inclusion of free mannose in the bathing medium (10 mM) completely abolished the effect of the mannose surface groups since it competed with the mannose surface groups for binding to the mannose receptors that were expressed on the membrane of the differentiated cells. Free (non-encapsulated) fluorescent probe (siRNA conjugated with alexaFluor 546) was not taken up by the cells at all and the peak of the cells that were exposed to the free fluorescent probe was identical to the peak of the control cells that were not exposed to neither vesicles and fluorescent probe. These data show that the vesicles with their encapsulated fluorescent probe, and not the free fluorescent probe, were taken up by the cells and that vesicles with mannose surface groups were taken up by the cells much more than vesicles without the targeting ligand on their surface. Altogether, these results indicate that vesicles with mannose surface groups target cells that express mannose receptors.

More specifically, the results obtained with GLH-64a were conclusive and showed that a bola which is bound to the mannose moiety via the primary hydroxyl which is situated on carbon 6 is capable of providing efficient targeting.

The results obtained with GLH-64b showed that targeting can be achieved with this bola, although the uptake of the vesicles that contain GLH-64b (uptake indicates targeting) was somewhat less than that obtained with GLH-64a (see FIG. 52). GLH-64b contains a mixture of bolas where the mannose is bound to the bola skeleton either via the primary or the secondary hydroxyls. Therefore, it was interesting to see whether a bola in which the mannose moiety is bound only via the secondary hydroxyl is capable of providing good targeting. GLH-64d is such a bola in which the mannose moiety is bound via the secondary hydroxyl, which is situated on carbon number 1 of the mannose. The results of the targeting experiment with GLH-64d are described in FIG. 54, which depicts the uptake of fluorescent vesicles that contain GLH-64d by differentiated and non-differentiated J774 cells. Cells were differentiated by exposing them to 1 μg/mL dexamethasone for 24 hours. Untreated cells were grown in parallel to the differentiated cells, but without dexamethasone. Cells were incubated with fluorescent vesicles that contained mannose moieties on their surface (by the including 5% GLH-64a in the vesicles formulation) for 4 hours and were examined by FACS. As noted above, a shift of the peak to the right indicates higher uptake (namely higher binding).

As can be seen from FIG. 54, vesicles that contain GLH-64d were taken up somewhat better by differentiated cells than by non-differentiated cells, but the shift was much smaller than that obtained with GLH-64a. Again, it is not yet clear whether this smaller shift is due to non-efficient differentiation which did not cause enough expression of mannose receptors, or because mannose which is bound via its secondary hydroxyl is not recognized by the mannose receptor as well as a mannose moiety which is bound to the bola's skeleton via the primary hydroxyl. Nonehtheless, it is safe to conclude, based upon all the results of the targeting experiments described above, that targeting can be achieved with vesicle that contain mannose surface moieties, particularly where the mannose is bound to the bola's skeleton via the primary hydroxyl situated on carbon 6 of the mannose.

### EXAMPLE 29

### Vesicle Stability During Storage

To study the effect of vesicle composition on stability, vesicles were prepared with encapsulated CF and studied the percent CF still encapsulated as a function of time in storage. Vesicles of the following formulations were prepared: a) a mixture of GLH-19 and GLH-20 (2:1) used as a control; and b) a formulation similar to that described in ‘a’ above with the addition of different amounts of GLH 64a (1%, 5% and 10% of the total bolas in the vesicle formulation). Each of the formulations, described in ‘a’ and ‘b’ above, was prepared with or without the bola that contains the chitosan head group (GLH-55b). All formulations contained also CHOL and CHEMS at a molar ratio of 1:1 as described.

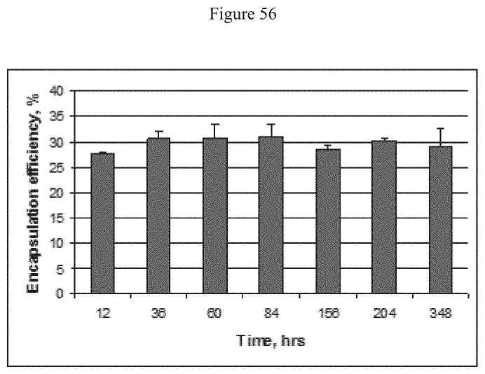

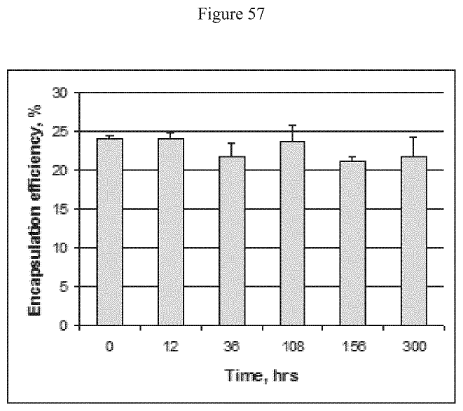

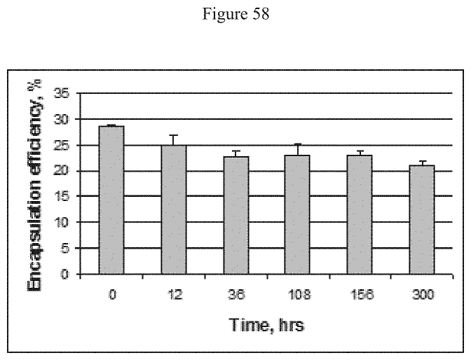

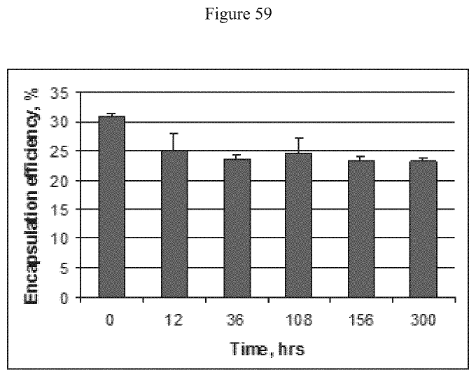

Vesicles were prepared from each of the above formulations by the method of film hydration followed by sonication as described above, and percent CF encapsulation was determined at different times in storage at 4° C. The results of the vesicle stability are shown in FIGS. 55-59. Vesicles that were made from the basic bolas (GLH-19 and GLH-20) were stable and maintained the amount of their encapsulated material for at least 14 days, which was the maximum period studied in this project (FIG. 55). Addition of CS surface groups, by including GLH-55b in the vesicle formulation, did not change vesicle stability and these vesicles were stable as well (FIG. 56). Addition of 1% GLH-64 to vesicle formulation that contained GLH-19, GLH-20 and GLH-55b did not affect significantly the stability of the resulting vesicles (FIG. 57), but 5% and 10% GLH-64 in the vesicle formulation reduced somewhat the stability of the resulting vesicles (25% less encapsulated CF was found after two weeks in storage), as can be seen from FIGS. 58 and 59, respectively. This reduction in the amount of CF encapsulation may be related to dissociation of negatively charged CF that was bound to the positively charged surface of the vesicles and not to disintegration of the vesicular structure or loss of encapsulated CF from the interior. Notably, dissociation of CF is expected to increase when more non-charged mannose groups are present on the vesicle surface and interfere with CF binding to the positively charged ACh groups.

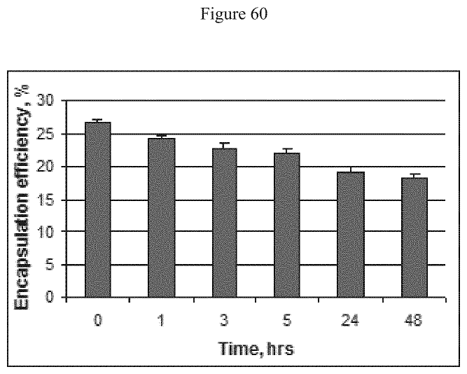

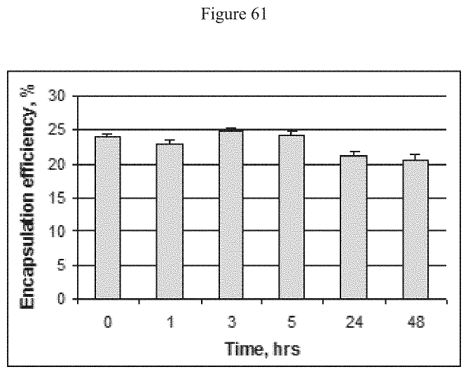

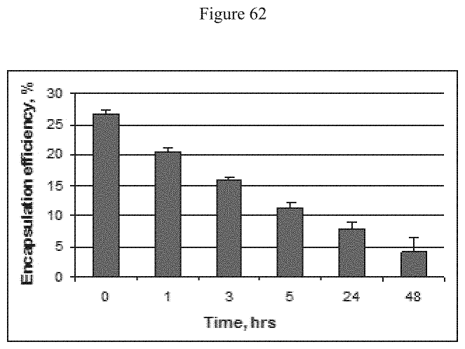

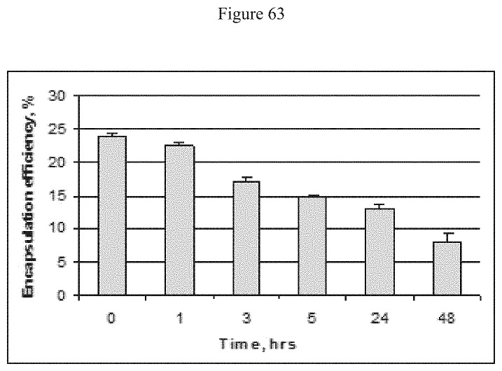

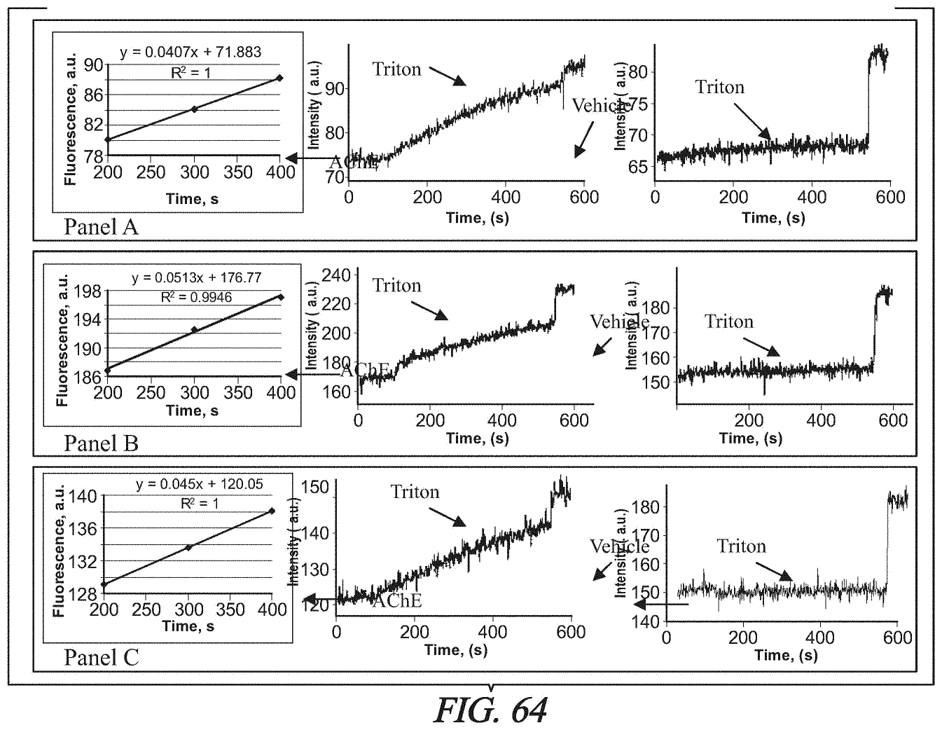

Vesicle stability in presence of 4% albumin was also studied. It is well known that proteins in the bathing medium may affect vesicle stability, and, since albumin is the major protein of the serum and, thus, when vesicles are injected into mice they will first circulate in the blood that contains serum proteins, we examined the effect of albumin on vesicle stability. The experiments employed 4% albumin, which is the concentration of this protein in serum. As can be seen in FIG. 60, albumin reduced somewhat the stability of vesicles made of GLH-19, GLH-20 and GLH-55b. By comparison, addition of 1% GLH-64 increased somewhat vesicle stability (FIG. 61). This was even more apparent when the vesicles were incubated at 25° C. instead of 4° C. (compare vesicle stability in FIG. 62, that show stability of vesicles without GLH-64 to FIG. 63 that shows stability of vesicles with GLH-64).

These results suggest that GLH-64a may increase vesicle stability in the blood. Previous data obtained upon IV administration of proteins, peptides and low molecular weight molecules, has also shown that the majority of the administered IV dose of vesicles of the disclosure delivers most of the encapsulated ingredients to the CNS within 2 hours after administration. Thus long term blodd circulatory stability may not be important. The reason why GLH-64a reduced vesicle stability in buffer and increased stability in buffer that contains albumin, may be attributed to interference of the mannose surface groups with the interaction of the protein with the vesicle surface. If this is the case, then 5% and 10% GLH-64a may even further increase stability. Vescicle stability can be maximized by fine tuning of the vesicle formulation. For example, increased stability of vesicles that contain GDNF in storage, may be obtained using freeze-dried GDNF-loaded vesicles that are maintained as solids in storage followed by reconstitution of the vesicles before injection.

Controlled release by AChE: The controlled release mechanism is based on the hydrolysis of the acetylcholine head groups of the matrix bolas (particularly GLH-20, the head groups of which are hydrolyzed by AChE). The hydrolyzing enzyme, AChE, is abundant in the CNS and can be inhibited selectively in peripheral tissues, without affecting its activity in the CNS, by pyridostigmine [Grauer et al. Stress does not enable pyridostigmine to inhibit brain cholinesterase after parenteral administration. Toxicol Appl Pharmacol. 2000; 164(3):301-304], a safe drug used in human for the treatment of myasthenia gravis [Bolourchian et al. Prolonged release matrix tablet of pyridostigmine bromide: formulation and optimization using statistical methods. Pak J Pharm Sci. 2012; 25(3):607-616]. Since, in addition to GLH-19 and GLH-20, GLH-55b and GLH-64a are also present in the vesicle formulation, experiments were carried out to determine if the addition of GLH-55b and GLH-64a influences the enzymatic hydrolysis of GLH-20's head groups, which controls the release of the active agent in the brain [Popov et al. (2012) Site-directed decapsulation of bolaamphiphilic vesicles with enzymatic cleavable surface groups. J. Controlled Release, June 10; 160(2):306-146]. To test this, vesicles were prepared with and without CS, and with different amounts of GLH-64a, and tested how vesicles that were prepared from these formulations release their encapsulated content upon exposure to AChE. The results that are shown in FIG. 20 indicate that neither GLH-55b nor GLH-64a inhibit the release rates induced by AChE.

FIG. 20 depicts the effect of GLH-55b and GLH-64 on the release of encapsulated CF from vesicles that contain encapsulated CF were incubated in PBS while monitoring their fluorescence. AChE dissolved in water (2 units/24), or water (24) was added while fluorescence was continuously monitored. Due to its high concentration inside the vesicles, the fluorescence of encapsulated CF is quenched. An increase in fluorescence indicates release of CF from the vesicles as the released drug is diluted in the bathing medium. Triton X100 was added at the end of the experiment to completely disrupt the vesicles and obtain the total fluorescence of the encapsulated CF. The graphs on the left side of FIG. 20 show the slope of the increase in fluorescence, representing the rate of the release. The graphs in middle column of Figurre 20 show the release induced by AChE and the graphs in the right column of FIG. 20 show the release induced by the vehicle (used as a control). Panel A: Vesicles made of GLH-19 and GLH-20, without GLH-55b and GLH-64a); Panel B: Vesicles made of GLH-19, GLH-20, GLH-55b and 1% GLH-64; Panel C: Vesicles made of GLH-19, GLH-20, GLH-55b and 10% GLH-64a.

Encapsulation studies: To learn how GLH-64a affects the encapsulation capacity of the vesicles, CF was used as the fluorescent probe and experiments compared the amount of CF encapsulation in vesicles without and with GLH-64a. The results are summarized in Table 15.

TABLE 15
CF encapsulation in vesicles containing different amounts of GLH-64

| | % CF | % CF |
|---|---|---|
| % GLH-64 in the | encapsulation | encapsulation 36 h |
| vesicle | immediately after | after vesicle |
| formulation | vesicle preparation | preparation |
| 0 | 27.6 ± 0.2 | 30.5 ± 1.5 |
| 1 | 27.9 ± 1.2 | 31.1 ± 2.6 |
| 5 | 34.1 ± 2.0 | 32.1 ± 3.7 |
| 8 | 35.3 ± 4.4 | 34.7 ± 0.5 |

As can be seen from Table 15, CF encapsulation ranged between 28-35% in all the vesicle formulations that were tested, with a tendency of increased encapsulation capacity with increased amount of GLH-64a in the vesicle formulation (the amount of GLH-64a was increased in the vesicle formulation from 1% to 8%). Yet, the difference in percent encapsulation among the various formulations was not significant and this led us to conclude that GLH-64a does not interfere with encapsulation, even though inclusion of GLH-64a in the vesicle formulation reduces somewhat vesicle size (see above). Therefore, from the vesicle properties and encapsulation points of view, relatively high concentrations of GLH-64 may be prepared in the vesicle formulation, to help ensure efficient targeting, since high concentrations of GLH-64 in the vesicle formulation will produce high number of targeting ligands on the vesicle's surface. The maximum amount of GLH-64 that will not interfere with the properties of the vesicles as drug carriers will be established, and used in targeting studies in vitro and in vivo.

Encapsulation of a protein similar in properties to GDNF was also examined. Initial encapsulation studies require relatively high amounts of protein and, since GDNF is expensive, initial studies were conducted with a model protein—trypsinogen, which has similar molecular weight and isoelectric point to that of GDNF. Trypsinogen has a similar isoelectric point (about 9) and close molecular weight (about 24 KDa) to that of GDNF and these two properties are most important for encapsulation. The initial studies are done with relatively large amounts of trypsinogen, which were needed for the determination of its concentration by UV absorbance. Then, once initial conditions for encapsulation have been worked out, it will be possible to use smaller amounts of the protein, similar to those that will be used with GDNF, with detection of these small amounts facilitated using fluorescence measurements. For this purpose, trypsinogen was labeled with a fluorescent probe (Alexa Flour™ 488) as described above. Note that GDNF can be labeled in this manner as well.

The determination of encapsulation with the labeled trypsinogen was carried out in the following way: fluorescently-labeled trypsinogen was dissolved in distilled water, at a concentration of up to 100 μg/ml. Then, empty vesicles were prepared by film hydration followed by sonication as described above. The trypsinogen solution was added to the vesicle suspension, and the mixture was sonicated on ice to form vesicles of 5 mg/ml of the bolas with encapsulated fluorescently-labeled protein. Then, non-encapsulated material was removed by running the vesicles over a Sephadex G-75 column. The fractions collected from the column were treated by Triton X-100 reduced form, and the fluorescence of each fraction was determined by fluorescence spectroscopy. Percent encapsulation was determined by dividing the AUC of the vesicle fractions by the total AUC, which is the sum of the AUC of the vesicle fractions and the AUC of the free trypsinogen. This approach was used to determine how the addition of the bola with the mannose head groups (GLH-64a) affects encapsulation. The amount of the encapsulated protein was determined right after vesicle formation and again, after 24 hrs.

Table 16 shows the percent encapsulation of labeled trypsinogen obtained with vesicles prepared from formulations without and with GLH-64a.

Percent encapsulation of trypsinogen conjugated with Alexa
FlourTM-488 in vesicles with and without mannose surface groups

| | No GLH-64a | | 1% GLH-64a | | 5% GLH-64a | | 10% GLH-64a | |
|---|---|---|---|---|---|---|---|---|
| Time after | With- | | With- | | With- | | With- | |
| | out | With | out | With | out | With | out | With |
| vesicle | GLH- | GLH- | GLH- | GLH- | GLH- | GLH- | GLH- | GLH- |
| formation | 55b | 55b | 55b | 55b | 55b | 55b | 55b | 55b |
| Immediate | 45.0 | 21.1 | 59.0 | 19.6 | 58.7 | 38.5 | 49.5 | — |
| 24 h | 43.4 | — | 42.2 | 23.4 | 49.1 | 34.2 | — | 37.5 |

As can be seen, the addition of chitosan bola (GLH-55b) to the vesicle formulation significantly reduced the amount of trypsinogen encapsulation. However, the inclusion of GLH-64a in the vesicle formulations (with GLH-55b) increased trypsinogen encapsulation (from 21.1% in the control to 34.2% and 37.5% in vesicles with 5% and 10% GLH-64a, respectively), although not to the same value, which was observed in vesicles without GLH-55b (about 59%). In other words, GLH-64a partially reversed the drop in encapsulation caused by GLH-55b. The trypsinogen-loaded vesicles without CS surface groups (no GLH-55b) were not completely stable and lost about 16-28% of the encapsulated trypsinogen within 24 h. By comparison, vesicles that contained GLH-55b, although starting with less trypsinogen encapsulation, were more stable and lost only about 11% of the encapsulated trypsinogen within 24 h.

As described herein, novel formulations of bolavesicles can be produced through co-assembly of GDNF with bolaamphiphile/lipid unilamellar vesicles. The formulations can be examined for their chemical and biophysical properties.

The incorporation of GDNF or NGF within the bolavesicles can be shown to significantly modulate interactions with membrane bilayers in model systems. This observation is important, suggesting that GDNF or NGF encapsulated in bolavesicles might be excellent candidates for targeting and transport of different molecular cargoes into the brain.

From the foregoing description, various modifications and changes in the compositions and methods provided herein will occur to those skilled in the art. All such modifications coming within the scope of the appended claims are intended to be included therein.

All publications, including but not limited to patents and patent applications, cited in this specification are herein incorporated by reference as if each individual publication were specifically and individually indicated to be incorporated by reference herein as though fully set forth.

At least some of the chemical names of compounds of the invention as given and set forth in this application, may have been generated on an automated basis by use of a commercially available chemical naming software program, and have not been independently verified. Representative programs performing this function include the Lexichem naming tool sold by Open Eye Software, Inc. and the Autonom Software tool sold by MDL, Inc. In the instance where the indicated chemical name and the depicted structure differ, the depicted structure will control.

Chemical structures shown herein were prepared using ISIS®/DRAW. Any open valency appearing on a carbon, oxygen or nitrogen atom in the structures herein indicates the presence of a hydrogen atom. Where a chiral center exists in a structure but no specific stereochemistry is shown for the chiral center, both enantiomers associated with the chiral structure are encompassed by the structure.

### REFERENCES

Abu Hammad I, Popov M, Linder C, Grinberg S, Heldman E, Stepensky D (2011) Bolaamphiphilic nanovesicles for the delivery of proteins to the brain, submitted to the Journal of Controlled Release.
Agyare, E K, Kandimalla K K, Poduslo J F, Yu C C, Ramakrishnan M, Curran G L (2008) Development of a smart nano-vesicle to target cerebrovascular amyloid deposits and brain parenchymal plaques observed in Alzheimer's disease and cerebral amyloid angiopathy. Pharm Res November; 25(11):2674-2684.
Ansorena E, Garbayo E, Lanciego J L, Aymerich M S, Blanco-Prieto M J. Production of highly pure human glycosylated GDNF in a mammalian cell line. Int J Pharm. 2010; 385(1-2):6-11.
Clarke R. L, Daum S. J, Gambino A/J, Aceto M. D, Pearl J, Levitt M, Cumiskey W. R. and Bogado E. F. Compounds Affecting the Central Nervous System. 4. 3β-Phenyltropane-2-carboxylic Esters and Anologs. J. Med. Chem. 1973; 16:1260-1267.
Dakwar G R, Abu Hammad I, Popov M, Linder C, Grinberg S, Heldman E, Stepensky D. Delivery of proteins to the brain by bolaamphiphilic nano-sized vesicles. J Control Release. 2012; 160(2):315-321.
Fazil M, Md S, Haque S, Kumar M, Baboota S, Sahni J K, Ali J. Development and evaluation of rivastigmine loaded chitosan nanoparticles for brain targeting. Eur J Pharm Sci. 2012; 47(1):6-15
Fuhrhop J. H. and Wang T. (2004) Bolaamphiphiles, Chem. Rev. 104:2901-2937.
Gash D M, Zhang Z, Ai Y, Grondin R, Coffey R, Gerhardt G A. Trophic factor distribution predicts functional recovery in parkinsonian monkeys. Ann Neurol. 2005; 58(2):224-233Gisslen M and Hagberg L and Hagberg (2001) Antiretroviral treatment of central nervous system HIV-linfection: a Review. HIV Medicine (2001) 2, 97-104.
G Gnanarajan, A K Gupta, V Juyal, P Kumar, P K Yadav, P Kailash “A validated method for development of tenofovir as API and tablet dosage forms by UV spectroscopy” Pharm Analysis 2009 Vol 1 Issue 4 pp 351-353.
Grinberg S, C. Linder, E. Heldman, Z. Weizman, and V. Kolot : EP1360168, 2003-11-12 and WO2002IL00043and 20020116, Filed by BG Negev “Amphiphilic Derivatives for the Production of Vesicles, Micelles, Complexants, and Uses Thereoff” in 2003
Grinberg S., Linder C., Kolot V., Waner T., Wiesman Z., Shaubi E., Heldman E. (2005) Novel cationic amphiphilic derivatives from vemonia oil: synthesis and self-aggregation into bilayer vesicles, nanoparticles, and DNA complexants. Langmuir. 21(17):7638-7645.
Grinberg S., Kolot V., Linder C., Shaubi E., Kas'yanov V., Deckelbaum R. J., Heldman E. (2008) Synthesis of novel cationic bolaamphiphiles from vemonia oil and their aggregated structures. Chem Phys Lipids 153(2):85-97.
Grinberg, S., Kipnis, N., Linder, C., Kolot, V. and Heldman, E., (2010) Assymetric bolaamphiphiles from veronica oil designed for drug delivery. Eur. J Lipid Sci. Technol., 112, 137-151.
Hamilton J F, Morrison P F, Chen M Y, Harvey-White J, Permute R S, Phillips H, Oldfield E, Bankiewicz K S. Heparin coinfusion during convection-enhanced delivery (CED) increases the distribution of the glial-derived neurotrophic factor (GDNF) ligand family in rat striatum and enhances the pharmacological activity of neurturin. Exp Neurol. 2001; 168(1):155-161
E. Heldman E, C. Linder, S. Grinberg Amphiphilic compounds and vesicles liposomes for organ-specified drug targeting” US patent Application 20060039962+WO03047499-2003-06-12.
Highleyman, L (2009) HIV and the Brain BETA. 2009 Summer-Fall;21(4):16-29. C. R.

Holmquist C. R, Keverline-Franz K. I, Abraham P, Boja J. W, Kukar M. J, Caroll F. I. 3alpha-(4″-Substituted Phenyl) Tropane-2beta-Carboxylic Acid Methyl Ester: Novel Ligands with High Affinity and Selectivity at the Doopamine Transporter. J. Med. Chem. 1996; 39:4139.
Huffer T, Linder C, Heldman E, Grinberg S (2011) Interfacial and self-assembly properties of bolaamphiphilic compounds derived from a multifunctional oil, Journal of Colloid and Interface Science, 2012; 365(1):53-62.
Jiang H, Jiang Q, Feng J. Parkin increases dopamine uptake by enhancing the cell surface expression of dopamine transporter. J Biol Chem. 2004; 279(52):54380-543806.
Jonasdottir T J, Fisher D R, Borrebaek J, Bruland O S, Larsen R H (2006) First in vivo evaluation of liposome-encapsulated 223Ra as a potential alpha-particle-emitting cancer therapeutic agent. Anticancer Res. 26(4B):2841-2848.
N. N. Kabal'nova, K. Yu. Murinov, I. R. Mullagaliev, N. N. Krasnogorskaya, V. V.

Shereshovets, Yu. B. Monakov, G. E. Zaikov , Oxidative Destruction of Chitosan Under the Effect of Ozone and Hydrogen Peroxide. Journal of Applied Polymer Science. 2001; 81:875-881.
T Kadota, T Yamaai, Y Saito, Y Akita, S Kawashima, K Moroi, N Inagaki and K Kadota. Expression of dopamine transporter at the tips of growing neurites of PC12 cells. J Histochem Cytochem 1996; 44: 989-996.
Kiyohito Shimura, Wang Zhi, Hiroyuki Matsumoto and Ken-ichi Kasai. Accuracy in the Determination of Isoelectric Points of Some Proteins and a Peptide by Capillary Isoelectric Focusing: Utility of Synthetic Peptides as Isoelectric Point Markers. Anal. Chem. 2000; 72:4747-4757.
Kordower J H, Emborg M E, Bloch J, Ma SY, Chu Y, Leventhal L, McBride J, Chen E Y, Palfi S, Roitberg B Z, Brown W D, Holden J E, Pyzalski R, Taylor M D, Carvey P, Ling Z, Trono D, Hantraye P, Deglon N, Aebischer P. Neurodegeneration prevented by lentiviral vector delivery of GDNF in primate models of Parkinson's disease. Science. 2000; 290(5492):767-773.
Lapidot Y., Rappaport S., Wolman Y. Use of esters of N-hydroxysuccinimide in the synthesis of N-acylamino acids. The Journal of Lipid Research. 1967; 8:142-145.
Letendre S, Marquie-Beck J, Capparelli E, Best B, Clifford D, Collier A C, Gelman B B, McArthur J C, McCutchan J A, Morgello S, Simpson D, Grant I, Ellis R J; CHARTER Group.(2008) Validation of the CNS Penetration-Effectiveness rank for quantifying antiretroviral penetration into the central nervous system. Arch Neurol. 2008 January; 65(1):65-70.
Linder C; Grinberg S; Heldman E “Nano-sized Particles Composing Multi-Headed Amphiphiles for Targeted Drug-Delivery” WO 2010128504 (A2) 2010.
Lu W, Tan Y Z, Hu K L and Jiang X G. (2005) Cationic albumin conjugated pegylated nanoparticle with its transcytosis ability and little toxicity against blood-brain barrier. Int J Pharm. May 13;295 (1-2); 247-260.
Madras B K, Fahey M A, Bergman J, Canfield D R, Spealman R D. Effects of cocaine and related drugs in nonhuman primates. I. [3H]cocaine binding sites in caudate-putamen. J Pharmacol Exp Ther. 1989; 251(1):131-141.
Melzer P. C, Liang A. Y, Brownell A. L. and Madras B. K. Substituted 3-Phenyltropane Analogs of Cocaine; Synthesis, Inhibition of binding at Cocaine Recognition Sites, and Positron Emmission Tomography Imaging. J. Med. Chem. 1993; 36:855-862.
Millius R. A, Saha J. K, Madras B. K. and Neumeyer J. L. Synthesis and Receptor Binding of N-Substituted Tropane derivatives. High Affinity ligands for the Cocaine Receptor. J. Med. Chem. 1991; 34:1728-1731.
Myers A. L, Williams H. E, Kraner J. C. K. and P. S. Callery J. Identification of Anhydroecgonine Ethyl Ester in the Urine of a Drug Overdose Victim. Forensic Sci. 2005; 50:1-5.
New R. R. C. (ed). (1997) Liposomes. A Practical Approach. IRL Press, Oxford.
Newton H B (2006) Advances in strategies to improve drug delivery to brain tumors. Expert Rev Neurother. 6(10): 1495-509.
Pickrell A M, Pinto M, Hida A, Moraes C T. Striatal dysfunctions associated with mitochondrial DNA damage in dopaminergic neurons in a mouse model of Parkinson's disease. J Neurosci. 2011; 31(48):17649-17658.
Popov M., Linder C., Deckelbaum R. J., Grinberg S., Hansen I. H., Shaubi E., Waner T., Heldman E. (2009) Cationic vesicles from novel bolaamphiphilic compounds. J Liposome Res. 20(2):147-159.
Popov M, Grinberg S, Linder C, Bachar Z, Waner T, Deckelbaum R, Heldman E. (2011) Site-directed decapsulation of bolaamphiphilic vesicles with enzymatic cleavable surface groups Journal of Controlled Release 2012; 160(2): 306-314.
Puri, A., Loomis, K., Smith, B., Lee, J., Yavlovich, A., Heldman, E. and Blumenthal, R. (2009) Lipid-Based Nanoparticles as Pharmaceutical Drug Carriers: From Concepts to Clinic. Crit Rev Ther Drug Carrier Syst, 26(6): 523-580.
Qin C.Q., Du Y. M., Xiao L. Effect of hydrogen peroxide treatment on the molecular weight and structure of chitosan. Polymer Degradation and Stability. 2002; 76:211-221.
Riss P J, Hummerich R, Schloss P. Synthesis and monoamine uptake inhibition of conformationally constrained 2beta-carbomethoxy-3beta-phenyl tropanes. Org Biomol Chem. 2009; 7(13):2688-2698
Saiyed Z, Gandhi N, and Nairi M (2010)Magnetic Nanoformulation of Azidothymidine 5′-triphosphate for Targeted Delivery across the Blood-Brain Barrier. International Journal of Nanomedicine 5 :157-166.
Salvatore M F, Ai Y, Fischer B, Zhang A M, Grondin R C, Zhang Z, Gerhardt G A, Gash D M. Point source concentration of GDNF may explain failure of phase II clinical trial. Exp Neurol. 2006; 202(2):497-505.
Slevin J T, Gash D M, Smith C D, Gerhardt G A, Kryscio R, Chebrolu H, Walton A, Wagner R, Young A B. Unilateral intraputamenal glial cell line-derived neurotrophic factor in patients with Parkinson disease: response to 1 year of treatment and 1 year of withdrawal. J Neurosurg. 2007; 106(4):614-620
Songjiang Z and Lixiang W. (2009) Amyloid-Beta Associated with Chitosan Nano-Carrier has Favorable Immunogenicity and Permeates the BBB. AAPS Pharm Sci Tech, 10(3):900-905.
Spudich S and Antses B (2011) Central Nervous System Complications of HIV Infection. Top. Antiviral Med 19(2), 48-57.
Steiner J M, Medinger T L, Williams D A. Purification and partial characterization of feline trypsin. Comp Biochem Physiol B Biochem Mol Biol. 1997; 116(1):87-93.
Stepanov V, Schou M, Järv J, Halldin C. Synthesis of 3H-labeled N-(3-iodoprop-2E-enyl)-2beta-carbomethoxy-3beta-(4-methylphenyl)nortropane (PE2I) and its interaction with mice striatal membrane fragments. Appl Radiat Isot. 2007; 65(3):293-300
Stern J, Freisleben H J, Janku S, Ring K. (1992) Black lipid membranes of tetraether lipids from Thermoplasma acidophilum, Biochim Biophys Acta 1128:227-236.
Uchegbu I. The biodistribution of novel 200-nm palmitoyl muramic acid vesicles International Journal of Pharmaceutics. 1998; 162:19-27.
Varatharajan L and Thomas S. (2009)The transport of anti-HIV drugs across blood-CNS interfaces: Summary of current knowledge and recommendations for further Research Antiviral Res. 2009 May; 82(2): A99-A109.
Wiesman Z., Dom N. B., Sharvit E., Grinberg S., Linder C., Heldman E., Zaccai M. (2007)

Novel cationic vesicle platform derived from vernonia oil for efficient delivery of DNA through plant cuticle membranes. J Biotechnol. 130(1):85-94.
Wu Y., Zheng Y. Yang W. Wang C., Hu J., Fu S. Synthesis and characterization of a novel amphiphilic chitosan-polylactide graft copolymer. Carbohydrate Polymers. 2005; 59:165-171.
Yagi S, Yoshikawa E, Futatsubashi M, Yokokura M, Yoshihara Y, Torizuka T, Ouchi Y. Progression from unilateral to bilateral parkinsonism in early Parkinson disease: implication of mesocortical dopamine dysfunction by PET. J Nucl Med. 2010; 51(8):1250-1257
Zabicky J; Linder C; Grinberg S; Heldman E “Nano—and Mesosized Particles Comprising an Inorganic Core, Process and Applications Thereof” US2009011002

1.-127. (canceled)

128. A pharmaceutical composition which comprises one or more bolaamphiphilic compounds selected from the group consisting of GLH-19, GLH-20, GLH-55b, GLH1-64a, GLH-64b, GLH-64c, GLH-64d, and GLH-64e, and a stabilizer which is cholesterol hemisuccinate, cholesterol, or cholesterol hemisuccinate and cholesterol.

129. A composition of claim 128, wherein NTF, NGF, GDNF, or IGF-1 is encapsulated within said composition.

130. A method of treating ALS which comprises administering to a person in need thereof a composition of claim 128.

131. A method of claim 130 wherein the method comprises intravenous, intraperitoneal, or oral administration.

132. A pharmaceutical composition which comprises one or more bolaamphiphilic compounds selected from the group consisting of GLH-19, GLH-20, GLH-55b, and a bolaamphiphile with a head group that is targeted to dopamine transporters, and a stabilizer which is cholesterol hemisuccinate, cholesterol, or cholesterol hemisuccinate and cholesterol.

133. A composition of claim 132, wherein the bo1aamphiphile with head group that is targeted to dopamine transporters is GLH 57.

134. A composition of claim 132, wherein NTF, NGF, GDNF, or IGF-1 is encapsulated within said composition.

135. A composition of claim 133, wherein NTF, NGF, GDNF, or IGF-1 is encapsulated within said composition.

136. A method of treating Parkinson's disease which comprises administering to a. person in need thereof a composition of claim 132.

137. A method of treating Parkinson's disease which comprises administering to a. person in need thereof a composition of claim 133.

138. A method of treating Parkinson's disease which comprises administering to a person in need thereof a composition of claim 134.

139. A method of treating Parkinson's disease which comprises administering to a person in need thereof a composition of claim 135.

140. A method of claim 136 wherein the method comprises intravenous, intraperitoneal, or oral administration.

141. A method of claim 137 wherein the method comprises intravenous, intraperitoneal, or oral administration.

142. A method of claim 138 wherein the method comprises intravenous, intraperitoneal, or oral administration.

143. A method of claim 139 wherein the method comprises intravenous, intraperitoneal, or oral administration.
